(19) United States
(12) Patent Application Publication
(10) Pub. No.: US 2025/0155429 A1
(43) Pub. Date: May 15, 2025

(54) MULTIVALENT LINKERS USED FOR ANTIBODY LABELING

(71) Applicant: Proteintech Group, Inc., Rosemont, IL (US)

(72) Inventors: Jianxun LI, Rosemont, IL (US); Deepa Shankar, Rosemont, IL (US); Michael Metterlein, Rosemont, IL (US); Sabrina Wendler, Rosemont, IL (US); Andrea Buchfellner, Rosemont, IL (US); Christian Linke-Winnebeck, Rosemont, IL (US); Larisa Yurlova, Rosemont, IL (US)

(21) Appl. No.: 19/032,745

(22) Filed: Jan. 21, 2025

Related U.S. Application Data
(63) Continuation of application No. PCT/US2023/071905, filed on Aug. 9, 2023.
(60) Provisional application No. 63/396,519, filed on Aug. 9, 2022.

(51) Int. Cl. G01N 33/542; C07K 16/00; C07K 16/44

(52) U.S. Cl. CPC G01N 33/542; C07K 16/005; C07K 16/44; C07K 2317/22; C07K 2317/524; C07K 2317/526; C07K 2317/528; C07K 2317/569; C07K 2317/92

(57) ABSTRACT

The disclosure relates to the use of multivalent linkers to label targets (e.g., primary antibodies). The disclosure provides methods of labeling antibody with a reporter/dye/enzyme via the multivalent linker. The disclosure further provides methods to use the multivalent linkers to label multiple primary antibodies of the same species in the same experiment.

### CROSS-REFERENCE TO RELATED APPLICATIONS

This application is a continuation of International Application No. PCT/US2023/071905, filed Aug. 9, 2023, which claims the benefit of U.S. Provisional Application No. 63/396,519, filed Aug. 9, 2022, the content of each of which is herein incorporated by reference in its entirety.

### REFERENCE TO AN ELECTRONIC SEQUENCE LISTING

The content of the electronic sequence listing (PTGI_001_01US_SeqList_ST26.xml; Size: 535,676 bytes; and Date of Creation: Jan. 21, 2025) is herein incorporated by reference in its entirety.

### BACKGROUND

Immunodetection assays leverage the antigen-binding abilities of antibodies to detect and quantify epitopes of interest. These assays typically use dual primary and secondary antibody systems, wherein specific primary antibodies are developed to bind to a target of interest, and secondary, labeled antibodies are designed to generically target conserved regions within primary antibodies. In some cases, primary antibodies are directly labeled. Current labeling and immunoassay technologies however suffer from several drawbacks, including inconsistent labeling, damage to epitope sites, antibody cross-reactions, and others.

To overcome these challenges, additional technologies are needed.

### SUMMARY

The current disclosure solves the aforementioned problems through the creation and utilization of artificial multivalent linkers capable of binding and labeling primary antibodies with high affinity.

Thus, in some embodiments, the present disclosure teaches a multivalent linker that specifically binds a target antigen, said multivalent linker comprising: a) a plurality of peptide binding arms, each binding arm capable of binding to an epitope in the same target antigen; and b) a linker segment covalently operably linked to the plurality of peptide binding arms. In some embodiments, the multivalent linker specifically binds a target antigen. In some embodiments, the target antigen is a constant domain of an antibody. In some embodiments, the protein binds to the antibody with a k_{off}(s⁻¹) rate of less than or equal to 1.0×10⁻⁴.

In some embodiments, the disclosure provide a method for detecting two or more target antigens in a sample comprising contacting the sample with a first antibody specific for a first target antigen and a second antibody specific for a second target antigen. In some embodiments, the first and second antibodies are linked or conjugated to a first multivalent linker and a second multivalent linker, respectively. In some embodiments, each multivalent linker specifically binds the constant region of the first or second antibody with a k_{off}(s⁻¹) rate of less than or equal to 1.0×10⁻⁴. In some embodiments, each multivalent linker is attached to a reporter. In some embodiments, the reporters are not the same.

In one aspect, the disclosure provides multivalent linkers that specifically bind a target antigen unit, said multivalent linker comprising: a) a plurality of peptide binding arms, each binding arm capable of binding to an epitope in the same target antigen unit; and b) at least one linker segment covalently operably linked to the plurality of peptide binding arms.

In one aspect, the disclosure provides molecular complexes comprising:
(i) a single target antigen unit; and
(ii) a multivalent linker comprising:
   a) a plurality of peptide binding arms, wherein each peptide binding arm binds to the single target antigen unit; and
   b) at least one linker segment covalently operably linked to the plurality of peptide binding arms.

In some embodiments, the multivalent linker binds to the target antigen unit with an apparent k_{off}(s⁻¹) rate of less than or equal to 1.0×10⁻⁴. In some embodiments, the multivalent linker dissociates from the single target antigen unit at an apparent k_{off}(s⁻¹) rate of less than or equal to 1.0×10⁻⁴. In some embodiments, the k_{off}(s⁻¹) rate is determined by Bio-Layer Interferometry (BLI).

In some embodiments, the target antigen unit comprises a constant region of an antibody. In some embodiments, the target antigen unit comprises an Fc region of an antibody. In some embodiments, the epitope is located on a CH2 domain, a CH3 domain, and/or a CH4 domain of the constant region or the Fc region. In some embodiments, the epitope(s) of the peptide binding arms are located on a CH1 domain or a CL domain of the target antigen unit.

In some embodiments, the target antigen unit is an antibody, F(ab′)2, Fab₂, Fab₃, or IgNAR. In some embodiments, the antibody is an IgG. In some embodiments, the IgG antibody is IgG1, IgG2, IgG3, or IgG4 subclass, optionally wherein the IgG antibody is a human antibody. In some embodiments, the IgG antibody is IgG1, IgG2a, IgG2b, IgG2c or IgG3 subclass; optionally wherein the IgG antibody is a murine antibody. In some embodiments, the IgG antibody is IgG1, IgG2a, IgG2b, or IgG2c subclass; optionally wherein the IgG antibody is a rat antibody. In some embodiments, the antibody is an IgM. In some embodiments, the antibody is a heavy-chain antibody. In some embodiments, the antibody is a guinea pig antibody, a mouse antibody, a rat antibody, a chicken antibody (e.g., IgY), a donkey antibody, a rabbit antibody, a human antibody, a goat antibody, a pig antibody, a horse antibody, or a cattle antibody.

In some embodiments, at least one of the peptide binding arms is cross-reactive and is capable of non-simultaneously binding to antibodies from two or more species; optionally wherein the two or more species are selected from human, mouse, rat, and rabbit. In some embodiments, at least one of the peptide binding arms is cross-reactive and is capable of non-simultaneously binding to antibodies from both rabbit and human.

In some embodiments, each peptide binding arm is specific for a different epitope of the same target antigen unit. In some embodiments, each peptide binding arm is specific for the same epitope, and wherein the target antigen unit comprises a plurality of the same epitopes.

In some embodiments, the plurality of peptide binding arms are capable of binding to the same target antigen unit. In some embodiments, the plurality of peptide binding arms do not bind to more than one target antigen unit.

In some embodiments, the multivalent linker is bivalent, comprising two peptide binding arms.

In some embodiments, less than 5%, 4%, 3%, 2%, or 1% of the plurality of peptide binding arms crosslink to different target antigen units. In some embodiments, the plurality of peptide binding arms are separated by a distance factor sufficiently long to prevent cross linking of the peptide binding arms with more than one target antigen units.

In some embodiments, the linker segment comprises a peptide. In some embodiments, the linker segment comprises between 5-50 amino acids. In some embodiments, the linker segment comprises between 10-40 amino acids. In some embodiments, the linker segment comprises between 20-30 amino acids. In some embodiments, the linker segment comprises about 25 amino acids, about 30 amino acids, or about 35 amino acids.

In some embodiments, the linker segment is between 1-400 Å in length in the extended conformation. In some embodiments, the linker segment is between about 50-350 Å in length in the extended conformation. In some embodiments, the linker segment is between about 70-300 Å in length in the extended conformation. In some embodiments, the linker segment is between about 70-140 Å in length in the extended conformation.

In some embodiments, the multivalent linker has an apparent k_{off}(s⁻¹) rate of less than or equal to 1.0×10⁻⁵. In some embodiments, the multivalent linker dissociates from the single target antigen unit at an apparent k_{off}(s⁻¹) rate of less than or equal to 1.0×10⁻⁵.

In some embodiments, the multivalent linker has an apparent k_{off}(s⁻¹) rate of less than or equal to 1.0×10⁻⁶. In some embodiments, the multivalent linker dissociates from the single target antigen unit at an apparent k_{off}(s⁻¹) rate of less than or equal to 1.0×10⁻⁶.

In some embodiments, the multivalent linker has an apparent k_{off}(s⁻¹) rate of less than or equal to 1.0×10⁻⁷. In some embodiments, the multivalent linker dissociates from the single target antigen unit at an apparent k_{off}(s⁻¹) rate of less than or equal to 1.0×10⁻⁷.

In some embodiments, the linker segment comprises a (G4S) unit. In some embodiments, the linker segment comprises more than 2 (G4S) units. In some embodiments, the linker segment comprises more than 3 (G4S) units. In some embodiments, the linker segment comprises more than 4 (G4S) units. In some embodiments, the linker segment comprises more than 5 (G4S) units. In some embodiments, the linker segment comprises more than 6 (G4S) units. In some embodiments, the linker segment comprises at most 4, at most 5, at most 6, at most 7, at most 8, or at most 9 (G4S) units.

In some embodiments, the linker segment comprises the amino acid sequence of GSTSGSGKSSEGKGEGSTSGSGKSG (SEQ ID NO: 495).

In some embodiments, at least 20-25% of the amino acids in the peptide of the linker segment are glycine. In some embodiments, between 60%-90% of the amino acids in the peptide of the linker segment are glycine. In some embodiments, between 10%-30% of the amino acids in the peptide of the linker segment are serine or threonine; more preferably, serine. In some embodiments, at least 70%, at least 75%, at least 80%, at least 85%, at least 90%, at least 95%, or 100%, of the amino acids in the linker segment are glycine (G), alanine (A), serine (S), threonine (T), glutamic acid (E), aspartic acid (D), lysine (K) and arginine (R). In some embodiments, at least 70%, at least 75%, at least 80%, at least 85%, at least 90%, at least 95%, or 100%, of the amino acids in the linker segment are glycine, alanine, serine and threonine. In some embodiments, at least 70%, at least 75%, at least 80%, at least 85%, at least 90%, at least 95%, or 100%, of the amino acids in the linker segment are glycine, serine and threonine. In some embodiments, at least 70%, at least 75%, at least 80%, at least 85%, at least 90%, at least 95%, or 100%, of the amino acids in the linker segment are glycine and serine. In some embodiments, the ratio of (i) glycine to (ii) serine and/or threonine is about 4:1 in the linker segment.

In some embodiments, the multivalent linker comprises at least one moiety for conjugation to a heterologous molecule. In some embodiments, the moiety for conjugation is a cysteine. In some embodiments, the moiety for conjugation is a lysine. In some embodiments, the moiety for conjugation comprises a biotin or a streptavidin. In some embodiments, the moiety for conjugation comprises a functional group for conjugation through click chemistry. In some embodiments, the functional group comprises dibenzocyclooctyne group (DBCO), azide, tetrazine and/or trans-cyclooctene (TCO).

In some embodiments, the linker segment comprises the moiety for conjugation. In some embodiments, the peptide in the linker segment comprises the moiety for conjugation.

In some embodiments, the heterologous molecule is a reporter, an oligonucleotide, a moiety functionalized for click chemistry, or an effector. In some embodiments, the multivalent linker comprises at least one reporter, oligonucleotide, moiety functionalized for click chemistry, or effector attached. In some embodiments, the reporter is a fluorescent reporter. In some embodiments, the fluorescent reporter is a fluorescein dye, a rhodamine dye, two or more fluorescent dyes that can act cooperatively with one another, or a protein that exhibits fluorescence. In some embodiments, the fluorescent reporter is green fluorescent protein, yellow fluorescent protein, orange fluorescent protein, cyan fluorescent protein, blue fluorescent protein, red fluorescent protein, mCherry, tdTomato, mStrawberry, mTangerine, and/or dsRed. In some embodiments, the reporter is an enzymatic reporter. In some embodiments, the enzymatic reporter is a horseradish peroxidase, a cathepsin, a matrix metalloprotease, a peptidase, a carboxypeptidase, a glycosidase, a lipase, a phospholipase, a phosphatase, a phosphodiesterase, a sulfatase, a reductase, a bacterial enzyme, a biotin ligase, a DNA transposase, or a nuclease. In some embodiments, the DNA transposase is Tn5 transposase. In some embodiments, the nuclease is micrococcal nuclease. In some embodiments, the effector is a magnetic effector. In some embodiments, the magnetic reporter is Gd(III), Dy(III), Fe(III), and Mn(II), DTPA, DOTA, DO3A, 2-benzyl-DOTA, alpha-(2-phenethyl) 1,4,7,10-tetraazacyclododecane-1-acetic-4,7,10-tris(methylacetic)acid, 2-benzyl-cyclohexyldiethylenetriaminepentaacetic acid, 2-benzyl-6-methyl-DTPA, or 6,6″-bis[N,N,N″,N″-tetra(carboxymethyl)aminomethyl)-4′-(3-amino-4-methoxyphenyl)-2,2′:6′,2″-terpyridine.

In some embodiments, the multivalent linker has an apparent K_{D} for the target antigen unit of less than 10,000 pM, less than 1,000 pM, less than 500 pM, less than 100 pM, less than 50 pM, less than 10 pM, or less than 1 pM. In some embodiments, the multivalent linker has an apparent K_{D} for the target antigen unit of 1 to 10 pM, 10 to 50 pM, 50 to 100 pM, 100 to 500 pM, or 500 to 1,000 pM. In some embodiments, the multivalent linker has an apparent K_{D} for the target antigen unit of less than about 50 pM. In some embodiments, the multivalent linker has an apparent K_{D} for the target antigen unit of less than about 25 pM. In some embodiments, the multivalent linker has an apparent K_{D} for the target antigen unit of less than about 10 pM.

In some embodiments, the plurality of peptide binding arms comprise a peptide binding arm comprising a sequence with at least 80%, at least 85%, at least 90%, at least 95%, at least 96%, at least 97%, at least 98%, at least 99%, or 100% identity with any one of SEQ ID Nos: 1-494. In some embodiments, the plurality of peptide binding arms comprise a peptide binding arm comprising sequence selected from the group comprising SEQ ID Nos 1-494.

In some embodiments, the multivalent linker or the molecular complex of the disclosure comprises more than one linker segment.

In some embodiments, the multivalent linker or the molecular complex of the disclosure comprises the structure: (peptide binding arm)-linker segment-(peptide binding arm).

In some embodiments, the multivalent linker or the molecular complex of the disclosure comprises the structure: (peptide binding arm)-linker segment-(peptide binding arm)-linker segment-(peptide binding arm).

In some embodiments, the peptide binding arm is a VHH of a camelid heavy chain antibody. In some embodiments, the peptide binding arm is a VH of an immunoglobulin. In some embodiments, the peptide binding arm comprises or consists of an immunoglobulin domain.

In some embodiments, the plurality of peptide binding arms are covalently linked to the linker segment. In some embodiments, the plurality of peptide binding arms and the linker segment form a continuous polypeptide.

In some embodiments, the plurality of peptide binding arms and the linker segment are operably linked via a translational fusion. In some embodiments, the plurality of peptide binding arms, the linker segment, and the reporter or effector are each operably linked via a translational fusion.

In some embodiments, each of the binding arm binds to an epitope of the target antigen unit. In some embodiments, the peptide binding arm binds to the epitope non-covalently.

In one aspect, the disclosure provides compositions comprising the multivalent linker of the disclosure or the molecular complex of the disclosure.

In some embodiments, the composition comprises a buffer.

In one aspect, the disclosure provides compositions comprising two or more different multivalent linkers of the disclosure or two or more different molecular complex of the disclosure, wherein each of the multivalent linker is linked to a different reporter.

In some embodiments, the target antigen unit comprises a binding domain capable of binding to a test antigen after the multivalent linker binds to the target antigen unit.

In some embodiments, the composition further comprises a decoy molecule that comprises an epitope of the peptide binding arm(s) but does not comprise the binding domain capable of binding to the test antigen.

In some embodiments, the comprises a cryoprotectant selected from glycerol, ethylene glycol, and dimethyl sulfoxide (DMSO). In some embodiments, the cryoprotectant is glycerol, and wherein the concentration of the glycerol is up to 50% by volume. In some embodiments, the glycerol concentration is less than 30% or less than 15% by volume. In some embodiments, the glycerol concentration is no less than 5% or no less than 10% by volume.

In one aspect, the disclosure provides methods for detecting a test antigen in a sample, comprising the steps of:
A) contacting the sample with the multivalent linker of the disclosure and a binding agent capable of specifically binding to the test antigen, and
B) removing unbound binding agent and multivalent linker, and
C) measuring the presence of the multivalent linker in the sample;
   wherein the binding agent comprises the target antigen unit, and wherein the binding agent specifically binds to the test antigen.

In one aspect, the disclosure provides methods for detecting two or more test antigens in a sample comprising contacting the sample with a first binding agent specific for a first test antigen and a second binding agent specific for a second test antigen, wherein the first and second binding agents are each bound to a first multivalent linker and a second multivalent linker, respectively, wherein the first and/or second multivalent linkers are the multivalent linkers of the disclosure, and wherein each multivalent linker is attached to a reporter, wherein the reporters are not the same.

In some embodiments, each multivalent linker specifically binds the constant region of the first or second binding agent with an apparent k_{off}(s⁻¹) rate of less than or equal to 1.0×10⁻⁵.

In some embodiments, the first and second binding agents are each non-covalently bound to a first multivalent linker and a second multivalent linker, respectively. In some embodiments, the first and second binding agents are each linked or conjugated to a first multivalent linker and a second multivalent linker, respectively.

In some embodiments, less than 5%, 4%, 3%, 2%, or 1% of the multivalent linkers bind to two or more of the binding agents. In some embodiments, less than 5%, 4%, 3%, 2%, or 1% of the first multivalent linker binds to the second binding agent, and wherein less than 5%, 4%, 3%, 2%, or 1% of the second multivalent linker binds to the first binding agent.

In one aspect, the disclosure provides methods for detecting one or more test antigens in a sample comprising contacting the sample with one or more of the molecular complexes of the disclosure, wherein the single target antigen unit within each of the molecular complexes is comprised within a binding agent, and wherein the binding agent is capable of specifically binding to the test antigen.

In some embodiments, the methods are for detecting two or more different test antigens with two or more of the molecular complexes, wherein the binding agent of each of the molecular complexes is capable of specifically binding to one of the test antigens.

In some embodiments, the binding agent is an antibody or comprises an antigen binding fragment thereof. In some embodiments, the first and second antibodies are of the same species. In some embodiments, the first and second antibodies are rabbit IgG antibodies. In some embodiments, the first and second antibodies are mouse IgG antibodies. In some embodiments, the first and second antibodies are rat IgG antibodies. In some embodiments, the first and second antibodies are human IgG antibodies.

In some embodiments, the first multivalent linker is incubated with the first binding agent prior to contacting the sample with the first binding agent. In some embodiments, the second multivalent linker is incubated with the second binding agent prior to contacting the sample with the second binding agent. In some embodiments, the first binding agent is incubated with the first multivalent linker at a molar ratio of about 1:2.5. In some embodiments, the second binding agent is incubated with the second multivalent linker at a molar ratio of about 1:2.5.

In some embodiments, the first binding agent stock concentration is at least 0.001 g/l. In some embodiments, the second binding agent stock concentration is at least 0.001 g/l.

In some embodiments, glycerol concentration in a solution containing the first binding agent and/or the second binding agent is between 0-50% by volume. In some embodiments, the glycerol concentration is less than 30%, or less than 15% by volume. In some embodiments, the glycerol concentration is no less than 5% or no less than 10% by volume.

In some embodiments, unbound multivalent linker are quenched by adding a decoy molecule that comprises the epitopes of the peptide binding arms but does not bind the test antigen(s).

In some embodiments, unbound multivalent linker are removed from multivalent linker-binding agent complexes.

In some embodiments, unbound multivalent linkers are removed by ultrafiltration.

In some embodiments, the unbound multivalent linkers are removed by bead depletion.

In some embodiments, unbound multivalent linkers are removed by adding unspecific polyclonal IgG, or fragments thereof.

In some embodiments, unbound multivalent linkers are removed by adding unspecific monoclonal IgG, or fragments thereof.

In some embodiments, the first multivalent linker and the first binding agent are incubated for about 30 minutes. In some embodiments, the first multivalent linker and the first binding agent are incubated for less than 10 minutes. In some embodiments, the second multivalent linker and the second binding agent are incubated for about 30 minutes. In some embodiments, the second multivalent linker and the second binding agent are incubated for less than 10 minutes.

In some embodiments, the method is for western blotting, enzyme linked immunosorbent assay (ELISA), immunofluorescence detection, immunohistochemistry, flow cytometry, fluorescence assisted cell sorting (FACS), screening of antibodies (e.g., using hybridomas), spatial genomic analysis, or mass spectroscopy. In some embodiments, the method is for cyclic immunofluorescence detection.

In one aspect, the disclosure provides molecular complexes comprising:
(a) a single target antigen unit comprising:
   i) a constant or Fc region of an antibody, said constant or FC region being selected from:
      1) a human IgG1, IgG2, IgG3, or IgG4;
      2) a murine IgG1, IgG2a, IgG2b, IgG2c or IgG3;
      3) a rat IgG1, IgG2a, IgG2b, or IgG2c; or
      4) a rabbit IgG; and
(b) a multivalent linker comprising:
   i) a plurality of peptide binding arms, wherein each peptide binding arm binds to the single target antigen unit, wherein the peptide binding arm is a VHH of a camelid heavy chain antibody; and
   ii) at least one peptide linker segment covalently operably linked to the plurality of peptide binding arms, wherein the peptide linker segment is between 10 and 40 amino acids long, and wherein at least 80%, at least 85%, at least 90%, at least 95%, or 100%, of the amino acids in the linker segment are selected from glycine (G), alanine (A), serine (S), threonine (T), glutamic acid (E), aspartic acid (D), lysine (K) and arginine (R).

In some embodiments, each of the peptide binding arms of the multivalent linker is non-covalently linked to a CH2 domain, a CH3 domain, and/or a CH4 domain of the constant region or the Fc region of the antibody. In some embodiments, the linker segment comprises a (G4S) unit. In some embodiments, the linker segment comprises more than 3 (G4S) units. In some embodiments, the plurality of peptide binding arms and the peptide linker segment are operably linked via a translational fusion.

### BRIEF DESCRIPTION OF THE FIGURES

The patent or application file contains at least one drawing executed in color.

FIG. 1 shows biolayer interferometry results depicting the comparative affinities of monovalent vs. multivalent linkers at various concentrations over time. In this case, a bivalent multivalent linker according to the present disclosure at a concentration of 1.25 nM has a higher apparent affinity compared to a monovalent linker at 20 nM. A bivalent multivalent linker at 20 nM is shown for comparison.

FIG. 2 shows biolayer interferometry results depicting the dissociation of a multivalent linker at 0.6, 1.2 and 2.5 nM concentrations. Multivalent linker binding remains strong, with little to no signal loss throughout the tested period.

FIG. 3 depicts the results of an immunofluorescence (IF) labeling experiment comparing the relative label signal strength and leaking of various multivalent linkers with different linker segment lengths compared to that of a control monovalent linker. Multivalent linkers of the present disclosure suffered from less signal leaking than control monovalent linkers. Multivalent linkers with shorter (G4S)₃ (SEQ ID NO: 527) linker segments exhibited some signal leaking, though at lower levels than monovalent controls. Multivalent linkers with longer (G4S)₄ (SEQ ID NO: 528) and (G4S)₅ (SEQ ID NO: 529) linker segment lengths exhibited essentially no leaking. A more detailed description of this experiment is provided in Example 6.

FIGS. 4A-F depict the results of a multiplexing Western Blot experiment using the multivalent linkers of the present disclosure multiplexed with multiple primary antibodies from the same species. FIG. 4A and FIG. 4C are scans Western Blots treated with anti-Cox and an anti-TDP primary antibodies of the same species labeled by two multivalent linkers comprising a reporter molecule detectable under a 488 channel or a reporter molecule detectable under a 647 channel, respectively. FIG. 4B and FIG. 4D are controls for single, non-multiplexed Western Blots of the same anti-Cox primary antibody labeled with a multivalent linker comprising a reporter molecule detectable under a 488 channel or an anti-TDP primary antibody labeled with a multivalent linker comprising a reporter molecule detectable under a 647 channel. The densitometry of each western is presented in FIG. 4E and FIG. 4F. The multivalent linkers of the present disclosure allow for multiplexing using antibodies from the same species with no signal leaking. A more detailed description of this experiment is provided in Example 7.

FIG. 5 shows a crystal structure of the Fc fragment of a mouse IgG1 antibody (PDB ID ligy). The distances are shown between equivalent symmetric positions (dashes) as used as a proxy for the distance between potential epitopes for a multivalent linker of the present disclosure. The Euclidean distances are given in Å (1 Å=0.1 nm). The epitope distances measured in the model correlate with empirical evidence demonstrating the consequences of various linker segment lengths within the presently claimed multivalent linkers. A more detailed description of the model and measurements made is provided in Example 10.

FIG. 6A is a flow cytometry scatterplot of a fluorescence-activated single cell sorting (FACS) experiment using a primary antibody labelled with multivalent linker of the present disclosure. Experiments were conducted with varying concentrations of glycerol (0-25%) to demonstrate functionality of the multivalent linkers under a wide range of glycerol concentrations. A more detailed description of the model and measurements made is provided in Example 8. FIG. 6B shows the ELISA result using GFP as antigen, a primary anti-GFP antibody, and a HRP-multivalent linker.

FIG. 7 depicts the results of a simulation evaluating the time it takes for 2% or 5% dissociation of an epitope-binding molecule with different K_{off} properties. In some embodiments, the multivalent linkers of the present disclosure are designed to exhibit strong binding via low K_{off} values.

FIG. 8 shows binding kinetics of the anti-rabbit IgG multivalent linker determined using biolayer interferometry, demonstrating near-absence of dissociation. The multivalent linker was titrated from 2.5 to 0.3 nM on immobilized rabbit IgG.

FIG. 9 shows the results of dynamic light scattering (DLS) analysis indicating homogenous labelling of antibodies using multivalent linkers. Test samples included a rabbit IgG primary antibody alone, or in complex with the anti-rabbit IgG multivalent linker or two conventional secondary antibodies (either in a Fab2 format or as intact IgG). The results indicate that the multivalent linkers leave the oligomeric state of the primary antibody unaffected.

FIG. 10 is a schematic illustrating an exemplary protocol for labeling antibodies using multivalent linkers.

FIGS. 11A-11C show co-staining of HeLa cells using two mouse IgG1 primary antibodies with the multivalent linkers. Anti-mouse IgG1 multivalent linker conjugated to 555 dye and anti-mouse IgG1 multivalent linker conjugated to 647 dye were used to label the mouse IgG1 primary antibodies anti-HSP60 (mitochondria, red) and anti-GORASP2 (Golgi, green). Nuclei were stained using DAPI (blue). FIG. 11A shows micrographs for the separate channels. FIG. 11B shows the merged image for all three channels. FIG. 11C is a profile plot for transect indicated in 11B (white straight line), as calculated using ImageJ Plugin RGB profiler. Confocal images were acquired using a 100× oil objective.

FIG. 12 shows labelling of primary antibodies at a wide range of concentrations. The primary antibodies were labelled using multivalent linkers at the indicated concentrations and used to stain HeLa cells.

FIG. 13A shows results of the complex stability of mouse IgG1 primary antibodies and multivalent linkers. Here, seven mouse IgG1 primary antibodies were labelled using the multivalent linker for mouse IgG1 and stored at 4° C. (Quencher was added during storage, too). After 7 days, 28 days and 3 months appropriate amounts of the complexes were used to stain HeLa cells, including freshly labelled positive controls, respectively. IF signals at each timepoint were normalized to the IF signals of freshly prepared samples showing 70%-100% normalized IF signals up to the maximal tested time period of 3 months. FIG. 13B shows immunofluorescence images demonstrating the stability of multivalent linker staining. HeLa cells were stained using the indicated primary antibodies and multivalent linkers and imaged the same day (Day 0) or 42 days after.

FIGS. 14A and 14B show multiplex immunostaining images using multivalent linkers. FIG. 14A shows an image of PFA-fixed HeLa cells stained using the following mouse IgG1 primary antibodies labelled with anti-mouse IgG1 multivalent linkers: anti-Lamin B1(green), anti-HSP60 (red), and anti-GORASP2 (cyan). Confocal image was acquired using a 100× oil objective and post-processed. FIG. 14B shows multiplex images of PFA-fixed HeLa cells stained using the following rabbit primary antibodies labelled with anti-rabbit IgG multivalent linkers: anti-TDP43 (green), anti-TOM20 (red), anti-Lamin B1 (magenta), and anti-CD147 (cyan).

FIGS. 15A and 15B show multiplex immunostaining images using multivalent linkers for different isotypes or together with chemically conjugated primary antibodies. FIG. 15A shows an image of PFA-fixed HeLa cells stained using the following primary antibodies and labelled with multivalent linkers: rabbit polyclonal IgG anti-TOM70 (green), mouse IgG2a anti-GNL3 (red), and mouse IgG2a anti-R-actin (magenta). Cell nuclei are in cyan.

FIG. 15B shows an image of PFA-fixed HeLa cells stained using mouse IgG2 anti-GLN3 chemically conjugated to 594 dye and using the following mouse IgG2a primary antibodies labelled with anti-mouse IgG2a multivalent linker: anti-Tubulin (green) and anti-Lamin A/C (magenta). Confocal images were acquired with a 100× oil objective and post-processed.

FIGS. 16A and 16B show multiplex immunostaining images of tissues using multivalent linkers. FIG. 16A shows an image of FFPE human kidney sections stained with rabbit polyclonal IgG anti-Calbindin labelled with anti-rabbit IgG multivalent linker conjugated to 555 dye (yellow), mouse IgG1 anti-ACE2 labelled with anti-mouse IgG1 multivalent linker conjugated to 647 dye (magenta), 488-conjugated rabbit polyclonal anti-podocalyxin (green) and DAPI (blue). FIG. 16B shows an image of FFPE rat brain tissue sections stained with mouse IgG1 anti-NeuN labelled with anti-mouse IgG1 multivalent linker conjugated to 647 dye (magenta), mouse IgG1 anti-TUBB3 labelled with anti-mouse IgG1 multivalent linker conjugated to 555 dye (orange), and 488-conjugated mouse IgG2a anti-GFAP (green).

FIG. 17 shows the imaging results of CyCIF multiplexing using multivalent linkers. Fixed HeLa were immunostained in three cycles using the indicated primary antibodies labelled with 750-conjugated multivalent linker and bleached using 4.5% H2O2+24 mM NaOH in PBS (1 hour at room temperature under light).

FIG. 18 shows the results of flow cytometry leaking assay for multivalent linkers. PBMCs were stained either with a mouse IgG1 isotype control labelled with anti-mouse IgG1 multivalent linker (left), a mouse IgG1 anti-CD3 monoclonal antibody (middle) labelled with anti-mouse IgG1 multivalent linker or the isotype control labelled with anti-mouse IgG1 multivalent linker and treated with a quencher in the presence of the anti-CD3 antibody (right).

FIG. 19 shows the results of flow cytometry multiplex analysis of surface markers of peripheral blood mononuclear cells (PBMCs) with primary antibodies of different isotypes. Mouse IgG1 anti-CD45, mouse IgG2a anti-CD3, and mouse IgG2b a-C4 were stained with the respective anti-mouse IgG1 multivalent linker conjugated to 488 dye, anti-IgG2a multivalent linker conjugated to 555 dye, or anti-IgG2b multivalent linker conjugated to 647 dye, and used to stain PBMCs.

FIG. 20 shows the results of flow cytometry multiplex analysis of surface markers of peripheral blood mononuclear cells (PBMCs) with primary antibodies of the same isotype. Multivalent linkers were used to label mouse IgG1 primaries anti-CD3 (FITC), anti-CD4 (555 dye), anti-CD8 (647 dye), and anti-CD45 (750 dye) and to stain PBMCs.

FIG. 21 shows the results of flow cytometry analysis of intracellular markers of HEK293T cells. Multivalent linkers were used to label rabbit polyclonal antibodies against the mitochondrial outer membrane proteins TOMM40 and TOMM20 and the inner membrane proteins COX2 and mitofilin and to stain HEK293T cells.

FIG. 22 shows flow cytometry results demonstrating the compatibility of the multivalent linkers with additives. Primary anti-CD3 antibodies stored in various concentrations of glycerol or bovine serum albumin (BSA) or in cell culture media comprising 15% fetal bovine serum (FBS) were labelled using multivalent linkers and used for staining of the T cell subpopulation of peripheral blood mononuclear cells (PBMCs). The primary antibody clones were mouse IgG1 in the glycerol and cell culture media experiments and mouse IgG2a for the BSA titration (co-staining with mouse IgG1 anti-CD45).

FIG. 23 shows flow cytometry results demonstrating the compatibility of the multivalent linkers for flow cytometry in the presence of additives. Primary mouse IgG1 anti-CD4 antibody was labelled using anti-mouse IgG1 multivalent linker conjugated to 647 dye and used for staining of the CD4+1 subpopulation of peripheral blood mononuclear cells (PBMCs) in the presence of increasing concentrations of bovine serum albumin (BSA), fetal bovine serum and the chelator EDTA.

FIGS. 24A-24C show the results of multivalent linker labelling of hybridoma supernatants. In FIG. 24A, hybridoma supernatant with varying amounts of mouse IgG1 anti-CD3 was simulated by adding the indicated amounts of primary antibody to 1×RPMI medium plus 15% FBS, labelled with anti-mouse IgG1 multivalent linker conjugated to 647 dye and used to stain PBMCs. In FIG. 24B, supernatant of hybridoma cells expressing mouse IgG2a anti-CD3 were diluted in IMDM medium plus 15% FBS as indicated, labelled with anti-mouse IgG2a multivalent linker conjugated to 647 dye and used to stain PBMCs. As a control, given amounts of purified antibody were treated similarly. In FIG. 24C, hybridoma cells expressing mouse IgG2a anti-CD3 were seeded at indicated numbers in a 96-well plate. After 72 hrs, the supernatant was labelled with anti-mouse IgG1 multivalent linker conjugated to 647 dye and used to stain PBMCs.

### DETAILED DESCRIPTION

### Definitions

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

All embodiments of any aspect of the disclosure can be used in combination unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Additionally, the words “herein,” “above,” “below,” and words of similar import, when used in this application, shall refer to this application as a whole and not to any particular portions of the application.

The term “about” means a quantity, level, value, number, frequency, percentage, dimension, size, amount, weight or length that varies by as much as 10% of a reference quantity, level, value, number, frequency, percentage, dimension, size, amount, weight or length, inclusive of the endpoints. When the term “about” is used in conjunction with a numerical range, it modifies that range by extending the boundaries above and below the numerical values set forth, unless otherwise apparent from context that it is impossible to extend the boundary beyond certain points (e.g., below 0% or above 100%).

The term “affinity” refers to the strength of the interaction between an antigen and a binding agent's (e.g., antibody's) antigen binding site. The affinity can be determined, for example, using the equation

K_{A}=[Ab:Ag]/[Ab][Ag];

Where K_{A}=affinity constant; [Ab]=molar concentration of unoccupied binding sites on the binding agent; [Ag]=molar concentration of unoccupied binding sites on the target antigen; and [Ab:Ag]=molar concentration of the binding agent-target antigen complex. The K_{A} describes how much binding agent-antigen complex exists at the point when equilibrium is reached. The time taken for this to occur depends on rate of diffusion and is similar for every antibody. However, high-affinity antibodies will bind a greater amount of antigen in a shorter period of time than low-affinity antibodies. The K_{A} of the antibodies produced can vary and range from between about 10⁵ mol⁻¹ to about 10¹² mol⁻¹ or more. The K_{A} can be influenced by factors including pH, temperature, and buffer composition.

The antibody affinity can be measured using any means commonly employed in the art, including but not limited to the use of biosensors, such as surface plasmon resonance (SPR), or Bio-Layer Interferometry (BLI). Resonance units are proportional to the degree of binding of soluble ligand (e.g., protein antigen) to the immobilized binding agent (or soluble binding agent to immobilized ligand such as protein antigen). Determining the amount of binding at equilibrium with different known concentrations of binding agent (e.g., antibody) and ligand (e.g., protein antigen) allows the calculation of equilibrium constants (K_{A}, K_{D}), and the rates of dissociation and association (k_{off}, kₒₙ). The K_{A} can be calculated using the formula K_{A}=kₒₙ/k_{off}.

Unless otherwise stated, the affinity measurements (K_{A} or K_{D}) are derived from the dissociation/association rates (k_{off} and kₒₙ) that are obtained using the BLI method.

The term “avidity” refers to the accumulated strength of multiple affinities of individual non-covalent binding interactions, such as between a multivalent linker and its antigen. Avidity is related to both the affinity of individual immunoglobulin molecules in the population with specific epitopes, and also the valencies of the multivalent linkers and the antigen. Therefore, for a multivalent binding molecule, the affinity might appear higher, and the off-rate might appear slower when compared to the monovalent molecule, although the affinity and off-rate of each individual binding domain of the multivalent molecule are identical to the monovalent affinity or off-rate, respectively.

The term “k_{off}” refers to the dissociation rate or off constant, or specific reaction rate, for dissociation of a binding agent from a binding agent/antigen complex, measured in units: 1/second (s⁻¹).

The term “kₒₙ” refers to the association rate constant or on, or specific reaction rate, of a direct or complex-forming reaction, measured in units: M⁻¹s⁻¹.

Unless stated otherwise, “k_{off}” or “kₒₙ” values are derived from the following condition using Bio-Layer Interferometry (BLI): phosphate buffered saline pH 7, 0.1% (m/v) BSA, 0.02% (v/v) Tween20, and 0.02% NaN₃; all samples are set up in a microplate (200 μl/well) at room temperature, and all experiments are run at 30° C., a shaking speed of 1000 rpm and a recording rate of 5 Hz. The target antigen unit of the multivalent linker is immobilized on the biosensor, preferably through a biotin tag to streptavidin on the biosensor when applicable.

The “equilibrium dissociation constant” or “dissociation constant” or “K_{D}” can be calculated using the formula K_{D}=k_{off}/kₒₙ. The K_{D} is a ratio of k_{off}/kₒₙ, between the binding agent and its antigen. K_{D} and affinity (K_{A}) are inversely related. The lower the K_{D} value (lower peptide concentration), the higher the affinity of the antibody. Most antibody-derived antigen binding domains have K_{D} values in the low micromolar (10⁻⁶ M) to nanomolar (10⁻⁷ to 10⁻⁹ M) range. High affinity antigen binding domains are generally considered to have a K_{D} in the low nanomolar range (10⁻⁹ M) with very high affinity being a K_{D} in the picomolar (10⁻¹² M) range or even lower (e.g. 10⁻¹³ to 10⁻¹⁴ M range). In one embodiment, the peptides disclosed herein have an apparent K_{D} ranging from about 10⁻⁶ to about 10⁻¹⁵ M, about 10⁻⁷ to about 10⁻¹⁵ M, about 10⁻⁸ to about 10⁻¹⁵ M, about 10⁻⁹ to about 10⁻¹⁵ M, about 10⁻¹⁰ to about 10⁻¹⁵ M, about 10⁻¹¹ to about 10⁻¹⁵ M, about 10⁻¹² to about 10⁻¹⁵ M, about 10⁻¹³ to about 10⁻¹⁴ M, about 10⁻¹³ to about 10⁻¹⁵ M, or about 10⁻¹⁴ to about 10⁻¹⁵ M.

The multivalent linkers produced by the methods disclosed herein have high avidity, indicating they bind tightly to the antigen. In some embodiments, the multivalent linkers of the disclosure have very low apparent dissociation rates (k_{off}) due to avidity effects.

The term “valent” refers to the presence of a specified number of binding sites of a binding agent (e.g., multivalent linker). As such, the terms “bivalent”, “tetravalent”, and “hexavalent” refer to the presence of two binding sites, four binding sites, and six binding sites, respectively, in a binding agent (e.g., multivalent linker_of the present disclosure. Multivalent linkers, according to the disclosure, may be at least “bivalent”, “trivalent”, “tetravalent” or “hexavalent”.

In some embodiments, the multivalent linker is bivalent, trivalent, tetravalent, or hexavalent. In some embodiments, the multivalent linker is bivalent. In some embodiments, the multivalent linker is trivalent. In some embodiments, the multivalent linker is tetravalent. In some embodiments, the multivalent linker is hexavalent.

The term “immunoglobulin” refers to a glycoprotein that may include at least two heavy (H) chains and two light (L) chains linked by disulfide bonds, or an antigen binding portion thereof. Each heavy chain has a heavy chain variable region (abbreviated herein as V_{H}) and a heavy chain constant region. The heavy chain constant region may include three domains, C_{H}1, C_{H}2 and C_{H}3. Each light chain has a light chain variable region (abbreviated herein as V_{L}) and a light chain constant region. The light chain constant region includes one domain, C_{L}. The V_{H} and V_{L} regions can be further subdivided into regions of hypervariability, termed complementarity determining regions (CDR), interspersed with regions that are more conserved, termed framework regions (FR). The CDRs contain most of the residues responsible for specific interactions of the antibody with the antigen. Each V_{H} and V_{L} has three CDRs and four FRs, arranged from amino-terminus (N-terminus) to carboxy-terminus (C-terminus) in the following order: FR1, CDR1, FR2, CDR2, FR3, CDR3, FR4. The variable regions of the heavy and light chains contain a binding domain that interacts with an epitope of an antigen. The term “immunoglobulin” may also include two heavy chains without light chains, such as e.g. an antibody devoid of light chains.

Each light chain of an immunoglobulin includes an N-terminal variable (V) domain (VL) and a constant I domain (CL). Each heavy chain includes an N-terminal V domain (VH), three or four C domains (CHs), and a hinge region.

An immunoglobulin may be a tetrameric glycosylated protein composed of two light (L) chains of approximately 25 kDa each and two heavy (H) chains of approximately 50 kDa each. Two types of light chain, termed lambda and kappa, may be found in immunoglobulins. Depending on the amino acid sequence of the constant domain of heavy chains, immunoglobulins can be assigned to five major classes: A, D, E, G, M, and Y, and several of these may be further divided into subclasses (isotypes), e.g., IgG1, IgG2, IgG3, IgG4, IgA1, and IgA2. An IgM immunoglobulin consists of 5 of the basic heterotetramer units along with an additional polypeptide called a J chain, and contains 10 antigen binding sites, while IgA immunoglobulins contain from 2-5 of the basic 4-chain units which can polymerize to form polyvalent assemblages in combination with the J chain.

An immunoglobulin is “specific to” or “specifically binds” (used interchangeably herein) to a target (e.g., HA) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An immunoglobulin “specifically binds” to a particular protein or substance if it binds with greater affinity, avidity, more readily, and/or with greater duration than it binds to alternative particular protein or substance. For example, an immunoglobulin that specifically or preferentially binds to HA is an immunoglobulin that binds HA with greater affinity, avidity, more readily, and/or with greater duration than it binds to other proteins. An immunoglobulin that specifically binds to a first protein or substance may or may not specifically or preferentially bind to a protein, cell, or substance. As such, “specific binding” does not necessarily require (although it can include) exclusive binding. Generally, but not necessarily, reference to binding means specific binding.

In some embodiments, “target antigen” refers to an antibody domain that the multivalent linker of the disclosure binds.

As used herein, the term “target antigen unit” refers to a single unit (e.g., molecule, peptide, or other structurally discernible particle) comprising the one or more epitopes bound by the multivalent linkers of the present disclosure. In some embodiments, binding agents (e.g., primary antibodies) are target antigen units.

As used herein the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs.

In some embodiments, identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described herein. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega® (Sievers et al., Mol Syst Biol. 2011 Oct. 11; 7:539) may be used. Unless noted otherwise, the term “sequence identity” in the claims refers to sequence identity as calculated by Clustal Omega® using default parameters.

The term “therapeutic agent” refers to a moiety that is directly or indirectly conjugated to the multivalent linker that has a specific biological effect. For example, a therapeutic agent may include, but is not limited to, a chemotherapeutic agent.

The term “reporter molecule” refers to a detectable moiety that is directly or indirectly conjugated to the multivalent linker. A reporter molecule includes, but is not limited to, fluorescent reporters, chemiluminescence reporters, radioactive reporters, and magnetic reporters.

### Traditional Immunoassay Limitations

Detection of a protein of interest by an antibody typically requires a primary antibody and a secondary antibody conjugated to a reporter molecule. For example, a primary antibody is typically obtained from one specific animal species, e.g. rabbit, and is used to bind the protein of interest. A secondary antibody specifically recognizes the primary antibody, and usually brings with it an attached label that is then used to detect the primary antibody (ideally bound to the epitope of interest). This secondary antibody is typically derived from another species than the primary antibody.

Labeling with reporter molecules on the secondary antibody is typically performed with non-directed chemistry. As a result, the number of reporter molecules present in every single secondary antibody is not precisely known. An average distribution of between 0.5 to ˜3.5 reporter molecules occurs per secondary antibody, which can vary from distributor-to distributor and batch-to-batch. Consequently, this variability in immunoassays leads to reduced reproducibility. For example, an average distribution of 0.5 to ˜3.5 reporter molecules per secondary antibody means that some secondary antibodies have no reporter molecules (no detection) and some have up to 5 or more reporter molecules (increased detection).

Another traditional technique is the direct labeling of the primary antibodies with reporter molecules, which is also typically performed with non-directed chemistry (i.e. random). The direct coupling of reporter molecules to primary antibodies is not often performed due to the risk of inactivating the binding capability of the antibody. This risk is especially high on monoclonal antibodies since adding a reporter molecule on the paratope (epitope binding region on the antibody) will completely impair the antibody binding function of all molecules. Polyclonal antibodies might not be completely inactivated by the coupling since there is a chance that some antibody molecules are not affected by the chemistry applied, but affinity of labeled antibodies can vary, thus introducing further variability into assays. Moreover, the chemical labeling of primary antibodies, e.g. via NHS ester chemistry, is hindered by incompatible buffer substances and additives commonly used in antibody formulation, such as TRIS, BSA, or azide, or may lead to loss of activity.

One drawback of classical immunoassays is that complexes of standard primary and secondary antibodies tend to form clusters. Furthermore, reporter molecules are displaced from the desired target molecule between 15-25 nm. As current microscopes can have a resolution power of ˜5 nm, the use of primary and secondary antibodies can result in an error of 10-20 nm in all three dimensions from the true location of the investigated target.

An additional drawback of classical immunoassays is the inability to use multiple primary antibodies of the same species in the same experiment (i.e., inability to multiplex). Specifically, the cross-reactivity and high k_{off} rates of traditional secondary antibodies makes it difficult/impossible to multiplex on primary antibodies from the same species, because labeled secondary antibodies may dissociate from their original target, and bind to another primary antibody (i.e., signal leaking). This inability to multiplex increases the time and cost of experiments, and often forces researchers to use inferior antibodies from a different species to avoid the leaking issues described above.

Further drawbacks of classical immunoassays stem from how antibodies are typically produced. For example, production of both primary and secondary antibodies require in vivo immune treatments in individual animals, followed by serum collection. Great variability in immune responses between individual animals lead to batch variability. Maintaining immunized animals is costly and can raise animal welfare issues (Reardon, S. US government issues historic $3.5-million fine over animal welfare. Nature (2016)). Hybridomas can be made for specific antibodies to reduce variability; however, these methods are time consuming and cost prohibitive for most antibodies.

Classical immunoassays further involve time consuming and error prone experimental protocols are further limitation of classical immunoassay methods. Current protocols typically apply the primary antibody for a particular time, typically ranging from 0.5 to 24 h, and then wash off the excess of primary antibody that did not find any target. After washing, the secondary antibody is incubated for a particular time, typically ranging from 0.5 to 18 h, and then washed several times, typically for 10 to 30 min, to make sure no free reporter molecule labeled secondary antibodies are left in the preparation. Additional washing steps associated with traditional immunoassays are prone to errors, which can easily result in a diminished primary signal that hampers experimental interpretation.

### Multivalent Linkers of the Present Disclosure

In some embodiments, the present disclosure solves the aforementioned problems associated with traditional immunoassays and treatments. Specifically, in some embodiments, the present disclosure teaches a multivalent linker that specifically binds a target antigen, said multivalent linker comprising: a) a plurality of peptide binding arms, each binding arm capable of binding to an epitope in the same target antigen; and b) a linker segment covalently operably linked to the plurality of peptide binding arms. In some embodiments the linker segments are sufficiently long so as to keep the peptide binding arms at c) a preselected distance factor, designed to reduce or avoid cross linking to other target antigen units. In some embodiments, the multivalent linkers of the present disclosure further comprise d) a reporter or effector.

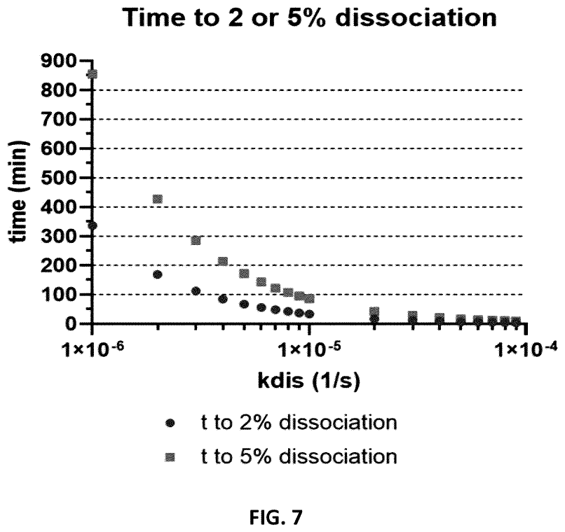

In some embodiments, the advantages of the multivalent linkers of the disclosure include: (1) high specificity for its target, (2) capable of being labeled with a fluorophore or other label of choice, and (3) binds the target with an extremely low dissociation rate (k_{off}). For example, a k_{off} of 10⁻⁵ s⁻¹ would result in only 5% dissociation over 90 min (FIG. 7)—i.e. approximately the time frame of a typical staining experiment. In practical terms, such a low k_{off} would allow the labelling of a primary antibody while minimizing the risk of cross-staining due to the multivalent linker's dissociation and rebinding to a different primary antibody of the same species or isotype.

As discussed above, one problem with immunoassays is the inability to use primary antibodies of the same species in the same assay. The present disclosure provides a solution to this problem by a labeling step that binds multivalent linkers to primary antibodies in a safe way that preserves the primary antibody's paratope. That is, in some embodiments, the multivalent linkers of the present disclosure have superior apparent k_{off} rates that allow the multivalent linker to bind a primary antibody without using the harsh conditions typically required during a direct labeling step.

The multivalent linker labeling step further allows the multiple primary antibodies of the same species to be used in the same assay. Many times, the “best” antibody will be known and used in a particular field of study. Therefore, classical immunoassays are limited to using antibodies of a different species to label a different protein of interest. This creates impossible experiments due to a lack of suitable combinations of antibodies from different species or the process of testing antibodies that can cost thousands of dollars and take weeks to find a suitable antibody.

Another advantage of the current disclosure over the prior art are the size of the multivalent linkers. For example, a typical secondary antibody is about ˜150 kDa. The large size of a secondary antibody introduces the aforementioned issues of an average distribution of 0.5 to ˜3.5 reporter molecules per secondary antibody and displacement from the desired target molecule by around 15-25 nm. In some embodiments, the multivalent linkers of the current disclosure are much smaller than typical antibodies. For example, bivalent peptides of the present disclosure can be around 25-35 kDa, thereby reducing these issues.

Another advantage of the multivalent linkers of the current disclosure relates to how the multivalent linkers are identified and produced. Traditional secondary antibodies require in vivo immune treatments in individual animals, followed by serum collection. Consequently, traditional techniques introduce batch variability in addition to animal welfare concerns. The in vitro techniques disclosed herein sidestep the need for in vivo production of these consumables, reducing batch variability and the need for housing immunized animals. Thus, in some embodiments, the multivalent linkers of the present disclosure exhibit superior properties to antibodies produced via traditional means.

Traditional immunoassays have multiple wash steps that can increase leaking of either the primary or secondary antibody. For example, application of the primary antibody ranges from 0.5 to 24 h, followed by several wash steps, then application of the secondary antibody that ranges from 0.5 to 18 h, finally followed by additional was steps. The additional wash steps associated with traditional immunoassays may result in a diminished primary signal that hampers experimental interpretation. The multivalent linkers of the disclosure may bypass several of these wash steps through labeling of primary antibodies via very strong, binding (e.g., non-covalent binding). Furthermore, the current disclosure may reduce the amount of time required for the assay since the secondary antibody step may be eliminated in favor of the linking step taught herein.

The multivalent linkers of the disclosure allow rapid labelling of primary antibodies for multiplex immunostaining techniques such as IF, IHC, flow cytometry and Western blotting. Importantly, labelling using the multivalent linkers is independent of the formulation or purification grade of the primary antibody. Furthermore, it is a highly scalable process, as antibody quantities ranging from sub-microgram volumes up to any volume can be labelled. This scalability may translate to significant cost efficiencies for laboratories.

Additional information regarding various aspects of the presently disclosed multivalent linkers is provided herein.

### Linker Segments

In some embodiments, the disclosure provides for a multivalent linker comprising two or more peptide binding arms covalently connected to a linker segment. In some embodiments, the disclosure provides for a multivalent linker comprising a first peptide binding arm; a second peptide binding arm; and a linker segment disposed between the first peptide binding arm and the second peptide binding arm.

In some embodiments, the linker segments of the present disclosure are polymeric molecules comprising repeating monomeric structures. Thus, in some embodiments, the multivalent linkers comprises the formula (Peptide Binding Arm)-(Linker Segment)-(Peptide Binding Arm). In some embodiments the linker segment has two reactive ends to bind separate peptide binding arms. In other embodiments, the linker segment has three or more reactive ends, each capable of binding their own peptide binding arms. In some embodiments, there is more than one linker segment, each linker segment located in between two peptide binding arms.

In some embodiments, the linker segment described herein is designed to connect (e.g., join, link) two peptide binding arms, wherein the linker segment is typically not disposed between the two binding arms in nature. In the context of the present disclosure, the phrase “linked” or “joined” or “connected” generally refers to a functional linkage between two contiguous or adjacent amino acid sequences to produce a multivalent linker that does not exist in nature. In certain embodiments, linkage may be used to refer to a covalent linkage of, for example, the amino acid sequences of the one or more peptide binding arms. Generally, linked peptide binding arms are contiguous or adjacent to one another and retain their respective operability and function when joined. In some embodiments, peptide binding arms comprised within the multivalent linkers disclosed herein are linked by means of an interposed linker segment. Such linker segments may provide desirable flexibility to permit the desired expression, activity and/or conformational positioning of the peptide binding arms within the multivalent linker.

In some embodiments, a linker segment comprises and/or consists essentially of amino acids. A typical amino acid linker segment is generally designed to be flexible or to interpose a structure, such as an alpha-helix, between the two protein moieties. In some embodiments, the terms “linker,” “linker segment” and “spacer” are interchangeably used.

In some embodiments, a linker segment may employ any one or more naturally-occurring amino acids, non-naturally occurring amino acid(s), amino acid analogs, and/or amino acid mimetics as described elsewhere herein and known in the art. Certain amino acid sequences which may be usefully employed as linker segments include those disclosed in Maratea et al., Gene 40:39-46, 1985; Murphy et al., PNAS USA. 83:8258-8262, 1986; U.S. Pat. Nos. 4,935,233 and 4,751,180. In some embodiments, the present disclosure teaches peptide linker segment sequences containing Gly, Ser, and/or Asn residues. In some embodiment, neutral amino acids, such as Thr and Ala may also be employed in the peptide linker segment sequence, if desired.

The linker segment peptide sequence can be of any appropriate length to connect one or more proteins of interest and is preferably designed to be sufficiently flexible so as to allow the proper folding and/or function and/or activity of one or both of the peptides it connects. In some embodiments, the linker segment peptide has a length of no more than 3, no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95 or no more than 100 amino acids along its longest axis, including all ranges and subranges therebetween. In some embodiments, the linker segment peptide can have a length of at least 3, at least 4, at least 5, at least 6, at least 7, at least 8, at least 9, at least 10, at least 12, at least 15, at least 18, at least 20, at least 25, at least 30, at least 35, at least 40, at least 45, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, or 100 amino acids along its longest edge, including all ranges and subranges therebetween.

In some embodiments, the linker segment comprises at least 10 and no more than 60 amino acids, at least 10 and no more than 55 amino acids, at least 10 and no more than 50 amino acids, at least 10 and no more than 45 amino acids, at least 10 and no more than 40 amino acids, at least 10 and no more 35 amino acids, at least 10 and no more than 30 amino acids, at least 10 and no more than 25 amino acids, at least 10 and no more than 20 amino acids or at least 10 and no more than 15 amino acids. In some embodiments, the linker segment comprises at least 15 and no more than 60 amino acids, at least 15 and no more than 55 amino acids, at least 15 and no more than 50 amino acids, at least 15 and no more than 45 amino acids, at least 15 and no more than 40 amino acids, at least 15 and no more 35 amino acids, at least 15 and no more than 30 amino acids, at least 15 and no more than 25 amino acids, or at least 15 and no more than 20 amino acids. In some embodiments, the linker segment comprises at least 20 and no more than 60 amino acids, at least 20 and no more than 55 amino acids, at least 20 and no more than 50 amino acids, at least 20 and no more than 45 amino acids, at least 20 and no more than 40 amino acids, at least 20 and no more 35 amino acids, at least 20 and no more than 30 amino acids, or at least 20 and no more than 25 amino acids. In some embodiments, the linker segment comprises at least 25 and no more than 60 amino acids, at least 25 and no more than 55 amino acids, at least 25 and no more than 50 amino acids, at least 25 and no more than 45 amino acids, at least 25 and no more than 40 amino acids, at least 25 and no more 35 amino acids, or at least 25 and no more than 30 amino acids. In some embodiments, the linker segment comprises at least 30 and no more than 60 amino acids, at least 30 and no more than 55 amino acids, at least 30 and no more than 50 amino acids, at least 30 and no more than 45 amino acids, at least 30 and no more than 40 amino acids, or at least 30 and no more 35 amino acids. In some embodiments, these are the number of the amino acids along the longest axis of the linker segment peptide sequence.

In some embodiments, in a polypeptide composition comprising a linker segment, the 5′ end (e.g., terminus) of the linker segment peptide sequence (e.g., amino acid sequence) is adjacent to and covalently linked to the 3′ end of one peptide sequence (e.g., full-length protein or protein domain, fragment or variant) and, further, the 3′ end of the linker segment amino acid sequence is adjacent to and covalently linked to the 5′ end of another peptide sequence.

In some embodiments, the linker segment comprises glycine. In some embodiments, the linker segment comprises glycine, alanine, serine and/or threonine amino acid residues. In some embodiments, the linker segment comprises glycine, alanine, and/or serine amino acid residues. In some embodiments, the linker segment comprises glycine, threonine, and/or serine amino acid residues. In some embodiments, the linker segment comprises glycine, alanine, and/or threonine amino acid residues. In some embodiments, the linker segment comprises glycine and/or alanine amino acid residues. In some embodiments, the linker segment comprises glycine and/or serine amino acid residues. In some embodiments, the linker segment comprises glycine and/or threonine amino acid residues.

In some embodiments, the linker segment comprises glycine. In some embodiments, at least 20% of the amino acids in the linker segment are glycine. In some embodiments, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the amino acids in the linker segment are glycine. In some embodiments, at least 50% of the amino acids in the linker segment are glycine. In some embodiments, at least 60% of the amino acids in the linker segment are glycine. In some embodiments, at least 70% of the amino acids in the linker segment are glycine. In some embodiments, at least 80% of the amino acids in the linker segment are glycine. In some embodiments, at least 90% of the amino acids in the linker segment are glycine. In some embodiments, 20-90% of the amino acids in the linker segment are glycine. In some embodiments, 20-30%, 30-40%, 40-50%, 50-60%, 60-70%, 70-80%, or 80-90% of the amino acids in the linker segment are glycine. In some embodiments, 20-40%, 30-50%, 40-60%, 50-70%, 60-80%, or 70-90% of the amino acids in the linker segment are glycine. In some embodiments, 20-50%, 30-60%, 40-70%, 50-80%, or 60-90% of the amino acids in the linker segment are glycine. In some embodiments, 60%-90% of the amino acids in the linker segment are glycine.

In some embodiments, the linker segment comprises alanine. In some embodiments, at least 5% of the amino acids in the linker segment are alanine. In some embodiments, at least 10%, at least 15%, at least 20%, at least 25%, at least 30%, at least 35%, at least 40%, at least 45%, or at least 50% of the amino acids in the linker segment are alanine. In some embodiments, 5-15%, 10-20%, 15-25%, 20-30%, 25-35%, 30-40%, 35-45%, or 40-50% of the amino acids in the linker segment are alanine. In some embodiments, 5-25%, 10-30%, 15-35%, 20-40%, 25-45%, or 30-50% of the amino acids in the linker segment are alanine. In some embodiments, 10-30%, 15-25%, or about 20% of the amino acids in the linker segment are alanine.

In some embodiments, the linker segment comprises (i) glycine and (ii) alanine. In some embodiments, the ratio of glycine to alanine is about 1:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1. In some embodiments, the ratio of glycine to alanine is at least 1:1, at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 9.5:1, or at least 10:1. In some embodiments, the ratio of glycine to alanine is at most 1:1, at most 1.5:1, at most 2:1, at most 2.5:1, at most 3:1, at most 3.5:1, at most 4:1, at most 4.5:1, at most 5:1, at most 5.5:1, at most 6:1, at most 6.5:1, at most 7:1, at most 7.5:1, at most 8:1, at most 8.5:1, at most 9:1, at most 9.5:1, or at most 10:1.

In some embodiments, the linker segment comprises serine and/or threonine. In some embodiments, at least 5% of the amino acids in the linker segment are serine and/or threonine. In some embodiments, at least 10%, at least 15%, at least 20%, at least 25%, at least 30%, at least 35%, at least 40%, at least 45%, or at least 50% of the amino acids in the linker segment are serine and/or threonine. In some embodiments, 5-15%, 10-20%, 15-25%, 20-30%, 25-35%, 30-40%, 35-45%, or 40-50% of the amino acids in the linker segment are serine and/or threonine. In some embodiments, 5-25%, 10-30%, 15-35%, 20-40%, 25-45%, or 30-50% of the amino acids in the linker segment are serine and/or threonine. In some embodiments, 10-30%, 15-25%, or about 20% of the amino acids in the linker segment are serine and/or threonine. In some embodiments, such amino acid are serine. In some embodiments, the serine to threonine ratio is at least 1:1, at least 2:1, at least 3:1, at least 4:1, at least 5:1, at least 6:1, at least 7:1, at least 8:1, at least 9:1, or at least 10:1.

In some embodiments, the linker segment comprises (i) glycine and (ii) serine and/or threonine. In some embodiments, the ratio of glycine to serine and/or threonine is about 1:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1. In some embodiments, the ratio of glycine to serine and/or threonine is at least 1:1, at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 9.5:1, or at least 10:1. In some embodiments, the ratio of glycine to serine and/or threonine is at most 1:1, at most 1.5:1, at most 2:1, at most 2.5:1, at most 3:1, at most 3.5:1, at most 4:1, at most 4.5:1, at most 5:1, at most 5.5:1, at most 6:1, at most 6.5:1, at most 7:1, at most 7.5:1, at most 8:1, at most 8.5:1, at most 9:1, at most 9.5:1, or at most 10:1.

In some embodiments, at least 70%, at least 75%, at least 80%, at least 85%, at least 90%, at least 95%, or 100%, of the amino acids in the linker segment are glycine (G), alanine (A), serine (S), threonine (T), glutamic acid (E), aspartic acid (D), lysine (K) and arginine (R). In some embodiments, at least 70%, at least 75%, at least 80%, at least 85%, at least 90%, at least 95%, or 100%, of the amino acids in the linker segment are glycine (G), alanine (A), serine (S), threonine (T), glutamic acid (E), aspartic acid (D), lysine (K). In some embodiments, at least 70%, at least 75%, at least 80%, at least 85%, at least 90%, at least 95%, or 100%, of the amino acids in the linker segment are glycine (G), serine (S), threonine (T), glutamic acid (E), aspartic acid (D), lysine (K) and arginine (R). In some embodiments, at least 70%, at least 75%, at least 80%, at least 85%, at least 90%, at least 95%, or 100%, of the amino acids in the linker segment are glycine (G), alanine (A), glutamic acid (E), aspartic acid (D), lysine (K) and arginine (R). In some embodiments, at least 70%, at least 75%, at least 80%, at least 85%, at least 90%, at least 95%, or 100%, of the amino acids in the linker segment are glycine (G), glutamic acid (E), aspartic acid (D), lysine (K) and arginine (R). In some embodiments, at least 70%, at least 75%, at least 80%, at least 85%, at least 90%, at least 95%, or 100%, of the amino acids in the linker segment are glycine (G), serine (S), threonine (T), glutamic acid (E), aspartic acid (D), and lysine (K). In some embodiments, at least 70%, at least 75%, at least 80%, at least 85%, at least 90%, at least 95%, or 100%, of the amino acids in the linker segment are glycine (G), alanine (A), glutamic acid (E), aspartic acid (D), and lysine (K). In some embodiments, at least 70%, at least 75%, at least 80%, at least 85%, at least 90%, at least 95%, or 100%, of the amino acids in the linker segment are glycine (G), glutamic acid (E), aspartic acid (D), and lysine (K). In some embodiments, at least 70%, at least 75%, at least 80%, at least 85%, at least 90%, at least 95%, or 100%, of the amino acids in the linker segment are glycine (G), alanine (A), serine (S), or threonine (T). In some embodiments, at least 70%, at least 75%, at least 80%, at least 85%, at least 90%, at least 95%, or 100%, of the amino acids in the linker segment are glycine (G), serine (S), or threonine (T). In some embodiments, at least 70%, at least 75%, at least 80%, at least 85%, at least 90%, at least 95%, or 100%, of the amino acids in the linker segment are glycine (G), or serine (S). In some embodiments, such amino acids represents at least 70% of the amino acids in the linker segment. In some embodiments, such amino acids represents at least 75% of the amino acids in the linker segment. In some embodiments, such amino acids represents at least 80% of the amino acids in the linker segment. In some embodiments, such amino acids represents at least 85% of the amino acids in the linker segment. In some embodiments, such amino acids represents at least 90% of the amino acids in the linker segment. In some embodiments, such amino acids represents at least 95% of the amino acids in the linker segment. In some embodiments, such amino acids represents 100% of the amino acids in the linker segment.

In some embodiments, the amino acids can alternate/repeat in any manner consistent with the linker segment remaining functional (e.g., resulting in expressed and/or active polypeptide(s)). In some embodiments, the linker segment may alternate/repeat in any manner that comprises the formula (G)nX (SEQ ID NO: 532), wherein n is 1-100 and X is any amino acid, such as alanine, serine, or glycine. For example, the amino acids in the linker segment can repeat every one (e.g., GAGA (SEQ ID NO: 510), GSGS (SEQ ID NO: 511)), every two (e.g., GGAGGA (SEQ ID NO: 512), GGSGGS (SEQ ID NO: 513)), every three (e.g., GGGAGGGA (SEQ ID NO: 514), GGGSGGGS (SEQ ID NO: 515)), every four (e.g., GGGGAGGGGA (SEQ ID NO: 516), GGGGSGGGGS (SEQ ID NO: 517)), every five, every 6, every 7, every 8, every 9 or every 10 or more amino acids, or the amino acids can repeat in any combination of the foregoing.

In some embodiments, the amino acids repeat every four amino acids and the linker segment consists of one or more glycine repeats. In some embodiments, the linker segment comprises the formula (G4X)n wherein n represents a number of repeats and X is any amino acid, such as alanine, serine, or glycine. For example, the linker segment can consist of a GGGGA (G4A) (SEQ ID NO: 518) or GGGGS (G4S) repeat (SEQ ID NO: 519).

In some embodiments, the linker segment sequence is GGGGAGGGGA (G4A)2 (SEQ ID NO: 520), GGGGAGGGGAGGGGA (G4A)3 (SEQ ID NO: 521), GGGGAGGGGAGGGGAGGGGA (G4A)4 (SEQ ID NO: 522), GGGGAGGGGAGGGGAGGGGAGGGGA (G4A)5 (SEQ ID NO: 523), GGGGAGGGGAGGGGAGGGGAGGGGAGGGGA (G4A)6 (SEQ ID NO: 524), or GGGGAGGGGAGGGGAGGGGAGGGGAGGGGAGGGGA (G4A)7 (SEQ ID NO: 525).

In some embodiments, the linker segment sequence is GGGGSGGGGS (G4S)2 (SEQ ID NO: 526), GGGGSGGGGSGGGGS (G4S)3 (SEQ ID NO: 527), GGGGSGGGGSGGGGSGGGGS (G4S)4 (SEQ ID NO: 528), GGGGSGGGGSGGGGSGGGGSGGGGS (G4S)5 (SEQ ID NO: 529), GGGGSGGGGSGGGGSGGGGSGGGGSGGGGS (G4S)6 (SEQ ID NO: 530), or GGGGSGGGGSGGGGSGGGGSGGGGSGGGGSGGGGS (G4S)7 (SEQ ID NO: 531).

In some embodiments, the linker segment sequence comprises or consists of the amino acid sequence of GSTSGSGKSSEGKGEGSTSGSGKSG (SEQ ID NO: 495), or an amino acid sequence having at most 1, at most 2, at most 3, at most 4, or at most 5 amino acid mutations (addition, deletion, or substitution) thereto.

### Distance Factors

The present invention is based, in part, on the inventors' unexpected discovery that the peptide binding arms of the multivalent linkers of the present disclosure are capable of all binding to the same target without crosslinking to other molecules. This results in superior binding properties due to the gained avidity achieved from the apparent (i.e. synergistic) affinity of the multiple peptide binding arms onto the target. In this binding mode, it is believed that one binding arm of the bivalent peptide binds the first of multiple epitopes in the target, followed by epitope engagement by additional (e.g., second) binding arms to the same target. For this to happen, the linker segment between the binding arms needs to be sufficiently long and flexible to reach additional epitopes within the same target.

Without wishing to be bound by any one theory, it is believed that if the linker segment between the binding arms is too short, the additional binding arms cannot reach the additional epitopes on the target (e.g., epitopes within the same primary antibody). Under these circumstances, the additional binding arms may eventually engage with a different target (e.g., another primary antibody), leading to cross-linking and thus aggregation of the target. Thus, in some embodiments, the present disclosure provides a minimum distance factor between binding arms.

In some embodiments, the distance is measured from the first amino acid of each peptide binding arm that is linked to the linker segment.

In some embodiments, the distance between two peptide binding arms of the multivalent linker created by the linker segment is about 1 Å, about 2 Å, about 3 Å, about 4 Å, about 5 Å, about 6 Å, about 7 Å, about 8 Å, about 9 Å, about 10 Å, about 11 Å, about 12 Å, about 13 Å, about 14 Å, about 15 Å, about 16 Å, about 17 Å, about 18 Å, about 19 Å, about 20 Å, about 21 Å, about 22 Å, about 23 Å, about 24 Å, about 25 Å, about 26 Å, about 27 Å, about 28 Å, about 29 Å, about 30 Å, about 31 Å, about 32 Å, about 33 Å, about 34 Å, about 35 Å, about 36 Å, about 37 Å, about 38 Å, about 39 Å, about 40 Å, about 41 Å, about 42 Å, about 43 Å, about 44 Å, about 45 Å, about 46 Å, about 47 Å, about 48 Å, about 49 Å, about 50 Å, about 51 Å, about 52 Å, about 53 Å, about 54 Å, about 55 Å, about 56 Å, about 57 Å, about 58 Å, about 59 Å, about 60 Å, about 61 Å, about 62 Å, about 63 Å, about 64 Å, about 65 Å, about 66 Å, about 67 Å, about 68 Å, about 69 Å, about 70 Å, about 71 Å, about 72 Å, about 73 Å, about 74 Å, about 75 Å, about 76 Å, about 77 Å, about 78 Å, about 79 Å, about 80 Å, about 81 Å, about 82 Å, about 83 Å, about 84 Å, about 85 Å, about 86 Å, about 87 Å, about 88 Å, about 89 Å, about 90 Å, about 91 Å, about 92 Å, about 93 Å, about 94 Å, about 95 Å, about 96 Å, about 97 Å, about 98 Å, about 99 Å, about 100 Å, about 101 Å, about 102 Å, about 103 Å, about 104 Å, about 105 Å, about 106 Å, about 107 Å, about 108 Å, about 109 Å, about 110 Å, about 111 Å, about 112 Å, about 113 Å, about 114 Å, about 115 Å, about 116 Å, about 117 Å, about 118 Å, about 119 Å, about 120 Å, about 121 Å, about 122 Å, about 123 Å, about 124 Å, about 125 Å, about 126 Å, about 127 Å, about 128 Å, about 129 Å, about 130 Å, about 131 Å, about 132 Å, about 133 Å, about 134 Å, about 135 Å, about 136 Å, about 137 Å, about 138 Å, about 139 Å, about 140 Å, about 141 Å, about 142 Å, about 143 Å, about 144 Å, about 145 Å, about 146 Å, about 147 Å, about 148 Å, about 149 Å, about 150 Å, about 151 Å, about 152 Å, about 153 Å, about 154 Å, about 155 Å, about 156 Å, about 157 Å, about 158 Å, about 159 Å, about 160 Å, about 161 Å, about 162 Å, about 163 Å, about 164 Å, about 165 Å, about 166 Å, about 167 Å, about 168 Å, about 169 Å, about 170 Å, about 171 Å, about 172 Å, about 173 Å, about 174 Å, about 175 Å, about 176 Å, about 177 Å, about 178 Å, about 179 Å, about 180 Å, about 181 Å, about 182 Å, about 183 Å, about 184 Å, about 185 Å, about 186 Å, about 187 Å, about 188 Å, about 189 Å, about 190 Å, about 191 Å, about 192 Å, about 193 Å, about 194 Å, about 195 Å, about 196 Å, about 197 Å, about 198 Å, about 199 Å, about 200 Å, about 201 Å, about 202 Å, about 203 Å, about 204 Å, about 205 Å, about 206 Å, about 207 Å, about 208 Å, about 209 Å, about 210 Å, about 211 Å, about 212 Å, about 213 Å, about 214 Å, about 215 Å, about 216 Å, about 217 Å, about 218 Å, about 219 Å, about 220 Å, about 221 Å, about 222 Å, about 223 Å, about 224 Å, about 225 Å, about 226 Å, about 227 Å, about 228 Å, about 229 Å, about 230 Å, about 231 Å, about 232 Å, about 233 Å, about 234 Å, about 235 Å, about 236 Å, about 237 Å, about 238 Å, about 239 Å, about 240 Å, about 241 Å, about 242 Å, about 243 Å, about 244 Å, about 245 Å, about 246 Å, about 247 Å, about 248 Å, about 249 Å, about 250 Å, about 251 Å, about 252 Å, about 253 Å, about 254 Å, about 255 Å, about 256 Å, about 257 Å, about 258 Å, about 259 Å, about 260 Å, about 261 Å, about 262 Å, about 263 Å, about 264 Å, about 265 Å, about 266 Å, about 267 Å, about 268 Å, about 269 Å, about 270 A, about 271 Å, about 272 Å, about 273 Å, about 274 Å, about 275 Å, about 276 Å, about 277 Å, about 278 Å, about 279 Å, about 280 Å, about 281 Å, about 282 Å, about 283 Å, about 284 Å, about 285 Å, about 286 Å, about 287 Å, about 288 Å, about 289 Å, about 290 Å, about 291 Å, about 292 Å, about 293 Å, about 294 Å, about 295 Å, about 296 Å, about 297 Å, about 298 Å, about 299 Å, about 300 Å, about 301 Å, about 302 Å, about 303 Å, about 304 Å, about 305 Å, about 306 Å, about 307 Å, about 308 Å, about 309 Å, about 310 Å, about 311 Å, about 312 Å, about 313 Å, about 314 Å, about 315 Å, about 316 Å, about 317 Å, about 318 Å, about 319 Å, about 320 Å, about 321 Å, about 322 Å, about 323 Å, about 324 Å, about 325 Å, about 326 Å, about 327 Å, about 328 Å, about 329 Å, about 330 Å, about 331 Å, about 332 Å, about 333 Å, about 334 Å, about 335 Å, about 336 Å, about 337 Å, about 338 Å, about 339 Å, about 340 Å, about 341 Å, about 342 Å, about 343 Å, about 344 Å, about 345 Å, about 346 Å, about 347 Å, about 348 Å, about 349 Å, about 350 Å, about 351 Å, about 352 Å, about 353 Å, about 354 Å, about 355 Å, about 356 Å, about 357 Å, about 358 Å, about 359 Å, about 360 Å, about 361 Å, about 362 Å, about 363 Å, about 364 Å, about 365 Å, about 366 Å, about 367 Å, about 368 Å, about 369 Å, about 370 Å, about 371 Å, about 372 Å, about 373 Å, about 374 Å, about 375 Å, about 376 Å, about 377 Å, about 378 Å, about 379 Å, about 380 Å, about 381 Å, about 382 Å, about 383 Å, about 384 Å, about 385 Å, about 386 Å, about 387 Å, about 388 Å, about 389 Å, about 390 Å, about 391 Å, about 392 Å, about 393 Å, about 394 Å, about 395 Å, about 396 Å, about 397 Å, about 398 Å, about 399 Å, or about 400 Å, including any ranges and subranges there between.

In some embodiments, the distance between two peptide binding arms of the multivalent linker created by the linker segment is between 1 Å to 10 Å, between 10 Å to 20 Å, between 20 Å to 30 Å, between 30 Å to 40 Å, between 40 Å to 50 Å, between 50 Å to 60 Å, between 60 Å to 70 Å, between 70 Å to 80 Å, between 80 Å to 90 Å, between 90 Å to 100 Å, between 100 Å to 110 Å, between 110 Å to 120 Å, between 120 Å to 130 Å, between 130 Å to 140 Å, between 140 Å to 150 Å, between 150 Å to 160 Å, between 160 Å to 170 Å, between 170 Å to 180 Å, between 180 Å to 190 Å, between 190 Å to 200 Å, between 200 Å to 210 Å, between 210 Å to 220 Å, between 220 Å to 230 Å, between 230 Å to 240 Å, between 240 Å to 250 Å, between 250 Å to 260 Å, between 260 Å to 270 Å, between 270 Å to 280 Å, between 280 Å to 290 Å, between 290 Å to 300 Å, between 300 Å to 310 Å, between 310 Å to 320 Å, between 320 Å to 330 Å, between 330 Å to 340 Å, between 340 Å to 350 Å, between 350 Å to 360 Å, between 360 Å to 370 Å, between 370 Å to 380 Å, between 380 Å to 390 Å, or between 390 Å to 300 Å.

In some embodiments, the minimum distance factor between the two peptides of the multivalent linker decreases crosslinking. In some embodiments, the crosslinking is decreased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all ranges and subranges therebetween.

In some embodiments, the crosslinking is decreased by between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 25%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, or between 95% to 100%, including all ranges and subranges therebetween.

In some embodiments, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, at most 45%, or at most 50% of the multivalent linkers crosslink to different antibodies.

In some embodiments, between 0% to 1%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 25%, between 25% to 30%, between 30% to 35%, between 35% to 40%, between 40% to 45%, or between 45% to 50% of the multivalent linkers crosslink to different antibodies, including all ranges and subranges therebetween.

In some embodiments, when distance between two peptides of the multivalent linker created by the linker segment is measured in angstroms, the distance is through space as determined by crystallography (e.g., X-ray crystallography) or 3-D modeling software.

In some embodiments, the distance between two peptide binding arms of the multivalent linker created by the linker segment is at most 1 (G4X) unit, at most 2 (G4X) units, at most 3 (G4X) units, at most 4 (G4X) units, at most 5 (G4X) units, at most 6 (G4X) units, at most 7 (G4X) units, at most 8 (G4X) units, at most 9 (G4X) units, or at most 10 (G4X) units, including any ranges and subranges therebetween.

In some embodiments, the distance between two peptide binding arms of the multivalent linker created by the linker segment is at most 1 (G4A) unit, at most 2 (G4A) units, at most 3 (G4A) units, at most 4 (G4A) units, at most 5 (G4A) units, at most 6 (G4A) units, at most 7 (G4A) units, at most 8 (G4A) units, at most 9 (G4A) units, or at most 10 (G4A) units, including any ranges and subranges therebetween.

In some embodiments, the distance between two peptide binding arms of the multivalent linker created by the linker segment is at most 1 (G4S) unit, at most 2 (G4S) units, at most 3 (G4S) units, at most 4 (G4S) units, at most 5 (G4S) units, at most 6 (G4S) units, at most 7 (G4S) units, at most 8 (G4S) units, at most 9 (G4S) units, or at most 10 (G4S) units, including any ranges and subranges therebetween.

### Moiety for Conjugation to a Heterologous Molecule

In some embodiments, the multivalent linker comprises at least one moiety for conjugation to a heterologous molecule. In some embodiments, the at least one moiety is a cysteine. In some embodiments, the at least one moiety is a lysine. In some embodiments, the at least one moiety is a biotin. In some embodiments, biotinylation of the multivalent linker is achieved through chemical methods. In some embodiments, biotinylation of the multivalent linker is achieved through enzymatic methods. In some embodiments, biotinylation of the multivalent linker is achieved in vitro. In some embodiments, biotinylation of the multivalent linker is achieved in vivo.

Exemplary methods of biotinylation, as well as other bioconjugation techniques, can be found in Hermanson GT (2008) Bioconjugate techniques, 2nd Edition. San Diego (CA): Academic Press, the content of which is incorporated by reference in its entirety for all purposes.

In some embodiments, the at least one moiety is a streptavidin.

In some embodiments, the at least one moiety is a non-natural amino acid.

In some embodiments, the at least one moiety comprises a functional group for conjugation through click chemistry. In some embodiments, the functional group comprises dibenzocyclooctyne group (DBCO), azide, tetrazine and/or trans-cyclooctene (TCO). Descriptions of click chemistry can be found, for example, in Devaraj and Finn, Chem. Rev. 2021, 121, 12, 6697-6698; and Hein et al., Pharm Res. 2008 October; 25(10): 2216-2230, the content of each of which is incorporated by reference in its entirety for all purposes.

In some embodiments, the multivalent linker comprise one moiety for conjugation to a heterologous molecule.

In some embodiments, the multivalent linker comprise two or more moieties for conjugation to a heterologous molecule. In some embodiments, the multivalent linker comprise two, three, four, five, six, seven, eight, nine, ten, or more than ten moieties for conjugation to a heterologous molecule. In some embodiments, the multivalent linker comprise two moieties for conjugation to a heterologous molecule. In some embodiments, the two or more moieties are the same. In some embodiments, the two or more moieties are different.

In some embodiments, the linker segment of the multivalent linker comprises at least one moiety for conjugation to a heterologous molecule. In some embodiments, the linker segment of the multivalent linker comprises all the moieties for conjugation to a heterologous molecule.

In some embodiments, the linker segment of the multivalent linker does not comprise any moiety for conjugation to a heterologous molecule.

In some embodiments, the peptide binding arm of the multivalent linker comprises at least one moiety for conjugation to a heterologous molecule. In some embodiments, the peptide binding arm of the multivalent linker comprises all the moieties for conjugation to a heterologous molecule.

In some embodiments, the peptide binding arm of the multivalent linker does not comprise any moiety for conjugation to a heterologous molecule.

In some embodiments, the heterologous molecule is a reporter, an oligonucleotide, a moiety functionalized for click chemistry, or an effector.

### Illustrative Multivalent Linker Structures

In some embodiments, the peptides of the present disclosure are multivalent, comprising multiple peptide binding arms.

In some embodiments, the multivalent peptides comprises the formula

(SEQ ID NO. 1-418)-(Linker Segment)-(SEQ ID NO. 1-418).

In some embodiments, the multivalent linkers comprises the formula

(SEQ ID NO. 1-418)-(G4S)˜-(SEQ ID NO. 1-418).

In some embodiments, the multivalent linkers comprises the same peptide sequence. In some embodiments, the multivalent linkers comprise the formula in Table 1.

TABLE 1
Multivalent Linkers that Comprise the Same Peptide Sequence

| SEQ | | SEQ |
|---|---|---|
| ID NO | Linker Segment | ID NO |
| 1 | Peptide Linker Segment | 1 |
| | (e.g., (G4S)n) | |
| 2 | Peptide Linker Segment | 2 |
| | (e.g., (G4S)n) | |
| 3 | Peptide Linker Segment | 3 |
| | (e.g., (G4S)n) | |
| 4 | Peptide Linker Segment | 4 |
| | (e.g., (G4S)n) | |
| 5 | Peptide Linker Segment | 5 |
| | (e.g., (G4S)n) | |
| 6 | Peptide Linker Segment | 6 |
| | (e.g., (G4S)n) | |
| 7 | Peptide Linker Segment | 7 |
| | (e.g., (G4S)n) | |
| 8 | Peptide Linker Segment | 8 |
| | (e.g., (G4S)n) | |
| 9 | Peptide Linker Segment | 9 |
| | (e.g., (G4S)n) | |
| 10 | Peptide Linker Segment | 10 |
| | (e.g., (G4S)n) | |
| 11 | Peptide Linker Segment | 11 |
| | (e.g., (G4S)n) | |
| 12 | Peptide Linker Segment | 12 |
| | (e.g., (G4S)n) | |
| 13 | Peptide Linker Segment | 13 |
| | (e.g., (G4S)n) | |
| 14 | Peptide Linker Segment | 14 |
| | (e.g., (G4S)n) | |
| 15 | Peptide Linker Segment | 15 |
| | (e.g., (G4S)n) | |
| 16 | Peptide Linker Segment | 16 |
| | (e.g., (G4S)n) | |
| 17 | Peptide Linker Segment | 17 |
| | (e.g., (G4S)n) | |
| 18 | Peptide Linker Segment | 18 |
| | (e.g., (G4S)n) | |
| 19 | Peptide Linker Segment | 19 |
| | (e.g., (G4S)n) | |
| 20 | Peptide Linker Segment | 20 |
| | (e.g., (G4S)n) | |
| 21 | Peptide Linker Segment | 21 |
| | (e.g., (G4S)n) | |
| 22 | Peptide Linker Segment | 22 |
| | (e.g., (G4S)n) | |
| 23 | Peptide Linker Segment | 23 |
| | (e.g., (G4S)n) | |
| 24 | Peptide Linker Segment | 24 |
| | (e.g., (G4S)n) | |
| 25 | Peptide Linker Segment | 25 |
| | (e.g., (G4S)n) | |
| 26 | Peptide Linker Segment | 26 |
| | (e.g., (G4S)n) | |
| 27 | Peptide Linker Segment | 27 |
| | (e.g., (G4S)n) | |
| 28 | Peptide Linker Segment | 28 |
| | (e.g., (G4S)n) | |
| 29 | Peptide Linker Segment | 29 |
| | (e.g., (G4S)n) | |
| 30 | Peptide Linker Segment | 30 |
| | (e.g., (G4S)n) | |
| 31 | Peptide Linker Segment | 31 |
| | (e.g., (G4S)n) | |
| 32 | Peptide Linker Segment | 32 |
| | (e.g., (G4S)n) | |
| 33 | Peptide Linker Segment | 33 |
| | (e.g., (G4S)n) | |
| 34 | Peptide Linker Segment | 34 |
| | (e.g., (G4S)n) | |
| 35 | Peptide Linker Segment | 35 |
| | (e.g., (G4S)n) | |
| 36 | Peptide Linker Segment | 36 |
| | (e.g., (G4S)n) | |
| 37 | Peptide Linker Segment | 37 |
| | (e.g., (G4S)n) | |
| 38 | Peptide Linker Segment | 38 |
| | (e.g., (G4S)n) | |
| 39 | Peptide Linker Segment | 39 |
| | (e.g., (G4S)n) | |
| 40 | Peptide Linker Segment | 40 |
| | (e.g., (G4S)n) | |
| 41 | Peptide Linker Segment | 41 |
| | (e.g., (G4S)n) | |
| 42 | Peptide Linker Segment | 42 |
| | (e.g., (G4S)n) | |
| 43 | Peptide Linker Segment | 43 |
| | (e.g., (G4S)n) | |
| 44 | Peptide Linker Segment | 44 |
| | (e.g., (G4S)n) | |
| 45 | Peptide Linker Segment | 45 |
| | (e.g., (G4S)n) | |
| 46 | Peptide Linker Segment | 46 |
| | (e.g., (G4S)n) | |
| 47 | Peptide Linker Segment | 47 |
| | (e.g., (G4S)n) | |
| 48 | Peptide Linker Segment | 48 |
| | (e.g., (G4S)n) | |
| 49 | Peptide Linker Segment | 49 |
| | (e.g., (G4S)n) | |
| 50 | Peptide Linker Segment | 50 |
| | (e.g., (G4S)n) | |
| 51 | Peptide Linker Segment | 51 |
| | (e.g., (G4S)n) | |
| 52 | Peptide Linker Segment | 52 |
| | (e.g., (G4S)n) | |
| 53 | Peptide Linker Segment | 53 |
| | (e.g., (G4S)n) | |
| 54 | Peptide Linker Segment | 54 |
| | (e.g., (G4S)n) | |
| 55 | Peptide Linker Segment | 55 |
| | (e.g., (G4S)n) | |
| 56 | Peptide Linker Segment | 56 |
| | (e.g., (G4S)n) | |
| 57 | Peptide Linker Segment | 57 |
| | (e.g., (G4S)n) | |
| 58 | Peptide Linker Segment | 58 |
| | (e.g., (G4S)n) | |
| 59 | Peptide Linker Segment | 59 |
| | (e.g., (G4S)n) | |
| 60 | Peptide Linker Segment | 60 |
| | (e.g., (G4S)n) | |
| 61 | Peptide Linker Segment | 61 |
| | (e.g., (G4S)n) | |
| 62 | Peptide Linker Segment | 62 |
| | (e.g., (G4S)n) | |
| 63 | Peptide Linker Segment | 63 |
| | (e.g., (G4S)n) | |
| 64 | Peptide Linker Segment | 64 |
| | (e.g., (G4S)n) | |
| 65 | Peptide Linker Segment | 65 |
| | (e.g., (G4S)n) | |
| 66 | Peptide Linker Segment | 66 |
| | (e.g., (G4S)n) | |
| 67 | Peptide Linker Segment | 67 |
| | (e.g., (G4S)n) | |
| 68 | Peptide Linker Segment | 68 |
| | (e.g., (G4S)n) | |
| 69 | Peptide Linker Segment | 69 |
| | (e.g., (G4S)n) | |
| 70 | Peptide Linker Segment | 70 |
| | (e.g., (G4S)n) | |
| 71 | Peptide Linker Segment | 71 |
| | (e.g., (G4S)n) | |
| 72 | Peptide Linker Segment | 72 |
| | (e.g., (G4S)n) | |
| 73 | Peptide Linker Segment | 73 |
| | (e.g., (G4S)n) | |
| 74 | Peptide Linker Segment | 74 |
| | (e.g., (G4S)n) | |
| 75 | Peptide Linker Segment | 75 |
| | (e.g., (G4S)n) | |
| 76 | Peptide Linker Segment | 76 |
| | (e.g., (G4S)n) | |
| 77 | Peptide Linker Segment | 77 |
| | (e.g., (G4S)n) | |
| 78 | Peptide Linker Segment | 78 |
| | (e.g., (G4S)n) | |
| 79 | Peptide Linker Segment | 79 |
| | (e.g., (G4S)n) | |
| 80 | Peptide Linker Segment | 80 |
| | (e.g., (G4S)n) | |
| 81 | Peptide Linker Segment | 81 |
| | (e.g., (G4S)n) | |
| 82 | Peptide Linker Segment | 82 |
| | (e.g., (G4S)n) | |
| 83 | Peptide Linker Segment | 83 |
| | (e.g., (G4S)n) | |
| 84 | Peptide Linker Segment | 84 |
| | (e.g., (G4S)n) | |
| 85 | Peptide Linker Segment | 85 |
| | (e.g., (G4S)n) | |
| 86 | Peptide Linker Segment | 86 |
| | (e.g., (G4S)n) | |
| 87 | Peptide Linker Segment | 87 |
| | (e.g., (G4S)n) | |
| 88 | Peptide Linker Segment | 88 |
| | (e.g., (G4S)n) | |
| 89 | Peptide Linker Segment | 89 |
| | (e.g., (G4S)n) | |
| 90 | Peptide Linker Segment | 90 |
| | (e.g., (G4S)n) | |
| 91 | Peptide Linker Segment | 91 |
| | (e.g., (G4S)n) | |
| 92 | Peptide Linker Segment | 92 |
| | (e.g., (G4S)n) | |
| 93 | Peptide Linker Segment | 93 |
| | (e.g., (G4S)n) | |
| 94 | Peptide Linker Segment | 94 |
| | (e.g., (G4S)n) | |
| 95 | Peptide Linker Segment | 95 |
| | (e.g., (G4S)n) | |
| 96 | Peptide Linker Segment | 96 |
| | (e.g., (G4S)n) | |
| 97 | Peptide Linker Segment | 97 |
| | (e.g., (G4S)n) | |
| 98 | Peptide Linker Segment | 98 |
| | (e.g., (G4S)n) | |
| 99 | Peptide Linker Segment | 99 |
| | (e.g., (G4S)n) | |
| 100 | Peptide Linker Segment | 100 |
| | (e.g., (G4S)n) | |
| 101 | Peptide Linker Segment | 101 |
| | (e.g., (G4S)n) | |
| 102 | Peptide Linker Segment | 102 |
| | (e.g., (G4S)n) | |
| 103 | Peptide Linker Segment | 103 |
| | (e.g., (G4S)n) | |
| 104 | Peptide Linker Segment | 104 |
| | (e.g., (G4S)n) | |
| 105 | Peptide Linker Segment | 105 |
| | (e.g., (G4S)n) | |
| 106 | Peptide Linker Segment | 106 |
| | (e.g., (G4S)n) | |
| 107 | Peptide Linker Segment | 107 |
| | (e.g., (G4S)n) | |
| 108 | Peptide Linker Segment | 108 |
| | (e.g., (G4S)n) | |
| 109 | Peptide Linker Segment | 109 |
| | (e.g., (G4S)n) | |
| 110 | Peptide Linker Segment | 110 |
| | (e.g., (G4S)n) | |
| 111 | Peptide Linker Segment | 111 |
| | (e.g., (G4S)n) | |
| 112 | Peptide Linker Segment | 112 |
| | (e.g., (G4S)n) | |
| 113 | Peptide Linker Segment | 113 |
| | (e.g., (G4S)n) | |
| 114 | Peptide Linker Segment | 114 |
| | (e.g., (G4S)n) | |
| 115 | Peptide Linker Segment | 115 |
| | (e.g., (G4S)n) | |
| 116 | Peptide Linker Segment | 116 |
| | (e.g., (G4S)n) | |
| 117 | Peptide Linker Segment | 117 |
| | (e.g., (G4S)n) | |
| 118 | Peptide Linker Segment | 118 |
| | (e.g., (G4S)n) | |
| 119 | Peptide Linker Segment | 119 |
| | (e.g., (G4S)n) | |
| 120 | Peptide Linker Segment | 120 |
| | (e.g., (G4S)n) | |
| 121 | Peptide Linker Segment | 121 |
| | (e.g., (G4S)n) | |
| 122 | Peptide Linker Segment | 122 |
| | (e.g., (G4S)n) | |
| 123 | Peptide Linker Segment | 123 |
| | (e.g., (G4S)n) | |
| 124 | Peptide Linker Segment | 124 |
| | (e.g., (G4S)n) | |
| 125 | Peptide Linker Segment | 125 |
| | (e.g., (G4S)n) | |
| 126 | Peptide Linker Segment | 126 |
| | (e.g., (G4S)n) | |
| 127 | Peptide Linker Segment | 127 |
| | (e.g., (G4S)n) | |
| 128 | Peptide Linker Segment | 128 |
| | (e.g., (G4S)n) | |
| 129 | Peptide Linker Segment | 129 |
| | (e.g., (G4S)n) | |
| 130 | Peptide Linker Segment | 130 |
| | (e.g., (G4S)n) | |
| 131 | Peptide Linker Segment | 131 |
| | (e.g., (G4S)n) | |
| 132 | Peptide Linker Segment | 132 |
| | (e.g., (G4S)n) | |
| 133 | Peptide Linker Segment | 133 |
| | (e.g., (G4S)n) | |
| 134 | Peptide Linker Segment | 134 |
| | (e.g., (G4S)n) | |
| 135 | Peptide Linker Segment | 135 |
| | (e.g., (G4S)n) | |
| 136 | Peptide Linker Segment | 136 |
| | (e.g., (G4S)n) | |
| 137 | Peptide Linker Segment | 137 |
| | (e.g., (G4S)n) | |
| 138 | Peptide Linker Segment | 138 |
| | (e.g., (G4S)n) | |
| 139 | Peptide Linker Segment | 139 |
| | (e.g., (G4S)n) | |
| 140 | Peptide Linker Segment | 140 |
| | (e.g., (G4S)n) | |
| 141 | Peptide Linker Segment | 141 |
| | (e.g., (G4S)n) | |
| 142 | Peptide Linker Segment | 142 |
| | (e.g., (G4S)n) | |
| 143 | Peptide Linker Segment | 143 |
| | (e.g., (G4S)n) | |
| 144 | Peptide Linker Segment | 144 |
| | (e.g., (G4S)n) | |
| 145 | Peptide Linker Segment | 145 |
| | (e.g., (G4S)n) | |
| 146 | Peptide Linker Segment | 146 |
| | (e.g., (G4S)n) | |
| 147 | Peptide Linker Segment | 147 |
| | (e.g., (G4S)n) | |
| 148 | Peptide Linker Segment | 148 |
| | (e.g., (G4S)n) | |
| 149 | Peptide Linker Segment | 149 |
| | (e.g., (G4S)n) | |
| 150 | Peptide Linker Segment | 150 |
| | (e.g., (G4S)n) | |
| 151 | Peptide Linker Segment | 151 |
| | (e.g., (G4S)n) | |
| 152 | Peptide Linker Segment | 152 |
| | (e.g., (G4S)n) | |
| 153 | Peptide Linker Segment | 153 |
| | (e.g., (G4S)n) | |
| 154 | Peptide Linker Segment | 154 |
| | (e.g., (G4S)n) | |
| 155 | Peptide Linker Segment | 155 |
| | (e.g., (G4S)n) | |
| 156 | Peptide Linker Segment | 156 |
| | (e.g., (G4S)n) | |
| 157 | Peptide Linker Segment | 157 |
| | (e.g., (G4S)n) | |
| 158 | Peptide Linker Segment | 158 |
| | (e.g., (G4S)n) | |
| 159 | Peptide Linker Segment | 159 |
| | (e.g., (G4S)n) | |
| 160 | Peptide Linker Segment | 160 |
| | (e.g., (G4S)n) | |
| 161 | Peptide Linker Segment | 161 |
| | (e.g., (G4S)n) | |
| 162 | Peptide Linker Segment | 162 |
| | (e.g., (G4S)n) | |
| 163 | Peptide Linker Segment | 163 |
| | (e.g., (G4S)n) | |
| 164 | Peptide Linker Segment | 164 |
| | (e.g., (G4S)n) | |
| 165 | Peptide Linker Segment | 165 |
| | (e.g., (G4S)n) | |
| 166 | Peptide Linker Segment | 166 |
| | (e.g., (G4S)n) | |
| 167 | Peptide Linker Segment | 167 |
| | (e.g., (G4S)n) | |
| 168 | Peptide Linker Segment | 168 |
| | (e.g., (G4S)n) | |
| 169 | Peptide Linker Segment | 169 |
| | (e.g., (G4S)n) | |
| 170 | Peptide Linker Segment | 170 |
| | (e.g., (G4S)n) | |
| 171 | Peptide Linker Segment | 171 |
| | (e.g., (G4S)n) | |
| 172 | Peptide Linker Segment | 172 |
| | (e.g., (G4S)n) | |
| 173 | Peptide Linker Segment | 173 |
| | (e.g., (G4S)n) | |
| 174 | Peptide Linker Segment | 174 |
| | (e.g., (G4S)n) | |
| 175 | Peptide Linker Segment | 175 |
| | (e.g., (G4S)n) | |
| 176 | Peptide Linker Segment | 176 |
| | (e.g., (G4S)n) | |
| 177 | Peptide Linker Segment | 177 |
| | (e.g., (G4S)n) | |
| 178 | Peptide Linker Segment | 178 |
| | (e.g., (G4S)n) | |
| 179 | Peptide Linker Segment | 179 |
| | (e.g., (G4S)n) | |
| 180 | Peptide Linker Segment | 180 |
| | (e.g., (G4S)n) | |
| 181 | Peptide Linker Segment | 181 |
| | (e.g., (G4S)n) | |
| 182 | Peptide Linker Segment | 182 |
| | (e.g., (G4S)n) | |
| 183 | Peptide Linker Segment | 183 |
| | (e.g., (G4S)n) | |
| 184 | Peptide Linker Segment | 184 |
| | (e.g., (G4S)n) | |
| 185 | Peptide Linker Segment | 185 |
| | (e.g., (G4S)n) | |
| 186 | Peptide Linker Segment | 186 |
| | (e.g., (G4S)n) | |
| 187 | Peptide Linker Segment | 187 |
| | (e.g., (G4S)n) | |
| 188 | Peptide Linker Segment | 188 |
| | (e.g., (G4S)n) | |
| 189 | Peptide Linker Segment | 189 |
| | (e.g., (G4S)n) | |
| 190 | Peptide Linker Segment | 190 |
| | (e.g., (G4S)n) | |
| 191 | Peptide Linker Segment | 191 |
| | (e.g., (G4S)n) | |
| 192 | Peptide Linker Segment | 192 |
| | (e.g., (G4S)n) | |
| 193 | Peptide Linker Segment | 193 |
| | (e.g., (G4S)n) | |
| 194 | Peptide Linker Segment | 194 |
| | (e.g., (G4S)n) | |
| 195 | Peptide Linker Segment | 195 |
| | (e.g., (G4S)n) | |
| 196 | Peptide Linker Segment | 196 |
| | (e.g., (G4S)n) | |
| 197 | Peptide Linker Segment | 197 |
| | (e.g., (G4S)n) | |
| 198 | Peptide Linker Segment | 198 |
| | (e.g., (G4S)n) | |
| 199 | Peptide Linker Segment | 199 |
| | (e.g., (G4S)n) | |
| 200 | Peptide Linker Segment | 200 |
| | (e.g., (G4S)n) | |
| 201 | Peptide Linker Segment | 201 |
| | (e.g., (G4S)n) | |
| 202 | Peptide Linker Segment | 202 |
| | (e.g., (G4S)n) | |
| 203 | Peptide Linker Segment | 203 |
| | (e.g., (G4S)n) | |
| 204 | Peptide Linker Segment | 204 |
| | (e.g., (G4S)n) | |
| 205 | Peptide Linker Segment | 205 |
| | (e.g., (G4S)n) | |
| 206 | Peptide Linker Segment | 206 |
| | (e.g., (G4S)n) | |
| 207 | Peptide Linker Segment | 207 |
| | (e.g., (G4S)n) | |
| 208 | Peptide Linker Segment | 208 |
| | (e.g., (G4S)n) | |
| 209 | Peptide Linker Segment | 209 |
| | (e.g., (G4S)n) | |
| 210 | Peptide Linker Segment | 210 |
| | (e.g., (G4S)n) | |
| 211 | Peptide Linker Segment | 211 |
| | (e.g., (G4S)n) | |
| 212 | Peptide Linker Segment | 212 |
| | (e.g., (G4S)n) | |
| 213 | Peptide Linker Segment | 213 |
| | (e.g., (G4S)n) | |
| 214 | Peptide Linker Segment | 214 |
| | (e.g., (G4S)n) | |
| 215 | Peptide Linker Segment | 215 |
| | (e.g., (G4S)n) | |
| 216 | Peptide Linker Segment | 216 |
| | (e.g., (G4S)n) | |
| 217 | Peptide Linker Segment | 217 |
| | (e.g., (G4S)n) | |
| 218 | Peptide Linker Segment | 218 |
| | (e.g., (G4S)n) | |
| 219 | Peptide Linker Segment | 219 |
| | (e.g., (G4S)n) | |
| 220 | Peptide Linker Segment | 220 |
| | (e.g., (G4S)n) | |
| 221 | Peptide Linker Segment | 221 |
| | (e.g., (G4S)n) | |
| 222 | Peptide Linker Segment | 222 |
| | (e.g., (G4S)n) | |
| 223 | Peptide Linker Segment | 223 |
| | (e.g., (G4S)n) | |
| 224 | Peptide Linker Segment | 224 |
| | (e.g., (G4S)n) | |
| 225 | Peptide Linker Segment | 225 |
| | (e.g., (G4S)n) | |
| 226 | Peptide Linker Segment | 226 |
| | (e.g., (G4S)n) | |
| 227 | Peptide Linker Segment | 227 |
| | (e.g., (G4S)n) | |
| 228 | Peptide Linker Segment | 228 |
| | (e.g., (G4S)n) | |
| 229 | Peptide Linker Segment | 229 |
| | (e.g., (G4S)n) | |
| 230 | Peptide Linker Segment | 230 |
| | (e.g., (G4S)n) | |
| 231 | Peptide Linker Segment | 231 |
| | (e.g., (G4S)n) | |
| 232 | Peptide Linker Segment | 232 |
| | (e.g., (G4S)n) | |
| 233 | Peptide Linker Segment | 233 |
| | (e.g., (G4S)n) | |
| 234 | Peptide Linker Segment | 234 |
| | (e.g., (G4S)n) | |
| 235 | Peptide Linker Segment | 235 |
| | (e.g., (G4S)n) | |
| 236 | Peptide Linker Segment | 236 |
| | (e.g., (G4S)n) | |
| 237 | Peptide Linker Segment | 237 |
| | (e.g., (G4S)n) | |
| 238 | Peptide Linker Segment | 238 |
| | (e.g., (G4S)n) | |
| 239 | Peptide Linker Segment | 239 |
| | (e.g., (G4S)n) | |
| 240 | Peptide Linker Segment | 240 |
| | (e.g., (G4S)n) | |
| 241 | Peptide Linker Segment | 241 |
| | (e.g., (G4S)n) | |
| 242 | Peptide Linker Segment | 242 |
| | (e.g., (G4S)n) | |
| 243 | Peptide Linker Segment | 243 |
| | (e.g., (G4S)n) | |
| 244 | Peptide Linker Segment | 244 |
| | (e.g., (G4S)n) | |
| 245 | Peptide Linker Segment | 245 |
| | (e.g., (G4S)n) | |
| 246 | Peptide Linker Segment | 246 |
| | (e.g., (G4S)n) | |
| 247 | Peptide Linker Segment | 247 |
| | (e.g., (G4S)n) | |
| 248 | Peptide Linker Segment | 248 |
| | (e.g., (G4S)n) | |
| 249 | Peptide Linker Segment | 249 |
| | (e.g., (G4S)n) | |
| 250 | Peptide Linker Segment | 250 |
| | (e.g., (G4S)n) | |
| 251 | Peptide Linker Segment | 251 |
| | (e.g., (G4S)n) | |
| 252 | Peptide Linker Segment | 252 |
| | (e.g., (G4S)n) | |
| 253 | Peptide Linker Segment | 253 |
| | (e.g., (G4S)n) | |
| 254 | Peptide Linker Segment | 254 |
| | (e.g., (G4S)n) | |
| 255 | Peptide Linker Segment | 255 |
| | (e.g., (G4S)n) | |
| 256 | Peptide Linker Segment | 256 |
| | (e.g., (G4S)n) | |
| 257 | Peptide Linker Segment | 257 |
| | (e.g., (G4S)n) | |
| 258 | Peptide Linker Segment | 258 |
| | (e.g., (G4S)n) | |
| 259 | Peptide Linker Segment | 259 |
| | (e.g., (G4S)n) | |
| 260 | Peptide Linker Segment | 260 |
| | (e.g., (G4S)n) | |
| 261 | Peptide Linker Segment | 261 |
| | (e.g., (G4S)n) | |
| 262 | Peptide Linker Segment | 262 |
| | (e.g., (G4S)n) | |
| 263 | Peptide Linker Segment | 263 |
| | (e.g., (G4S)n) | |
| 264 | Peptide Linker Segment | 264 |
| | (e.g., (G4S)n) | |
| 265 | Peptide Linker Segment | 265 |
| | (e.g., (G4S)n) | |
| 266 | Peptide Linker Segment | 266 |
| | (e.g., (G4S)n) | |
| 267 | Peptide Linker Segment | 267 |
| | (e.g., (G4S)n) | |
| 268 | Peptide Linker Segment | 268 |
| | (e.g., (G4S)n) | |
| 269 | Peptide Linker Segment | 269 |
| | (e.g., (G4S)n) | |
| 270 | Peptide Linker Segment | 270 |
| | (e.g., (G4S)n) | |
| 271 | Peptide Linker Segment | 271 |
| | (e.g., (G4S)n) | |
| 272 | Peptide Linker Segment | 272 |
| | (e.g., (G4S)n) | |
| 273 | Peptide Linker Segment | 273 |
| | (e.g., (G4S)n) | |
| 274 | Peptide Linker Segment | 274 |
| | (e.g., (G4S)n) | |
| 275 | Peptide Linker Segment | 275 |
| | (e.g., (G4S)n) | |
| 276 | Peptide Linker Segment | 276 |
| | (e.g., (G4S)n) | |
| 277 | Peptide Linker Segment | 277 |
| | (e.g., (G4S)n) | |
| 278 | Peptide Linker Segment | 278 |
| | (e.g., (G4S)n) | |
| 279 | Peptide Linker Segment | 279 |
| | (e.g., (G4S)n) | |
| 280 | Peptide Linker Segment | 280 |
| | (e.g., (G4S)n) | |
| 281 | Peptide Linker Segment | 281 |
| | (e.g., (G4S)n) | |
| 282 | Peptide Linker Segment | 282 |
| | (e.g., (G4S)n) | |
| 283 | Peptide Linker Segment | 283 |
| | (e.g., (G4S)n) | |
| 284 | Peptide Linker Segment | 284 |
| | (e.g., (G4S)n) | |
| 285 | Peptide Linker Segment | 28.5 |
| | (e.g., (G4S)n) | |
| 286 | Peptide Linker Segment | 286 |
| | (e.g., (G4S)n) | |
| 287 | Peptide Linker Segment | 287 |
| | (e.g., (G4S)n) | |
| 288 | Peptide Linker Segment | 288 |
| | (e.g., (G4S)n) | |
| 289 | Peptide Linker Segment | 289 |
| | (e.g., (G4S)n) | |
| 290 | Peptide Linker Segment | 290 |
| | (e.g., (G4S)n) | |
| 291 | Peptide Linker Segment | 291 |
| | (e.g., (G4S)n) | |
| 292 | Peptide Linker Segment | 292 |
| | (e.g., (G4S)n) | |
| 293 | Peptide Linker Segment | 293 |
| | (e.g., (G4S)n) | |
| 294 | Peptide Linker Segment | 294 |
| | (e.g., (G4S)n) | |
| 295 | Peptide Linker Segment | 295 |
| | (e.g., (G4S)n) | |
| 296 | Peptide Linker Segment | 296 |
| | (e.g., (G4S)n) | |
| 297 | Peptide Linker Segment | 297 |
| | (e.g., (G4S)n) | |
| 298 | Peptide Linker Segment | 298 |
| | (e.g., (G4S)n) | |
| 299 | Peptide Linker Segment | 299 |
| | (e.g., (G4S)n) | |
| 300 | Peptide Linker Segment | 300 |
| | (e.g., (G4S)n) | |
| 301 | Peptide Linker Segment | 301 |
| | (e.g., (G4S)n) | |
| 302 | Peptide Linker Segment | 302 |
| | (e.g., (G4S)n) | |
| 303 | Peptide Linker Segment | 303 |
| | (e.g., (G4S)n) | |
| 304 | Peptide Linker Segment | 304 |
| | (e.g., (G4S)n) | |
| 305 | Peptide Linker Segment | 305 |
| | (e.g., (G4S)n) | |
| 306 | Peptide Linker Segment | 306 |
| | (e.g., (G4S)n) | |
| 307 | Peptide Linker Segment | 307 |
| | (e.g., (G4S)n) | |
| 308 | Peptide Linker Segment | 308 |
| | (e.g., (G4S)n) | |
| 309 | Peptide Linker Segment | 309 |
| | (e.g., (G4S)n) | |
| 310 | Peptide Linker Segment | 310 |
| | (e.g., (G4S)n) | |
| 311 | Peptide Linker Segment | 311 |
| | (e.g., (G4S)n) | |
| 312 | Peptide Linker Segment | 312 |
| | (e.g., (G4S)n) | |
| 313 | Peptide Linker Segment | 313 |
| | (e.g., (G4S)n) | |
| 314 | Peptide Linker Segment | 314 |
| | (e.g., (G4S)n) | |
| 315 | Peptide Linker Segment | 315 |
| | (e.g., (G4S)n) | |
| 316 | Peptide Linker Segment | 316 |
| | (e.g., (G4S)n) | |
| 317 | Peptide Linker Segment | 317 |
| | (e.g., (G4S)n) | |
| 318 | Peptide Linker Segment | 318 |
| | (e.g., (G4S)n) | |
| 319 | Peptide Linker Segment | 319 |
| | (e.g., (G4S)n) | |
| 320 | Peptide Linker Segment | 320 |
| | (e.g., (G4S)n) | |
| 321 | Peptide Linker Segment | 321 |
| | (e.g., (G4S)n) | |
| 322 | Peptide Linker Segment | 322 |
| | (e.g., (G4S)n) | |
| 323 | Peptide Linker Segment | 323 |
| | (e.g., (G4S)n) | |
| 324 | Peptide Linker Segment | 324 |
| | (e.g., (G4S)n) | |
| 325 | Peptide Linker Segment | 325 |
| | (e.g., (G4S)n) | |
| 326 | Peptide Linker Segment | 326 |
| | (e.g., (G4S)n) | |
| 327 | Peptide Linker Segment | 327 |
| | (e.g., (G4S)n) | |
| 328 | Peptide Linker Segment | 328 |
| | (e.g., (G4S)n) | |
| 329 | Peptide Linker Segment | 329 |
| | (e.g., (G4S)n) | |
| 330 | Peptide Linker Segment | 330 |
| | (e.g., (G4S)n) | |
| 331 | Peptide Linker Segment | 331 |
| | (e.g., (G4S)n) | |
| 332 | Peptide Linker Segment | 332 |
| | (e.g., (G4S)n) | |
| 333 | Peptide Linker Segment | 333 |
| | (e.g., (G4S)n) | |
| 334 | Peptide Linker Segment | 334 |
| | (e.g., (G4S)n) | |
| 335 | Peptide Linker Segment | 335 |
| | (e.g., (G4S)n) | |
| 336 | Peptide Linker Segment | 336 |
| | (e.g., (G4S)n) | |
| 337 | Peptide Linker Segment | 337 |
| | (e.g., (G4S)n) | |
| 338 | Peptide Linker Segment | 338 |
| | (e.g., (G4S)n) | |
| 339 | Peptide Linker Segment | 339 |
| | (e.g., (G4S)n) | |
| 340 | Peptide Linker Segment | 340 |
| | (e.g., (G4S)n) | |
| 341 | Peptide Linker Segment | 341 |
| | (e.g., (G4S)n) | |
| 342 | Peptide Linker Segment | 342 |
| | (e.g., (G4S)n) | |
| 343 | Peptide Linker Segment | 343 |
| | (e.g., (G4S)n) | |
| 344 | Peptide Linker Segment | 344 |
| | (e.g., (G4S)n) | |
| 345 | Peptide Linker Segment | 345 |
| | (e.g., (G4S)n) | |
| 346 | Peptide Linker Segment | 346 |
| | (e.g., (G4S)n) | |
| 347 | Peptide Linker Segment | 347 |
| | (e.g., (G4S)n) | |
| 348 | Peptide Linker Segment | 348 |
| | (e.g., (G4S)n) | |
| 349 | Peptide Linker Segment | 349 |
| | (e.g., (G4S)n) | |
| 350 | Peptide Linker Segment | 350 |
| | (e.g., (G4S)n) | |
| 351 | Peptide Linker Segment | 351 |
| | (e.g., (G4S)n) | |
| 352 | Peptide Linker Segment | 352 |
| | (e.g., (G4S)n) | |
| 353 | Peptide Linker Segment | 353 |
| | (e.g., (G4S)n) | |
| 354 | Peptide Linker Segment | 354 |
| | (e.g., (G4S)n) | |
| 355 | Peptide Linker Segment | 355 |
| | (e.g., (G4S)n) | |
| 356 | Peptide Linker Segment | 356 |
| | (e.g., (G4S)n) | |
| 357 | Peptide Linker Segment | 357 |
| | (e.g., (G4S)n) | |
| 358 | Peptide Linker Segment | 358 |
| | (e.g., (G4S)n) | |
| 359 | Peptide Linker Segment | 359 |
| | (e.g., (G4S)n) | |
| 360 | Peptide Linker Segment | 360 |
| | (e.g., (G4S)n) | |
| 361 | Peptide Linker Segment | 361 |
| | (e.g., (G4S)n) | |
| 362 | Peptide Linker Segment | 362 |
| | (e.g., (G4S)n) | |
| 363 | Peptide Linker Segment | 363 |
| | (e.g., (G4S)n) | |
| 364 | Peptide Linker Segment | 364 |
| | (e.g., (G4S)n) | |
| 365 | Peptide Linker Segment | 365 |
| | (e.g., (G4S)n) | |
| 366 | Peptide Linker Segment | 366 |
| | (e.g., (G4S)n) | |
| 367 | Peptide Linker Segment | 367 |
| | (e.g., (G4S)n) | |
| 368 | Peptide Linker Segment | 368 |
| | (e.g., (G4S)n) | |
| 369 | Peptide Linker Segment | 369 |
| | (e.g., (G4S)n) | |
| 370 | Peptide Linker Segment | 370 |
| | (e.g., (G4S)n) | |
| 371 | Peptide Linker Segment | 371 |
| | (e.g., (G4S)n) | |
| 372 | Peptide Linker Segment | 372 |
| | (e.g., (G4S)n) | |
| 373 | Peptide Linker Segment | 373 |
| | (e.g., (G4S)n) | |
| 374 | Peptide Linker Segment | 374 |
| | (e.g., (G4S)n) | |
| 375 | Peptide Linker Segment | 375 |
| | (e.g., (G4S)n) | |
| 376 | Peptide Linker Segment | 376 |
| | (e.g., (G4S)n) | |
| 377 | Peptide Linker Segment | 377 |
| | (e.g., (G4S)n) | |
| 378 | Peptide Linker Segment | 378 |
| | (e.g., (G4S)n) | |
| 379 | Peptide Linker Segment | 379 |
| | (e.g., (G4S)n) | |
| 380 | Peptide Linker Segment | 380 |
| | (e.g., (G4S)n) | |
| 381 | Peptide Linker Segment | 381 |
| | (e.g., (G4S)n) | |
| 382 | Peptide Linker Segment | 382 |
| | (e.g., (G4S)n) | |
| 383 | Peptide Linker Segment | 383 |
| | (e.g., (G4S)n) | |
| 384 | Peptide Linker Segment | 384 |
| | (e.g., (G4S)n) | |
| 385 | Peptide Linker Segment | 385 |
| | (e.g., (G4S)n) | |
| 386 | Peptide Linker Segment | 386 |
| | (e.g., (G4S)n) | |
| 387 | Peptide Linker Segment | 387 |
| | (e.g., (G4S)n) | |
| 388 | Peptide Linker Segment | 388 |
| | (e.g., (G4S)n) | |
| 389 | Peptide Linker Segment | 389 |
| | (e.g., (G4S)n) | |
| 390 | Peptide Linker Segment | 390 |
| | (e.g., (G4S)n) | |
| 391 | Peptide Linker Segment | 391 |
| | (e.g., (G4S)n) | |
| 392 | Peptide Linker Segment | 392 |
| | (e.g., (G4S)n) | |
| 393 | Peptide Linker Segment | 393 |
| | (e.g., (G4S)n) | |
| 394 | Peptide Linker Segment | 394 |
| | (e.g., (G4S)n) | |
| 395 | Peptide Linker Segment | 395 |
| | (e.g., (G4S)n) | |
| 396 | Peptide Linker Segment | 396 |
| | (e.g., (G4S)n) | |
| 397 | Peptide Linker Segment | 397 |
| | (e.g., (G4S)n) | |
| 398 | Peptide Linker Segment | 398 |
| | (e.g., (G4S)n) | |
| 399 | Peptide Linker Segment | 399 |
| | (e.g., (G4S)n) | |
| 400 | Peptide Linker Segment | 400 |
| | (e.g., (G4S)n) | |
| 401 | Peptide Linker Segment | 401 |
| | (e.g., (G4S)n) | |
| 402 | Peptide Linker Segment | 402 |
| | (e.g., (G4S)n) | |
| 403 | Peptide Linker Segment | 403 |
| | (e.g., (G4S)n) | |
| 404 | Peptide Linker Segment | 404 |
| | (e.g., (G4S)n) | |
| 405 | Peptide Linker Segment | 405 |
| | (e.g., (G4S)n) | |
| 406 | Peptide Linker Segment | 406 |
| | (e.g., (G4S)n) | |
| 407 | Peptide Linker Segment | 407 |
| | (e.g., (G4S)n) | |
| 408 | Peptide Linker Segment | 408 |
| | (e.g., (G4S)n) | |
| 409 | Peptide Linker Segment | 409 |
| | (e.g., (G4S)n) | |
| 410 | Peptide Linker Segment | 410 |
| | (e.g., (G4S)n) | |
| 411 | Peptide Linker Segment | 411 |
| | (e.g., (G4S)n) | |
| 412 | Peptide Linker Segment | 412 |
| | (e.g., (G4S)n) | |
| 413 | Peptide Linker Segment | 413 |
| | (e.g., (G4S)n) | |
| 414 | Peptide Linker Segment | 414 |
| | (e.g., (G4S)n) | |
| 415 | Peptide Linker Segment | 415 |
| | (e.g., (G4S)n) | |
| 416 | Peptide Linker Segment | 416 |
| | (e.g., (G4S)n) | |
| 417 | Peptide Linker Segment | 417 |
| | (e.g., (G4S)n) | |
| 418 | Peptide Linker Segment | 418 |
| | (e.g., (G4S)n) | |

In some embodiments, the multivalent linkers comprises different peptide sequences. In some embodiments, the multivalent linkers comprise a formula in Table 2.

TABLE 2
Multivalent Linkers that Comprise Different Peptide Sequences.

| SEQ | | Any one of |
|---|---|---|
| ID NO | Linker Segment | SEQ ID NOS |
| 1 | Peptide Linker Segment | 2-418 |
| | (e.g., (G4S)n) | |
| 2 | Peptide Linker Segment | 1, 3-418 |
| | (e.g., (G4S)n) | |
| 3 | Peptide Linker Segment | 1-2, 4-418 |
| | (e.g., (G4S)n) | |
| 4 | Peptide Linker Segment | 1-3, 5-418 |
| | (e.g., (G4S)n) | |
| 5 | Peptide Linker Segment | 1-4, 6-418 |
| | (e.g., (G4S)n) | |
| 6 | Peptide Linker Segment | 1-5, 7-418 |
| | (e.g., (G4S)n) | |
| 7 | Peptide Linker Segment | 1-6, 8-418 |
| | (e.g., (G4S)n) | |
| 8 | Peptide Linker Segment | 1-7, 9-418 |
| | (e.g., (G4S)n) | |
| 9 | Peptide Linker Segment | 1-8, 10-418 |
| | (e.g., (G4S)n) | |
| 10 | Peptide Linker Segment | 1-9, 11-418 |
| | (e.g., (G4S)n) | |
| 11 | Peptide Linker Segment | 1-10, 12-418 |
| | (e.g., (G4S)n) | |
| 12 | Peptide Linker Segment | 1-11, 13-418 |
| | (e.g., (G4S)n) | |
| 13 | Peptide Linker Segment | 1-12, 14-418 |
| | (e.g., (G4S)n) | |
| 14 | Peptide Linker Segment | 1-13, 15-418 |
| | (e.g., (G4S)n) | |
| 15 | Peptide Linker Segment | 1-14, 16-418 |
| | (e.g., (G4S)n) | |
| 16 | Peptide Linker Segment | 1-15, 17-418 |
| | (e.g., (G4S)n) | |
| 17 | Peptide Linker Segment | 1-16, 18-418 |
| | (e.g., (G4S)n) | |
| 18 | Peptide Linker Segment | 1-17, 19-418 |
| | (e.g., (G4S)n) | |
| 19 | Peptide Linker Segment | 1-18, 20-418 |
| | (e.g., (G4S)n) | |
| 20 | Peptide Linker Segment | 1-19, 21-418 |
| | (e.g., (G4S)n) | |
| 21 | Peptide Linker Segment | 1-20, 22-418 |
| | (e.g., (G4S)n) | |
| 22 | Peptide Linker Segment | 1-21, 23-418 |
| | (e.g., (G4S)n) | |
| 23 | Peptide Linker Segment | 1-22, 24-418 |
| | (e.g., (G4S)n) | |
| 24 | Peptide Linker Segment | 1-23, 25-418 |
| | (e.g., (G4S)n) | |
| 25 | Peptide Linker Segment | 1-24, 26-418 |
| | (e.g., (G4S)n) | |
| 26 | Peptide Linker Segment | 1-25, 27-418 |
| | (e.g., (G4S)n) | |
| 27 | Peptide Linker Segment | 1-26, 28-418 |
| | (e.g., (G4S)n) | |
| 28 | Peptide Linker Segment | 1-27, 29-418 |
| | (e.g., (G4S)n) | |
| 29 | Peptide Linker Segment | 1-28, 30-418 |
| | (e.g., (G4S)n) | |
| 30 | Peptide Linker Segment | 1-29, 31-418 |
| | (e.g., (G4S)n) | |
| 31 | Peptide Linker Segment | 1-30, 32-418 |
| | (e.g., (G4S)n) | |
| 32 | Peptide Linker Segment | 1-31, 33-418 |
| | (e.g., (G4S)n) | |
| 33 | Peptide Linker Segment | 1-32, 34-418 |
| | (e.g., (G4S)n) | |
| 34 | Peptide Linker Segment | 1-33, 35-418 |
| | (e.g., (G4S)n) | |
| 35 | Peptide Linker Segment | 1-34, 36-418 |
| | (e.g., (G4S)n) | |
| 36 | Peptide Linker Segment | 1-35, 37-418 |
| | (e.g., (G4S)n) | |
| 37 | Peptide Linker Segment | 1-36, 38-418 |
| | (e.g., (G4S)n) | |
| 38 | Peptide Linker Segment | 1-37, 39-418 |
| | (e.g., (G4S)n) | |
| 39 | Peptide Linker Segment | 1-38, 40-418 |
| | (e.g., (G4S)n) | |
| 40 | Peptide Linker Segment | 1-39, 41-418 |
| | (e.g., (G4S)n) | |
| 41 | Peptide Linker Segment | 1-40, 42-418 |
| | (e.g., (G4S)n) | |
| 42 | Peptide Linker Segment | 1-41, 43-418 |
| | (e.g., (G4S)n) | |
| 43 | Peptide Linker Segment | 1-42, 44-418 |
| | (e.g., (G4S)n) | |
| 44 | Peptide Linker Segment | 1-43, 45-418 |
| | (e.g., (G4S)n) | |
| 45 | Peptide Linker Segment | 1-44, 46-418 |
| | (e.g., (G4S)n) | |
| 46 | Peptide Linker Segment | 1-45, 47-418 |
| | (e.g., (G4S)n) | |
| 47 | Peptide Linker Segment | 1-46, 48-418 |
| | (e.g., (G4S)n) | |
| 48 | Peptide Linker Segment | 1-47, 49-418 |
| | (e.g., (G4S)n) | |
| 49 | Peptide Linker Segment | 1-48, 50-418 |
| | (e.g., (G4S)n) | |
| 50 | Peptide Linker Segment | 1-49, 51-418 |
| | (e.g., (G4S)n) | |
| 51 | Peptide Linker Segment | 1-50, 52-418 |
| | (e.g., (G4S)n) | |
| 52 | Peptide Linker Segment | 1-51, 53-418 |
| | (e.g., (G4S)n) | |
| 53 | Peptide Linker Segment | 1-52, 54-418 |
| | (e.g., (G4S)n) | |
| 54 | Peptide Linker Segment | 1-53, 55-418 |
| | (e.g., (G4S)n) | |
| 55 | Peptide Linker Segment | 1-54, 56-418 |
| | (e.g., (G4S)n) | |
| 56 | Peptide Linker Segment | 1-55, 57-418 |
| | (e.g., (G4S)n) | |
| 57 | Peptide Linker Segment | 1-56, 58-418 |
| | (e.g., (G4S)n) | |
| 58 | Peptide Linker Segment | 1-57, 59-418 |
| | (e.g., (G4S)n) | |
| 59 | Peptide Linker Segment | 1-58, 60-418 |
| | (e.g., (G4S)n) | |
| 60 | Peptide Linker Segment | 1-59, 61-418 |
| | (e.g., (G4S)n) | |
| 61 | Peptide Linker Segment | 1-60, 62-418 |
| | (e.g., (G4S)n) | |
| 62 | Peptide Linker Segment | 1-61, 63-418 |
| | (e.g., (G4S)n) | |
| 63 | Peptide Linker Segment | 1-62, 64-418 |
| | (e.g., (G4S)n) | |
| 64 | Peptide Linker Segment | 1-63, 65-418 |
| | (e.g., (G4S)n) | |
| 65 | Peptide Linker Segment | 1-64, 66-418 |
| | (e.g., (G4S)n) | |
| 66 | Peptide Linker Segment | 1-65, 67-418 |
| | (e.g., (G4S)n) | |
| 67 | Peptide Linker Segment | 1-66, 68-418 |
| | (e.g., (G4S)n) | |
| 68 | Peptide Linker Segment | 1-67, 69-418 |
| | (e.g., (G4S)n) | |
| 69 | Peptide Linker Segment | 1-68, 70-418 |
| | (e.g., (G4S)n) | |
| 70 | Peptide Linker Segment | 1-69, 71-418 |
| | (e.g., (G4S)n) | |
| 71 | Peptide Linker Segment | 1-70, 72-418 |
| | (e.g., (G4S)n) | |
| 72 | Peptide Linker Segment | 1-71, 73-418 |
| | (e.g., (G4S)n) | |
| 73 | Peptide Linker Segment | 1-72, 74-418 |
| | (e.g., (G4S)n) | |
| 74 | Peptide Linker Segment | 1-73, 75-418 |
| | (e.g., (G4S)n) | |
| 75 | Peptide Linker Segment | 1-74, 76-418 |
| | (e.g., (G4S)n) | |
| 76 | Peptide Linker Segment | 1-75, 77-418 |
| | (e.g., (G4S)n) | |
| 77 | Peptide Linker Segment | 1-76, 78-418 |
| | (e.g., (G4S)n) | |
| 78 | Peptide Linker Segment | 1-77, 79-418 |
| | (e.g., (G4S)n) | |
| 79 | Peptide Linker Segment | 1-78, 80-418 |
| | (e.g., (G4S)n) | |
| 80 | Peptide Linker Segment | 1-79, 81-418 |
| | (e.g., (G4S)n) | |
| 81 | Peptide Linker Segment | 1-80, 82-418 |
| | (e.g., (G4S)n) | |
| 82 | Peptide Linker Segment | 1-81, 83-418 |
| | (e.g., (G4S)n) | |
| 83 | Peptide Linker Segment | 1-82, 84-418 |
| | (e.g., (G4S)n) | |
| 84 | Peptide Linker Segment | 1-83, 85-418 |
| | (e.g., (G4S)n) | |
| 85 | Peptide Linker Segment | 1-84, 86-418 |
| | (e.g., (G4S)n) | |
| 86 | Peptide Linker Segment | 1-85, 87-418 |
| | (e.g., (G4S)n) | |
| 87 | Peptide Linker Segment | 1-86, 88-418 |
| | (e.g., (G4S)n) | |
| 88 | Peptide Linker Segment | 1-87, 89-418 |
| | (e.g., (G4S)n) | |
| 89 | Peptide Linker Segment | 1-88, 90-418 |
| | (e.g., (G4S)n) | |
| 90 | Peptide Linker Segment | 1-89, 91-418 |
| | (e.g., (G4S)n) | |
| 91 | Peptide Linker Segment | 1-90, 92-418 |
| | (e.g., (G4S)n) | |
| 92 | Peptide Linker Segment | 1-91, 93-418 |
| | (e.g., (G4S)n) | |
| 93 | Peptide Linker Segment | 1-92, 94-418 |
| | (e.g., (G4S)n) | |
| 94 | Peptide Linker Segment | 1-93, 95-418 |
| | (e.g., (G4S)n) | |
| 95 | Peptide Linker Segment | 1-94, 96-418 |
| | (e.g., (G4S)n) | |
| 96 | Peptide Linker Segment | 1-95, 97-418 |
| | (e.g., (G4S)n) | |
| 97 | Peptide Linker Segment | 1-96, 98-418 |
| | (e.g., (G4S)n) | |
| 98 | Peptide Linker Segment | 1-97, 99-418 |
| | (e.g., (G4S)n) | |
| 99 | Peptide Linker Segment | 1-98, 100-418 |
| | (e.g., (G4S)n) | |
| 100 | Peptide Linker Segment | 1-99, 101-418 |
| | (e.g., (G4S)n) | |
| 101 | Peptide Linker Segment | 1-100, 102- |
| | (e.g., (G4S)n) | 418 |
| 102 | Peptide Linker Segment | 1-101, 103- |
| | (e.g., (G4S)n) | 418 |
| 103 | Peptide Linker Segment | 1-102, 104- |
| | (e.g., (G4S)n) | 418 |
| 104 | Peptide Linker Segment | 1-103, 105- |
| | (e.g., (G4S)n) | 418 |
| 105 | Peptide Linker Segment | 1-104, 106- |
| | (e.g., (G4S)n) | 418 |
| 106 | Peptide Linker Segment | 1-105, 107- |
| | (e.g., (G4S)n) | 418 |
| 107 | Peptide Linker Segment | 1-106, 108- |
| | (e.g., (G4S)n) | 418 |
| 108 | Peptide Linker Segment | 1-107, 109- |
| | (e.g., (G4S)n) | 418 |
| 109 | Peptide Linker Segment | 1-108, 110- |
| | (e.g., (G4S)n) | 418 |
| 110 | Peptide Linker Segment | 1-109, 111- |
| | (e.g., (G4S)n) | 418 |
| 111 | Peptide Linker Segment | 1-110, 112- |
| | (e.g., (G4S)n) | 418 |
| 112 | Peptide Linker Segment | 1-111, 113- |
| | (e.g., (G4S)n) | 418 |
| 113 | Peptide Linker Segment | 1-112, 114- |
| | (e.g., (G4S)n) | 418 |
| 114 | Peptide Linker Segment | 1-113, 115- |
| | (e.g., (G4S)n) | 418 |
| 115 | Peptide Linker Segment | 1-114, 116- |
| | (e.g., (G4S)n) | 418 |
| 116 | Peptide Linker Segment | 1-115, 117- |
| | (e.g., (G4S)n) | 418 |
| 117 | Peptide Linker Segment | 1-116, 118- |
| | (e.g., (G4S)n) | 418 |
| 118 | Peptide Linker Segment | 1-117, 119- |
| | (e.g., (G4S)n) | 418 |
| 119 | Peptide Linker Segment | 1-118, 120- |
| | (e.g., (G4S)n) | 418 |
| 120 | Peptide Linker Segment | 1-119, 121- |
| | (e.g., (G4S)n) | 418 |
| 121 | Peptide Linker Segment | 1-120, 122- |
| | (e.g., (G4S)n) | 418 |
| 122 | Peptide Linker Segment | 1-121, 123- |
| | (e.g., (G4S)n) | 418 |
| 123 | Peptide Linker Segment | 1-122, 124- |
| | (e.g., (G4S)n) | 418 |
| 124 | Peptide Linker Segment | 1-123, 125- |
| | (e.g., (G4S)n) | 418 |
| 125 | Peptide Linker Segment | 1-124, 126- |
| | (e.g., (G4S)n) | 418 |
| 126 | Peptide Linker Segment | 1-125, 127- |
| | (e.g., (G4S)n) | 418 |
| 127 | Peptide Linker Segment | 1-126, 128- |
| | (e.g., (G4S)n) | 418 |
| 128 | Peptide Linker Segment | 1-127, 129- |
| | (e.g., (G4S)n) | 418 |
| 129 | Peptide Linker Segment | 1-128, 130- |
| | (e.g., (G4S)n) | 418 |
| 130 | Peptide Linker Segment | 1-129, 131- |
| | (e.g., (G4S)n) | 418 |
| 131 | Peptide Linker Segment | 1-130, 132- |
| | (e.g., (G4S)n) | 418 |
| 132 | Peptide Linker Segment | 1-131, 133- |
| | (e.g., (G4S)n) | 418 |
| 133 | Peptide Linker Segment | 1-132, 134- |
| | (e.g., (G4S)n) | 418 |
| 134 | Peptide Linker Segment | 1-133, 135- |
| | (e.g., (G4S)n) | 418 |
| 135 | Peptide Linker Segment | 1-134, 136- |
| | (e.g., (G4S)n) | 418 |
| 136 | Peptide Linker Segment | 1-135, 137- |
| | (e.g., (G4S)n) | 418 |
| 137 | Peptide Linker Segment | 1-136, 138- |
| | (e.g., (G4S)n) | 418 |
| 138 | Peptide Linker Segment | 1-137, 139- |
| | (e.g., (G4S)n) | 418 |
| 139 | Peptide Linker Segment | 1-138, 140- |
| | (e.g., (G4S)n) | 418 |
| 140 | Peptide Linker Segment | 1-139, 141- |
| | (e.g., (G4S)n) | 418 |
| 141 | Peptide Linker Segment | 1-140, 142- |
| | (e.g., (G4S)n) | 418 |
| 142 | Peptide Linker Segment | 1-141, 143- |
| | (e.g., (G4S)n) | 418 |
| 143 | Peptide Linker Segment | 1-142, 144- |
| | (e.g., (G4S)n) | 418 |
| 144 | Peptide Linker Segment | 1-143, 145- |
| | (e.g., (G4S)n) | 418 |
| 145 | Peptide Linker Segment | 1-144, 146- |
| | (e.g., (G4S)n) | 418 |
| 146 | Peptide Linker Segment | 1-145, 147- |
| | (e.g., (G4S)n) | 418 |
| 147 | Peptide Linker Segment | 1-146, 148- |
| | (e.g., (G4S)n) | 418 |
| 148 | Peptide Linker Segment | 1-147, 149- |
| | (e.g., (G4S)n) | 418 |
| 149 | Peptide Linker Segment | 1-148, 150- |
| | (e.g., (G4S)n) | 418 |
| 150 | Peptide Linker Segment | 1-149, 151- |
| | (e.g., (G4S)n) | 418 |
| 151 | Peptide Linker Segment | 1-150, 152- |
| | (e.g., (G4S)n) | 418 |
| 152 | Peptide Linker Segment | 1-151, 153- |
| | (e.g., (G4S)n) | 418 |
| 153 | Peptide Linker Segment | 1-152, 154- |
| | (e.g., (G4S)n) | 418 |
| 154 | Peptide Linker Segment | 1-153, 155- |
| | (e.g., (G4S)n) | 418 |
| 155 | Peptide Linker Segment | 1-154, 156- |
| | (e.g., (G4S)n) | 418 |
| 156 | Peptide Linker Segment | 1-155, 157- |
| | (e.g., (G4S)n) | 418 |
| 157 | Peptide Linker Segment | 1-156, 158- |
| | (e.g., (G4S)n) | 418 |
| 158 | Peptide Linker Segment | 1-157, 159- |
| | (e.g., (G4S)n) | 418 |
| 159 | Peptide Linker Segment | 1-158, 160- |
| | (e.g., (G4S)n) | 418 |
| 160 | Peptide Linker Segment | 1-159, 161- |
| | (e.g., (G4S)n) | 418 |
| 161 | Peptide Linker Segment | 1-160, 162- |
| | (e.g., (G4S)n) | 418 |
| 162 | Peptide Linker Segment | 1-161, 163- |
| | (e.g., (G4S)n) | 418 |
| 163 | Peptide Linker Segment | 1-162, 164- |
| | (e.g., (G4S)n) | 418 |
| 164 | Peptide Linker Segment | 1-163, 165- |
| | (e.g., (G4S)n) | 418 |
| 165 | Peptide Linker Segment | 1-164, 166- |
| | (e.g., (G4S)n) | 418 |
| 166 | Peptide Linker Segment | 1-165, 167- |
| | (e.g., (G4S)n) | 418 |
| 167 | Peptide Linker Segment | 1-166, 168- |
| | (e.g., (G4S)n) | 418 |
| 168 | Peptide Linker Segment | 1-167, 169- |
| | (e.g., (G4S)n) | 418 |
| 169 | Peptide Linker Segment | 1-168, 170- |
| | (e.g., (G4S)n) | 418 |
| 170 | Peptide Linker Segment | 1-169, 171- |
| | (e.g., (G4S)n) | 418 |
| 171 | Peptide Linker Segment | 1-170, 172- |
| | (e.g., (G4S)n) | 418 |
| 172 | Peptide Linker Segment | 1-171, 173- |
| | (e.g., (G4S)n) | 418 |
| 173 | Peptide Linker Segment | 1-172, 174- |
| | (e.g., (G4S)n) | 418 |
| 174 | Peptide Linker Segment | 1-173, 175- |
| | (e.g., (G4S)n) | 418 |
| 175 | Peptide Linker Segment | 1-174, 176- |
| | (e.g., (G4S)n) | 418 |
| 176 | Peptide Linker Segment | 1-175, 177- |
| | (e.g., (G4S)n) | 418 |
| 177 | Peptide Linker Segment | 1-176, 178- |
| | (e.g., (G4S)n) | 418 |
| 178 | Peptide Linker Segment | 1-177, 179- |
| | (e.g., (G4S)n) | 418 |
| 179 | Peptide Linker Segment | 1-178, 180- |
| | (e.g., (G4S)n) | 418 |
| 180 | Peptide Linker Segment | 1-179, 181- |
| | (e.g., (G4S)n) | 418 |
| 181 | Peptide Linker Segment | 1-180, 182- |
| | (e.g., (G4S)n) | 418 |
| 182 | Peptide Linker Segment | 1-181, 183- |
| | (e.g., (G4S)n) | 418 |
| 183 | Peptide Linker Segment | 1-182, 184- |
| | (e.g., (G4S)n) | 418 |
| 184 | Peptide Linker Segment | 1-183, 185- |
| | (e.g., (G4S)n) | 418 |
| 185 | Peptide Linker Segment | 1-184, 186- |
| | (e.g., (G4S)n) | 418 |
| 186 | Peptide Linker Segment | 1-185, 187- |
| | (e.g., (G4S)n) | 418 |
| 187 | Peptide Linker Segment | 1-186, 188- |
| | (e.g., (G4S)n) | 418 |
| 188 | Peptide Linker Segment | 1-187, 189- |
| | (e.g., (G4S)n) | 418 |
| 189 | Peptide Linker Segment | 1-188, 190- |
| | (e.g., (G4S)n) | 418 |
| 190 | Peptide Linker Segment | 1-189, 191- |
| | (e.g., (G4S)n) | 418 |
| 191 | Peptide Linker Segment | 1-190, 192- |
| | (e.g., (G4S)n) | 418 |
| 192 | Peptide Linker Segment | 1-191, 193- |
| | (e.g., (G4S)n) | 418 |
| 193 | Peptide Linker Segment | 1-192, 194- |
| | (e.g., (G4S)n) | 418 |
| 194 | Peptide Linker Segment | 1-193, 195- |
| | (e.g., (G4S)n) | 418 |
| 195 | Peptide Linker Segment | 1-194, 196- |
| | (e.g., (G4S)n) | 418 |
| 196 | Peptide Linker Segment | 1-195, 197- |
| | (e.g., (G4S)n) | 418 |
| 197 | Peptide Linker Segment | 1-196, 198- |
| | (e.g., (G4S)n) | 418 |
| 198 | Peptide Linker Segment | 1-197, 199- |
| | (e.g., (G4S)n) | 418 |
| 199 | Peptide Linker Segment | 1-198, 200- |
| | (e.g., (G4S)n) | 418 |
| 200 | Peptide Linker Segment | 1-199, 201- |
| | (e.g., (G4S)n) | 418 |
| 201 | Peptide Linker Segment | 1-200, 202- |
| | (e.g., (G4S)n) | 418 |
| 202 | Peptide Linker Segment | 1-201, 203- |
| | (e.g., (G4S)n) | 418 |
| 203 | Peptide Linker Segment | 1-202, 204- |
| | (e.g., (G4S)n) | 418 |
| 204 | Peptide Linker Segment | 1-203, 205- |
| | (e.g., (G4S)n) | 418 |
| 205 | Peptide Linker Segment | 1-204, 206- |
| | (e.g., (G4S)n) | 418 |
| 206 | Peptide Linker Segment | 1-205, 207- |
| | (e.g., (G4S)n) | 418 |
| 207 | Peptide Linker Segment | 1-206, 208- |
| | (e.g., (G4S)n) | 418 |
| 208 | Peptide Linker Segment | 1-207, 209- |
| | (e.g., (G4S)n) | 418 |
| 209 | Peptide Linker Segment | 1-208, 210- |
| | (e.g., (G4S)n) | 418 |
| 210 | Peptide Linker Segment | 1-209, 211- |
| | (e.g., (G4S)n) | 418 |
| 211 | Peptide Linker Segment | 1-210, 212- |
| | (e.g., (G4S)n) | 418 |
| 212 | Peptide Linker Segment | 1-211, 213- |
| | (e.g., (G4S)n) | 418 |
| 213 | Peptide Linker Segment | 1-212, 214- |
| | (e.g., (G4S)n) | 418 |
| 214 | Peptide Linker Segment | 1-213, 215- |
| | (e.g., (G4S)n) | 418 |
| 215 | Peptide Linker Segment | 1-214, 216- |
| | (e.g., (G4S)n) | 418 |
| 216 | Peptide Linker Segment | 1-215, 217- |
| | (e.g., (G4S)n) | 418 |
| 217 | Peptide Linker Segment | 1-216, 218- |
| | (e.g., (G4S)n) | 418 |
| 218 | Peptide Linker Segment | 1-217, 219- |
| | (e.g., (G4S)n) | 418 |
| 219 | Peptide Linker Segment | 1-218, 220- |
| | (e.g., (G4S)n) | 418 |
| 220 | Peptide Linker Segment | 1-219, 221- |
| | (e.g., (G4S)n) | 418 |
| 221 | Peptide Linker Segment | 1-220, 222- |
| | (e.g., (G4S)n) | 418 |
| 222 | Peptide Linker Segment | 1-221, 223- |
| | (e.g., (G4S)n) | 418 |
| 223 | Peptide Linker Segment | 1-222, 224- |
| | (e.g., (G4S)n) | 418 |
| 224 | Peptide Linker Segment | 1-223, 225- |
| | (e.g., (G4S)n) | 418 |
| 225 | Peptide Linker Segment | 1-224, 226- |
| | (e.g., (G4S)n) | 418 |
| 226 | Peptide Linker Segment | 1-225, 227- |
| | (e.g., (G4S)n) | 418 |
| 227 | Peptide Linker Segment | 1-226, 228- |
| | (e.g., (G4S)n) | 418 |
| 228 | Peptide Linker Segment | 1-227, 229- |
| | (e.g., (G4S)n) | 418 |
| 229 | Peptide Linker Segment | 1-228, 230- |
| | (e.g., (G4S)n) | 418 |
| 230 | Peptide Linker Segment | 1-229, 231- |
| | (e.g., (G4S)n) | 418 |
| 231 | Peptide Linker Segment | 1-230, 232- |
| | (e.g., (G4S)n) | 418 |
| 232 | Peptide Linker Segment | 1-231, 233- |
| | (e.g., (G4S)n) | 418 |
| 233 | Peptide Linker Segment | 1-232, 234- |
| | (e.g., (G4S)n) | 418 |
| 234 | Peptide Linker Segment | 1-233, 235- |
| | (e.g., (G4S)n) | 418 |
| 235 | Peptide Linker Segment | 1-234, 236- |
| | (e.g., (G4S)n) | 418 |
| 236 | Peptide Linker Segment | 1-235, 237- |
| | (e.g., (G4S)n) | 418 |
| 237 | Peptide Linker Segment | 1-236, 238- |
| | (e.g., (G4S)n) | 418 |
| 238 | Peptide Linker Segment | 1-237, 239- |
| | (e.g., (G4S)n) | 418 |
| 239 | Peptide Linker Segment | 1-238, 240- |
| | (e.g., (G4S)n) | 418 |
| 240 | Peptide Linker Segment | 1-239, 241- |
| | (e.g., (G4S)n) | 418 |
| 241 | Peptide Linker Segment | 1-240, 242- |
| | (e.g., (G4S)n) | 418 |
| 242 | Peptide Linker Segment | 1-241, 243- |
| | (e.g., (G4S)n) | 418 |
| 243 | Peptide Linker Segment | 1-242, 244- |
| | (e.g., (G4S)n) | 418 |
| 244 | Peptide Linker Segment | 1-243, 245- |
| | (e.g., (G4S)n) | 418 |
| 245 | Peptide Linker Segment | 1-244, 246- |
| | (e.g., (G4S)n) | 418 |
| 246 | Peptide Linker Segment | 1-245, 247- |
| | (e.g., (G4S)n) | 418 |
| 247 | Peptide Linker Segment | 1-246, 248- |
| | (e.g., (G4S)n) | 418 |
| 248 | Peptide Linker Segment | 1-247, 249- |
| | (e.g., (G4S)n) | 418 |
| 249 | Peptide Linker Segment | 1-248, 250- |
| | (e.g., (G4S)n) | 418 |
| 250 | Peptide Linker Segment | 1-249, 251- |
| | (e.g., (G4S)n) | 418 |
| 251 | Peptide Linker Segment | 1-250, 252- |
| | (e.g., (G4S)n) | 418 |
| 252 | Peptide Linker Segment | 1-251, 253- |
| | (e.g., (G4S)n) | 418 |
| 253 | Peptide Linker Segment | 1-252, 254- |
| | (e.g., (G4S)n) | 418 |
| 254 | Peptide Linker Segment | 1-253, 255- |
| | (e.g., (G4S)n) | 418 |
| 255 | Peptide Linker Segment | 1-254, 256- |
| | (e.g., (G4S)n) | 418 |
| 256 | Peptide Linker Segment | 1-255, 257- |
| | (e.g., (G4S)n) | 418 |
| 257 | Peptide Linker Segment | 1-256, 258- |
| | (e.g., (G4S)n) | 418 |
| 258 | Peptide Linker Segment | 1-257, 259- |
| | (e.g., (G4S)n) | 418 |
| 259 | Peptide Linker Segment | 1-258, 260- |
| | (e.g., (G4S)n) | 418 |
| 260 | Peptide Linker Segment | 1-259, 261- |
| | (e.g., (G4S)n) | 418 |
| 261 | Peptide Linker Segment | 1-260, 262- |
| | (e.g., (G4S)n) | 418 |
| 262 | Peptide Linker Segment | 1-261, 263- |
| | (e.g., (G4S)n) | 418 |
| 263 | Peptide Linker Segment | 1-262, 264- |
| | (e.g., (G4S)n) | 418 |
| 264 | Peptide Linker Segment | 1-263, 265- |
| | (e.g., (G4S)n) | 418 |
| 265 | Peptide Linker Segment | 1-264, 266- |
| | (e.g., (G4S)n) | 418 |
| 266 | Peptide Linker Segment | 1-265, 267- |
| | (e.g., (G4S)n) | 418 |
| 267 | Peptide Linker Segment | 1-266, 268- |
| | (e.g., (G4S)n) | 418 |
| 268 | Peptide Linker Segment | 1-267, 269- |
| | (e.g., (G4S)n) | 418 |
| 269 | Peptide Linker Segment | 1-268, 270- |
| | (e.g., (G4S)n) | 418 |
| 270 | Peptide Linker Segment | 1-269, 271- |
| | (e.g., (G4S)n) | 418 |
| 271 | Peptide Linker Segment | 1-270, 272- |
| | (e.g., (G4S)n) | 418 |
| 272 | Peptide Linker Segment | 1-271, 273- |
| | (e.g., (G4S)n) | 418 |
| 273 | Peptide Linker Segment | 1-272, 274- |
| | (e.g., (G4S)n) | 418 |
| 274 | Peptide Linker Segment | 1-273, 275- |
| | (e.g., (G4S)n) | 418 |
| 275 | Peptide Linker Segment | 1-274, 276- |
| | (e.g., (G4S)n) | 418 |
| 276 | Peptide Linker Segment | 1-275, 277- |
| | (e.g., (G4S)n) | 418 |
| 277 | Peptide Linker Segment | 1-276, 278- |
| | (e.g., (G4S)n) | 418 |
| 278 | Peptide Linker Segment | 1-277, 279- |
| | (e.g., (G4S)n) | 418 |
| 279 | Peptide Linker Segment | 1-278, 280- |
| | (e.g., (G4S)n) | 418 |
| 280 | Peptide Linker Segment | 1-279, 281- |
| | (e.g., (G4S)n) | 418 |
| 281 | Peptide Linker Segment | 1-280, 282- |
| | (e.g., (G4S)n) | 418 |
| 282 | Peptide Linker Segment | 1-281, 283- |
| | (e.g., (G4S)n) | 418 |
| 283 | Peptide Linker Segment | 1-282, 284- |
| | (e.g., (G4S)n) | 418 |
| 284 | Peptide Linker Segment | 1-283, 285- |
| | (e.g., (G4S)n) | 418 |
| 285 | Peptide Linker Segment | 1-284, 286- |
| | (e.g., (G4S)n) | 418 |
| 286 | Peptide Linker Segment | 1-285, 287- |
| | (e.g., (G4S)n) | 418 |
| 287 | Peptide Linker Segment | 1-286, 288- |
| | (e.g., (G4S)n) | 418 |
| 288 | Peptide Linker Segment | 1-287, 289- |
| | (e.g., (G4S)n) | 418 |
| 289 | Peptide Linker Segment | 1-288, 290- |
| | (e.g., (G4S)n) | 418 |
| 290 | Peptide Linker Segment | 1-289, 291- |
| | (e.g., (G4S)n) | 418 |
| 291 | Peptide Linker Segment | 1-290, 292- |
| | (e.g., (G4S)n) | 418 |
| 292 | Peptide Linker Segment | 1-291, 293- |
| | (e.g., (G4S)n) | 418 |
| 293 | Peptide Linker Segment | 1-292, 294- |
| | (e.g., (G4S)n) | 418 |
| 294 | Peptide Linker Segment | 1-293, 295- |
| | (e.g., (G4S)n) | 418 |
| 295 | Peptide Linker Segment | 1-294, 296- |
| | (e.g., (G4S)n) | 418 |
| 296 | Peptide Linker Segment | 1-295, 297- |
| | (e.g., (G4S)n) | 418 |
| 297 | Peptide Linker Segment | 1-296, 298- |
| | (e.g., (G4S)n) | 418 |
| 298 | Peptide Linker Segment | 1-297, 299- |
| | (e.g., (G4S)n) | 418 |
| 299 | Peptide Linker Segment | 1-298, 300- |
| | (e.g., (G4S)n) | 418 |
| 300 | Peptide Linker Segment | 1-299, 301- |
| | (e.g., (G4S)n) | 418 |
| 301 | Peptide Linker Segment | 1-300, 302- |
| | (e.g., (G4S)n) | 418 |
| 302 | Peptide Linker Segment | 1-301, 303- |
| | (e.g., (G4S)n) | 418 |
| 303 | Peptide Linker Segment | 1-302, 304- |
| | (e.g., (G4S)n) | 418 |
| 304 | Peptide Linker Segment | 1-303, 305- |
| | (e.g., (G4S)n) | 418 |
| 305 | Peptide Linker Segment | 1-304, 306- |
| | (e.g., (G4S)n) | 418 |
| 306 | Peptide Linker Segment | 1-305, 307- |
| | (e.g., (G4S)n) | 418 |
| 307 | Peptide Linker Segment | 1-306, 308- |
| | (e.g., (G4S)n) | 418 |
| 308 | Peptide Linker Segment | 1-307, 309- |
| | (e.g., (G4S)n) | 418 |
| 309 | Peptide Linker Segment | 1-308, 310- |
| | (e.g., (G4S)n) | 418 |
| 310 | Peptide Linker Segment | 1-309, 311- |
| | (e.g., (G4S)n) | 418 |
| 311 | Peptide Linker Segment | 1-310, 312- |
| | (e.g., (G4S)n) | 418 |
| 312 | Peptide Linker Segment | 1-311, 313- |
| | (e.g., (G4S)n) | 418 |
| 313 | Peptide Linker Segment | 1-312, 314- |
| | (e.g., (G4S)n) | 418 |
| 314 | Peptide Linker Segment | 1-313, 315- |
| | (e.g., (G4S)n) | 418 |
| 315 | Peptide Linker Segment | 1-314, 316- |
| | (e.g., (G4S)n) | 418 |
| 316 | Peptide Linker Segment | 1-315, 317- |
| | (e.g., (G4S)n) | 418 |
| 317 | Peptide Linker Segment | 1-316, 318- |
| | (e.g., (G4S)n) | 418 |
| 318 | Peptide Linker Segment | 1-317, 319- |
| | (e.g., (G4S)n) | 418 |
| 319 | Peptide Linker Segment | 1-318, 320- |
| | (e.g., (G4S)n) | 418 |
| 320 | Peptide Linker Segment | 1-319, 321- |
| | (e.g., (G4S)n) | 418 |
| 321 | Peptide Linker Segment | 1-320, 322- |
| | (e.g., (G4S)n) | 418 |
| 322 | Peptide Linker Segment | 1-321, 323- |
| | (e.g., (G4S)n) | 418 |
| 323 | Peptide Linker Segment | 1-322, 324- |
| | (e.g., (G4S)n) | 418 |
| 324 | Peptide Linker Segment | 1-323, 325- |
| | (e.g., (G4S)n) | 418 |
| 325 | Peptide Linker Segment | 1-324, 326- |
| | (e.g., (G4S)n) | 418 |
| 326 | Peptide Linker Segment | 1-325, 327- |
| | (e.g., (G4S)n) | 418 |
| 327 | Peptide Linker Segment | 1-326, 328- |
| | (e.g., (G4S)n) | 418 |
| 328 | Peptide Linker Segment | 1-327, 329- |
| | (e.g., (G4S)n) | 418 |
| 329 | Peptide Linker Segment | 1-328, 330- |
| | (e.g., (G4S)n) | 418 |
| 330 | Peptide Linker Segment | 1-329, 331- |
| | (e.g., (G4S)n) | 418 |
| 331 | Peptide Linker Segment | 1-330, 332- |
| | (e.g., (G4S)n) | 418 |
| 332 | Peptide Linker Segment | 1-331, 333- |
| | (e.g., (G4S)n) | 418 |
| 333 | Peptide Linker Segment | 1-332, 334- |
| | (e.g., (G4S)n) | 418 |
| 334 | Peptide Linker Segment | 1-333, 335- |
| | (e.g., (G4S)n) | 418 |
| 335 | Peptide Linker Segment | 1-334, 336- |
| | (e.g., (G4S)n) | 418 |
| 336 | Peptide Linker Segment | 1-335, 337- |
| | (e.g., (G4S)n) | 418 |
| 337 | Peptide Linker Segment | 1-336, 338- |
| | (e.g., (G4S)n) | 418 |
| 338 | Peptide Linker Segment | 1-337, 339- |
| | (e.g., (G4S)n) | 418 |
| 339 | Peptide Linker Segment | 1-338, 340- |
| | (e.g., (G4S)n) | 418 |
| 340 | Peptide Linker Segment | 1-339, 341- |
| | (e.g., (G4S)n) | 418 |
| 341 | Peptide Linker Segment | 1-340, 342- |
| | (e.g., (G4S)n) | 418 |
| 342 | Peptide Linker Segment | 1-341, 343- |
| | (e.g., (G4S)n) | 418 |
| 343 | Peptide Linker Segment | 1-342, 344- |
| | (e.g., (G4S)n) | 418 |
| 344 | Peptide Linker Segment | 1-343, 345- |
| | (e.g., (G4S)n) | 418 |
| 345 | Peptide Linker Segment | 1-344, 346- |
| | (e.g., (G4S)n) | 418 |
| 346 | Peptide Linker Segment | 1-345, 347- |
| | (e.g., (G4S)n) | 418 |
| 347 | Peptide Linker Segment | 1-346, 348- |
| | (e.g., (G4S)n) | 418 |
| 348 | Peptide Linker Segment | 1-347, 349- |
| | (e.g., (G4S)n) | 418 |
| 349 | Peptide Linker Segment | 1-348, 350- |
| | (e.g., (G4S)n) | 418 |
| 350 | Peptide Linker Segment | 1-349, 351- |
| | (e.g., (G4S)n) | 418 |
| 351 | Peptide Linker Segment | 1-350, 352- |
| | (e.g., (G4S)n) | 418 |
| 352 | Peptide Linker Segment | 1-351, 353- |
| | (e.g., (G4S)n) | 418 |
| 353 | Peptide Linker Segment | 1-352, 354- |
| | (e.g., (G4S)n) | 418 |
| 354 | Peptide Linker Segment | 1-353, 355- |
| | (e.g., (G4S)n) | 418 |
| 355 | Peptide Linker Segment | 1-354, 356- |
| | (e.g., (G4S)n) | 418 |
| 356 | Peptide Linker Segment | 1-355, 357- |
| | (e.g., (G4S)n) | 418 |
| 357 | Peptide Linker Segment | 1-356, 358- |
| | (e.g., (G4S)n) | 418 |
| 358 | Peptide Linker Segment | 1-357, 359- |
| | (e.g., (G4S)n) | 418 |
| 359 | Peptide Linker Segment | 1-358, 360- |
| | (e.g., (G4S)n) | 418 |
| 360 | Peptide Linker Segment | 1-359, 361- |
| | (e.g., (G4S)n) | 418 |
| 361 | Peptide Linker Segment | 1-360, 362- |
| | (e.g., (G4S)n) | 418 |
| 362 | Peptide Linker Segment | 1-361, 363- |
| | (e.g., (G4S)n) | 418 |
| 363 | Peptide Linker Segment | 1-362, 364- |
| | (e.g., (G4S)n) | 418 |
| 364 | Peptide Linker Segment | 1-363, 365- |
| | (e.g., (G4S)n) | 418 |
| 365 | Peptide Linker Segment | 1-364, 366- |
| | (e.g., (G4S)n) | 418 |
| 366 | Peptide Linker Segment | 1-365, 367- |
| | (e.g., (G4S)n) | 418 |
| 367 | Peptide Linker Segment | 1-366, 368- |
| | (e.g., (G4S)n) | 418 |
| 368 | Peptide Linker Segment | 1-367, 369- |
| | (e.g., (G4S)n) | 418 |
| 369 | Peptide Linker Segment | 1-368, 370- |
| | (e.g., (G4S)n) | 418 |
| 370 | Peptide Linker Segment | 1-369, 371- |
| | (e.g., (G4S)n) | 418 |
| 371 | Peptide Linker Segment | 1-370, 372- |
| | (e.g., (G4S)n) | 418 |
| 372 | Peptide Linker Segment | 1-371, 373- |
| | (e.g., (G4S)n) | 418 |
| 373 | Peptide Linker Segment | 1-372, 374- |
| | (e.g., (G4S)n) | 418 |
| 374 | Peptide Linker Segment | 1-373, 375- |
| | (e.g., (G4S)n) | 418 |
| 375 | Peptide Linker Segment | 1-374, 376- |
| | (e.g., (G4S)n) | 418 |
| 376 | Peptide Linker Segment | 1-375, 377- |
| | (e.g., (G4S)n) | 418 |
| 377 | Peptide Linker Segment | 1-376, 378- |
| | (e.g., (G4S)n) | 418 |
| 378 | Peptide Linker Segment | 1-377, 379- |
| | (e.g., (G4S)n) | 418 |
| 379 | Peptide Linker Segment | 1-378, 380- |
| | (e.g., (G4S)n) | 418 |
| 380 | Peptide Linker Segment | 1-379, 381- |
| | (e.g., (G4S)n) | 418 |
| 381 | Peptide Linker Segment | 1-380, 382- |
| | (e.g., (G4S)n) | 418 |
| 382 | Peptide Linker Segment | 1-381, 383- |
| | (e.g., (G4S)n) | 418 |
| 383 | Peptide Linker Segment | 1-382, 384- |
| | (e.g., (G4S)n) | 418 |
| 384 | Peptide Linker Segment | 1-383, 385- |
| | (e.g., (G4S)n) | 418 |
| 385 | Peptide Linker Segment | 1-384, 386- |
| | (e.g., (G4S)n) | 418 |
| 386 | Peptide Linker Segment | 1-385, 387- |
| | (e.g., (G4S)n) | 418 |
| 387 | Peptide Linker Segment | 1-386, 388- |
| | (e.g., (G4S)n) | 418 |
| 388 | Peptide Linker Segment | 1-387, 389- |
| | (e.g., (G4S)n) | 418 |
| 389 | Peptide Linker Segment | 1-388, 390- |
| | (e.g., (G4S)n) | 418 |
| 390 | Peptide Linker Segment | 1-389, 391- |
| | (e.g., (G4S)n) | 418 |
| 391 | Peptide Linker Segment | 1-390, 392- |
| | (e.g., (G4S)n) | 418 |
| 392 | Peptide Linker Segment | 1-391, 393- |
| | (e.g., (G4S)n) | 418 |
| 393 | Peptide Linker Segment | 1-392, 394- |
| | (e.g., (G4S)n) | 418 |
| 394 | Peptide Linker Segment | 1-393, 395- |
| | (e.g., (G4S)n) | 418 |
| 395 | Peptide Linker Segment | 1-394, 396- |
| | (e.g., (G4S)n) | 418 |
| 396 | Peptide Linker Segment | 1-395, 397- |
| | (e.g., (G4S)n) | 418 |
| 397 | Peptide Linker Segment | 1-396, 398- |
| | (e.g., (G4S)n) | 418 |
| 398 | Peptide Linker Segment | 1-397, 399- |
| | (e.g., (G4S)n) | 418 |
| 399 | Peptide Linker Segment | 1-398, 400- |
| | (e.g., (G4S)n) | 418 |
| 400 | Peptide Linker Segment | 1-399, 401- |
| | (e.g., (G4S)n) | 418 |
| 401 | Peptide Linker Segment | 1-400, 402- |
| | (e.g., (G4S)n) | 418 |
| 402 | Peptide Linker Segment | 1-401, 403- |
| | (e.g., (G4S)n) | 418 |
| 403 | Peptide Linker Segment | 1-402, 404- |
| | (e.g., (G4S)n) | 418 |
| 404 | Peptide Linker Segment | 1-403, 405- |
| | (e.g., (G4S)n) | 418 |
| 405 | Peptide Linker Segment | 1-404, 406- |
| | (e.g., (G4S)n) | 418 |
| 406 | Peptide Linker Segment | 1-405, 407- |
| | (e.g., (G4S)n) | 418 |
| 407 | Peptide Linker Segment | 1-406, 408- |
| | (e.g., (G4S)n) | 418 |
| 408 | Peptide Linker Segment | 1-407, 409- |
| | (e.g., (G4S)n) | 418 |
| 409 | Peptide Linker Segment | 1-408, 410- |
| | (e.g., (G4S)n) | 418 |
| 410 | Peptide Linker Segment | 1-409, 411- |
| | (e.g., (G4S)n) | 418 |
| 411 | Peptide Linker Segment | 1-410, 412- |
| | (e.g., (G4S)n) | 418 |
| 412 | Peptide Linker Segment | 1-411, 413- |
| | (e.g., (G4S)n) | 418 |
| 413 | Peptide Linker Segment | 1-412, 414- |
| | (e.g., (G4S)n) | 418 |
| 414 | Peptide Linker Segment | 1-413, 415- |
| | (e.g., (G4S)n) | 418 |
| 415 | Peptide Linker Segment | 1-414, 416- |
| | (e.g., (G4S)n) | 418 |
| 416 | Peptide Linker Segment | 1-415, 417- |
| | (e.g., (G4S)n) | 418 |
| 417 | Peptide Linker Segment | 1-416, 418 |
| | (e.g., (G4S)n) | |
| 418 | Peptide Linker Segment | 1-417 |
| | (e.g., (G4S)n) | |

The multivalent linker of the present disclosure are at least “bivalent”, and may at least be “trivalent” or “tetravalent” or “hexavalent”. In some embodiments, the multivalent linker is bivalent, trivalent, tetravalent, pentavalent, hexavalent, heptavalent, or greater valency (e.g., containing 2, 3, 4, 5, 6, 7 or more peptide binding arms selected from SEQ ID NO: 1-418).

For example, in some embodiments, a trivalent peptide of the current disclosure comprises the formula

(peptide binding arm)-(linker segment)-(peptide binding arm 2)-(linker segment)-(Peptide binding arm)

For example, in some embodiments, a trivalent peptide of the current disclosure comprises the formula

(SEQ ID NO. 1-418)-(G4S)ₙ-(SEQ ID NO. 1-418)-(G4S)ₙ-(SEQ ID NO. 1-418).

In some embodiments, the multivalent linkers of the present disclosure are less than 90 kDa, less than 85 kDa, less than 80 kDa, less than 75 kDa, less than 70 kDa, less than 65 kDa, less than 60 kDa, less than 55 kDa, less than 50 kDa, less than 45 kDa, less than 40 kDa, less than 35 kDa, less than 30 kDa, less than less than 25 kDa, less than less than 20 kDa, or less than 15 kDa.

In some embodiments, the multivalent linkers are between 15-20 kDa, between 20-25 kDa, between 25-30 kDa, between 30-35 kDa, between 35-40 kDa, between 40-45 kDa, between 45-50 kDa, between 50-55 kDa, between 55-60 kDa, between 60-65 kDa, between 65-70 kDa, between 70-75 kDa, between 75-80 kDa, between 80-85 kDa, or between 85-90 kDa.

K_{D} and k_{off} Rates

The invention and development of the presently-claimed multivalent linkers is based, in part, on the finding that the additional binding strength provided by multiple peptide binding arms resulted in vast technical improvements in a variety of immunoassays and treatment.

As discussed above, one of the important technical limitations of traditional dual-antibody detection technologies is signal bleeding, which occurs due to dissociation of an antibody from its primary binding agent, and potential re-binding to another agent.

In some embodiments, the multivalent linkers of the present disclosure achieve low apparent K_{D} (pM) values (for binding to the target antigen unit).

In some embodiments, the apparent K_{D} (pM) of the multivalent linkers is less than 5,000, less than 4,000, less than 3,000, less than 2,000, less than 1,000, less than 900, less than 800, less than 700, less than 600, less than 500, less than 400, less than 300, less than 200, less than 100, less than 50, less than 10, or less than 1 pM, including all ranges and subranges therebetween.

In some embodiments, the apparent K_{D} (pM) of the multivalent linkers is between 1 to 10, between 10 to 50, between 50 to 100, between 100 to 200, between 200 to 300, between 300 to 400, between 400-500, between 500 to 600, between 600 to 700, between 700 to 800, between 800 to 900, between 900 to 1,000, between 1,000 to 2,000, between 2,000 to 3,000, between 3,000 to 4,000, or between 4,000 to 5,000 pM, including all ranges and subranges therebetween.

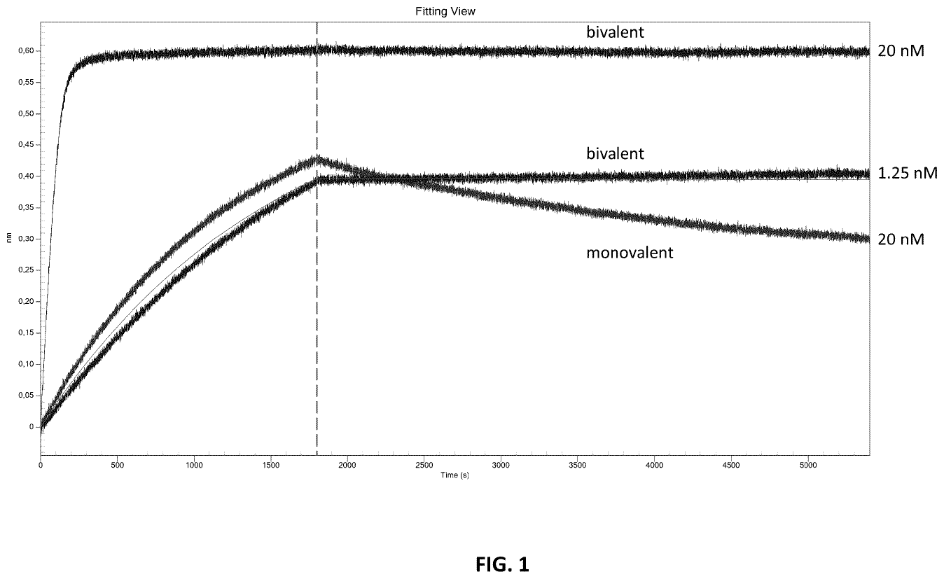

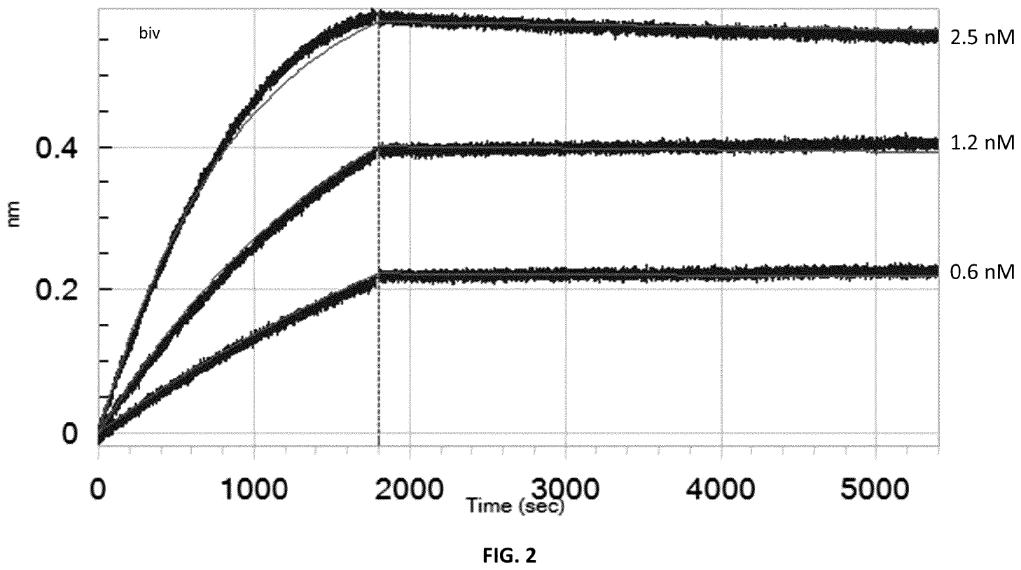

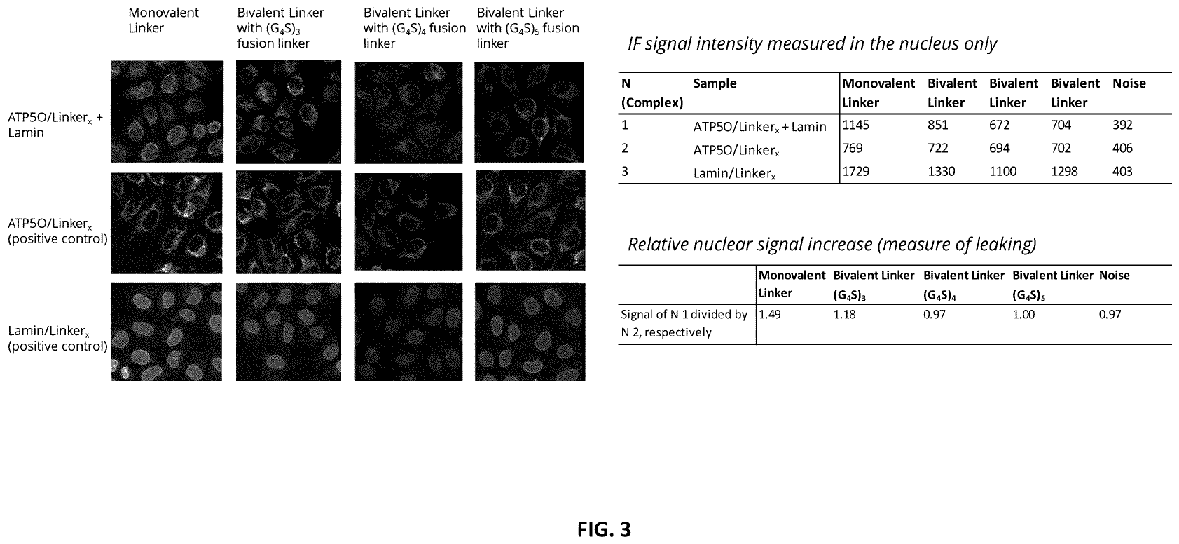

In some embodiments, the multivalent linkers of the present disclosure achieve strong apparent k_{off} rates that were not achievable by prior art secondary antibodies. For example, commercially-available secondary antibodies may exhibit k_{off} rates of greater than 10⁻⁴ s⁻¹, as measured by Bio-Layer Interferometry (BLI). Other monovalent peptide arms are similarly limited, as shown in FIG. 1 and FIG. 3. Thus, in some embodiments, the multivalent linkers of the present disclosure exhibit superior properties to even similar monovalent peptides, such as those disclosed in EP3596464, the content of which is incorporated herein in its entirety.

In some embodiments, the multivalent linker of the present disclosure exhibit superior (lower) k_{off} rates.

In some embodiments, the multivalent linkers disclosed herein have a k_{off}(s⁻¹) rate ranging from about 10⁻⁴ to about 10⁻¹⁰, from about 10⁻⁵ to about 10⁻¹⁰, from about 10⁻⁶ to about 10⁻¹⁰, from about 10⁻⁷ to about 10⁻¹⁰, from about 10⁻⁸ to about 10⁻¹⁰, from about 10⁻⁹ to about 10⁻¹⁰, or about 10⁻¹⁰.

In some embodiments, the multivalent linkers of the present disclosure exhibit less than 5% dissociation after 100 minutes (the average time to conduct an antibody-based assay). Thus, in some embodiments, the multivalent linkers of the present disclosure have a k_{off} (s⁻¹) rate smaller than 10⁻⁴, 10⁻⁵, 10⁻⁶, 10⁻⁷, 10⁻⁸, 10⁻⁹, 10⁻¹⁰, or 10⁻¹¹.

One of the advantages of the presently disclosed multivalent linkers is that they achieve such low k_{off} rates, while in some embodiments still not requiring a covalent link to the target antigen (e.g., to the primary antibody). Thus, at least in some embodiments, the presently disclosed multivalent linkers are distinct from covalent linkers described in U.S. Pat. No. 11,123,440 and WO2022115791.

In one embodiment, the multivalent linkers disclosed herein exhibit a k_{off}(s⁻¹) rate of at most 10⁻⁴, at most 10⁻⁵, at most 10⁻⁶, at most 10⁻⁷, at most 10⁻⁸, at most 10⁻⁹ or at most 10⁻¹⁰.

In some embodiments, the multivalent linkers of the present disclosure are designed to specifically bind a single species of Immunoglobulins, while exhibiting no cross-reactivity to immunoglobulins from other species. For example, in some embodiments the multivalent linkers of the present disclosure specifically bind immunoglobulin from a species selected from the group consisting of human, guinea pig, mouse, rat, chicken, rabbit, goat, donkey, pig, horse, and cattle (e.g., cow), while not exhibiting cross reactivity to immunoglobulins of other species.

As noted throughout this document the multivalent linkers of the present disclosure comprise multiple binding arms, which are believed to impart several advantages to the multivalent linkers over other known antibody labeling technologies. In some embodiments, the multivalent linkers of the present disclosure are not (and do not contain) Fragment Antigen-Binding (FAB) molecules. That is, the multivalent linkers of the present disclosure are distinct and superior to FAB-based technologies, such as those disclosed on the world wide web at thermofisher.com/us/en/home/references/molecular-probes-the-handbook/antibodies-avidins-lectins-and-related-products/56olyv-technology-versatile-reagents-for-immunolabeling.html.

In some embodiments, the multivalent linker specifically binds human immunoglobulin and is not cross-reactive to guinea pig immunoglobulin, mouse immunoglobulin, rat immunoglobulin, chicken immunoglobulin, rabbit immunoglobulin, goat immunoglobulin, donkey immunoglobulin, pig immunoglobulin, horse immunoglobulin, or cattle immunoglobulin.

In some embodiments, the multivalent linker specifically binds rabbit immunoglobulin and is not cross-reactive to guinea pig immunoglobulin, mouse immunoglobulin, rat immunoglobulin, chicken immunoglobulin, human immunoglobulin, goat immunoglobulin, donkey immunoglobulin, pig immunoglobulin, horse immunoglobulin, or cattle immunoglobulin.

In some embodiments, the multivalent linker specifically binds goat immunoglobulin and is not cross-reactive to guinea pig immunoglobulin, mouse immunoglobulin, rat immunoglobulin, human immunoglobulin, chicken immunoglobulin, rabbit immunoglobulin, donkey immunoglobulin, pig immunoglobulin, horse immunoglobulin, or cattle immunoglobulin.

In some embodiments, the multivalent linker specifically binds rat immunoglobulin and is not cross-reactive to guinea pig immunoglobulin, mouse immunoglobulin, chicken immunoglobulin, rabbit immunoglobulin, human immunoglobulin, goat immunoglobulin, donkey immunoglobulin, pig immunoglobulin, horse immunoglobulin, or cattle immunoglobulin.

In some embodiments, the multivalent linker specifically binds mouse immunoglobulin and is not cross-reactive to guinea pig immunoglobulin, rat immunoglobulin, chicken immunoglobulin, rabbit immunoglobulin, human immunoglobulin, goat immunoglobulin, donkey immunoglobulin, pig immunoglobulin, horse immunoglobulin, or cattle immunoglobulin.

In some embodiments, the multivalent linker specifically binds donkey immunoglobulin and is not cross-reactive to guinea pig immunoglobulin, mouse immunoglobulin, rat immunoglobulin, chicken immunoglobulin, rabbit immunoglobulin, human immunoglobulin, goat immunoglobulin, pig immunoglobulin, horse immunoglobulin, or cattle immunoglobulin.

In some embodiments, the multivalent linker specifically binds sheep immunoglobulin and is not cross-reactive to guinea pig immunoglobulin, mouse immunoglobulin, rat immunoglobulin, chicken immunoglobulin, rabbit immunoglobulin, human immunoglobulin, goat immunoglobulin, donkey immunoglobulin, pig immunoglobulin, horse immunoglobulin, or cattle immunoglobulin.

In some embodiments, the multivalent linker specifically binds pig immunoglobulin and is not cross-reactive to guinea pig immunoglobulin, mouse immunoglobulin, rat immunoglobulin, chicken immunoglobulin, rabbit immunoglobulin, human immunoglobulin, goat immunoglobulin, donkey immunoglobulin, horse immunoglobulin, cattle immunoglobulin, sheep immunoglobulin, or rabbit immunoglobulin.

In some embodiments, the multivalent linker specifically horse immunoglobulin and is not cross-reactive to guinea pig immunoglobulin, mouse immunoglobulin, rat immunoglobulin, chicken immunoglobulin, rabbit immunoglobulin, human immunoglobulin, goat immunoglobulin, donkey immunoglobulin, sheep immunoglobulin, pig immunoglobulin, or cattle immunoglobulin.

In some embodiments, the multivalent linker specifically binds cattle immunoglobulin and is not cross-reactive to guinea pig immunoglobulin, mouse immunoglobulin, rat immunoglobulin, chicken immunoglobulin, rabbit immunoglobulin, human immunoglobulin, goat immunoglobulin, donkey immunoglobulin, sheep immunoglobulin, pig immunoglobulin, or horse immunoglobulin.

In some embodiments, the multivalent linker specifically binds chicken immunoglobulin and is not cross-reactive to guinea pig immunoglobulin, mouse immunoglobulin, rat immunoglobulin, rabbit immunoglobulin, human immunoglobulin, goat immunoglobulin, donkey immunoglobulin, sheep immunoglobulin, pig immunoglobulin, horse immunoglobulin, or cattle immunoglobulin.

In some embodiments, the multivalent linker specifically binds guinea pig immunoglobulin and is not cross-reactive to mouse immunoglobulin, rat immunoglobulin, chicken immunoglobulin, rabbit immunoglobulin, human immunoglobulin, goat immunoglobulin, donkey immunoglobulin, sheep immunoglobulin, pig immunoglobulin, horse immunoglobulin, or cattle immunoglobulin.

In some embodiments, the multivalent linker specifically binds rabbit immunoglobulin and/or guinea pig immunoglobulin and is not cross-reactive to mouse immunoglobulin, rat immunoglobulin, chicken immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds rabbit immunoglobulin and/or mouse immunoglobulin and is not cross-reactive to guinea pig immunoglobulin, rat immunoglobulin, chicken immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds rabbit immunoglobulin and/or rat immunoglobulin and is not cross-reactive to mouse immunoglobulin, guinea pig immunoglobulin, chicken immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds rabbit immunoglobulin and/or chicken immunoglobulin and is not cross-reactive to mouse immunoglobulin, rat immunoglobulin, guinea pig immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds rabbit immunoglobulin and/or human immunoglobulin and is not cross-reactive to mouse immunoglobulin, rat immunoglobulin, chicken immunoglobulin, guinea pig immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds rabbit immunoglobulin and/or goat immunoglobulin and is not cross-reactive to mouse immunoglobulin, rat immunoglobulin, chicken immunoglobulin, human immunoglobulin, guinea pig immunoglobulin, donkey immunoglobulin, pig immunoglobulin, horse immunoglobulin, cattle immunoglobulin, or sheep immunoglobulin.

In some embodiments, the multivalent linker specifically binds rabbit immunoglobulin and/or donkey immunoglobulin and is not cross-reactive to mouse immunoglobulin, rat immunoglobulin, chicken immunoglobulin, human immunoglobulin, goat immunoglobulin, guinea pig immunoglobulin, pig immunoglobulin, horse immunoglobulin, cattle immunoglobulin, or sheep immunoglobulin.

In some embodiments, the multivalent linker specifically binds rabbit immunoglobulin and/or pig immunoglobulin and is not cross-reactive to mouse immunoglobulin, rat immunoglobulin, chicken immunoglobulin, human immunoglobulin, goat immunoglobulin, donkey immunoglobulin, guinea pig immunoglobulin, horse immunoglobulin, cattle immunoglobulin, or sheep immunoglobulin.

In some embodiments, the multivalent linker specifically binds rabbit immunoglobulin and/or horse immunoglobulin and is not cross-reactive to mouse immunoglobulin, rat immunoglobulin, chicken immunoglobulin, human immunoglobulin, goat immunoglobulin, donkey immunoglobulin, pig immunoglobulin, guinea pig immunoglobulin, cattle immunoglobulin, or sheep immunoglobulin.

In some embodiments, the multivalent linker specifically binds rabbit immunoglobulin and/or cattle immunoglobulin and is not cross-reactive to mouse immunoglobulin, rat immunoglobulin, chicken immunoglobulin, human immunoglobulin, goat immunoglobulin, donkey immunoglobulin, pig immunoglobulin, horse immunoglobulin, guinea pig immunoglobulin, or sheep immunoglobulin.

In some embodiments, the multivalent linker specifically binds rabbit immunoglobulin and/or sheep immunoglobulin and is not cross-reactive to mouse immunoglobulin, rat immunoglobulin, chicken immunoglobulin, human immunoglobulin, goat immunoglobulin, donkey immunoglobulin, pig immunoglobulin, horse immunoglobulin, guinea pig immunoglobulin, or cattle immunoglobulin.

In some embodiments, the multivalent linker specifically binds guinea pig immunoglobulin and/or mouse immunoglobulin and is not cross-reactive to rabbit immunoglobulin, rat immunoglobulin, chicken immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds guinea pig immunoglobulin and/or rat immunoglobulin and is not cross-reactive to rabbit immunoglobulin, mouse immunoglobulin, chicken immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds guinea pig immunoglobulin and/or chicken immunoglobulin and is not cross-reactive to rabbit immunoglobulin, rat immunoglobulin, mouse immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds guinea pig immunoglobulin and/or human immunoglobulin and is not cross-reactive to rabbit immunoglobulin, rat immunoglobulin, chicken immunoglobulin, mouse immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds guinea pig immunoglobulin and/or goat immunoglobulin and is not cross-reactive to rabbit immunoglobulin, rat immunoglobulin, chicken immunoglobulin, human immunoglobulin, mouse immunoglobulin, donkey immunoglobulin, pig immunoglobulin, horse immunoglobulin, cattle immunoglobulin, or sheep immunoglobulin.

In some embodiments, the multivalent linker specifically binds guinea pig immunoglobulin and/or donkey immunoglobulin and is not cross-reactive to rabbit immunoglobulin, rat immunoglobulin, chicken immunoglobulin, human immunoglobulin, goat immunoglobulin, mouse immunoglobulin, pig immunoglobulin, horse immunoglobulin, cattle immunoglobulin, or sheep immunoglobulin.

In some embodiments, the multivalent linker specifically binds guinea pig immunoglobulin and/or pig immunoglobulin and is not cross-reactive to rabbit immunoglobulin, rat immunoglobulin, chicken immunoglobulin, human immunoglobulin, goat immunoglobulin, donkey immunoglobulin, mouse immunoglobulin, horse immunoglobulin, cattle immunoglobulin, or sheep immunoglobulin.

In some embodiments, the multivalent linker specifically binds guinea pig immunoglobulin and/or horse immunoglobulin and is not cross-reactive to rabbit immunoglobulin, rat immunoglobulin, chicken immunoglobulin, human immunoglobulin, goat immunoglobulin, donkey immunoglobulin, pig immunoglobulin, mouse immunoglobulin, cattle immunoglobulin, or sheep immunoglobulin.

In some embodiments, the multivalent linker specifically binds guinea pig immunoglobulin and/or cattle immunoglobulin and is not cross-reactive to rabbit immunoglobulin, rat immunoglobulin, chicken immunoglobulin, human immunoglobulin, goat immunoglobulin, donkey immunoglobulin, pig immunoglobulin, horse immunoglobulin, mouse immunoglobulin, or sheep immunoglobulin.

In some embodiments, the multivalent linker specifically binds guinea pig immunoglobulin and/or sheep immunoglobulin and is not cross-reactive to rabbit immunoglobulin, rat immunoglobulin, chicken immunoglobulin, human immunoglobulin, goat immunoglobulin, donkey immunoglobulin, pig immunoglobulin, horse immunoglobulin, mouse immunoglobulin, or cattle immunoglobulin.

In some embodiments, the multivalent linker specifically binds mouse immunoglobulin and/or rat immunoglobulin and is not cross-reactive to rabbit immunoglobulin, guinea pig immunoglobulin, chicken immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds mouse immunoglobulin and/or chicken immunoglobulin and is not cross-reactive to rabbit immunoglobulin, guinea pig immunoglobulin, rat immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds mouse immunoglobulin and/or human immunoglobulin and is not cross-reactive to rabbit immunoglobulin, guinea pig immunoglobulin, rat immunoglobulin, chicken immunoglobulin, goat immunoglobulin, donkey immunoglobulin, pig immunoglobulin, horse immunoglobulin, cattle immunoglobulin, or sheep immunoglobulin.

In some embodiments, the multivalent peptide specifically binds mouse immunoglobulin and/or goat immunoglobulin and is not cross-reactive to rabbit immunoglobulin, guinea pig immunoglobulin, rat immunoglobulin, human immunoglobulin, chicken immunoglobulin, donkey immunoglobulin, pig immunoglobulin, horse immunoglobulin, cattle immunoglobulin, or sheep immunoglobulin.

In some embodiments, the multivalent linker specifically binds mouse immunoglobulin and/or donkey immunoglobulin and is not cross-reactive to rabbit immunoglobulin, guinea pig immunoglobulin, rat immunoglobulin, human immunoglobulin, goat immunoglobulin, chicken immunoglobulin, pig immunoglobulin, horse immunoglobulin, cattle immunoglobulin, or sheep immunoglobulin.

In some embodiments, the multivalent linker specifically binds mouse immunoglobulin and/or pig immunoglobulin and is not cross-reactive to rabbit immunoglobulin, guinea pig immunoglobulin, rat immunoglobulin, human immunoglobulin, goat immunoglobulin, donkey immunoglobulin, chicken immunoglobulin, horse immunoglobulin, cattle immunoglobulin, or sheep immunoglobulin.

In some embodiments, the multivalent linker specifically binds mouse immunoglobulin and/or horse immunoglobulin and is not cross-reactive to rabbit immunoglobulin, guinea pig immunoglobulin, rat immunoglobulin, human immunoglobulin, goat immunoglobulin, donkey immunoglobulin, pig immunoglobulin, chicken immunoglobulin, cattle immunoglobulin, or sheep immunoglobulin.

In some embodiments, the multivalent linker specifically binds mouse immunoglobulin and/or cattle immunoglobulin and is not cross-reactive to rabbit immunoglobulin, guinea pig immunoglobulin, rat immunoglobulin, human immunoglobulin, goat immunoglobulin, donkey immunoglobulin, pig immunoglobulin, horse immunoglobulin, chicken immunoglobulin, or sheep immunoglobulin.

In some embodiments, the multivalent linker specifically binds mouse immunoglobulin and/or sheep immunoglobulin and is not cross-reactive to rabbit immunoglobulin, guinea pig immunoglobulin, rat immunoglobulin, human immunoglobulin, goat immunoglobulin, donkey immunoglobulin, pig immunoglobulin, horse immunoglobulin, chicken immunoglobulin, or cattle immunoglobulin.

In some embodiments, the multivalent linker specifically binds rat immunoglobulin and/or chicken immunoglobulin and is not cross-reactive to rabbit immunoglobulin, guinea pig immunoglobulin, mouse immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds rat immunoglobulin and/or human immunoglobulin and is not cross-reactive to rabbit immunoglobulin, guinea pig immunoglobulin, mouse immunoglobulin, chicken immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds rat immunoglobulin and/or goat immunoglobulin and is not cross-reactive to rabbit immunoglobulin, guinea pig immunoglobulin, mouse immunoglobulin, human immunoglobulin, chicken immunoglobulin, donkey immunoglobulin, pig immunoglobulin, horse immunoglobulin, cattle immunoglobulin, or sheep immunoglobulin.

In some embodiments, the multivalent linker specifically binds rat immunoglobulin and/or donkey immunoglobulin and is not cross-reactive to rabbit immunoglobulin, guinea pig immunoglobulin, mouse immunoglobulin, human immunoglobulin, goat immunoglobulin, chicken immunoglobulin, pig immunoglobulin, horse immunoglobulin, cattle immunoglobulin, or sheep immunoglobulin.

In some embodiments, the multivalent linker specifically binds rat immunoglobulin and/or pig immunoglobulin and is not cross-reactive to rabbit immunoglobulin, guinea pig immunoglobulin, mouse immunoglobulin, human immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds rat immunoglobulin and/or horse immunoglobulin and is not cross-reactive to rabbit immunoglobulin, guinea pig immunoglobulin, mouse immunoglobulin, human immunoglobulin, goat immunoglobulin, donkey immunoglobulin, chicken immunoglobulin, chicken immunoglobulin, cattle immunoglobulin, or sheep immunoglobulin.

In some embodiments, the multivalent linker specifically binds rat immunoglobulin and/or cattle immunoglobulin and is not cross-reactive to rabbit immunoglobulin, guinea pig immunoglobulin, mouse immunoglobulin, human immunoglobulin, goat immunoglobulin, donkey immunoglobulin, pig immunoglobulin, horse immunoglobulin, chicken immunoglobulin, or sheep immunoglobulin.

In some embodiments, the multivalent linker specifically binds rat immunoglobulin and/or sheep immunoglobulin and is not cross-reactive to rabbit immunoglobulin, guinea pig immunoglobulin, mouse immunoglobulin, human immunoglobulin, goat immunoglobulin, donkey immunoglobulin, pig immunoglobulin, horse immunoglobulin, chicken immunoglobulin, or cattle immunoglobulin.

In some embodiments, the multivalent linker specifically binds chicken immunoglobulin and/or human immunoglobulin and is not cross-reactive to rabbit immunoglobulin, guinea pig immunoglobulin, mouse immunoglobulin, rat immunoglobulin, goat immunoglobulin, donkey immunoglobulin, pig immunoglobulin, horse immunoglobulin, cattle immunoglobulin, or sheep immunoglobulin.

In some embodiments, the multivalent linker specifically binds chicken immunoglobulin and/or goat immunoglobulin and is not cross-reactive to rabbit immunoglobulin, guinea pig immunoglobulin, mouse immunoglobulin, rat immunoglobulin, human immunoglobulin, donkey immunoglobulin, pig immunoglobulin, horse immunoglobulin, cattle immunoglobulin, or sheep immunoglobulin.

In some embodiments, the multivalent linker specifically binds chicken immunoglobulin and/or donkey immunoglobulin and is not cross-reactive to rabbit immunoglobulin, guinea pig immunoglobulin, mouse immunoglobulin, rat immunoglobulin, goat immunoglobulin, human immunoglobulin, pig immunoglobulin, horse immunoglobulin, cattle immunoglobulin, or sheep immunoglobulin.

In some embodiments, the multivalent linker specifically binds chicken immunoglobulin and/or pig immunoglobulin and is not cross-reactive to rabbit immunoglobulin, guinea pig immunoglobulin, mouse immunoglobulin, rat immunoglobulin, goat immunoglobulin, donkey immunoglobulin, human immunoglobulin, horse immunoglobulin, cattle immunoglobulin, or sheep immunoglobulin.

In some embodiments, the multivalent linker specifically binds chicken immunoglobulin and/or horse immunoglobulin and is not cross-reactive to rabbit immunoglobulin, guinea pig immunoglobulin, mouse immunoglobulin, rat immunoglobulin, goat immunoglobulin, donkey immunoglobulin, pig immunoglobulin, human immunoglobulin, cattle immunoglobulin, or sheep immunoglobulin.

In some embodiments, the multivalent linker specifically binds chicken immunoglobulin and/or cattle immunoglobulin and is not cross-reactive to rabbit immunoglobulin, guinea pig immunoglobulin, mouse immunoglobulin, rat immunoglobulin, goat immunoglobulin, donkey immunoglobulin, pig immunoglobulin, horse immunoglobulin, or human immunoglobulin, or sheep immunoglobulin.

In some embodiments, the multivalent linker specifically binds chicken immunoglobulin and/or sheep immunoglobulin and is not cross-reactive to rabbit immunoglobulin, guinea pig immunoglobulin, mouse immunoglobulin, rat immunoglobulin, goat immunoglobulin, donkey immunoglobulin, pig immunoglobulin, horse immunoglobulin, human immunoglobulin, or cattle immunoglobulin.

In some embodiments, the multivalent linker specifically binds human immunoglobulin and/or goat immunoglobulin and is not cross-reactive to rabbit immunoglobulin, guinea pig immunoglobulin, mouse immunoglobulin, rat immunoglobulin, chicken immunoglobulin, donkey immunoglobulin, pig immunoglobulin, horse immunoglobulin, cattle immunoglobulin, or sheep immunoglobulin.

In some embodiments, the multivalent linker specifically binds human immunoglobulin and/or donkey immunoglobulin and is not cross-reactive to rabbit immunoglobulin, guinea pig immunoglobulin, mouse immunoglobulin, rat immunoglobulin, chicken immunoglobulin, goat immunoglobulin, pig immunoglobulin, horse immunoglobulin, cattle immunoglobulin, or sheep immunoglobulin.

In some embodiments, the multivalent linker specifically binds human immunoglobulin and/or pig immunoglobulin and is not cross-reactive to rabbit immunoglobulin, guinea pig immunoglobulin, mouse immunoglobulin, rat immunoglobulin, chicken immunoglobulin, donkey immunoglobulin, goat immunoglobulin, horse immunoglobulin, cattle immunoglobulin, or sheep immunoglobulin.

In some embodiments, the multivalent linker specifically binds human immunoglobulin and/or horse immunoglobulin and is not cross-reactive to rabbit immunoglobulin, guinea pig immunoglobulin, mouse immunoglobulin, rat immunoglobulin, chicken immunoglobulin, donkey immunoglobulin, pig immunoglobulin, goat immunoglobulin, cattle immunoglobulin, or sheep immunoglobulin.

In some embodiments, the multivalent linker specifically binds human immunoglobulin and/or cattle immunoglobulin and is not cross-reactive to rabbit immunoglobulin, guinea pig immunoglobulin, mouse immunoglobulin, rat immunoglobulin, chicken immunoglobulin, donkey immunoglobulin, pig immunoglobulin, horse immunoglobulin, goat immunoglobulin, or sheep immunoglobulin.

In some embodiments, the multivalent linker specifically binds human immunoglobulin and/or sheep immunoglobulin and is not cross-reactive to rabbit immunoglobulin, guinea pig immunoglobulin, mouse immunoglobulin, rat immunoglobulin, chicken immunoglobulin, donkey immunoglobulin, pig immunoglobulin, horse immunoglobulin, goat immunoglobulin, or cattle immunoglobulin.

In some embodiments, the multivalent linker specifically binds goat immunoglobulin and/or donkey immunoglobulin and is not cross-reactive to rabbit immunoglobulin, guinea pig immunoglobulin, mouse immunoglobulin, rat immunoglobulin, chicken immunoglobulin, human immunoglobulin, pig immunoglobulin, horse immunoglobulin, cattle immunoglobulin, or sheep immunoglobulin.

In some embodiments, the multivalent linker specifically binds goat immunoglobulin and/or pig immunoglobulin and is not cross-reactive to rabbit immunoglobulin, guinea pig immunoglobulin, mouse immunoglobulin, rat immunoglobulin, chicken immunoglobulin, human immunoglobulin, donkey immunoglobulin, horse immunoglobulin, cattle immunoglobulin, or sheep immunoglobulin.

In some embodiments, the multivalent linker specifically binds goat immunoglobulin and/or horse immunoglobulin and is not cross-reactive to rabbit immunoglobulin, guinea pig immunoglobulin, mouse immunoglobulin, rat immunoglobulin, chicken immunoglobulin, human immunoglobulin, pig immunoglobulin, donkey immunoglobulin, cattle immunoglobulin, or sheep immunoglobulin.

In some embodiments, the multivalent linker specifically binds goat immunoglobulin and/or cattle immunoglobulin and is not cross-reactive to rabbit immunoglobulin, guinea pig immunoglobulin, mouse immunoglobulin, rat immunoglobulin, chicken immunoglobulin, human immunoglobulin, pig immunoglobulin, horse immunoglobulin, donkey immunoglobulin, or sheep immunoglobulin.

In some embodiments, the multivalent linker specifically binds goat immunoglobulin and/or sheep immunoglobulin and is not cross-reactive to rabbit immunoglobulin, guinea pig immunoglobulin, mouse immunoglobulin, rat immunoglobulin, chicken immunoglobulin, human immunoglobulin, pig immunoglobulin, horse immunoglobulin, donkey immunoglobulin, or cattle immunoglobulin.

In some embodiments, the multivalent linker specifically binds donkey immunoglobulin and/or pig immunoglobulin and is not cross-reactive to rabbit immunoglobulin, guinea pig immunoglobulin, mouse immunoglobulin, rat immunoglobulin, chicken immunoglobulin, human immunoglobulin, goat immunoglobulin, horse immunoglobulin, cattle immunoglobulin, or sheep immunoglobulin.

In some embodiments, the multivalent linker specifically binds donkey immunoglobulin and/or horse immunoglobulin and is not cross-reactive to rabbit immunoglobulin, guinea pig immunoglobulin, mouse immunoglobulin, rat immunoglobulin, chicken immunoglobulin, human immunoglobulin, goat immunoglobulin, pig immunoglobulin, cattle immunoglobulin, or sheep immunoglobulin.

In some embodiments, the multivalent linker specifically binds donkey immunoglobulin and/or cattle immunoglobulin and is not cross-reactive to rabbit immunoglobulin, guinea pig immunoglobulin, mouse immunoglobulin, rat immunoglobulin, chicken immunoglobulin, human immunoglobulin, goat immunoglobulin, horse immunoglobulin, pig immunoglobulin, or sheep immunoglobulin.

In some embodiments, the multivalent linker specifically binds donkey immunoglobulin and/or sheep immunoglobulin and is not cross-reactive to rabbit immunoglobulin, guinea pig immunoglobulin, mouse immunoglobulin, rat immunoglobulin, chicken immunoglobulin, human immunoglobulin, goat immunoglobulin, horse immunoglobulin, or pig immunoglobulin, or cattle immunoglobulin.

In some embodiments, the multivalent linker specifically binds pig immunoglobulin and/or horse immunoglobulin and is not cross-reactive to rabbit immunoglobulin, guinea pig immunoglobulin, mouse immunoglobulin, rat immunoglobulin, chicken immunoglobulin, human immunoglobulin, goat immunoglobulin, donkey immunoglobulin, cattle immunoglobulin, or sheep immunoglobulin.

In some embodiments, the multivalent linker specifically binds pig immunoglobulin and/or cattle immunoglobulin and is not cross-reactive to rabbit immunoglobulin, guinea pig immunoglobulin, mouse immunoglobulin, rat immunoglobulin, chicken immunoglobulin, human immunoglobulin, goat immunoglobulin, donkey immunoglobulin, horse immunoglobulin, or sheep immunoglobulin.

In some embodiments, the multivalent linker specifically binds pig immunoglobulin and/or sheep immunoglobulin and is not cross-reactive to rabbit immunoglobulin, guinea pig immunoglobulin, mouse immunoglobulin, rat immunoglobulin, chicken immunoglobulin, human immunoglobulin, goat immunoglobulin, donkey immunoglobulin, horse immunoglobulin, or cattle immunoglobulin.

In some embodiments, the multivalent linker specifically binds horse immunoglobulin and/or cattle immunoglobulin and is not cross-reactive to rabbit immunoglobulin, guinea pig immunoglobulin, mouse immunoglobulin, rat immunoglobulin, chicken immunoglobulin, human immunoglobulin, goat immunoglobulin, donkey immunoglobulin, pig immunoglobulin, or sheep immunoglobulin.

In some embodiments, the multivalent linker specifically binds horse immunoglobulin and/or sheep immunoglobulin and is not cross-reactive to rabbit immunoglobulin, guinea pig immunoglobulin, mouse immunoglobulin, rat immunoglobulin, chicken immunoglobulin, human immunoglobulin, goat immunoglobulin, donkey immunoglobulin, or pig immunoglobulin, or cattle immunoglobulin.

In some embodiments, the multivalent linker specifically binds cattle immunoglobulin and/or sheep immunoglobulin and is not cross-reactive to rabbit immunoglobulin, guinea pig immunoglobulin, mouse immunoglobulin, rat immunoglobulin, chicken immunoglobulin, human immunoglobulin, goat immunoglobulin, donkey immunoglobulin, or pig immunoglobulin, or horse immunoglobulin.

In some embodiments, the multivalent linker specifically binds the constant region of the heavy chain of an immunoglobulin.

In some embodiments, the multivalent linker specifically binds the hinge region of the heavy chain of an immunoglobulin.

In some embodiments, the multivalent linker specifically binds the constant region of the light chain of an immunoglobulin.

### Peptide Binding Arms of the Present Disclosure

In some embodiments, the multivalent linkers of the present disclosure comprise a plurality of peptide binding arms. In some embodiments, the peptide binding arm comprises at least one sequence from SEQ ID NO: 1-418. In some embodiments, the multivalent linker comprises two or more peptide binding arms with sequences selected from SEQ ID NO: 1-418.

In some embodiments, the peptide binding arms are single domain antibodies or antigen-binding fragments thereof. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, cattle, and/or alpaca.

According to the disclosure, a single domain antibody as used herein is derived from a naturally occurring antibody known as heavy chain antibody devoid of light chains. A variable domain derived from a heavy chain antibody naturally devoid of light chain is known herein as a VHH molecule to distinguish it from the conventional VH of four chain immunoglobulins.

In some embodiments, a VHH molecule may be derived from antibodies raised in Camelidae species, for example in camel, dromedary, llama, vicuna, alpaca and guanaco.

Other species besides Camelidae may produce heavy chain antibodies naturally devoid of light chain. For example, sharks produce heavy chain antibodies naturally devoid of light chains (commonly named IgNAR), which comprise a VNAR domain. In addition, single domain antibodies (sdAbs) may be obtained from synthetic libraries. Designed ankyrin repeat proteins (DARPins) are antibody mimetic proteins that may also be generated from synthetic libraries. Other examples may include, but are not limited to, anticalins (lipocalins), single-chain variable fragment (scFv), Fibronectin type III (FN3) domains, monobodies, and/or affibodies. All such sdAbs, DARPins, anticalins, scFvs, FN3 domains, monobodies, and affibodies are within the scope of the disclosure.

In some embodiments, the peptide binding arm of the present disclosure comprises a sequence identity with any of SEQ ID NO. 1-494.

In some embodiments, the multivalent sequence comprises a sequence at least 80%, at least 85%, at least 90%, at least 95%, or 100% identical with any of SEQ ID NO. 1-494.

In some embodiments, the multivalent sequence comprises a sequence between 80%-85%, between 85%-90%, between 90%-95%, or between 95%-100% identical with any of SEQ ID NO. 1-494.

In some embodiments, the C-terminus of a first peptide selected from SEQ ID NO: 1-418 is conjugated to the N-terminus of a second peptide to create a multivalent linker via a linker segment. In some embodiments, the linker segment includes the sequence GGGGS, or G4S, or any other linker segment disclosed in this disclosure.

In some embodiments, the multivalent linker comprises multiple G4S linker segments. For example, a multivalent linker may comprise (G4S)n, wherein n=1-10.

In some embodiments, the first peptide and the second peptides have different sequences (e.g., comprise different SEQ ID NOs disclosed herein).

In some embodiments, the first and second peptides have the same sequence (e.g., are copies of any of the same SEQ ID NO. sequence disclosed herein).

In some embodiments, the peptide binding arm binds to an epitope of the target antigen unit non-covalently. In some embodiments, the peptide binding arm binds to an epitope of the target antigen unit covalently.

### Molecular Complex

In one aspect, the disclosure provides molecular complexes comprising i) a single target antigen unit and a ii) multivalent linker of the disclosure. In some embodiments, the molecular complex comprises a single multivalent linker, such as those of the present disclosure.

In some embodiments, the single target antigen unit comprises at least two identical, or substantially the same, epitopes of the peptide binding arms of the multivalent linker (e.g., a multivalent linker that has at least two identical, or substantially the same, peptide binding arms).

In some embodiments, the single target antigen unit comprises at least two different epitopes for the peptide binding arms of the multivalent linker (e.g., a multivalent linker that has at least two different peptide binding arms).

In some embodiments, the number of epitopes comprised within the single target antigen unit is the same as the number of the peptide binding arms within the multivalent linker. In some embodiments, the number of epitopes/peptide binding arms is both two. In some embodiments, the number of epitopes/peptide binding arms is both three. In some embodiments, the number of epitopes/peptide binding arms is both four. In some embodiments, the number of epitopes/peptide binding arms is both five. In some embodiments, the number of epitopes/peptide binding arms is both six, seven, eight, nine, ten, or more than ten.

In some embodiments, the multivalent linker dissociates from the single target antigen unit at an apparent k_{off}(s⁻¹) rate of less than or equal to 1.0×10⁻⁴. In some embodiments, the multivalent linker dissociates from the single target antigen unit at an apparent k_{off} (s⁻¹) rate smaller than 10⁻⁴, 10⁻⁵, 10⁻⁶, 10⁻⁷, 10⁻⁸, 10⁻⁹, 10⁻¹⁰, or 10⁻¹¹. In some embodiments, the multivalent linker dissociates from the single target antigen unit at an apparent k_{off}(s⁻¹) rate of less than or equal to 1.0×10⁻⁵. In some embodiments, the multivalent linker dissociates from the single target antigen unit at an apparent k_{off}(s⁻¹) rate of less than or equal to 1.0×10⁻⁶. In some embodiments, the multivalent linker dissociates from the single target antigen unit at an apparent k_{off}(s⁻¹) rate of less than or equal to 1.0×10⁻⁷. In some embodiments, the multivalent linker dissociates from the single target antigen unit at an apparent k_{off}(s⁻¹) rate of less than or equal to 1.0×10⁻⁸. In some embodiments, the k_{off}(s⁻¹) rate is determined by Bio-Layer Interferometry (BLI).

In one aspect, the disclosure provides a population of the molecular complex. In some embodiments, the disclosure provides a composition comprising at least 1 g, at least 1 mg, at least 1 ug, or at least 1 ng of the population of the molecular complex. In some embodiments, the disclosure provides a composition comprising at least 1 ng of the population of the molecular complex. In some embodiments, within the composition comprising the population of the molecular complex, less than 5%, 4%, 3%, 2%, or 1% of the plurality of peptide binding arms of the multivalent linker crosslink to different target antigen units.

### Target Antigen Unit

In some embodiments, the multivalent linker binds to a single target antigen unit. In some embodiments, a single target antigen unit is comprised within a molecular complex with the multivalent linker.

In some embodiments, the target antigen unit comprises a constant region of an antibody. In some embodiments, the single target antigen unit comprises an Fc region of an antibody. In some embodiments, the epitope is located on the CH2 domain of the constant region of the antibody. In some embodiments, the epitope is located on the CH3 domain of the constant region of the antibody. In some embodiments, the epitope is located on the CH4 domain of the constant region of the antibody. In some embodiments, the number of the epitope is two within the single target antigen unit.

In some embodiments, the epitope is located at a position not in the Fc region of the antibody. In some embodiments, the epitope is located within the Fab region of the antibody. In some embodiments, the epitope is located within the CH1 domain of the antibody. In some embodiments, the epitope is located within the CL domain (of the light chain) of the antibody.

In some embodiments, the target antigen unit comprises or consists of an antibody, F(ab′)2, Fab₂, Fab₃, or IgNAR. In some embodiments, the target antigen unit comprises or consists of an antibody. In some embodiments, the target antigen unit comprises or consists of an F(ab′)2. In some embodiments, the target antigen unit comprises or consists of an Fab₂. In some embodiments, the target antigen unit comprises or consists of an Fab₃. In some embodiments, the target antigen unit comprises or consists of an IgNAR. In some embodiments, the target antigen unit comprises the antigen binding fragment of an antibody.

In some embodiments, the target antigen unit comprises or consists of an scFv.

In some embodiments, the antibody is an IgG. In some embodiments, the IgG antibody is IgG1, IgG2, IgG3, or IgG4 subclass. In some embodiments, the IgG antibody is IgG1 subclass. In some embodiments, the IgG antibody is IgG2 subclass. In some embodiments, the IgG antibody is IgG3 subclass. In some embodiments, the IgG antibody is IgG4 subclass.

In some embodiments, the IgG antibody is a human antibody. In some embodiments, the human IgG antibody is IgG1, IgG2, IgG3, or IgG4 subclass. In some embodiments, the human IgG antibody is IgG1 subclass. In some embodiments, the human IgG antibody is IgG2 subclass. In some embodiments, the human IgG antibody is IgG3 subclass. In some embodiments, the human IgG antibody is IgG4 subclass. In some embodiments, the constant region of the IgG1 antibody comprises or consists of a sequence having at least 80%, at least 85%, at least 90%, at least 95%, at least 98%, at least 99%, or 100% identity to SEQ ID NO: 496 (Uniprot ID: P01857). In some embodiments, the constant region of the IgG2 antibody comprises or consists of a sequence having at least 80%, at least 85%, at least 90%, at least 95%, at least 98%, at least 99%, or 100% identity to SEQ ID NO: 497 (Uniprot ID: P01859). In some embodiments, the constant region of the IgG3 antibody comprises or consists of a sequence having at least 80%, at least 85%, at least 90%, at least 95%, at least 98%, at least 99%, or 100% identity to SEQ ID NO: 498 (Uniprot ID: P01860). In some embodiments, the constant region of the IgG4 antibody comprises or consists of a sequence having at least 80%, at least 85%, at least 90%, at least 95%, at least 98%, at least 99%, or 100% identity to SEQ ID NO: 499 (Uniprot ID: P01861).

In some embodiments, the IgG antibody is a mouse antibody. In some embodiments, the mouse IgG antibody is IgG1, IgG2a, IgG2b, IgG2c or IgG3 subclass. In some embodiments, the mouse IgG antibody is IgG1 subclass. In some embodiments, the mouse IgG antibody is IgG2a subclass. In some embodiments, the mouse IgG antibody is IgG2b subclass. In some embodiments, the mouse IgG antibody is IgG2c subclass. In some embodiments, the mouse IgG antibody is IgG3 subclass. In some embodiments, the mouse IgG1 antibody is an IgG1* variant. In some embodiments, the mouse IgG2b antibody is an IgG2b* variant. In some embodiments, the mouse IgG2a antibody is the IgG2a A allotype (IgG2aA). In some embodiments, the mouse IgG2a antibody is the IgG2a B allotype (IgG2aB). Descriptions of murine antibodies can be found, for example, in Han et al., ACS Omega. 2020 Apr. 21; 5(15): 8564-8571, the content of which is incorporated by reference in its entirety for all purposes. In some embodiments, the constant region of the IgG1 antibody comprises or consists of a sequence having at least 80%, at least 85%, at least 90%, at least 95%, at least 98%, at least 99%, or 100% identity to SEQ ID NO: 500 (Uniprot ID: P01868). In some embodiments, the constant region of the IgG2a antibody comprises or consists of a sequence having at least 80%, at least 85%, at least 90%, at least 95%, at least 98%, at least 99%, or 100% identity to SEQ ID NO: 501 (Uniprot ID: P01864). In some embodiments, the constant region of the IgG2b antibody comprises or consists of a sequence having at least 80%, at least 85%, at least 90%, at least 95%, at least 98%, at least 99%, or 100% identity to SEQ ID NO: 502 (Uniprot ID: P01867). In some embodiments, the constant region of the IgG2c antibody comprises or consists of a sequence having at least 80%, at least 85%, at least 90%, at least 95%, at least 98%, at least 99%, or 100% identity to SEQ ID NO: 503 (Uniprot ID: A0A0A6YY53). In some embodiments, the constant region of the IgG3 antibody comprises or consists of a sequence having at least 80%, at least 85%, at least 90%, at least 95%, at least 98%, at least 99%, or 100% identity to SEQ ID NO: 504 (Uniprot ID: A0A075B5P5).

In some embodiments, the IgG antibody is a rat antibody. In some embodiments, the rat IgG antibody is IgG1, IgG2a, IgG2b, or IgG2c subclass. In some embodiments, the rat IgG antibody is IgG1 subclass. In some embodiments, the rat IgG antibody is IgG2a subclass. In some embodiments, the rat IgG antibody is IgG2b subclass. In some embodiments, the rat IgG antibody is IgG2c subclass. Descriptions of rat antibodies can be found, for example, in Kinoshita and Ross, J Immunoassay. 1993 September; 14(3):149-66, the content of which is incorporated by reference in its entirety for all purposes. In some embodiments, the constant region of the IgG1 antibody comprises or consists of a sequence having at least 80%, at least 85%, at least 90%, at least 95%, at least 98%, at least 99%, or 100% identity to SEQ ID NO: 505 (Uniprot ID: P20759). In some embodiments, the constant region of the IgG2a antibody comprises or consists of a sequence having at least 80%, at least 85%, at least 90%, at least 95%, at least 98%, at least 99%, or 100% identity to SEQ ID NO: 506 (Uniprot ID: P20760). In some embodiments, the constant region of the IgG2b antibody comprises or consists of a sequence having at least 80%, at least 85%, at least 90%, at least 95%, at least 98%, at least 99%, or 100% identity to SEQ ID NO: 507 (Uniprot ID: P20761). In some embodiments, the constant region of the IgG2c antibody comprises or consists of a sequence having at least 80%, at least 85%, at least 90%, at least 95%, at least 98%, at least 99%, or 100% identity to SEQ ID NO: 508 (Uniprot ID: P20762).

In some embodiments, the IgG antibody is a rabbit antibody. In some embodiments, the constant region of the IgG antibody comprises or consists of a sequence having at least 80%, at least 85%, at least 90%, at least 95%, at least 98%, at least 99%, or 100% identity to SEQ ID NO: 509 (Uniprot ID: P01870).

In some embodiments, the antibody is an IgM. In some embodiments, the antibody is an IgA. In some embodiments, the antibody is an IgD. In some embodiments, the antibody is an IgE.

In some embodiments, the antibody is a chicken antibody. In some embodiments, the antibody is an IgY antibody.

In some embodiments, the antibody is a heavy-chain antibody.

In some embodiments, the antibody is a mouse antibody, a rat antibody, a rabbit antibody, a chicken antibody (e.g., IgY), a guinea pig antibody, a donkey antibody, a human antibody, a goat antibody, a pig antibody, a horse antibody, or a cattle antibody. In some embodiments, the antibody is a mouse antibody. In some embodiments, the antibody is a rat antibody. In some embodiments, the antibody is a rabbit antibody. In some embodiments, the antibody is a chicken antibody.

### Production of Multivalent Linkers

The present disclosure teaches methods of producing multivalent linkers. In some embodiments, the multivalent linkers of the present disclosure comprise the general structure (Peptide Binding arm)-(Linker Segment)-(Peptide Binding arm). In some embodiments the linker segment is a polypeptide. Thus, in some embodiments, the multivalent linker is a polypeptide that can be encoded in and expressed from nucleic acids. Thus, in some embodiments, the multivalent linkers of the present disclosure are made via in vivo or in vitro expression (e.g., translation). Persons having skill in the art will be familiar with techniques for creating translational fusions, (see for example, Sambrook et al., Molecular Cloning: A Laboratory Manual, Cold Springs Harbor Laboratory, 1992) New York which is incorporated by reference herein in its entirety).

Briefly, in some embodiments, a vector is designed to express, in a linear and, in frame fashion, a polypeptide comprising [a peptide binding arm selected from SEQ ID NO: 1-418]-[an amino acid linker segment as described herein]-[a peptide binding arm selected from SEQ ID NO: 1-418]. In some embodiments the plurality of peptide binding arms can be the same. In some embodiments, the plurality of peptide binding arms can be different.

### Methods of Use

The multivalent linkers of the present disclosure can generally be used for any method that requires attachment of a compound to an antibody or other epitope.

For example, the method may comprise the detection of a target antigen by optical detection, isotopic detection, or detection by electron microscopy. For example, the methods of the disclosure may be a microscopy method, such as a fluorescent microscopy method or an immunofluorescence method.

The methods of the disclosure may also comprise immunofluorescence detection. In some embodiments, the method comprises cyclic immunofluorescence detection.

The methods of the disclosure may also comprise spatial genomic analysis.

The methods of the disclosure may also comprise a flow cytometry or a fluorescence-activated cell sorting (FACS) method. The methods of the disclosure may also comprise a Western Blot.

The methods of the disclosure may also be an immunohistochemistry method. The methods of the disclosure may also be an ELISA method.

In some embodiments, the method of the disclosure encompasses contacting a second monovalent antibody with a first antibody (e.g., when performing ELISA).

The methods of the disclosure may also comprise screening of antibodies or molecules comprising an antigen binding fragment thereof. In some embodiments, the method comprises screening of hybridomas. In some embodiments, the method comprises labeling the supernatant (e.g., hybridoma supernatant) with the multivalent linker of this disclosure.

The methods of the disclosure may also comprise mass spectroscopy.

In some embodiments, the method comprises detecting a test antigen in a sample. In some embodiments, the method comprising the steps of:
A) contacting the sample with the multivalent linker and a binding agent capable of specifically binding to the test antigen,
B) removing unbound binding agent and multivalent linker, and
C) measuring the presence of the multivalent linker in the sample.

In some embodiments, the multivalent linker forms a molecular complex with the binding agent through its target antigen unit.

In some embodiments, the method comprises detecting one or more test antigens in a sample comprising contacting the sample with one or more of the molecular complexes, wherein the molecular complexes are formed by a single binding agent with the corresponding multivalent linker, wherein the binding agent is capable of specifically binding to the test antigen. In some embodiments, the method comprises detecting at least 2, at least 3, at least 4, at least 5, at least 6, at least 7, at least 8, at least 9, or at least 10, test antigens.

In some embodiments, the method comprises detecting two or more test antigens. In some embodiments, the method comprises contacting the sample with a first binding agent specific for a first test antigen, a second binding agent specific for a second test antigen, optionally a third binding agent specific for a third test antigen, and so on, and add multivalent linkers that can specifically bind to the each of the binding agents, respectively. In some embodiments, a multivalent linker can be added earlier, simultaneously, or later than the corresponding binding agent. In some embodiments, the method comprises contacting the sample with a first molecular complex specific for a first test antigen and a second molecular complex specific for a second test antigen, and optionally a third molecular complex for a third test antigen, and so on. In some embodiments, the method comprises contacting the sample with a mixture of molecular complex and binding agents, and adding the corresponding multivalent linker for the binding agents later. Thus, in some embodiments, a first test antigen may be detected by directly adding the corresponding molecular complex to the sample, and the second test antigen may be detected by adding, separately, the binding agent and the corresponding multivalent linker to the sample, or vice versa.

In some embodiments, less than 5%, 4%, 3%, 2%, or 1% of the multivalent linkers bind to two or more of the binding agents (cross-linker two or more binding agents).

In some embodiments, less than 5%, 4%, 3%, 2%, or 1% of the first multivalent linker binds to the second binding agent, and at the same time less than 5%, 4%, 3%, 2%, or 1% of the second multivalent linker binds to the first binding agent. In some embodiments, less than 5%, 4%, 3%, 2%, or 1% of each type of the different multivalent linkers bind to an off-target binding agent. In some embodiment, the ratio is less than 1% for each type of the different multivalent linkers.

In some embodiments, the binding agent is an antibody. In some embodiments, the binding agent comprises an antigen binding fragment of an antibody.

In some embodiments, the two or more binding agent comprises the same target antigen unit (or epitope) that the same multivalent linker specifically binding to, but pre-mixing a binding agent with the multivalent linker that has a unique reporter before adding it to the sample, the method prevents or minimizes the binding of each multivalent linker with a unique reporter to off-target binding agent within the time frame of the method because of the slow dissociation rate.

In some embodiments, the method of the disclosure needs quenching the unbound multivalent linkers so that they do not bind to unintended targets. In some embodiments, the quenching is accomplished by adding a decoy molecule (i.e., quencher) that can bind to the multivalent linker. In some embodiments, the decoy molecule comprises the epitopes or the target antigen unit recognized by the multivalent linker but the decoy molecule lacks the ability to bind to any of the test antigens. In some embodiments, the decoy molecule is an unspecific antibody, or a fragment thereof (e.g., an Fc fragment). In some embodiments, the decoy molecule is an Fc fragment. In some embodiments, the unspecific antibody is an IgG. In some embodiments, the unspecific antibody is polyclonal. In some embodiments, the unspecific antibody is monoclonal.

In some embodiments, the decoy molecule may contain a tag or a moiety that facilitates the removal of itself (and along with the excess multivalent linker).

In some embodiments, the method comprises removal of unbound multivalent linkers from multivalent linker-binding agent complexes.

In some embodiments, the unbound multivalent linkers are removed by ultrafiltration. In some embodiments, the unbound multivalent linkers are removed by bead depletion.

### Therapeutic Agents

In some embodiments, the multivalent linkers of the present disclosure can be labeled with cargo that includes but is not limited to fluorescent dyes, haptens (e.g. biotin), contrast agents (e.g. gadolinium, radionuclides), chelated metals, therapeutic agents, sensitizers, small molecules, or combinations thereof. In some embodiments, a therapeutic agent is attached to the multivalent linker.

In some embodiments, the therapeutic agent is chemotherapeutic agent, a therapeutic antibody, a nucleic acid molecule, a radioisotope, a thymidylate synthase inhibitor, or a platinum compound. Exemplary examples of each are found in U.S. Pat. No. 10,888,618, which is incorporated by reference in its entirety.

### Buffers

In some embodiments, the multivalent linkers are prepared in pharmaceutically acceptable carriers, buffers, or excipients that are nontoxic and/or stabilize the multivalent linker. In some embodiments, the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

Reporter and/or Effectuator Molecules

In some embodiments, a reporter molecule is attached to the multivalent linker. Suitable substances for attachment to multivalent linkers include, but are not limited to, an amino acid, a peptide, a protein, a polysaccharide, a nucleoside, a nucleotide, an oligonucleotide, a nucleic acid, a hapten, a drug, a hormone, a lipid, a lipid assembly, a synthetic polymer, a polymeric microparticle, a biological cell, a virus, a fluorophore, a chromophore, a dye, a toxin, a hapten, an enzyme, an antibody, an antibody fragment, a radioisotope, solid matrixes, semi-solid matrixes and combinations thereof. Patents teaching the use of such labels include U.S. Pat. Nos. 3,817,837; 3,850,752; 3,939,350; 3,996,345; 4,277,437; 4,275,149; and 4,366,241 and are incorporated herein by reference in their entirety.

In some embodiments, the multivalent linker of the present disclosure is labeled with a radioactive isotope, including, but not limited to At211, Cu64, 1131, 1125, Y90, Re186, Re188, Sm153, Bi212, P32, Zr89 and radioactive isotopes of Lu.

In some embodiments, the reporter molecule is genetically linked with the multivalent linker. For example, the reporter molecule may be a fluorescent molecule, such as green fluorescent protein (GFP). To be genetically linked, the sequence for GFP will be in-frame with the sequence for the multivalent linker. The expression or transcription of the coding sequence may be under the influence or control of the same promoter. Therefore, expression from a promoter will express the reporter molecule and multivalent linker in the same polypeptide.

In some embodiments, the reporter molecule is on the c-terminus of the multivalent linker. In some embodiments, a sequence encoding for multivalent linker does not comprise a stop codon at the end of the peptide binding arm, but instead includes one after a sequence encoding a reporter molecule.

In some embodiments, the reporter molecule is on the n-terminus of the multivalent linker.

In some embodiments, reporter/effectuator molecules are attached via other means. For example, in some embodiments reporters and effectuators can be attached via Light Activated Site-Specific Conjugation of Native IgGs (Bioconjugate Chem. 2015, 26, 8, 1456-1460).

### Fluorescent Reporters

In some embodiments, the multivalent peptide is conjugated to a fluorescent reporter. In some embodiments, the fluorescent reporter includes, but is not limited to, coumarins, cyanines, benzofurans, quinolines, quinazolinones, indoles, benzazoles, borapolyazaindacenes, and xanthenes, including fluoresceins, rhodamines, and rhodols.

In some embodiments, the fluorescent reporter is green fluorescent protein (GFP). GFP refers to a polypeptide having a peak in the emission spectrum at 510 nm or about 510 nm. Various fluorescent proteins (FPs) that emit at various wavelengths are known in the art. FPs of interest are green fluorescent protein (GFP), yellow fluorescent protein (YFP), orange fluorescent protein (OFP), cyan fluorescent protein (CFP), blue fluorescent protein (BFP), red fluorescent protein (RFP), far red including but not limited to fluorescent proteins, or near infrared fluorescent proteins. As used herein, Aequorea GFP refers to GFP from the genus Aequorea and mutants or variants thereof. Such mutants and other species such as Anthozoa reef coral, Anemonia sea anemone, Renilla, Galaxea coral, Acropora brown coral, Trachyphyllia and peti GFPs from Pectimidae coral and other species are well known and available and known to those skilled in the art. Additional GFP variants include, but are not limited to, BFP, CFP, YFP and OFP. Examples of fluorescent proteins and variants thereof include GFP proteins such as Emerald (Invitrogen, Carlsbad, Calif.), EGFP (Clontech, Palo Alto, Calif.), Azami-Green (MBL International, Woburn, Mass.), Kaede (MBL International, Woburn, Mass.), ZsGreenl (Clontech, Palo Alto, California), and CopGFP (Evrogen/Axxora, LLC, San Diego, CA); CFP proteins such as Cerulean (Rizzo, Nat Biotechnol. 22 (4): 445-9 (2004)), mCFP (Wang et al., PNAS USA. 101 (48): 16745-9 (2004)), AmCyan1 (Clontech, Palo Alto, CA), MiCy (MBL International, Woburn, Massachusetts) State), and CyPet (Nguyen and Daugherty, Nat Biotechnol. 23 (3): 355-60 (2005)); BFP proteins such as EBFP (Clontech Palo Alto, California); YFP proteins such as EYFP (Clontech, Palo Alto, California), Ypet (Nguyen and Daugherty, Nat Biotechnol. 23 (3): 355-60 (2005)), Venus (Nagai et al., Nat.Biotechnol. 20 (1): 87-90 (2002)), Zs Yellow (Clontech, Palo Alto, CA), and mCitrine (Wang et al., PNAS USA. 101 (48): 16745-9 (2004) OFP proteins such as cOFP (Strategene, La Jolla, CA), mKO (MBL International, Woburn, Mass.), And mOrange; and others (Shaner NC, Steinbach P A, and Tsien R Y., Nat Methods. 2 (12): 905-9 (2005)).

In some embodiments, the fluorescent reporter is red fluorescent protein (RFP). In some embodiments the RFP is Discosoma RFP (DsRed) isolated from corallimorph Discosoma (Matz et al., Nature Biotechnology 17: 969-973 (1999)), and any other optional species, such as red or far-red fluorescent proteins from Heteractis reef corals and Actinia or Entacmaea sea anemones, and variants thereof. RFP is, for example, monomeric red fluorescent protein 1 (mRFP1), mCherry, tdTomato, mStrawberry, mTangerine (Wang et al., PNAS USA. 101 (48): 16745-9 (2004)), DsRed2 (Clontech, Palo Alto, California), and DsRed-T1 (Bevis and Glick, Nat. Biotechnol., 20: 83-87 (2002)), Anthomedusa J-Red (Evrogen) and Anemonia AsRed2 (Clontech, Palo Alto, California) Includes discosome variants. Far-red fluorescent proteins include, for example, Actinia AQ143 (Shkrob et al., Biochem J. 392 (Pt 3): 649-54 (2005)), Entacmaea eqFP611 (Wiedenmann et al. Proc Natl Acad Sci USA. 99 (18): 1 1646-51 (2002)), discosomal variants such as mPlum and mRasberry (Wang et al., PNAS USA. 101 (48): 16745-9 (2004)), and Heteractis HcRedl and t-HcRed (Clontech, Palo Alto, California).

### Non-Fluorescent Reporters

The term “non-fluorescent reporter” as used herein, refers to a chemical moiety that is not fluorescent but which can be used to provide the contrast or signal in imaging and is detectable by a non-fluorescent imaging technique. In certain embodiments, other non-fluorescent reporters can be chemically linked with the imaging agents, or can be administered to a subject simultaneously or sequentially with the imaging agents of the disclosure. Such reporters can include photoluminescent nanoparticles, radioisotopes, superparamagnetic agents, X-ray contrast agents, and ultrasound agents. A reporter may also comprise therapeutic reporters such as porphyrins, Photofrin®, Lutrin®, Antrin®, aminolevulinic acid, hypericin, benzoporphryrin derivatives used in photodynamic therapy, and radionuclides used for radiotherapy.

### Enzymatic Reporters

The reporter molecule can include one or more enzymatic reporters. In some embodiments, the reporter is horseradish peroxidase. In some embodiments, the reporter is β-galactosidase or alkaline phosphatase. In some embodiments, the enzymatic reporter is acetylcholinesterase. In some embodiments, the enzymatic reporter is a binding pair capable of forming a complex. In some embodiments, the binding pair is streptavidin and biotin. In some embodiments, the binding pair is avidin and biotin. Other enzymatic reporters are well known in the art and are further described in van Rossum et al., which is incorporated by reference in its entirety (van Rossum, T., Kengen, S. W. M. and van der Oost, J. (2013), Reporter-based screening and selection of enzymes. FEBS J, 280: 2979-2996.)

### Radioactive Reporters

The reporter molecule can include one or more radioactive labels. Radioisotopic forms of metals such as copper, gallium, indium, technetium, yttrium, and lutetium can be chemically linked to the multivalent linker. Exemplary radioactive labels include, without limitation, ³H, ³⁵S, ¹⁴C, ³²P, ^{99m}TC, ¹¹¹In, ⁶⁴Cu, ⁶⁷Ga, ¹⁸⁶Re, ¹⁸⁸Re, ¹⁵³Sm, ¹⁷⁷Lu, and ⁶⁷Cu.

Additional labels can include, for example, ¹²³I, ¹²⁴I, ¹²⁵I, ¹¹C, ¹³N, ¹⁵O, and ¹⁸F. Additional labels can be therapeutic radiopharmaceuticals including for example ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, and ¹⁹²Ir.

Chelators or bonding moieties can be chemically associated with the multivalent linker. Chelators can be selected to form stable complexes with radioisotopes that have imagable gamma ray or positron emissions, such as ^{99m}Tc, ¹¹¹In, ⁶⁴Cu, and ⁶⁷Ga. Exemplary chelators include diaminedithiols, monoamine-monoamidedithiols, triamide-monothiols, monoamine-diamide-monothiols, diaminedioximes, and hydrazines. Chelators generally are tetradentate with donor atoms selected from nitrogen, oxygen and sulfur, and may include for example, cyclic and acyclic polyaminocarboxylates such as diethylenetriaminepentaacetic acid (DTPA), 1,4,7,10-tetraazacyclododecane-1,4,7,10-tetraacetic acid (DOTA), (DO3A), 2-benzyl-DOTA, alpha-(2-phenethyl)1,4,7,10-tetraazazcyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl]-4′-(3-amino-4-methoxyphenyl)-2,2′:6′,2″-terpyridine.

Chelators or bonding moieties can be selected to form stable complexes with the radioisotopes that have alpha particle, beta particle, Auger or Coster-Kronig electron emissions, such as ¹⁸⁶Re, ¹⁸⁸Re, ¹⁵³Sm, ¹⁷⁷Lu, and ⁶⁷Cu. Chelators can be selected from diaminedithiols, monoamine-monoamidedithiols, triamide-monothiols, monoamine-diamide-monothiols, diaminedioximes, and hydrazines, cyclic and acyclic polyaminocarboxylates such as DTPA, DOTA, DO3A, 2-benzyl-DOTA, alpha-(2-phenethyl)1,4,7,10-tetraazacyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl]-4′-(3-amino-4-methoxyphenyl)-2,2′:6′,2″-terpyridine.

### Metal Reporters/Effectuators

In some embodiments, the reporter molecule can include one or more magnetic labels. Other exemplary reporters can include a chelating agent for magnetic agents. Such chelators can include for example, polyamine-polycarboxylate chelators or iminoacetic acid chelators that can be chemically linked to the multivalent linker.

Magnetic reporters can be selected to form stable complexes with paramagnetic metal ions, such as Gd(III), Dy(III), Fe(III), and Mn(II), are selected from cyclic and acyclic polyaminocarboxylates such as DTPA, DOTA, DO3A, 2-benzyl-DOTA, alpha-(2-phenethyl)1,4,7,10-tetraazacyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl]-4′-(3-amino-4-methoxyphenyl)-2,2′:6′,2″-terpyridine.

In some embodiments, the multivalent linkers are chemically linked to superparamagnetic metal oxide nanoparticles that are either (a) non-fluorescent or (b) are fluorescent and can be used in a variety of in vitro and in vivo applications.

### Oligo Reporters

In some embodiments the multivalent linkers of the present disclosure comprise one or more oligonucleotide reporters. Persons having skill in the art will be familiar with various natural and synthetic oligonucleotides that can be attached to antibodies, or multivalent linkers of the present disclosure alike, including those discussed in Evaluation of oligonucleotide conjugated antibodies as reporter molecules in single-cell assays, Takahashi et al., The Journal of Immunology May 1, 2020, 204 (1 Supplement) 86.35; Pharmaceutics. 2020 June; 12(6): 545. Published online 2020 Jun. 12. Doi: 10.3390/pharmaceutics12060545; and Kennedy-Darling et al, (2021), Highly multiplexed tissue imaging using repeated oligonucleotide exchange reaction. Eur. J. Immunol., 51: 1262-1277.

### Compositions Comprising Multivalent Linkers

In some embodiments, the multivalent linkers or the molecular complexes are prepared in pharmaceutically acceptable carriers, buffers, or excipients that are nontoxic and/or stabilize the multivalent linker or the molecular complex. In some embodiments, the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, the composition may be administered in a form suitable for any route of administration, including e.g., orally in the form tablets, capsules, or liquid, or in sterile aqueous solution for injection. The composition may be administered orally in the form of tablets, capsules, ovules, elixirs, solutions or suspensions, gels, syrups, mouth washes, or a dry powder for constitution with water or other suitable vehicle before use, optionally with flavoring and coloring agents for immediate-, delayed-, modified-, sustained-, pulsed- or controlled-release applications. Solid compositions such as tablets, capsules, lozenges, pastilles, pills, boluses, powder, pastes, granules, bullets, or premix preparations can also be used. Solid and liquid compositions for oral use can be prepared according to methods well known in the art. Such compositions can also contain one or more pharmaceutically acceptable carriers and excipients which can be in solid or liquid form. When the composition is formulated for oral administration, the tablets or capsules can be prepared by conventional means with pharmaceutically acceptable excipients such as binding agents (e.g., pregelatinized starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art.

The pharmaceutically acceptable excipients also include, but are not limited to, microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropyl ethylcellulose (HPMC), hydroxypropyl cellulose (HPC), sucrose, gelatin, and acacia. Additionally, lubricating agents such as magnesium stearate, stearic acid, glyceryl behenate and talc can be included.

In some embodiments, the composition comprises a multivalent linker and a binding agent (e.g., a primary antibody).

In some embodiments, the composition comprises a first multivalent linker and a first binding agent (e.g., a primary antibody). In some embodiments, the composition comprises a second multivalent linker and a second binding agent (e.g., a primary antibody). In some embodiments, the first and second binding agents (e.g., primary antibodies) are from the same species. In some embodiments, the first and second binding agents (e.g., primary antibodies) are from different species.

In some embodiments, the composition comprises a multivalent linker, a binding agent (e.g., a primary antibody), and an antigen.

In some embodiments, the composition comprises a first multivalent linker, a first binding agent (e.g., a primary antibody), and a first antigen. In some embodiments, the composition comprises a second multivalent linker, a second binding agent (e.g., a second primary antibody), and a second antigen. In some embodiments, the first and second binding agent (e.g., primary antibodies) are from the same species. In some embodiments, the first and second binding agents (e.g., primary antibodies) are from different species.

In some embodiments, the binding agent is or comprises a target antigen unit, as described in the present disclosure.

Labeling Step with Multivalent Linker

In some embodiments, the present disclosure teaches a labeling step of the multivalent linker with a primary binding agent (e.g., a primary antibody) prior to conducting the experimental assay. In some embodiments, labeling allows for use of primary binding agents from the same species, or with shared epitopes.

In some embodiments, the multivalent linker labels a primary antibody. In some embodiments, the primary antibody is a monoclonal antibody. In some embodiments, the antibody is not a polyclonal antibody. In some embodiments, the antibody is a polyclonal antibody.

In some embodiments, the primary antibody and multivalent linker are mixed at specific molar ratios.

In some embodiments, the primary binding agent (e.g., primary antibody) to multivalent linker molar ratio is about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, or 1:10.

In some embodiments, the primary binding agent (e.g., primary antibody) to multivalent linker molar ratio is between 1:1-1:1.5, between 1:1.5-1:2, between 1:2-1:2.5, between 1:2.5-1:3, between 1:3-1:3.5, between 1:3.5-1:4, between 1:4-1:4.5, between 1:4.5-1:5, between 1:5-1:5.5, between 1:5.5-1:6, between 1:6-1:6.5, between 1:6.5-1.7, between 1:7-1:7.5, between 1:7.5-1:8, between 1:8-1:8.5, between 1:8.5-1:9, between 1:9-1:9.5, or between 1:9.5-1:10.

In some embodiments, the concentration of the primary binding agent (e.g., primary antibody) should be maintained at a high concentration during the pre-conjugation step.

In some embodiments, the concentration of the primary antibody is at least 0.00001 g/l, at least 0.0001 g/l, at least 0.001 g/l, at least 0.01 g/l, at least 0.05 g/l, at least 0.1 g/l, at least 0.15 g/l, at least 0.2 g/l, at least 0.25 g/l, at least 0.3 g/l, at least 0.35 g/l, at least 0.4 g/l, at least 0.45 g/l, at least 0.5 g/l, at least 0.55 g/l, at least 0.6 g/l, at least 0.65 g/l, at least 0.7 g/l, at least 0.75 g/l, at least 0.8 g/l, at least 0.85 g/l, at least 0.9 g/l, at least 0.95 g/l, or at least 1 g/l.

In some embodiments, the concentration of the primary antibody is between 0.00001 g/l to 0.0001 g/l, between 0.0001 g/l to 0.001 g/l, between 0.001 g/l to 0.01 g/l, between 0.01 g/l to 0.05 g/l, between 0.05 g/l to 0.1 g/l, between 0.1 g/l to 0.15 g/l, between 0.15 g/l to 0.2 g/1, between 0.2 g/l to 0.25 g/l, between 0.25 g/l to 0.3 g/l, between 0.3 g/l to 0.35 g/l, between 0.35 g/l to 0.4 g/l, between 0.4 g/l to 0.45 g/l, between 0.45 g/l to 0.5 g/l, between 0.5 g/l to 0.55 g/l, between 0.55 g/l to 0.6 g/l, between 0.6 g/l to 0.65 g/l, between 0.65 g/l to 0.7 g/l, between 0.7 g/l to 0.75 g/l, between 0.75 g/l to 0.8 g/l, between 0.8 g/l to 0.85 g/l, between 0.85 g/l to 0.9 g/l, between 0.9 g/l to 0.95 g/l, or between 0.95 g/l to 1 g/l, including all ranges and subranges therebetween.

In some embodiments, the binding agent (e.g., primary antibody) is incubated with the multivalent linker for at least 1 second, at least 1 minute, at least 5 minutes, at least 10 minutes, at least 20 minutes, at least 30 minutes, at least 40 minutes, at least 50 minutes, at least 60 minutes, at least 120 minutes, at least 180 minutes, at least 240 minutes, or at least 300 minutes.

In some embodiments, the binding agent (e.g., primary antibody) is incubated with the multivalent linker for at most 1 second, at most 1 minute, at most 5 minutes, at most 10 minutes, at most 20 minutes, at most 30 minutes, at most 40 minutes, at most 50 minutes, at most 60 minutes, at most 120 minutes, at most 180 minutes, at most 240 minutes, or at most 300 minutes.

In some embodiments, the binding agent (e.g., primary antibody) is incubated with the multivalent linker for between 1 second to 1 minute, between 1 minute to 5 minutes, between 5 minutes to 10 minutes, between 10 minutes to 20 minutes, between 20 minutes to 30 minutes, between 30 minutes to 40 minutes, between 40 minutes to 50 minutes, between 50 minutes to 60 minutes, between 60 minutes to 120 minutes, between 120 minutes to 180 minutes, between 180 minutes to 240 minutes, or between 240 minutes to 300 minutes, including all ranges and subranges therebetween.

In some embodiments, the glycerol content is less than 90%, is less than 80%, is less than 70%, is less than 60%, is less than 50%, is less than 40%, is less than 30%, is less than 20%, is less than 10%, is less than 5%, is less than 4%, is less than 3%, is less than 2%, is less than 1%, is less than 0.5%, or is less than 0.1%.

### EXAMPLES

### Example 1: Synthesizing Phage Display Library

As noted above, in some embodiments, the peptide binding arms can be sourced from antibodies (e.g. VHH or single domain antibodies (sdAbs)). Peptide binding arms of the present disclosure can be generated via the immunization of mammals, including camelids or rodents, with target antigens. Depending on the antigen, peptide binding arms with even picomolar affinities can be identified without additional in vitro maturation steps. The binding regions of the identified antibodies (e.g., from camels), can then be used as peptide binding arms in the methods and compositions of the present disclosure.

Methods for isolating antigen-specific antibodies from immune antibody libraries by phage display and other display techniques are known since the 1990s, well established and the protocols are routinely used. For immune library generation, a mammal (e.g. a camelid or rodent) will be immunized with a defined amount of the target antigen over a certain period. After the immunization phase antigen secreting B cells will be isolated from the peripheral blood, total mRNA will be extracted and transcribed into cDNA. Subsequently several PCR steps will be conducted to amplify the binding variants and to generate a large and diverse phage library representing the full immune antibody repertoire (Pleiner et al., 2015).

### Example 2: Phage Display Panning and Screening Process

The phage display screening method described above was utilized to provide a high-throughput and unbiased method to identify peptide binding arms for use in the multivalent linkers.

Phage display assays were conducted according to methods known in the art and further as described in Pande et al. and Wu et al. (Pande, J et al. Phage display: Concept, innovations, applications and future. Biotechnology Advances 28 (2010) 849-858; Wu, C H., Liu, I J., Lu, R M. Et al. Advancement and applications of peptide phage display technology in biomedical science. J Biomed Sci 23, 8 (2016)).

Briefly, Phage display is based on genotype-phenotype coupling, i.e. a plasmid+phage (phagemid) encodes for a multivalent linker fused to one of the coat proteins of the phage (e.g. pIII). The expressed multivalent linker is linked to the coat protein and presented by the phage on its surface. During the panning process, multivalent linker fragments from a diverse library binding to an antigen of interest are enriched and selected. In general, the selection process is done as follows:
1. Binding of the multivalent linker-phage complex to an antigen in solution or immobilized on a plate.
2. Non-binding multivalent linker-phage complexes are washed away with several washing steps, whereby only the bound antigen-binding complexes remain.
3. The antigen-specific candidates are then eluted, e.g. via a pH-shift.
4. The free multivalent linker-phage complexes can then be used for infection of bacteria and phage amplification, which can be used as input for a subsequent selection round to further enrich for candidates with desired properties.

After several selection rounds, single clones are used for high throughput screening of the encoded multivalent linker. For screening, single colonies are picked, the multivalent linker are expressed by E. coli in 96-well format and tested for antigen binding as bacterial lysate, e.g. in ELISA. Positive, so-called “primary” hits can then be further characterized in additional screenings, e.g. for cell binding, cross-reactivity, or binding strength.

For in-depth characterization of promising candidates, the multivalent linkers are expressed and purified. For example, accurate affinities can be determined with the purified candidates, and final characterization in the various assays and applications can be conducted.

The screen successfully identified several hundred candidates (see, e.g., SEQ ID NO: 1-418), which were further characterized.

### Example 3: Production of Multivalent Linkers

Nucleotide sequences from the phage display library described above were utilized to clone, express and purify peptide binding arms for use in the multivalent linkers.

Cloning, protein expression and protein purification were conducted according to methods known in the art and further as described in Sambrook et al., Molecular Cloning: A Laboratory Manual, Cold Springs Harbor Laboratory, 1992) New York.

Nucleotides encoding a protein binding head as produced in Examples 1 and 2 with a peptide linker segment (e.g., from SEQ ID NO: 1-418) were cloned into a mammalian expression vector and transfected into mammalian cells. Stable or transient transfection into mammalian cells are both suitable. Alternatively, the nucleotide may be cloned into a bacterial expression vector. Both stable and transient transfection into bacterial cells and/or yeast cells or any other system are suitable.

The mammalian cells were incubated at 37° C. with 5% CO₂ for several days, including daily media changes.

Supernatants containing the multivalent linkers were purified through affinity chromatography. Other methods, such as cation/anion exchange, size exclusion and/or hydrophobic interaction chromatography, are additionally suitable. The multivalent linkers were concentrated by selective filtration and formulated in a buffer that stabilizes free tetramers.

Several dozen multivalent proteins from Table 1 and Table 2 were successfully cloned, expressed, purified, and further characterized.

### Example 4: Attachment of Labels to Multivalent Linkers

Selected multivalent linkers were covalently linked with a fluorophore label using standard maleimide labeling chemistry. (See e.g., Jagpreet S. Nanda, Jon R. Lorsch, Chapter Seven—Labeling of a Protein with Fluorophores Using Maleimide Derivitization).

Persons having skill in the art will recognize that other labels will be compatible with the multivalent linkers of the present disclosure, including those disclosed herein and in EP3596464 and WO2022115791A1, which are incorporated by reference in their entirety.

### Example 5: Labeling of Primary IgG with Multivalent Linkers

### Initial Labeling

In some embodiments, the present disclosure teaches multivalent linkers that are already bound to primary antibodies. Thus in some embodiments, the present disclosure teaches methods of labeling primary antibodies with multivalent linkers.

For example, in order to use multivalent linkers with multiple primary antibodies of the same species, an initial labeling step is carried out to bind each multivalent linker to the desired a primary IgG. The primary IgG may be from any species.

The multivalent linkers disclosed in Tables 1 and 2 are incubated with the corresponding primary IgG were incubated in a ratio of IgG:multivalent peptide=1:2.5. The multivalent linkers bind to the Fc region of each primary antibody. (Note multivalent linkers can be—and have been—targeted to other conserved regions of primary antibodies, such as conserved light chain regions.) Incubation was performed in PBS and carried out for 0.5 h at RT in the dark under shaking (700 rpm). In general, the IgG concentration during the pre-conjugation step should be kept as high as possible (>0.01 g/l). The primary antibody concentration in a specific reaction has been shown to work at concentrations as low as 0.5 ug/ul. The reactions can be performed with antibodies in any formulations, including glycerol or sodium azide.

Next, the multivalent linkers that bind specifically to the Fc domain of a primary IgG were incubated in a ratio of IgG:multivalent linker=1:2.5. Incubation was performed in PBS and carried out for 5 minutes at RT in the dark. The primary antibody concentration in a specific reaction can be as low as 0.5 ug/ul.

### Removal of Unbound Multivalent Linker

Any unbound multivalent linker may bind an off-target antigen in an immunoassay, resulting in increased background. Consequently, any unbound multivalent linker should be removed (e.g., via filtration as discussed below), depleted (as discussed below), or quenched (as discussed below).

After labeling as discussed above, unbound multivalent linkers were removed by 1) either using Ultrafiltration (with e.g. Amicon Ultra 0.5 ml, 50 MWCO, Merck Millipore, cat #UFC505024), 2) In other experiments, unbound multivalent linkers were removed by bead depletion (with agarose beads conjugated to the respective IgG: e.g. Rabbit IgG agarose, Sigma, cat #A2909, or Mouse IgG1 Flag M2 agarose, Sigma, cat #A2220). In other experiments, unbound multivalent linkers were quenched by adding unspecific polyclonal IgG containing the same subtype as the used primary IgG, as discussed in more detail below.

### Ultrafiltration

In some instances, the primary antibodies labeled with the multivalent linker were purified before packaging/use. In some instances, the labeled antibodies are subjected ultrafiltration. Labeled mixes of primary antibody and multivalent linkers (50-500 μl) were added to a column, respectively, and centrifuged at 14000 g for 1 min at 4° C. Elution fractions were roughly quantified using a pipette and discarded. Then, the same volume of PBS as the discarded fractions was pipetted to each column again. This purification step was repeated four times. After the last (5^{th}) round, columns were inverted and put into a new tube. Centrifugation was done at 1000 g for 2 min at 4° C. The elution fractions were quantified, and the missing volume compared to the original volume (before purification round 1) was added finally.

### Depletion

For bead depletion, the pre-conjugated mixes were processed by five sequential rounds of IgG-bead incubation in spin columns and centrifugation. In detail, 10 μl of PBS pre-equilibrated IgG agarose slurry was pipetted into a spin column, respectively. Then, the pre-conjugated mixes were added to the spin columns and incubated for 2 min on a thermomixer at RT under shaking (700 rpm). Further, the mixes were centrifuged (1 min, 2500 g @RT) and the eluate used for the next purification round. This purification step was repeated for four rounds for each sample.

### Quenching

For quenching of unbound multivalent linker, nonspecific IgG (e.g., Mouse IgG1 isotype control monoclonal antibody, or IgG control rabbit polyclonal antibody, was added to the pre-conjugated mixes. In detail, each pre-conjugated mix was incubated for 5 min with 10× the amount of the applied primary IgG (e.g. 10 μg unspecific IgG for 1 μg specific primary IgG, deviating amounts are also expected to work.

This removal and quenching step provides several methods to remove unbound multivalent linker, which will decrease the background in the following experiments.

### Example 6: Multivalent Linkers of the Present Disclosure Permit Multiplex Labeling of Primary Antibodies from the Same Species in Immunomicroscopy

### Leaking Experiment

Traditional immunoassays rely on labeled secondary antibodies that bind and label primary antibodies targeting epitopes of interest. These secondary antibodies however, target shared regions within primary antibodies of the same species, which restricts the ability to multiplex using primary antibodies from the same species. The technical issue that arises is that secondary antibodies that were pre-conjugated to a first primary antibody become detached from that antibody, and can then re-bind to another primary antibody of the same species, that may be targeting a different epitope in the sample (i.e., leaking of the label from one primary antibody to another primary antibody of the same species). This essentially causes a label that was originally intended to mark one epitope, to instead mark for a different epitope.

To demonstrate the superior multiplexing functionality of the multivalent linkers of the present disclosure, a leaking experiment was performed that compared the signal leaking from a sample labeled with control monovalent linker versus several multivalent linkers with different linker segment lengths. The results of this experiment are depicted in FIG. 3, and are further described, below.

5 μg of anti-ATP5O primary antibody was pre-labeled with either, i) control monovalent linker (column 1), ii) a bivalent multivalent linker with a (G4S)₃ linker segment (column 2), iii) a bivalent multivalent linker with a (G4S)₄ linker segment (column 3), or iv) a bivalent multivalent linker (G4S)₅ linker segment (column 4). Each of the monovalent linker and multivalent linkers were labeled with AF488 fluorophore.

Selected samples also received 10 μg of unlabeled anti-LaminB1 primary antibody from the same species as the anti-ATP50 antibody. A positive control with labeled anti-laminB1 was also included in the experimental design. The incubation volume was 50 μl which corresponds to a primary dilution of 1:10. To remove unbound multivalent linkers, ultrafiltration was applied as described above (1:20 dilutions of pre-conjugated mixes were used).

The experimental design for this experiment was as follows. Samples labeled ATP50/Linker+Lamin contained the anti-ATP50 primary antibody labeled with the corresponding monovalent or multivalent linker with the AF488 fluorophore; as well as unlabeled anti-lamin primary antibody. Samples labeled ATP50/Linker (positive control) contained only the anti-ATP50 primary antibody labeled with the corresponding monovalent or multivalent linker with the AF488 fluorophore, and no other antibodies. Samples Labeled Lamin/Linker (positive control) contained only anti-Lamin primary antibody labeled with the corresponding monovalent or multivalent linker with the AF488 fluorophore, and no other antibodies.

Lamin is a nuclear-envelope localized protein, such that samples containing fluorophore-labeled anti-Lamin showed a strong nuclear fluorescent signal. (See bottom row of microscopy images of FIG. 3). ATP50 is a mitochondrial protein, such that fluorophore-labeled anti-ATP50 only showed an extra-nuclear signal. (See bottom row of microscopy images of FIG. 3). The top sample in this experiment contained the fluorophore-labeled anti-ATP50, and an unlabeled anti-Lamin primary antibody. The only way in which the anti-Lamin antibody could generate a fluorescent signal in the nuclear envelope would be if the fluorophore label detached itself from the anti-ATP50, and re-attached itself to the Lamin antibody (i.e., if the signal leaked).

By comparing the nuclear fluorescence signal of the various samples, the amount of label leaking could be quantified. Specifically, the nuclear fluorescent signal from the ATP50/Linker+Lamin was divided by the nuclear fluorescent signal of the ATP50/linker (positive control) to create a “relative nuclear signal increase.” A relative nuclear signal increase of 1 would indicate no signal leakage, while a value greater than 1 would indicate that at least a portion of the fluorophore label from the anti-ATP50 had “leaked” to the anti-Lamin antibody. The Lamin/Linker (positive control) sample provided verification that the anti-Lamin antibody could properly target and fluorescently label the nuclear envelope.

As can be seen in FIG. 3, monovalent linkers exhibited significant leaking, showing a relative nuclear signal increase of 1.49 (about 49% signal leaking). Among the multivalent linkers, the (G4S)₄ and (G4S)₅ linker segments provided the best results, with near zero leaking. The multivalent linker with the (G4S)₃ linker segment exhibited slightly higher background, suggesting that perhaps the linker was too short to maintain contact with the primary antibody.

These results demonstrate that the multivalent linkers of the present disclosure are markedly superior to monovalent linkers, and further show the importance of linker segment length.

### Example 7: Multivalent Linkers Permit Multiplex Labeling of Primary Antibodies in Westerns

Multiplexing the multivalent linkers are not limited to immunofluorescence assays and can be used in different types of immunoassays, such as SDS-PAGE Western blots.

Persons having skill in the art will be familiar with the process for preparing western blots. Briefly, desired cell lysate can be separated by SDS-PAGE and blotted on nitrocellulose or PVDF in advance. On the day of the experiment, the blots can be incubated in quenching buffer (e.g. 5% milk powder, 0.075% Tween20 in PBS) until the final mixes are added for incubation.

In our standard WB procedure, we used nitrocellulose membranes blotted with HEK293T cell lysate. After pre-conjugation and purification or quenching of the IgG:multivalent linker mixes they were pre-diluted in the assay buffer (here: 5% BSA, 0.075% Tween20 solution in PBS) at a dilution twice as concentrated as the final dilution was (if the final concentration is 1:1000, they were diluted at 1:500 at this step). Then, the pre-diluted mixes were mixed 1:1 with either unconjugated, competitive IgG (leaking experiment) or conjugated, competitive IgG (multiplexing experiment) to give the final dilution for WB incubation. Mixing pre-diluted IgG:multivalent linker mix 1:1 with buffer alone were used as controls.

The blots were incubated for 0.5-1.0 h at room temperature on a wheel. After incubation, blots were washed three-times for 5 minutes with 0.075% Tween20 solution in PBS. Prior to imaging blots were dried for at least 30 minutes using Whatman filter paper.

Several membranes were blotted with different combinations of pre-labeled multivalent linker+primary antibody complexes.

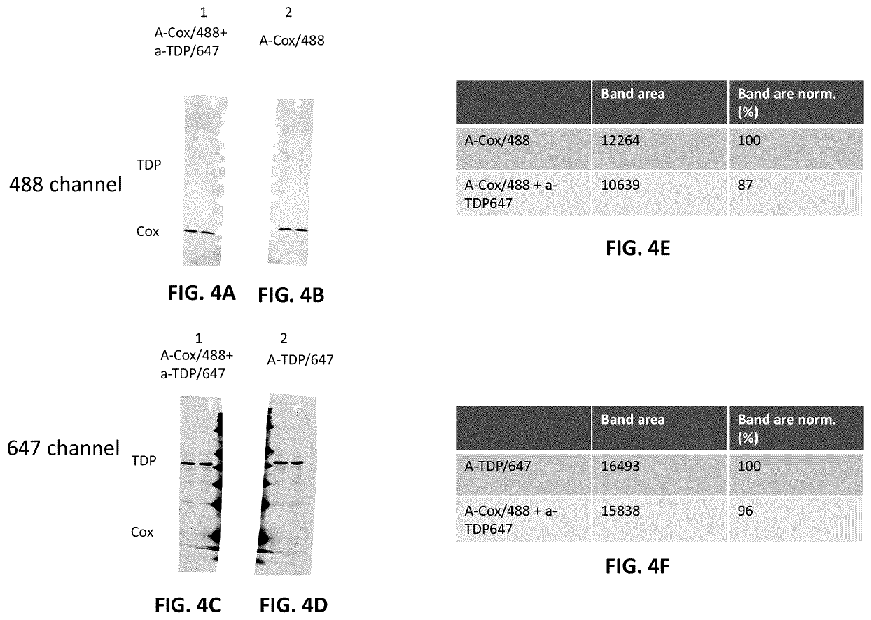

FIG. 4A and FIG. 4C depict a membrane treated with anti-CoxIV primary antibody linked to a multivalent linker with a 488 fluorophore and also a second primary anti-TDP antibody from the same species, labeled with a multivalent linker with a 647 fluorophore. FIG. 4B and FIG. 4D are controls treated with only the anti-CoxIV primary antibody linked to a multivalent linker with a 488 fluorophore or with a primary anti-TDP antibody labeled with a multivalent linker with a 647 fluorophore, respectively.

FIG. 4A and FIG. 4B show the signal as viewed through the 488 excitation frequency channel, while FIG. 4C and FIG. 4D shows the signal as viewed through the 647 excitation frequency channel. The results shows that membranes treated with both labeled primary antibodies from the same species did not exhibit any signal bleed.

This example thus further demonstrates the use of the present multivalent linkers in multiplexing assays.

### Example 8: Multivalent Linkers in Fluorescence-Activated Cell Sorting

Primary antibodies are often stored in glycerol to prevent antibody degradation; however, glycerol concentration can inhibit various applications for the primary antibody. For example, glycerol may inhibit a reporter molecule function, such as peroxidase, or inhibit recognition of the target antigen. Several methods exist to remove glycerol from the primary antibody stock; however, the concentration of the primary antibody may be significantly reduced through these processes. Therefore, it is often preferred to work with reagents that are not negatively impacted by glycerol. This Example demonstrates the multivalent linkers are suitable for flow cytometry experiments at wide range of different glycerol concentrations.

A standard flow cytometry procedure was performed. Briefly, the cells were washed (single cell suspension) and the cell number as adjusted to a concentration of 1-5×10⁶ cells/ml in ice cold FACS Buffer (PBS, 0.5-1% BSA or 5-10% FBS, 0.1% NaN₃ sodium azide). Cells were stained in polystyrene round-bottom 12×75 mm BD Falcon tubes (cat #352052). Next, 100 μl of cell suspension was added to each tube. Next, 100 μl of Fc block was added to each sample (Fc block diluted in FACS buffer at 1:50 ratio) and incubated on ice for 20 min, followed by centrifugation at 1500 rpm for 5 min at 4° C.

To determine if glycerol concentration affects primary antibody-multivalent linker mix formation, six different glycerol concentrations of the primary antibody stock were used: 0%, 5%, 10%, 15%, 20%, and 25%. The multivalent linker was first conjugated to an allophycocyanin (APC) reporter. The multivalent linker-APC reporter were then incubated with a primary antibody targeting an epitope of interest within the cells, thereby labeling that antibody. Next, 10 μg/ml of a primary antibody-multivalent linker complex was added to the cells and incubated for 30 min in the dark. The cells were washed 3 times by centrifugation at 1500 rpm for 5 minutes and resuspend in 1 ml of ice cold FACS buffer. The cells were kept in the dark on ice until the scheduled analysis time.

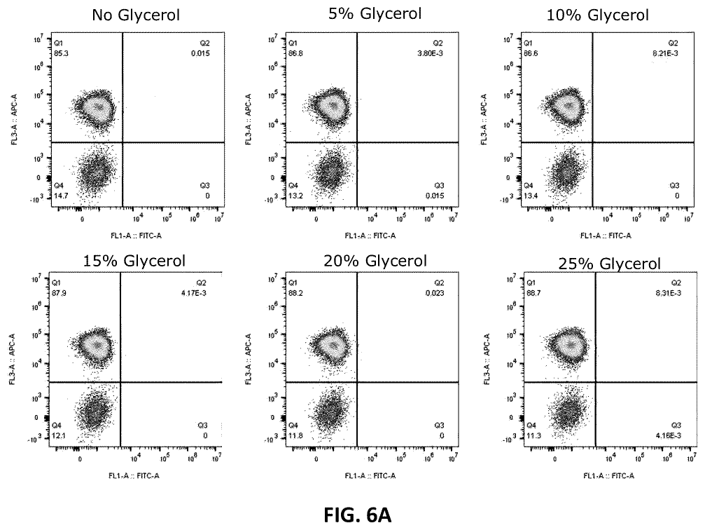

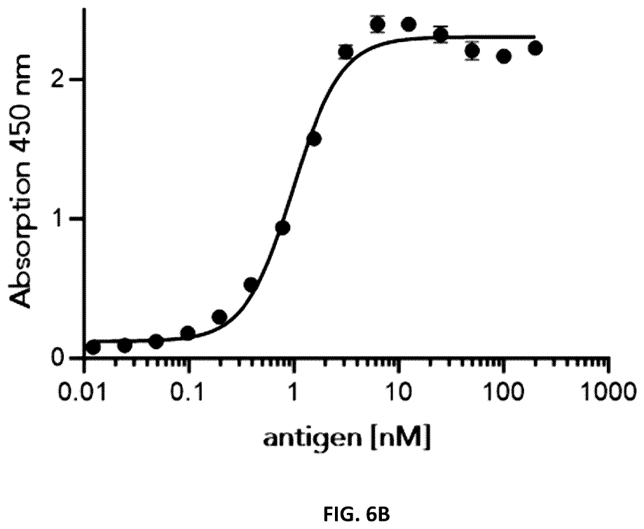

As shown in FIG. 6A and Table 3, various concentrations of glycerol (0-25%) in the primary antibody stock do not affect the number of positive cells, or the ability to distinguish between labeled and unlabeled cell populations. Therefore, forming the primary antibody-multivalent linker mix is suitable at several concentrations of glycerol in FACs experiments.

TABLE 3
Various Primary Antibody Stock Glycerol
Concentrations in FACs Experiment

| % (w/w) Glycerol | Q1% (+) |
|---|---|
| 0 | 85.3 |
| 5 | 86.8 |
| 10 | 86.6 |
| 15 | 87.9 |
| 20 | 86.2 |
| 25 | 88.7 |

This experiment demonstrates that primary antibody-multivalent linker mixes are suitable for flow cytometry using a wide range of primary antibody glycerol concentrations.

### Example 9: Other Envisioned Uses of the Multivalent Linkers

### Immunohistochemistry

Multiplexing experiments using the multivalent linkers may also be performed in immunocytochemistry experiments.

Using a standard immunohistochemistry protocol, tissues will be fixed in paraformaldehyde (4% in buffer) and incubated for around 2 hours at room temperature.

Next, the paraformaldehyde is discarded and the tissue will be washed 5 times for 10 minutes each in 0.1M phosphate buffer solution+0.3% Triton-X. The tissue will then be embedded in paraffin. Thin slices of paraffin-embedded tissue will be sectioned using a microtome. The paraffin-embedded tissue sections will next be transferred to a vial containing a first primary antibody-multivalent linker mix and a second primary antibody-multivalent linker mix, which will be incubated overnight. Following overnight incubation, the paraffin-embedded tissue will be washed in Triton buffer 6 times for 10 minutes each. The paraffin-embedded tissue sections will be kept in the dark on ice or at 4° C. until the scheduled analysis time.

Experiments will be conducted using single linker-antibody combinations, or multiplexed primary antibodies of the same species with multivalent linkers. No signal bleeding or cross-reactivity is expected.

### Flow Cytometry

A multiple flow cytometry example will be conducted to demonstrate the multivalent linker's use in flow cytometry. In a standard flow cytometry procedure, the cells will be washed (single cell suspension) and the cell number will be adjusted to a concentration of 1-5×10⁶ cells/ml in ice cold FACS Buffer (PBS, 0.5-1% BSA or 5-10% FBS, 0.1% NaN₃ sodium azide). Cells will be stained in polystyrene round-bottom 12×75 mm BD Falcon tubes (cat #352052). Next, 100 μl of cell suspension will be added to each tube. Next, 100 μl of Fc block will be added to each sample (Fc block diluted in FACS buffer at 1:50 ratio) and incubated on ice for 20 min, followed by centrifugation at 1500 rpm for 5 min at 4° C.

Next, 0.1-10 μg/ml of a first primary antibody-multivalent linker mix and 0.1-10 μg/ml of a second primary antibody-multivalent linker mix will be added and incubated for at least 30 min in the dark. Dilutions, if necessary, can be made in FACS buffer.

The cells will then be washed 3 times by centrifugation at 1500 rpm for 5 minutes and resuspend them in 200 μl to 1 ml of ice cold FACS buffer. The cells are kept in the dark on ice or at 4° C. until the scheduled analysis time.

Experiments will be conducted using single linker-antibody combinations, or multiplexed primary antibodies of the same species with multivalent linkers. No signal bleeding or cross-reactivity is expected.

### Enzyme-Linked Immunosorbent Assay (ELISA)

An Enzyme-Linked Immunosorbent Assay (ELISA) was to demonstrate use of the multivalent linker's use in this context.

In this ELISA study, the sample antigen, biotinylated GFP, was diluted in varying concentration in coating buffer. The plates were coated with 100 μL per well of coating solution. The plates were then covered and incubate one hour at room temperature or overnight (12-18 hours) at 2-8° C.

The contents was then aspirated and the wells were washed one time with >300 μL of Wash buffer per well. Following the wash, the plate was inverted and tapped on absorbent paper to remove excess liquid.

Next, 300 μL of blocking buffer was added per well for one hour at room temperature, followed by aspirating the blocking buffer, inverting the plate, and tapping the plate on absorbent paper to remove excess liquid.

Next, the primary anti-GFP antibody plus HRP-multivalent linker was diluted in blocking buffer, added to each well, and incubated for two hours at room temperature with gentle continual shaking (˜500 rpm).

The contents were then aspirated and washed five times with >300 μL of wash buffer per well. Following the wash, the plate was inverted and taped on absorbent paper to remove excess liquid. The relative fluorescent units (RFU) was then measured on a standard plate reader. The result (FIG. 6B) demonstrates excellent antigen binding measurements using the primary antibody labeled with the multivalent linker.

Another standard sandwich ELISA study will be conducted to demonstrate use of the multivalent linker's use in this context. In this study, a solution will be prepared by diluting capture antibodies in coating buffer. The plates will be coated with 100 μL per well of coating solution. The plates will then be covered and incubate one hour at room temperature or overnight (12-18 hours) at 2-8° C.

The contents will then be aspirated and the wells will be washed one time with >300 μL of Wash buffer per well. Following the wash, the plate will be inverted and tapped on absorbent paper to remove excess liquid.

Next, 300 μL of blocking buffer will be added per well for one hour at room temperature, followed by aspirating the blocking buffer, inverting the plate, and tapping the plate on absorbent paper to remove excess liquid.

Standards and sample dilutions will be prepared in blocking buffer. 100 μL of standards and samples (in duplicate) will be pipetted into designated wells. The plate will be incubated for one to two hours at room temperature with gentle continual shaking (˜500 rpm).

The contents will then be aspirated and washed wells five times with >300 μL of wash buffer per well. Following the wash, the plate will be inverted and tapped on absorbent paper to remove excess liquid.

Next, a first primary antibody-multivalent linker mix and a second primary antibody-multivalent linker mix were diluted in blocking buffer, added to each well, and incubated for two hours at room temperature with gentle continual shaking (˜500 rpm).

The contents will then be aspirated and washed five times with >300 μL of wash buffer per well. Following the wash, the plate will be inverted and taped on absorbent paper to remove excess liquid.

For fluorescence-based reporters, the relative fluorescent units (RFU) will be measured on a standard plate reader.

Experiments will be conducted using single linker-antibody combinations, or multiplexed primary antibodies of the same species with multivalent linkers. No signal bleeding or cross-reactivity is expected.

### Example 10: Linker Segment Distance Measurements

Knowing the exact epitopes targeted by the multivalent linkers facilitates the calculation of linker segment lengths/distance factors in vivo. However, in the absence of such data, the epitope distances may be approximated by measuring the distance between the equivalent positions on the target structure. Here, we used the crystal structure of an intact mouse IgG1 (PDB ID ligy; Harris et al. 1998, J. Mol. Biol. 275:861-872) as a model to provide non-limiting, illustrative distance factors for the multivalent linkers of the present disclosure. The focus was on the Fc fragment of the IgG.

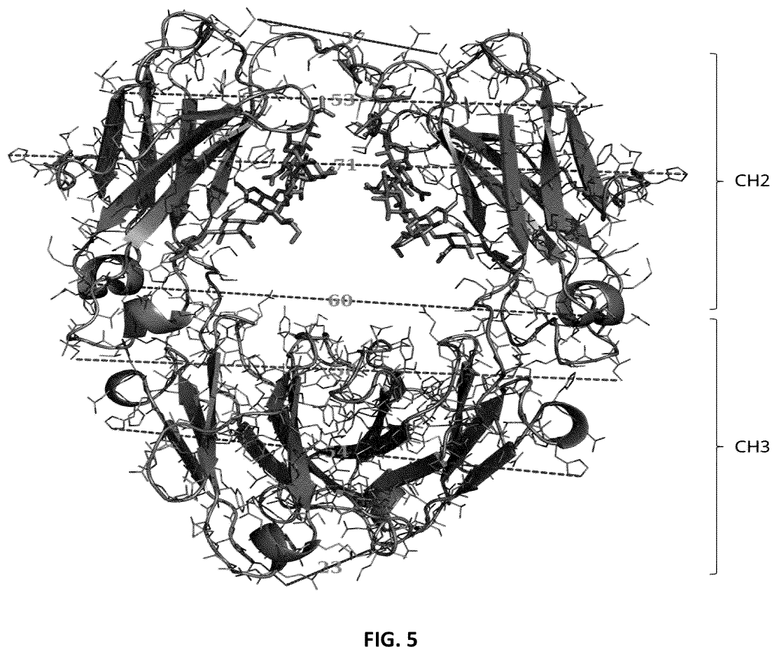

As shown in FIG. 5, the Fc fragment is an O-shaped homodimer with C2 symmetry, comprising the CH2 and CH3 domains of two Y heavy chains. The distance of symmetry points varies significantly over the length of the Fc fragment, with minimal (Euclidean) distances of ca 2-3 nm at the N- and C-terminal tips, but 5-7 nm for most other areas. Thus, for the majority of binding peptides, the linker segment may need to cover a Euclidean distance of at least 5 nm.

Allowing for additional flexibility to avoid steric hindrances (while accounting for epitope bridging by the multivalent linker), we designed linker segments of a length of about 5-9 nm based on Gly-/Ser-rich sequences. We chose Gly and Ser, as they confer maximal flexibility (Gly) and hydrophilicity (Ser). Calculating with a peptide bond length of 0.38 nm, we designed linker segments with 15, 20 and 25 amino acids, which is the equivalent of 5.7 nm, 7.6 nm and 9.5 nm, respectively.

Multivalent linkers were produced with three linker segment lengths. As shown experimentally, a linker segment with 25 amino acids (9.5 nm) falls within this calculated range, and was tested in other sections of the specification (see e.g., FIG. 3).

As discussed above, an insufficiently sized linker segment may lead to aggregation of the primary antibody.

### Example 11: Biophysical Characterization of Multivalent Linkers

In the following examples, multivalent linkers were constructed based on camelid anti-antibody VHHs (also known as single domain antibodies), the variable domains of camelid heavy-chain antibodies. VHHs that bind IgG with very high affinity was selected to construct a bivalent format of multivalent linkers with a (G4S)₅ linker segment: VHH1-(G4S)₅-VHH2. This format is compatible with a 1:1 interaction of one IgG and one multivalent linker, as the two multivalent linker VHH units can occupy both equivalent epitopes on the IgG heteromer, e.g. on both heavy chains of the Fc part. The multivalent linkers also contain one or two cysteine handles for the reproducible, site-directed maleimide conjugation of fluorophores or other labels. Two groups of multivalent linkers were designed, one was specific for rabbit IgG, which comprises only one IgG isotype, and the other was specific for mouse IgG1, the most common mouse IgG isotype.

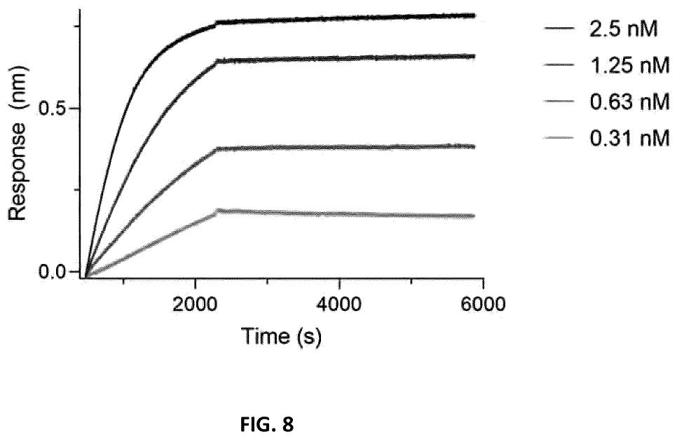

The multivalent linkers described bound to antibodies with picomolar affinities and extremely slow dissociation rates. The binding kinetics of multivalent linkers were studied using biolayer interferometry (BLI), by titrating multivalent linkers to biotinylated binding agent immobilized on streptavidin biosensors (FIG. 8). The observed dissociation rates k_{off} were up to 10⁻⁷ or 10⁻⁶ s⁻¹, which translates to less than 5% dissociation of the multivalent linker from its antibody over several hours to days. Of note, these dissociation rates were recorded at 30° C.; as any other chemical rate, these rates further decrease with lower temperatures, such as typically used in an multiplex immunostaining experiment setting. In addition, the association rates of the multivalent linkers were in the range of 10⁵ Mol⁻¹ s⁻¹, implicating fast binding to IgG. Thus, multivalent linkers rapidly decorated their target primary antibody and, crucially, stayed bound to the same antibody for extended periods of time.

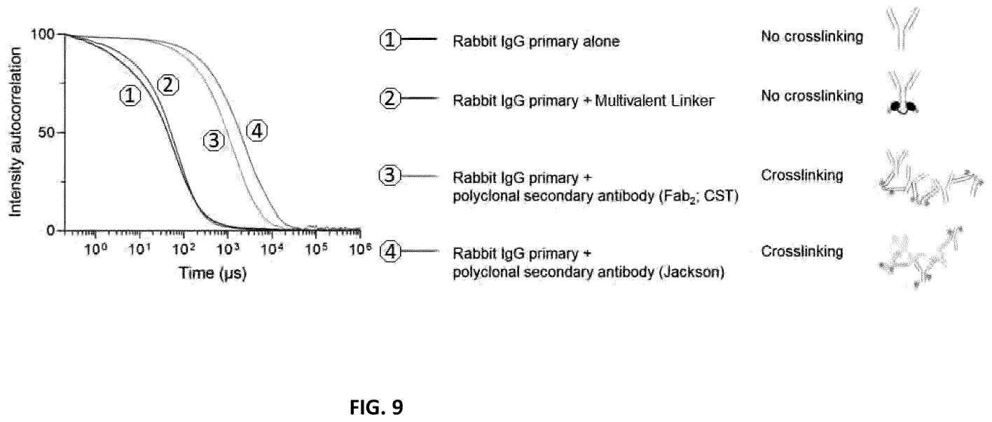

The 1:1 binding mode of multivalent linkers to IgGs allows homogenous labelling and preserves the oligomeric state of their target antibody. Dynamic light scattering (DLS) was used to analyze the solution state of a rabbit IgG in isolation and bound to an anti-rabbit IgG multivalent linker. As shown in FIG. 9, the addition of the multivalent linker increased the hydrodynamic radius Rh for this antibody only incrementally, as expected for the formation of a 1:1 complex without aggregation or crosslinking. In comparison, a significant increase of Rh occurred when the rabbit IgG was mixed with a conventional secondary goat anti-rabbit IgG, indicating aggregation and thus likely loss of activity of the primary antibody when conventional secondary antibody was present. Thus, the DLS results illustrated why conventional secondary antibodies must only be used in sequential staining protocols. In contrast, the multivalent linker binding mode is optimal for the use of antibody labelling prior to target engagement and thus multiplex immunostaining experiments.

### Example 12: Multiplex Immunostainings Using Multivalent Linkers

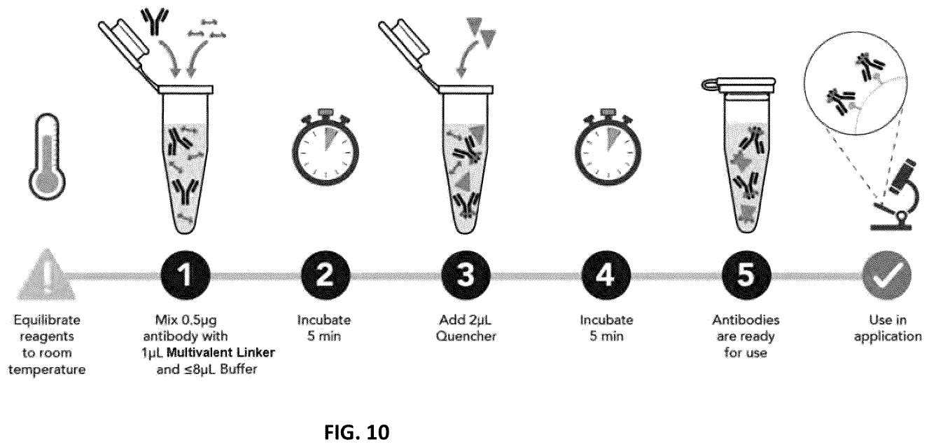

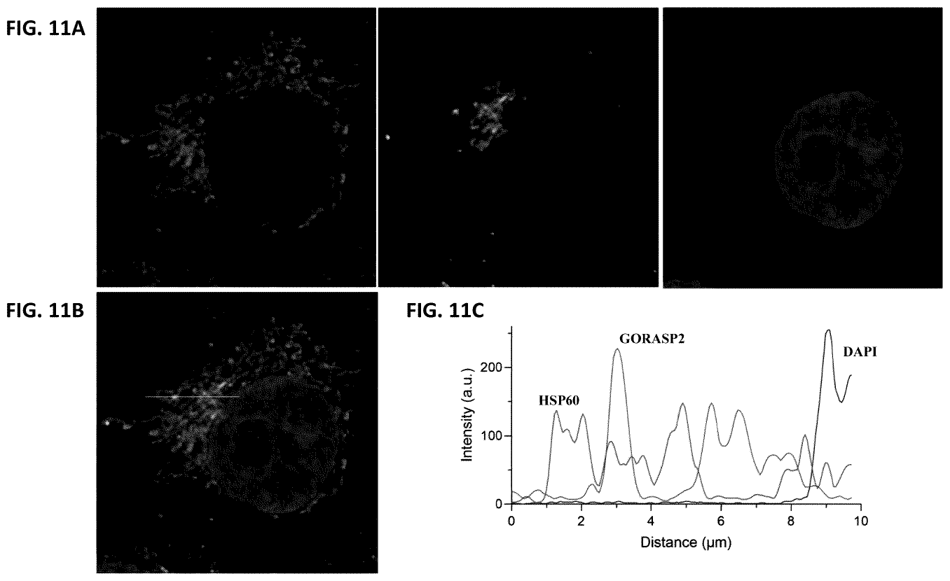

A fast and reproducible multiplex immunostainings protocol was developed using the high-affinity multivalent linkers for rabbit IgG and mouse IgG1. As summarized in FIG. 10, this protocol comprises just two 5-min steps, i.e. the incubation of the multivalent linker with (1) its primary antibody and (2) a quencher. The quencher is an intact IgG or Fc fragment that serves as decoy for excess multivalent linkers. In a first example, this protocol was applied to the simultaneous immunofluorescence co-staining of HeLa cells using mouse monoclonal IgG1 primary antibodies against the mitochondrial protein HSP60 and the Golgi protein GORASP2 labelled with anti-mouse IgG1 multivalent linker conjugated to 555 dye and anti-mouse IgG1 conjugated to 647 dye, respectively. The resulting micrograph (FIG. 11A and FIG. 11B) displayed perfectly resolved mitochondria and Golgi cisternae. A two-dimensional transect of this image, a line intensity plot, demonstrates that the two signals were clearly separated (FIG. 11C), indicating the absence of any cross-reaction between the two anti-mouse IgG1 multivalent linkers and the two mouse IgG1 primary antibodies.

To challenge the labeling protocol, a quantitative and highly sensitive leaking assay was developed to check whether a multivalent linker bound to a first primary antibody would dissociate (leak) from the first primary antibody and then bind to the second, hitherto unlabeled primary antibody. A systematic screening was performed to test the general applicability of multivalent linkers to multiplex immunostaining. Twenty-seven Rabbit IgG and twenty-two mouse IgG1 primary antibodies were sourced from a total of ten vendors, covering monoclonal, polyclonal and recombinant antibodies produced and purified by different methods (see Table 4 below). Notably, these primary antibodies were kept at the condition as provided by their vendors, without any further purification, buffer exchange, or other treatment. Each primary antibody was then successfully labeled with an anti-rabbit IgG or anti-mouse IgG1 multivalent linker as described above and analyzed cross-reactivity with a different primary antibody of the same species/isotype in immunofluorescence using multiplexing assays. In all multiplex assays, we observed staining of the intended target in the absence of any detectable cross-reaction, demonstrating the superiority of multivalent linker-based labeling of primary antibodies.

TABLE 4
Overview of primary antibodies successfully tested with multivalent
linkers in a leaking assay combined with immunofluorescence

| | Rabbit IgG | Mouse IgG1 |
|---|---|---|
| Number of vendors | 5 | 6 |
| Polyclonal | 15 | 0 |
| Monoclonal (recombinant) | 12 (12) | 22 (3) |
| Concentration range | 0.006-1.0 g/l | 0.2-2.0 g/l |
| Purified (antigen) | 19 | 0 |
| Purified (Prot A/G) | n.a. | 14 |
| Supernatant | 0 | 2 |
| Target cytosolic | 7 | 9 |
| Target cytoskeleton | 5 | 4 |
| Target plasma membrane | 6 | 2 |
| Target nuclear | 8 | 7 |

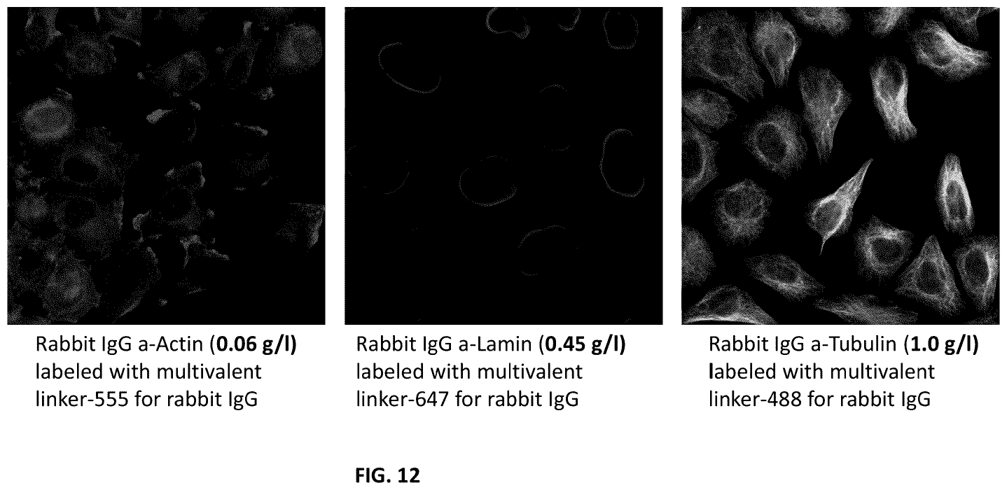

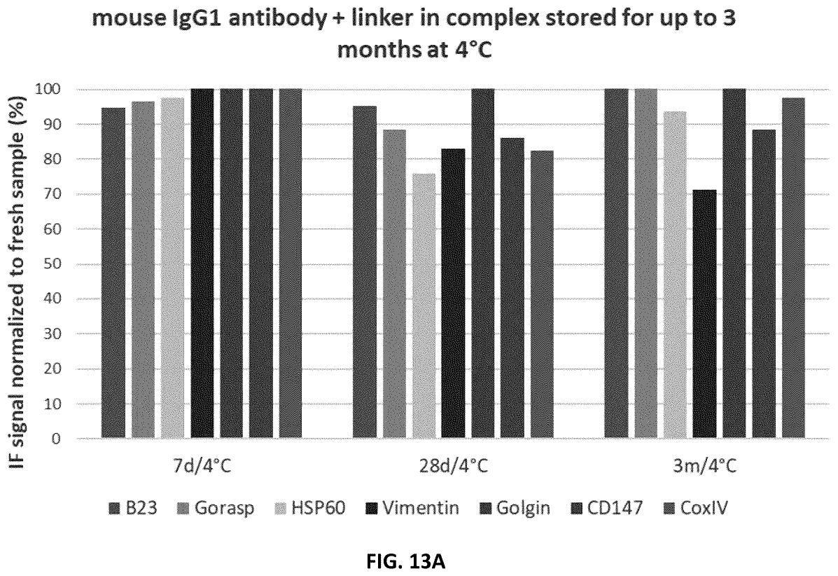

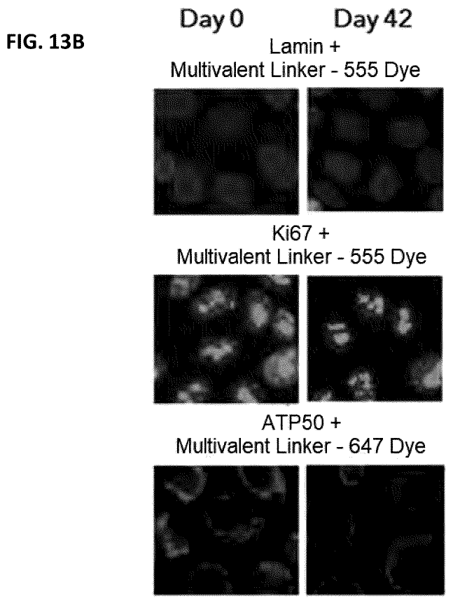

The protocol provides wide-ranging experimental flexibility. For example, the amount of primary antibody can be adjusted according to assay needs. Commercial antibodies are supplied at a very broad range of concentrations, if this information is given at all. As illustrated in FIG. 12, primary antibodies provided at a concentration from 0.05 to 1 mg/ml can be labelled using multivalent linkers and be used to detect their target protein successfully. In addition, for some primary antibodies, adjusting the ratio of multivalent linker to antibody can further improve the overall strength of the staining signal. Furthermore, the molecular complex formed by primary antibody and the multivalent linker may be stable for extended periods of time (FIG. 13A), as long as the primary antibody itself is sufficiently stable. Finally, limited access to a suitable microscopy often delays the visualization of cell or tissue staining. Thus, micrographs of multivalent linker immunofluorescence staining was recorded at the same day as the staining (day 0) and 42 days after and observed no significant decrease in signal strength (FIG. 13B). In summary, the protocol may be tailored to the specific need of the experiment, allowing the use of multivalent linkers in a broad range of applications.

### Example 13: Multivalent Linkers Labeling of Antibodies is Compatible with a Broad Range of Multiplex Immunostaining Applications

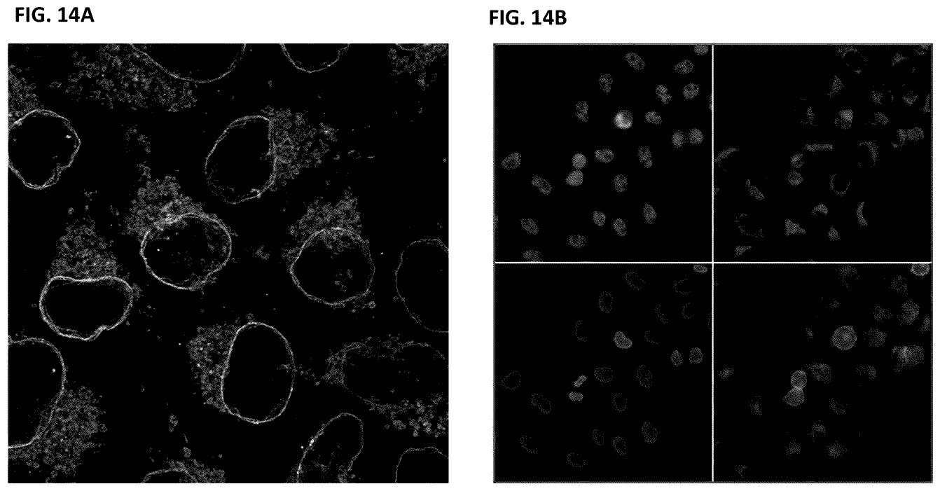

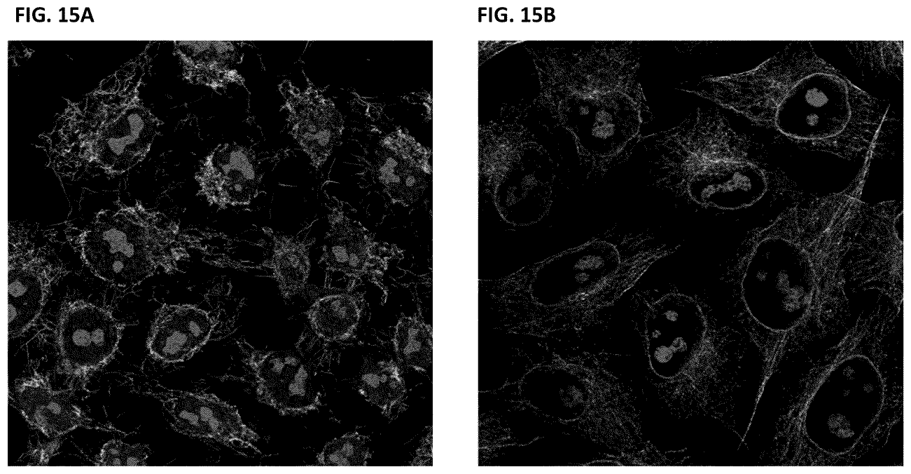

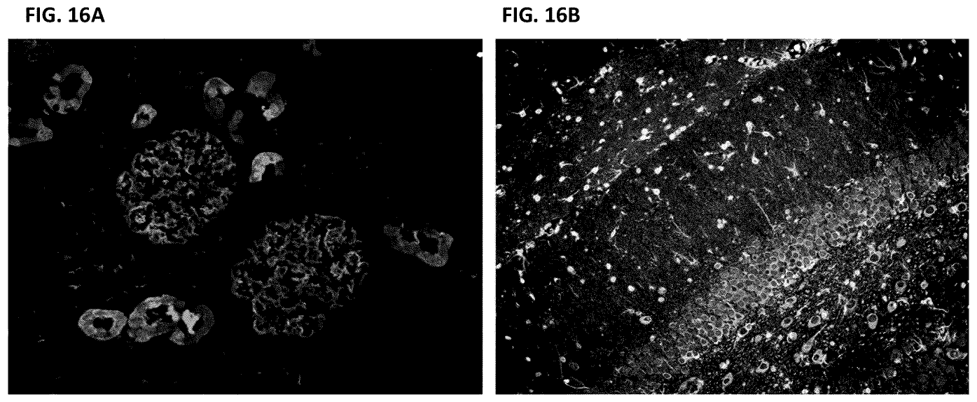

Multivalent linker labeling of antibodies was tested in a range of multiplex immunostaining applications. First, anti-mouse IgG1 or anti-rabbit IgG multivalent linkers were tested for immunofluorescence analysis of cells with three (FIG. 14A) or four (FIG. 14B) primary antibodies of the same species/isotype simultaneously, which visualized markers of the nucleolus, nuclear lamina, Golgi apparatus and mitochondria. Primary antibodies of different species or isotype can also be labelled with their respective multivalent linkers, as shown in FIG. 15A. In addition, co-stainings using multivalent linkers and chemically conjugated primary antibodies illustrated that antibody-multivalent linker complexes are functionally equivalent to and can be used together with chemically labelled antibodies (FIG. 15B). In addition, multiplex IHC/IF stainings were successfully performed on diverse tissues, highlighting various cell types such as human kidney tubules and glomeruli or rat brain neurons (FIG. 16). Of note, the extreme stability of the complex of primary antibody and multivalent linker abrogates any cross-reaction with endogenous IgG contained within the tissue. Thus, prior Fc-blocking is not required.

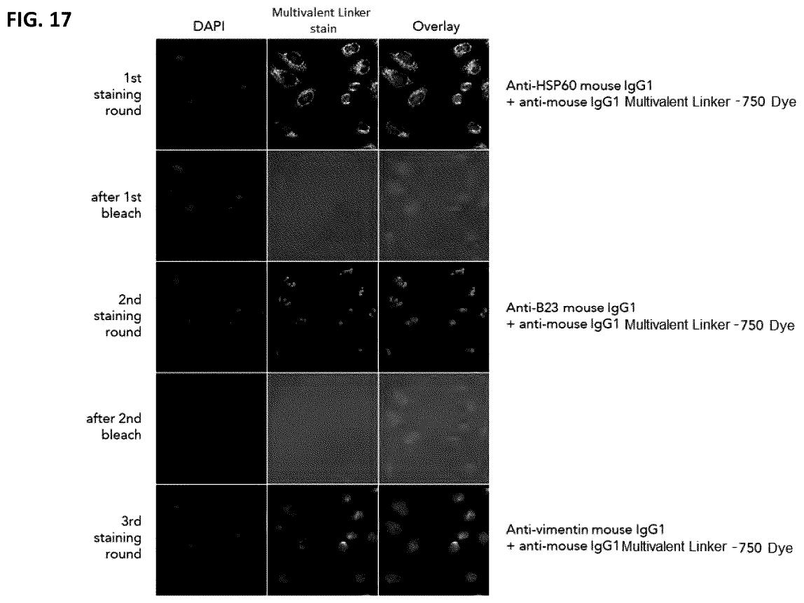

The examples above show multiplexed immunostainings of cultured cells or tissue slices with up to four different fluorophores introduced using our multivalent linkers. The multivalent linker biotin conjugates allow the experimenter to add additional fluorophores using Streptavidin conjugates. Also, the multiplexing level may be raised further using cyclic IF (CyCIF, an overview is provided in world wide web address: www.cycif.org). Briefly, CyCIF subjects samples to repeating cycles of staining using primary antibodies that are either labelled with fluorophores or detected using secondary antibodies followed by chemical bleaching (see, e.g., the description in Lin et al., Elfe. 2018 Jul. 11; 7:e31657, the content of which is incorporated by reference in its entirety). In a proof-of-principle experiment, the conjugated dyes were shown to be efficiently erased using the CyCIF procedure, allowing for several cycles of immunostaining (FIG. 17).

For super-resolution microscopy, primary antibodies labeled with dye-conjugated multivalent linkers will position fluorophores closer to their target, because they are significantly smaller than the complexes of primary and full-length IgG secondary antibodies, thus likely resulting in significant improvement of image resolution.

### Example 14: Applications of Multivalent Linkers in Flow Cytometry

The application of multivalent linkers to flow cytometry was validated. Flow cytometry and the related fluorescence-activated cell sorting (FACS) benefit from using pre-labelled primary antibodies to minimize the incubation time of live cells. Especially, multivalent linkers enable direct labelling of primary antibodies that are currently unavailable as chemical conjugates with a suitable dye.

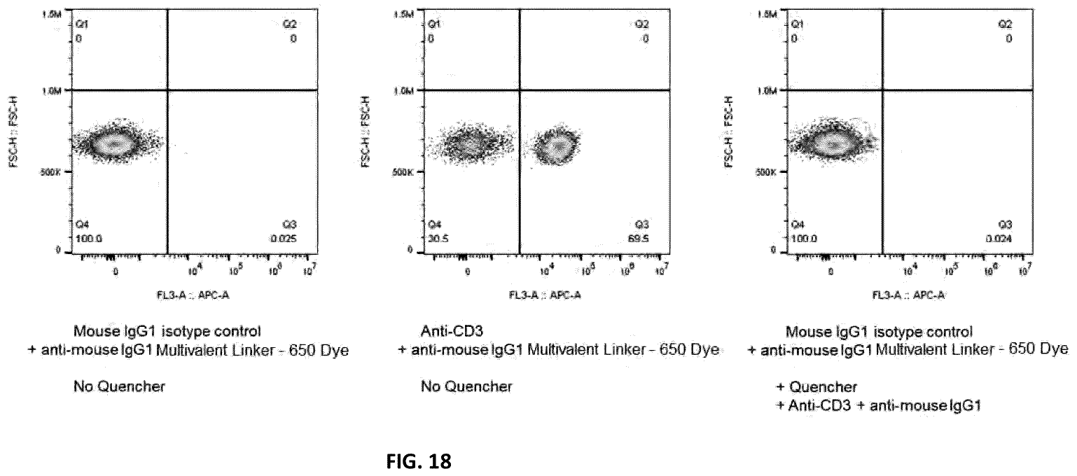

In a first approach, multivalent linkers were confirmed to label their target primary antibody without any cross-reactivity in a modified leaking assay. Using the protocol described in the previous Examples, a mouse IgG1 isotype control (a non-binding monoclonal antibody) was labelled with the anti-mouse IgG1 multivalent linker conjugated to 650 dye, optionally treated with the quencher, and incubated with peripheral blood mononuclear cells (PBMCs) in the absence or presence of an anti-CD3 mouse IgG1 monoclonal antibody. While multivalent linker labelling of the anti-CD3 antibody led to a strong peak for the T cell subpopulation, i.e. CD3⁺ positive cells (FIG. 18), there was no signal, i.e. no leaking for multivalent linker labelling of the isotype control in the presence of anti-CD3 antibody. Thus, the multivalent linker system is compatible with flow cytometry.

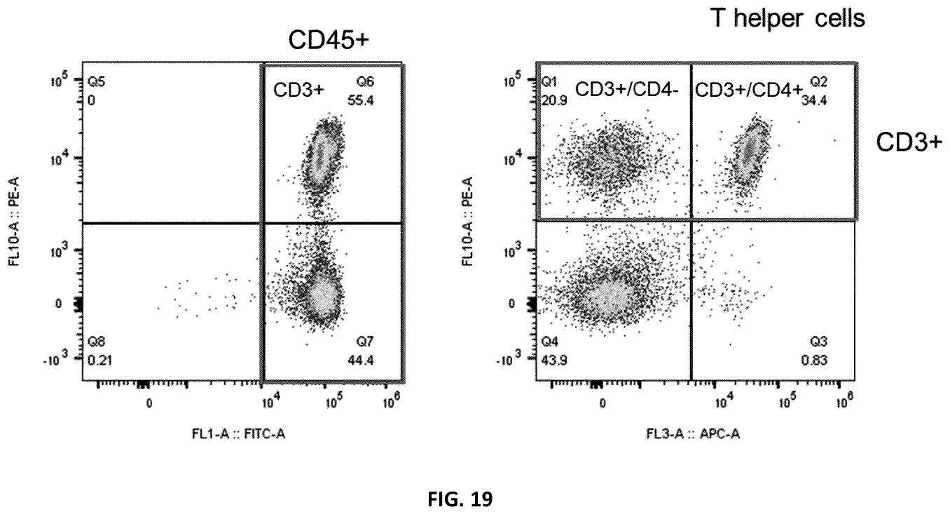

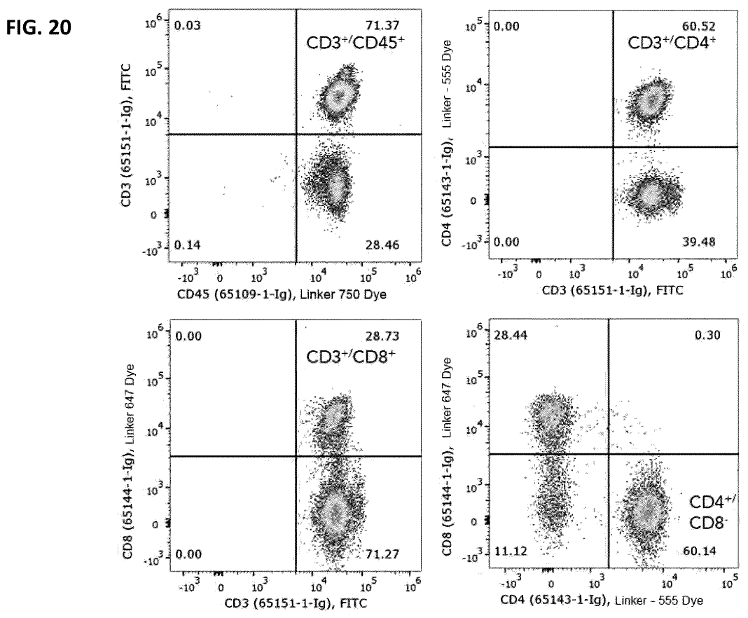

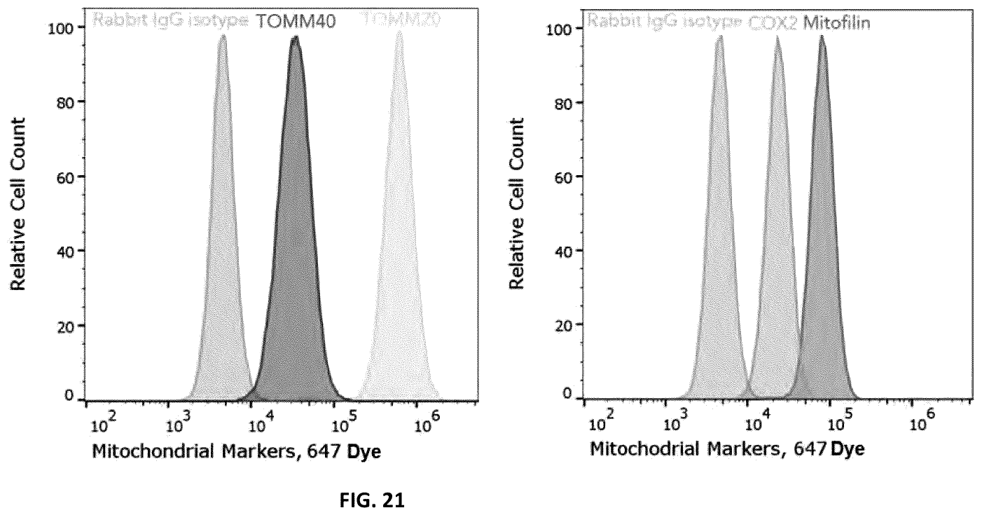

Multivalent linkers enable multiplex staining of cell surface receptors and intracellular markers in flow cytometry. Several scenarios of flow cytometry were tested with the multivalent linkers. In the first study, three primary antibodies of a different mouse isotype each (mouse IgG1, IgG2a, and IgG2b) were labelled with the respective anti-mouse IgG1, IgG2a, or IgG2b multivalent linker labeled with 488, 555, or 647 dye and used to stain PBMCs. As shown in FIG. 19, this staining resulted in clear separation of CD45+/CD3+CD3/CD4+(T helper cells) or CD3/CD4-subpopulations. In the second study, four primary antibodies of the same isotype of mouse IgG1 were labelled with anti-mouse IgG1 multivalent linker conjugated to FITC or 555, 647 or 750 dye and used to stain PBMCs. Again, various populations such as T helper cells or cytotoxic T cells can be clearly identified (FIG. 20). To quantify intracellular markers, rabbit polyclonal primary antibodies against human proteins of the outer and inner mitochondrial membranes were labelled with anti-rabbit IgG multivalent linker conjugated to 647 dye and used to stain permeabilized HEK293T cells (FIG. 21). These examples demonstrate that, in flow cytometry, labelling of primary antibodies using multivalent linkers provides outcomes comparable with directly conjugated antibodies, but with the benefit of the flexibility of the system.

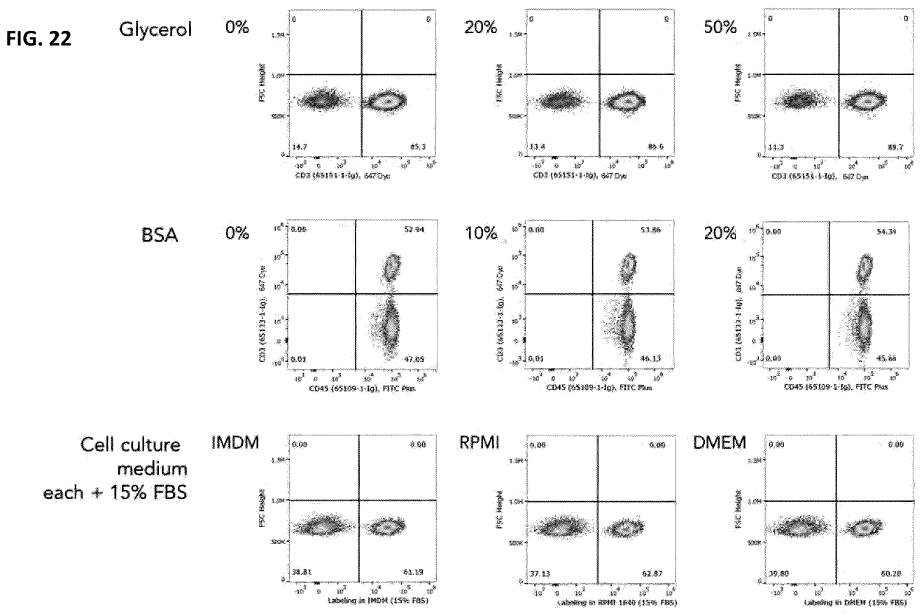

The multivalent linker protocol is highly robust in a wide range of conditions. The quantitative nature of flow cytometry was exploited to study the compatibility of multivalent linkers with various additives that may occur in antibody formulations. For example, the anti-mouse IgG1 multivalent linker was used to label the anti-CD3 antibody stored in 0%, 20% or 50% glycerol without removal of the glycerol (FIG. 22). When using the labelled UCTH1 antibody to detect the T cell subpopulation of PBMCs, no effect of the glycerol was observed. Similarly, multivalent linker labelling of the anti-CD3 antibody OKT3 was also unaffected by high concentrations of BSA up to 20%. As primary antibodies may often be stored in the cell culture medium they were produced in, we also tested the influence of three different cell culture media comprising fetal bovine serum (FBS) on multivalent linker labelling of an anti-CD3 antibody. Again, multivalent linker labelling was highly effective under all tested conditions (FIG. 22). As additives such as glycerol, BSA, cell culture media and FBS are incompatible with traditional antibody labelling methods such as NHS conjugation, the high tolerance of the multivalent linker labelling protocol towards these additives underlines its wide applicability.

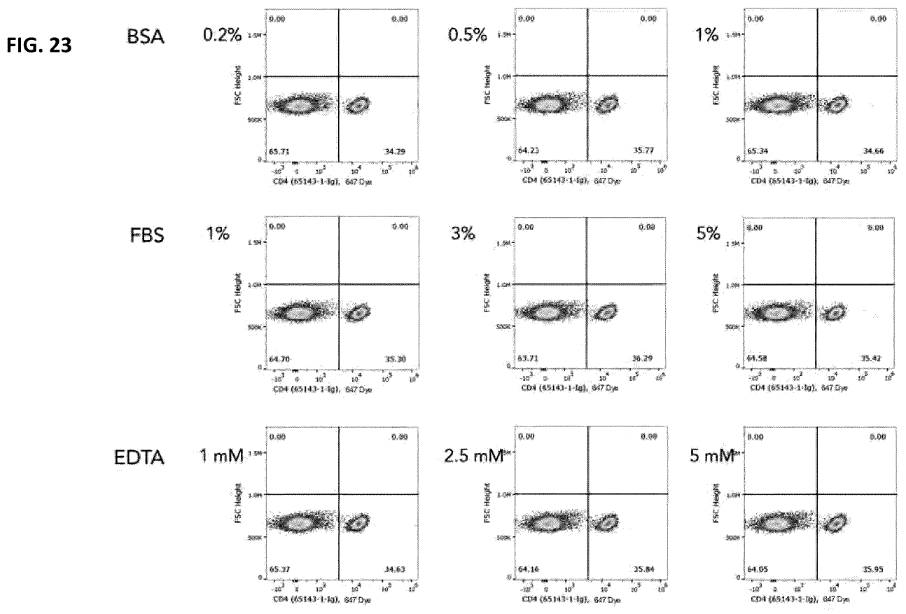

In addition to the superior compatibility of various additives, the multivalent linker labelling technique was studied for factors that may influence the efficiency of cell staining for flow cytometry. Additives such as BSA, FBS or EDTA may be included in flow cytometry or cell sorting experiments to sustain the cells to be analyzed. Accordingly, the mouse IgG1 anti-CD4 was labeled with the anti-mouse IgG1 multivalent linker conjugated to 647 dye to stain CD4⁺ PBMCs. When the staining buffer was supplemented with increasing concentrations of BSA, FBS or EDTA or a combination of thereof, the multivalent linker staining remained unaffected (FIG. 23).

### Example 15: Application of Multivalent Linkers in Hybridoma Screening

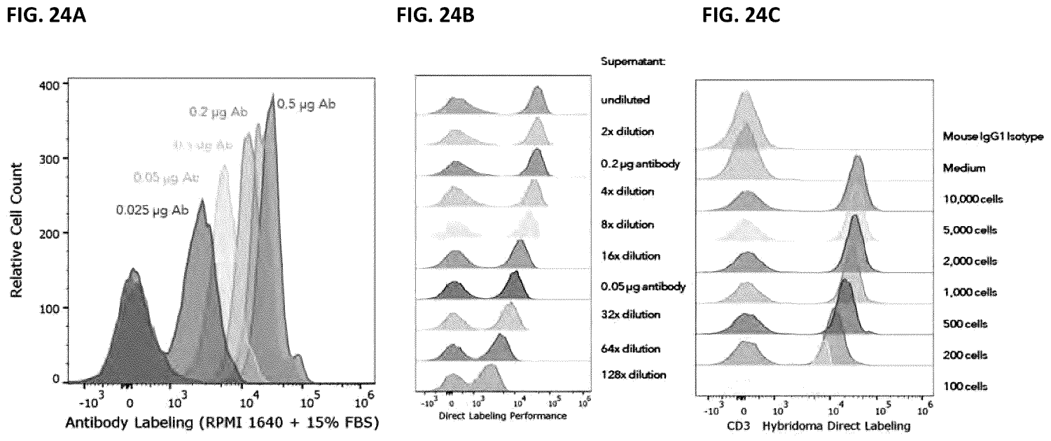

Multivalent linker labelling was found to be a robust technique for accelerating the screening of hybridoma cells. An essential and often time-consuming step of the process of creating new hybridoma cell lines is the screening for target-specific hybridoma cells. Chemical labelling of antibodies within a hybridoma supernatant is virtually impossible owing to media composition. it is hypothesized that multivalent linker labelling circumvents this issue and allows the direct labelling of hybridoma supernatant antibodies. In a first approach, a hybridoma supernatant was stimulated by titrating 0.0025-0.5 μg purified mouse IgG1 anti-CD3 primary antibody into 100 μl of 1×RPMI cell culture medium supplemented with 15% FBS. The mock hybridoma supernatant was labeled using 1 μl anti-mouse IgG1 multivalent linker conjugated to with 647 dye for 5 min and used it to stain PBMCs. The resulting flow cytometry signal increased proportionally with the amount of primary antibody (FIG. 24A). Of note, this reaction volume is one order of magnitude higher than that recommended by the standard protocol, emphasizing once more the flexibility of the system. Second, the culture supernatant from hybridoma cells expressing the mouse IgG2a anti-CD3 was diluted up to 128-fold in IMDM medium supplemented with 15% FBS and labelled using anti-mouse IgG2a multivalent linker conjugated to 647 dye. All dilutions yielded substantial staining of PBMCs in flow cytometry (FIG. 24B). Similarly, multivalent linker labelling of supernatants from 100 to 10,000 hybridoma cells expressing mouse IgG1 anti-CD3 returned successful staining of PBMCs for all cell numbers (FIG. 24C). These data demonstrate that multivalent linkers allow the straightforward labelling and detection of hybridoma supernatant antibodies for a wide range of cell numbers and expression levels. Thus, multivalent linkers enable rapid flow cytometry screening of hybridoma supernatants, using the simple protocol of supplementing a supernatant (e.g. 100 μl) with 1 μl multivalent linker, incubating it for 5 min at room temperature and using it to stain cells.

### NUMBERED EMBODIMENTS

Embodiment 1. A multivalent linker that specifically binds a target antigen unit, said multivalent linker comprising:
a) a plurality of peptide binding arms, each binding arm capable of binding to an epitope in the same target antigen unit; and
b) at least one linker segment covalently operably linked to the plurality of peptide binding arms.

Embodiment 2. A molecular complex, comprising:
(i) a single target antigen unit; and
(ii) a multivalent linker comprising:
   a) a plurality of peptide binding arms, wherein each peptide binding arm binds to the single target antigen unit; and
   b) at least one linker segment covalently operably linked to the plurality of peptide binding arms.

Embodiment 3. The multivalent linker of Embodiment 1 or the molecular complex of Embodiment 2, wherein the multivalent linker binds to the target antigen unit with an apparent k_{off} (s⁻¹) rate of less than or equal to 1.0×10⁻⁴, or wherein the multivalent linker dissociates from the single target antigen unit at an apparent k_{off}(s⁻¹) rate of less than or equal to 1.0×10⁻⁴.

Embodiment 4. The multivalent linker or the molecular complex of Embodiment 3, wherein the k_{off} (s⁻¹) rate is determined by Bio-Layer Interferometry (BLI).

Embodiment 5. The multivalent linker of any one of Embodiments 1 and 3-4 or the molecular complex of any one of Embodiments 2-4, wherein the target antigen unit comprises a constant region of an antibody.

Embodiment 6. The multivalent linker of any one of Embodiments 1 and 3-5 or the molecular complex of any one of Embodiments 2-5, wherein the target antigen unit comprises an Fc region of an antibody.

Embodiment 7. The multivalent linker or the molecular complex of any one of Embodiments 5-6, wherein the epitope is located on a CH2 domain, a CH3 domain, and/or a CH4 domain of the constant region or the Fc region.

Embodiment 8. The multivalent linker of any one of Embodiments 1 and 3-7 or the molecular complex of any one of Embodiments 2-7, wherein the epitope(s) of the peptide binding arms are located on a CH1 domain or a CL domain of the target antigen unit.

Embodiment 9. The multivalent linker of any one of Embodiments 1 and 3-8 or the molecular complex of any one of Embodiments 2-8, wherein the target antigen unit is an antibody, F(ab′)2, Fab2, Fab3, or IgNAR.

Embodiment 10. The multivalent linker or the molecular complex of Embodiment 9, wherein the antibody is an IgG.

Embodiment 11. The multivalent linker or the molecular complex of Embodiment 10, wherein the IgG antibody is
(i) IgG1, IgG2, IgG3, or IgG4 subclass; optionally wherein the IgG antibody is a human antibody;
(ii) IgG1, IgG2a, IgG2b, IgG2c or IgG3 subclass; optionally wherein the IgG antibody is a murine antibody; or
(iii) IgG1, IgG2a, IgG2b, or IgG2c subclass; optionally wherein the IgG antibody is a rat antibody.

Embodiment 12. The multivalent linker or the molecular complex of Embodiment 9, wherein the antibody is an IgM.

Embodiment 13. The multivalent linker or the molecular complex of any one of Embodiments 9-12, wherein the antibody is a heavy-chain antibody.

Embodiment 14. The multivalent linker or the molecular complex of any one of Embodiments 9-13, wherein the antibody is a guinea pig antibody, a mouse antibody, a rat antibody, a chicken antibody (e.g., IgY), a donkey antibody, a rabbit antibody, a human antibody, a goat antibody, a pig antibody, a horse antibody, or a cattle antibody.

Embodiment 15. The multivalent linker or the molecular complex of any one of Embodiments 9-14, wherein at least one of the peptide binding arms is cross-reactive and is capable of non-simultaneously binding to antibodies from two or more species; optionally wherein the two or more species are selected from human, mouse, rat, and rabbit.

Embodiment 16. The multivalent linker or the molecular complex of Embodiment 15, wherein at least one of the peptide binding arms is cross-reactive and is capable of non-simultaneously binding to antibodies from both rabbit and human.

Embodiment 17. The multivalent linker of any one of Embodiments 1 and 3-16 or the molecular complex of any one of Embodiments 2-16, wherein each peptide binding arm is specific for a different epitope of the same target antigen unit.

Embodiment 18. The multivalent linker of any one of Embodiments 1 and 3-16 or the molecular complex of any one of Embodiments 2-16, wherein each peptide binding arm is specific for the same epitope, and wherein the target antigen unit comprises a plurality of the same epitopes.

Embodiment 19. The multivalent linker of any one of Embodiments 1 and 3-18 or the molecular complex of any one of Embodiments 2-18, wherein the plurality of peptide binding arms are capable of binding to the same target antigen unit.

Embodiment 20. The multivalent linker of any one of Embodiments 1 and 3-19 or the molecular complex of any one of Embodiments 2-19, wherein the plurality of peptide binding arms do not bind to more than one target antigen unit.

Embodiment 21. The multivalent linker of any one of Embodiments 1 and 3-20 or the molecular complex of any one of Embodiments 2-20, wherein the multivalent linker is bivalent, comprising two peptide binding arms.

Embodiment 22. The multivalent linker of any one of Embodiments 1 and 3-21 or the molecular complex of any one of Embodiments 2-21, wherein less than 5%, 4%, 3%, 2%, or 1% of the plurality of peptide binding arms crosslink to different target antigen units.

Embodiment 23. The multivalent linker of any one of Embodiments 1 and 3-22 or the molecular complex of any one of Embodiments 2-22, wherein the plurality of peptide binding arms are separated by a distance factor sufficiently long to prevent cross linking of the peptide binding arms with more than one target antigen units.

Embodiment 24. The multivalent linker of any one of Embodiments 1 and 3-23 or the molecular complex of any one of Embodiments 2-23, wherein the linker segment comprises a peptide.

Embodiment 25. The multivalent linker of any one of Embodiments 1 and 3-24 or the molecular complex of any one of Embodiments 2-24, wherein the linker segment comprises between 5-50 amino acids.

Embodiment 26. The multivalent linker or the molecular complex of Embodiment 25, wherein the linker segment comprises between 10-40 amino acids.

Embodiment 27. The multivalent linker or the molecular complex of Embodiment 25, wherein the linker segment comprises between 20-30 amino acids.

Embodiment 28. The multivalent linker or the molecular complex of any one of Embodiments 25-27, wherein the linker segment comprises about 25 amino acids, about 30 amino acids, or about 35 amino acids.

Embodiment 29. The multivalent linker of any one of Embodiments 1 and 3-28 or the molecular complex of any one of Embodiments 2-28, wherein the linker segment is between 1-400 Å in length in the extended conformation.

Embodiment 30. The multivalent linker or the molecular complex of Embodiment 29, wherein the linker segment is between about 50-350 Å in length in the extended conformation.

Embodiment 31. The multivalent linker or the molecular complex of Embodiment 29, wherein the linker segment is between about 70-300 Å in length in the extended conformation.

Embodiment 32. The multivalent linker or the molecular complex of Embodiment 29, wherein the linker segment is between about 70-140 Å in length in the extended conformation.

Embodiment 33. The multivalent linker of any one of Embodiments 1 and 3-32 or the molecular complex of any one of Embodiments 2-32, wherein the multivalent linker has an apparent k_{off}(s⁻¹) rate of less than or equal to 1.0×10⁻⁵, or wherein the multivalent linker dissociates from the single target antigen unit at an apparent k_{off}(s⁻¹) rate of less than or equal to 1.0×10⁻⁵.

Embodiment 34. The multivalent linker or the molecular complex of Embodiment 33, wherein the multivalent linker has an apparent k_{off}(s⁻¹) rate of less than or equal to 1.0×10⁻⁶, or wherein the multivalent linker dissociates from the single target antigen unit at an apparent k_{off}(s⁻¹) rate of less than or equal to 1.0×10⁻⁶.

Embodiment 35. The multivalent linker or the molecular complex of Embodiment 33, wherein the multivalent linker has an apparent k_{off}(s⁻¹) rate of less than or equal to 1.0×10⁻⁷, or wherein the multivalent linker dissociates from the single target antigen unit at an apparent k_{off} (s⁻¹) rate of less than or equal to 1.0×10⁻⁷.

Embodiment 36. The multivalent linker of any one of Embodiments 1 and 3-35 or the molecular complex of any one of Embodiments 2-35, wherein the linker segment comprises a (G4S) unit.

Embodiment 37. The multivalent linker or the molecular complex of Embodiment 36, wherein the linker segment comprises more than 2 (G4S) units.

Embodiment 38. The multivalent linker or the molecular complex of Embodiment 36, wherein the linker segment comprises more than 3 (G4S) units.

Embodiment 39. The multivalent linker or the molecular complex of Embodiment 36, wherein the linker segment comprises more than 4 (G4S) units.

Embodiment 40. The multivalent linker or the molecular complex of Embodiment 36, wherein the linker segment comprises more than 5 (G4S) units.

Embodiment 41. The multivalent linker or the molecular complex of Embodiment 36, wherein the linker segment comprises more than 6 (G4S) units.

Embodiment 42. The multivalent linker or the molecular complex of any one of Embodiments 36-41, wherein the linker segment comprises at most 4, at most 5, at most 6, at most 7, at most 8, or at most 9 (G4S) units.

Embodiment 43. The multivalent linker or the molecular complex of any one of Embodiments 24-42, wherein the linker segment comprises the amino acid sequence of GSTSGSGKSSEGKGEGSTSGSGKSG (SEQ ID NO: 495).

Embodiment 44. The multivalent linker or the molecular complex of any one of Embodiments 24-43, wherein at least 20-25% of the amino acids in the peptide of the linker segment are glycine.

Embodiment 45. The multivalent linker or the molecular complex of any one of Embodiments 24-44, wherein between 60%-90% of the amino acids in the peptide of the linker segment are glycine.

Embodiment 46. The multivalent linker of any one of Embodiments 24-45, wherein between 10%-30% of the amino acids in the peptide of the linker segment are serine or threonine; more preferably, serine.

Embodiment 47. The multivalent linker of any one of Embodiments 24-46, wherein at least 70%, at least 75%, at least 80%, at least 85%, at least 90%, at least 95%, or 100%, of the amino acids in the linker segment are glycine (G), alanine (A), serine (S), threonine (T), glutamic acid (E), aspartic acid (D), lysine (K) and arginine (R); more preferably, glycine, alanine, serine and threonine; more preferably, glycine, serine and threonine; more preferably, glycine and serine.

Embodiment 48. The multivalent linker of any one of Embodiments 24-47, wherein the ratio of (i) glycine to (ii) serine and/or threonine is about 4:1 in the linker segment.

Embodiment 49. The multivalent linker of any one of Embodiments 1 and 3-48 or the molecular complex of any one of Embodiments 2-48, wherein the multivalent linker comprises at least one moiety for conjugation to a heterologous molecule.

Embodiment 50. The multivalent linker or the molecular complex of Embodiment 49, wherein the moiety for conjugation is a cysteine.

Embodiment 51. The multivalent linker or the molecular complex of Embodiment 49, wherein the moiety for conjugation is a lysine.

Embodiment 52. The multivalent linker or the molecular complex of Embodiment 49, wherein the moiety for conjugation comprises a biotin or a streptavidin.

Embodiment 53. The multivalent linker or the molecular complex of Embodiment 49, wherein the moiety for conjugation comprises a functional group for conjugation through click chemistry.

Embodiment 54. The multivalent linker or the molecular complex of Embodiment 53, wherein the functional group comprises dibenzocyclooctyne group (DBCO), azide, tetrazine and/or trans-cyclooctene (TCO).

Embodiment 55. The multivalent linker or the molecular complex of any one of Embodiments 49-54, wherein the linker segment, or the peptide in the linker segment, comprises the moiety for conjugation.

Embodiment 56. The multivalent linker or the molecular complex of any one of Embodiments 49-55, wherein the heterologous molecule is a reporter, an oligonucleotide, a moiety functionalized for click chemistry, or an effector.

Embodiment 57. The multivalent linker of any one of Embodiments 1 and 3-56 or the molecular complex of any one of Embodiments 2-56, wherein the multivalent linker comprises at least one reporter, oligonucleotide, moiety functionalized for click chemistry, or effector attached.

Embodiment 58. The multivalent linker or the molecular complex of Embodiment 57, wherein the reporter is a fluorescent reporter.

Embodiment 59. The multivalent linker or the molecular complex of Embodiment 58, wherein the fluorescent reporter is a fluorescein dye, a rhodamine dye, two or more fluorescent dyes that can act cooperatively with one another, or a protein that exhibits fluorescence.

Embodiment 60. The multivalent linker or the molecular complex of Embodiment 58, wherein the fluorescent reporter is green fluorescent protein, yellow fluorescent protein, orange fluorescent protein, cyan fluorescent protein, blue fluorescent protein, red fluorescent protein, mCherry, tdTomato, mStrawberry, mTangerine, and/or dsRed.

Embodiment 61. The multivalent linker or the molecular complex of Embodiment 57, wherein the reporter is an enzymatic reporter.

Embodiment 62. The multivalent linker or the molecular complex of Embodiment 61, wherein the enzymatic reporter is a horseradish peroxidase, a cathepsin, a matrix metalloprotease, a peptidase, a carboxypeptidase, a glycosidase, a lipase, a phospholipase, a phosphatase, a phosphodiesterase, a sulfatase, a reductase, a bacterial enzyme, a biotin ligase, a DNA transposase, or a nuclease.

Embodiment 63. The multivalent linker or the molecular complex of Embodiment 62, wherein the DNA transposase is Tn5 transposase, or wherein the nuclease is micrococcal nuclease.

Embodiment 64. The multivalent linker or the molecular complex of Embodiment 57, wherein the effector is a magnetic effector.

Embodiment 65. The multivalent linker or the molecular complex of Embodiment 64, wherein the magnetic reporter is Gd(III), Dy(III), Fe(III), and Mn(II), DTPA, DOTA, DO3A, 2-benzyl-DOTA, alpha-(2-phenethyl) 1,4,7,10-tetraazacyclododecane-1-acetic-4,7,10-tris(methylacetic)acid, 2-benzyl-cyclohexyldiethylenetriaminepentaacetic acid, 2-benzyl-6-methyl-DTPA, or 6,6″-bis[N,N,N″,N″-tetra(carboxymethyl)aminomethyl)-4′-(3-amino-4-methoxyphenyl)-2,2′:6′,2″-terpyridine.

Embodiment 66. The multivalent linker of any one of Embodiments 1 and 3-65 or the molecular complex of any one of Embodiments 2-65, wherein the multivalent linker has an apparent K_{D} for the target antigen unit of less than 10,000 pM, less than 1,000 pM, less than 500 pM, less than 100 pM, less than 50 pM, less than 10 pM, or less than 1 pM.

Embodiment 67. The multivalent linker or the molecular complex of Embodiment 66, wherein the multivalent linker has an apparent K_{D} for the target antigen unit of 1 to 10 pM, 10 to 50 pM, 50 to 100 pM, 100 to 500 pM, or 500 to 1,000 pM.

Embodiment 68. The multivalent linker or the molecular complex of Embodiment 66 or 67, wherein the multivalent linker has an apparent K_{D} for the target antigen unit of less than about 50 pM.

Embodiment 69. The multivalent linker or the molecular complex of Embodiment 68, wherein the multivalent linker has an apparent K_{D} for the target antigen unit of less than about 25 pM.

Embodiment 70. The multivalent linker or the molecular complex of Embodiment 69, wherein the multivalent linker has an apparent K_{D} for the target antigen unit of less than about 10 pM.

Embodiment 71. The multivalent linker of any one of Embodiments 1 and 3-70 or the molecular complex of any one of Embodiments 2-70, wherein the plurality of peptide binding arms comprise a peptide binding arm comprising a sequence with at least 80%, at least 85%, at least 90%, at least 95%, at least 96%, at least 97%, at least 98%, at least 99%, or 100% identity with any one of SEQ ID Nos: 1-494.

Embodiment 72. The multivalent linker or the molecular complex of Embodiment 71, wherein the plurality of peptide binding arms comprise a peptide binding arm comprising sequence selected from the group comprising SEQ ID Nos 1-494.

Embodiment 73. The multivalent linker of any one of Embodiments 1 and 3-72 or the molecular complex of any one of Embodiments 2-72, comprising more than one linker segment.

Embodiment 74. The multivalent linker of any one of Embodiments 1 and 3-73 or the molecular complex of any one of Embodiments 2-73, comprising the structure: (peptide binding arm)-linker segment-(peptide binding arm).

Embodiment 75. The multivalent linker of any one of Embodiments 1 and 3-73 or the molecular complex of any one of Embodiments 2-73, comprising the structure: (peptide binding arm)-linker segment-(peptide binding arm)-linker segment-(peptide binding arm).

Embodiment 76. The multivalent linker of any one of Embodiments 1 and 3-75 or the molecular complex of any one of Embodiments 2-75, wherein the peptide binding arm is a VHH of a camelid heavy chain antibody.

Embodiment 77. The multivalent linker of any one of Embodiments 1 and 3-75 or the molecular complex of any one of Embodiments 2-75, wherein the peptide binding arm is a VH of an immunoglobulin.

Embodiment 78. The multivalent linker of any one of Embodiments 1 and 3-77 or the molecular complex of any one of Embodiments 2-77, wherein the plurality of peptide binding arms are covalently linked to the linker segment.

Embodiment 79. The multivalent linker of any one of Embodiments 1 and 3-78 or the molecular complex of any one of Embodiments 2-78, wherein the plurality of peptide binding arms and the linker segment form a continuous polypeptide.

Embodiment 80. The multivalent linker of any one of Embodiments 1 and 3-79 or the molecular complex of any one of Embodiments 2-79, wherein the plurality of peptide binding arms and the linker segment are operably linked via a translational fusion.

Embodiment 81. The multivalent linker or the molecular complex of any one of Embodiments 56-80, wherein the plurality of peptide binding arms, the linker segment, and the reporter or effector are each operably linked via a translational fusion.

Embodiment 82. The multivalent linker of any one of Embodiments 1 and 3-81 or the molecular complex of any one of Embodiments 2-81, wherein each of the binding arm binds to an epitope of the target antigen unit.

Embodiment 83. The multivalent linker of any one of Embodiments 1 and 3-82 or the molecular complex of any one of Embodiments 2-82, wherein the peptide binding arm binds to the epitope non-covalently.

Embodiment 84. A composition comprising the multivalent linker of any one of Embodiments 1 and 3-83 or the molecular complex of any one of Embodiments 2-83.

Embodiment 85. The composition of Embodiment 84, comprising a buffer.

Embodiment 86. A composition comprising two or more different multivalent linkers according to any one of Embodiments 1 and 3-83 or two or more different molecular complex of any one of Embodiments 2-83, wherein each of the multivalent linker is linked to a different reporter.

Embodiment 87. The composition of Embodiment any one of Embodiments 84-86, wherein the target antigen unit comprises a binding domain capable of binding to a test antigen after the multivalent linker binds to the target antigen unit.

Embodiment 88. The composition of Embodiment 87, further comprising a decoy molecule that comprises an epitope of the peptide binding arm(s) but does not comprise the binding domain capable of binding to the test antigen.

Embodiment 89. The composition of any one of Embodiment 84-88, comprising a cryoprotectant selected from glycerol, ethylene glycol, and dimethyl sulfoxide (DMSO).

Embodiment 90. The composition of Embodiment 89, wherein the cryoprotectant is glycerol, and wherein the concentration of the glycerol is up to 50% by volume.

Embodiment 91. The composition of Embodiment 89, wherein the glycerol concentration is less than 30% or less than 15% by volume.

Embodiment 92. The composition of any one of Embodiment 89-91, wherein the glycerol concentration is no less than 5% or no less than 10% by volume.

Embodiment 93. A method for detecting a test antigen in a sample, the method comprising the steps of:
A) contacting the sample with the multivalent linker of any one of Embodiments 1 and 3-83 and a binding agent capable of specifically binding to the test antigen, and
B) removing unbound binding agent and multivalent linker, and
C) measuring the presence of the multivalent linker in the sample;
wherein the binding agent comprises the target antigen unit, and wherein the binding agent specifically binds to the test antigen.

Embodiment 94. A method for detecting two or more test antigens in a sample comprising contacting the sample with a first binding agent specific for a first test antigen and a second binding agent specific for a second test antigen, wherein the first and second binding agents are each bound to a first multivalent linker and a second multivalent linker, respectively, wherein the first and/or second multivalent linkers are the multivalent linkers according to any one of Embodiments 1 and 3-83, and wherein each multivalent linker is attached to a reporter, wherein the reporters are not the same.

Embodiment 95. The method of Embodiment 94, wherein each multivalent linker specifically binds the constant region of the first or second binding agent with an apparent k_{off} (s⁻¹) rate of less than or equal to 1.0×10⁻⁵.

Embodiment 96. The method of Embodiment 94 or 95, wherein the first and second binding agents are each non-covalently bound to a first multivalent linker and a second multivalent linker, respectively.

Embodiment 97. The method of Embodiment 94 or 95, wherein the first and second binding agents are each linked or conjugated to a first multivalent linker and a second multivalent linker, respectively.

Embodiment 98. The method of any one of Embodiments 93-97, wherein less than 5%, 4%, 3%, 2%, or 1% of the multivalent linkers bind to two or more of the binding agents.

Embodiment 99. The method of any one of Embodiments 93-97, wherein less than 5%, 4%, 3%, 2%, or 1% of the first multivalent linker binds to the second binding agent, and wherein less than 5%, 4%, 3%, 2%, or 1% of the second multivalent linker binds to the first binding agent.

Embodiment 100. A method for detecting one or more test antigens in a sample comprising contacting the sample with one or more of the molecular complexes of any one of Embodiments 2-83, wherein the single target antigen unit within each of the molecular complexes is comprised within a binding agent, and wherein the binding agent is capable of specifically binding to the test antigen.

Embodiment 101. The method of Embodiment 100, for detecting two or more different test antigens with two or more of the molecular complexes, wherein the binding agent of each of the molecular complexes is capable of specifically binding to one of the test antigens.

Embodiment 102. The method of any one of Embodiments 93-101, wherein the binding agent is an antibody or comprises an antigen binding fragment thereof.

Embodiment 103. The method of Embodiment 102, wherein the first and second antibodies are of the same species.

Embodiment 104. The method of Embodiment 102, wherein the first and second antibodies are rabbit IgG antibodies.

Embodiment 105. The method of Embodiment 102, wherein the first and second antibodies are mouse IgG antibodies.

Embodiment 106. The method of Embodiment 102, wherein the first and second antibodies are rat IgG antibodies.

Embodiment 107. The method of Embodiment 102, wherein the first and second antibodies are human IgG antibodies.

Embodiment 108. The method of any one of Embodiments 94-107, wherein the first multivalent linker is incubated with the first binding agent prior to contacting the sample with the first binding agent.

Embodiment 109. The method of Embodiment 108, wherein the second multivalent linker is incubated with the second binding agent prior to contacting the sample with the second binding agent.

Embodiment 110. The method of Embodiment 108 or 109, wherein the first binding agent is incubated with the first multivalent linker at a molar ratio of about 1:2.5.

Embodiment 111. The method of any one of Embodiments 108-110, wherein the second binding agent is incubated with the second multivalent linker at a molar ratio of about 1:2.5.

Embodiment 112. The method of any one of Embodiments 108-111, wherein the first binding agent stock concentration is at least 0.001 g/l.

Embodiment 113. The method of any one of Embodiments 108-112, wherein the second binding agent stock concentration is at least 0.001 g/l.

Embodiment 114. The method of any one of Embodiments 94-113, wherein glycerol concentration in a solution containing the first binding agent and/or the second binding agent is between 0-50% by volume.

Embodiment 115. The method of Embodiment 114, wherein the glycerol concentration is less than 30%, or less than 15% by volume.

Embodiment 116. The method of Embodiment 114 or 115, wherein the glycerol concentration is no less than 5% or no less than 10% by volume.

Embodiment 117. The method of any one of Embodiments 93-116, wherein unbound multivalent linker are quenched by adding a decoy molecule that comprises the epitopes of the peptide binding arms but does not bind the test antigen(s).

Embodiment 118. The method of any one of Embodiments 93-117, wherein unbound multivalent linker are removed from multivalent linker-binding agent complexes.

Embodiment 119. The method of Embodiment 118, wherein the unbound multivalent linkers are removed by ultrafiltration.

Embodiment 120. The method of Embodiment 119, wherein the unbound multivalent linkers are removed by bead depletion.

Embodiment 121. The method of any one of Embodiments 118-120, wherein the unbound multivalent linkers are removed by adding unspecific polyclonal IgG, or fragments thereof.

Embodiment 122. The method of any one of Embodiments 118-120, wherein the unbound multivalent linkers are removed by adding unspecific monoclonal IgG, or fragments thereof.

Embodiment 123. The method of any one of Embodiments 94-122, the first multivalent linker and the first binding agent are incubated for about 30 minutes.

Embodiment 124. The method of any one of Embodiments 94-122, wherein the first multivalent linker and the first binding agent are incubated for less than 10 minutes.

Embodiment 125. The method of any one of Embodiments 94-124, wherein the second multivalent linker and the second binding agent are incubated for about 30 minutes.

Embodiment 126. The method of any one of Embodiments 94-124, wherein the second multivalent linker and the second binding agent are incubated for less than 10 minutes.

Embodiment 127. The method of any one of Embodiments 93-126, wherein the method is for western blotting, enzyme linked immunosorbent assay (ELISA), immunofluorescence detection, immunohistochemistry, flow cytometry, fluorescence assisted cell sorting (FACS), screening of antibodies (e.g., using hybridomas), spatial genomic analysis, or mass spectroscopy.

Embodiment 128. The method of Embodiment 127, wherein the method is for cyclic immunofluorescence detection.

Embodiment 129. A molecular complex, comprising:
(a) a single target antigen unit comprising:
   i) a constant or Fc region of an antibody, said constant or FC region being selected from:
      1) a human IgG1, IgG2, IgG3, or IgG4;
      2) a murine IgG1, IgG2a, IgG2b, IgG2c or IgG3;
      3) a rat IgG1, IgG2a, IgG2b, or IgG2c; or
      4) a rabbit IgG; and
(b) a multivalent linker comprising:
   i) a plurality of peptide binding arms, wherein each peptide binding arm binds to the single target antigen unit, wherein the peptide binding arm is a VHH of a camelid heavy chain antibody; and
   ii) at least one peptide linker segment covalently operably linked to the plurality of peptide binding arms, wherein the peptide linker segment is between 10 and 40 amino acids long, and wherein at least 80%, at least 85%, at least 90%, at least 95%, or 100%, of the amino acids in the linker segment are selected from glycine (G), alanine (A), serine (S), threonine (T), glutamic acid (E), aspartic acid (D), lysine (K) and arginine (R).

Embodiment 130. The molecular complex of Embodiment 129, wherein each of the peptide binding arms of the multivalent linker is non-covalently linked to a CH2 domain, a CH3 domain, and/or a CH4 domain of the constant region or the Fc region of the antibody.

Embodiment 131. The molecular complex of any one of Embodiments 129-130, wherein the linker segment comprises a (G4S) unit.

Embodiment 132. The molecular complex of any one of Embodiments 129-130, wherein the linker segment comprises more than 3 (G4S) units.

Embodiment 133. The molecular complex of any one of Embodiments 129-132, wherein the plurality of peptide binding arms and the peptide linker segment are operably linked via a translational fusion.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgement or any form of suggestion that they constitute valid prior art or form part of the common general knowledge in any country in the world.

1. A multivalent linker that specifically binds a target antigen unit, said multivalent linker comprising:
a) a plurality of peptide binding arms, each binding arm capable of binding to an epitope in the same target antigen unit; and
b) at least one linker segment covalently operably linked to the plurality of peptide binding arms.

2. A molecular complex, comprising:
(i) a single target antigen unit; and
(ii) a multivalent linker comprising:
a) a plurality of peptide binding arms, wherein each peptide binding arm binds to the single target antigen unit; and
b) at least one linker segment covalently operably linked to the plurality of peptide binding arms.

3. The multivalent linker of claim 1 or the molecular complex of claim 2, wherein the multivalent linker binds to the target antigen unit with an apparent k_{off} (s⁻¹) rate of less than or equal to 1.0×10⁻⁴, or wherein the multivalent linker dissociates from the single target antigen unit at an apparent k_{off}(s⁻¹) rate of less than or equal to 1.0×10⁻⁴.

4. The multivalent linker or the molecular complex of claim 3, wherein the k_{off}(s⁻¹) rate is determined by Bio-Layer Interferometry (BLI).

5. The multivalent linker of any one of claims 1 and 3-4 or the molecular complex of any one of claims 2-4, wherein the target antigen unit comprises a constant region of an antibody.

6. The multivalent linker of any one of claims 1 and 3-5 or the molecular complex of any one of claims 2-5, wherein the target antigen unit comprises an Fc region of an antibody.

7. The multivalent linker or the molecular complex of any one of claims 5-6, wherein the epitope is located on a CH2 domain, a CH3 domain, and/or a CH4 domain of the constant region or the Fc region.

8. The multivalent linker of any one of claims 1 and 3-7 or the molecular complex of any one of claims 2-7, wherein the epitope(s) of the peptide binding arms are located on a C_{H}1 domain or a CL domain of the target antigen unit.

9. The multivalent linker of any one of claims 1 and 3-8 or the molecular complex of any one of claims 2-8, wherein the target antigen unit is an antibody, F(ab′)2, Fab₂, Fab₃, or IgNAR.

10. The multivalent linker or the molecular complex of claim 9, wherein the antibody is an IgG.

11. The multivalent linker or the molecular complex of claim 10, wherein the IgG antibody is
(i) IgG1, IgG2, IgG3, or IgG4 subclass; optionally wherein the IgG antibody is a human antibody;
(ii) IgG1, IgG2a, IgG2b, IgG2c or IgG3 subclass; optionally wherein the IgG antibody is a murine antibody; or
(iii) IgG1, IgG2a, IgG2b, or IgG2c subclass; optionally wherein the IgG antibody is a rat antibody.

12. The multivalent linker or the molecular complex of claim 9, wherein the antibody is an IgM.

13. The multivalent linker or the molecular complex of any one of claims 9-12, wherein the antibody is a heavy-chain antibody.

14. The multivalent linker or the molecular complex of any one of claims 9-13, wherein the antibody is a guinea pig antibody, a mouse antibody, a rat antibody, a chicken antibody (e.g., IgY), a donkey antibody, a rabbit antibody, a human antibody, a goat antibody, a pig antibody, a horse antibody, or a cattle antibody.

15. The multivalent linker or the molecular complex of any one of claims 9-14, wherein at least one of the peptide binding arms is cross-reactive and is capable of non-simultaneously binding to antibodies from two or more species; optionally wherein the two or more species are selected from human, mouse, rat, and rabbit.

16. The multivalent linker or the molecular complex of claim 15, wherein at least one of the peptide binding arms is cross-reactive and is capable of non-simultaneously binding to antibodies from both rabbit and human.

17. The multivalent linker of any one of claims 1 and 3-16 or the molecular complex of any one of claims 2-16, wherein each peptide binding arm is specific for a different epitope of the same target antigen unit.

18. The multivalent linker of any one of claims 1 and 3-16 or the molecular complex of any one of claims 2-16, wherein each peptide binding arm is specific for the same epitope, and wherein the target antigen unit comprises a plurality of the same epitopes.

19. The multivalent linker of any one of claims 1 and 3-18 or the molecular complex of any one of claims 2-18, wherein the plurality of peptide binding arms are capable of binding to the same target antigen unit.

20. The multivalent linker of any one of claims 1 and 3-19 or the molecular complex of any one of claims 2-19, wherein the plurality of peptide binding arms do not bind to more than one target antigen unit.

21. The multivalent linker of any one of claims 1 and 3-20 or the molecular complex of any one of claims 2-20, wherein the multivalent linker is bivalent, comprising two peptide binding arms.

22. The multivalent linker of any one of claims 1 and 3-21 or the molecular complex of any one of claims 2-21, wherein less than 5%, 4%, 3%, 2%, or 1% of the plurality of peptide binding arms crosslink to different target antigen units.

23. The multivalent linker of any one of claims 1 and 3-22 or the molecular complex of any one of claims 2-22, wherein the plurality of peptide binding arms are separated by a distance factor sufficiently long to prevent cross linking of the peptide binding arms with more than one target antigen units.

24. The multivalent linker of any one of claims 1 and 3-23 or the molecular complex of any one of claims 2-23, wherein the linker segment comprises a peptide.

25. The multivalent linker of any one of claims 1 and 3-24 or the molecular complex of any one of claims 2-24, wherein the linker segment comprises between 5-50 amino acids.

26. The multivalent linker or the molecular complex of claim 25, wherein the linker segment comprises between 10-40 amino acids.

27. The multivalent linker or the molecular complex of claim 25, wherein the linker segment comprises between 20-30 amino acids.

28. The multivalent linker or the molecular complex of any one of claims 25-27, wherein the linker segment comprises about 25 amino acids, about 30 amino acids, or about 35 amino acids.

29. The multivalent linker of any one of claims 1 and 3-28 or the molecular complex of any one of claims 2-28, wherein the linker segment is between 1-400 Å in length in the extended conformation.

30. The multivalent linker or the molecular complex of claim 29, wherein the linker segment is between about 50-350 Å in length in the extended conformation.

31. The multivalent linker or the molecular complex of claim 29, wherein the linker segment is between about 70-300 Å in length in the extended conformation.

32. The multivalent linker or the molecular complex of claim 29, wherein the linker segment is between about 70-140 Å in length in the extended conformation.

33. The multivalent linker of any one of claims 1 and 3-32 or the molecular complex of any one of claims 2-32, wherein the multivalent linker has an apparent k_{off}(s⁻¹) rate of less than or equal to 1.0×10⁻⁵, or wherein the multivalent linker dissociates from the single target antigen unit at an apparent k_{off}(s⁻¹) rate of less than or equal to 1.0×10⁻⁵.

34. The multivalent linker or the molecular complex of claim 33, wherein the multivalent linker has an apparent k_{off}(s⁻¹) rate of less than or equal to 1.0×10⁻⁶, or wherein the multivalent linker dissociates from the single target antigen unit at an apparent k_{off}(s⁻¹) rate of less than or equal to 1.0×10⁻⁶.

35. The multivalent linker or the molecular complex of claim 33, wherein the multivalent linker has an apparent k_{off}(s⁻¹) rate of less than or equal to 1.0×10⁻⁷, or wherein the multivalent linker dissociates from the single target antigen unit at an apparent k_{off}(s⁻¹) rate of less than or equal to 1.0×10⁻⁷.

36. The multivalent linker of any one of claims 1 and 3-35 or the molecular complex of any one of claims 2-35, wherein the linker segment comprises a (G4S) unit.

37. The multivalent linker or the molecular complex of claim 36, wherein the linker segment comprises more than 2 (G4S) units.

38. The multivalent linker or the molecular complex of claim 36, wherein the linker segment comprises more than 3 (G4S) units.

39. The multivalent linker or the molecular complex of claim 36, wherein the linker segment comprises more than 4 (G4S) units.

40. The multivalent linker or the molecular complex of claim 36, wherein the linker segment comprises more than 5 (G4S) units.

41. The multivalent linker or the molecular complex of claim 36, wherein the linker segment comprises more than 6 (G4S) units.

42. The multivalent linker or the molecular complex of any one of claims 36-41, wherein the linker segment comprises at most 4, at most 5, at most 6, at most 7, at most 8, or at most 9 (G4S) units.

43. The multivalent linker or the molecular complex of any one of claims 24-42, wherein the linker segment comprises the amino acid sequence of GSTSGSGKSSEGKGEGSTSGSGKSG (SEQ ID NO: 495).

44. The multivalent linker or the molecular complex of any one of claims 24-43, wherein at least 20-25% of the amino acids in the peptide of the linker segment are glycine.

45. The multivalent linker or the molecular complex of any one of claims 24-44, wherein between 60%-90% of the amino acids in the peptide of the linker segment are glycine.

46. The multivalent linker of any one of claims 24-45, wherein between 10%-30% of the amino acids in the peptide of the linker segment are serine or threonine; more preferably, serine.

47. The multivalent linker of any one of claims 24-46, wherein at least 70%, at least 75%, at least 80%, at least 85%, at least 90%, at least 95%, or 100%, of the amino acids in the linker segment are glycine (G), alanine (A), serine (S), threonine (T), glutamic acid (E), aspartic acid (D), lysine (K) and arginine (R); more preferably, glycine, alanine, serine and threonine; more preferably, glycine, serine and threonine; more preferably, glycine and serine.

48. The multivalent linker of any one of claims 24-47, wherein the ratio of (i) glycine to (ii) serine and/or threonine is about 4:1 in the linker segment.

49. The multivalent linker of any one of claims 1 and 3-48 or the molecular complex of any one of claims 2-48, wherein the multivalent linker comprises at least one moiety for conjugation to a heterologous molecule.

50. The multivalent linker or the molecular complex of claim 49, wherein the moiety for conjugation is a cysteine.

51. The multivalent linker or the molecular complex of claim 49, wherein the moiety for conjugation is a lysine.

52. The multivalent linker or the molecular complex of claim 49, wherein the moiety for conjugation comprises a biotin or a streptavidin.

53. The multivalent linker or the molecular complex of claim 49, wherein the moiety for conjugation comprises a functional group for conjugation through click chemistry.

54. The multivalent linker or the molecular complex of claim 53, wherein the functional group comprises dibenzocyclooctyne group (DBCO), azide, tetrazine and/or trans-cyclooctene (TCO).

55. The multivalent linker or the molecular complex of any one of claims 49-54, wherein the linker segment, or the peptide in the linker segment, comprises the moiety for conjugation.

56. The multivalent linker or the molecular complex of any one of claims 49-55, wherein the heterologous molecule is a reporter, an oligonucleotide, a moiety functionalized for click chemistry, or an effector.

57. The multivalent linker of any one of claims 1 and 3-56 or the molecular complex of any one of claims 2-56, wherein the multivalent linker comprises at least one reporter, oligonucleotide, moiety functionalized for click chemistry, or effector attached.

58. The multivalent linker or the molecular complex of claim 57, wherein the reporter is a fluorescent reporter.

59. The multivalent linker or the molecular complex of claim 58, wherein the fluorescent reporter is a fluorescein dye, a rhodamine dye, two or more fluorescent dyes that can act cooperatively with one another, or a protein that exhibits fluorescence.

60. The multivalent linker or the molecular complex of claim 58, wherein the fluorescent reporter is green fluorescent protein, yellow fluorescent protein, orange fluorescent protein, cyan fluorescent protein, blue fluorescent protein, red fluorescent protein, mCherry, tdTomato, mStrawberry, mTangerine, and/or dsRed.

61. The multivalent linker or the molecular complex of claim 57, wherein the reporter is an enzymatic reporter.

62. The multivalent linker or the molecular complex of claim 61, wherein the enzymatic reporter is a horseradish peroxidase, a cathepsin, a matrix metalloprotease, a peptidase, a carboxypeptidase, a glycosidase, a lipase, a phospholipase, a phosphatase, a phosphodiesterase, a sulfatase, a reductase, a bacterial enzyme, a biotin ligase, a DNA transposase, or a nuclease.

63. The multivalent linker or the molecular complex of claim 62, wherein the DNA transposase is Tn5 transposase, or wherein the nuclease is micrococcal nuclease.

64. The multivalent linker or the molecular complex of claim 57, wherein the effector is a magnetic effector.

65. The multivalent linker or the molecular complex of claim 64, wherein the magnetic reporter is Gd(III), Dy(III), Fe(III), and Mn(II), DTPA, DOTA, DO3A, 2-benzyl-DOTA, alpha-(2-phenethyl) 1,4,7,10-tetraazacyclododecane-1-acetic-4,7,10-tris(methylacetic)acid, 2-benzyl-cyclohexyldiethylenetriaminepentaacetic acid, 2-benzyl-6-methyl-DTPA, or 6,6″-bis[N,N,N″,N″-tetra(carboxymethyl)aminomethyl)-4′-(3-amino-4-methoxyphenyl)-2,2′:6′,2″-terpyridine.

66. The multivalent linker of any one of claims 1 and 3-65 or the molecular complex of any one of claims 2-65, wherein the multivalent linker has an apparent K_{D} for the target antigen unit of less than 10,000 pM, less than 1,000 pM, less than 500 pM, less than 100 pM, less than 50 pM, less than 10 pM, or less than 1 pM.

67. The multivalent linker or the molecular complex of claim 66, wherein the multivalent linker has an apparent K_{D} for the target antigen unit of 1 to 10 pM, 10 to 50 pM, 50 to 100 pM, 100 to 500 pM, or 500 to 1,000 pM.

68. The multivalent linker or the molecular complex of claim 66 or 67, wherein the multivalent linker has an apparent K_{D} for the target antigen unit of less than about 50 pM.

69. The multivalent linker or the molecular complex of claim 68, wherein the multivalent linker has an apparent K_{D} for the target antigen unit of less than about 25 pM.

70. The multivalent linker or the molecular complex of claim 69, wherein the multivalent linker has an apparent K_{D} for the target antigen unit of less than about 10 pM.

71. The multivalent linker of any one of claims 1 and 3-70 or the molecular complex of any one of claims 2-70, wherein the plurality of peptide binding arms comprise a peptide binding arm comprising a sequence with at least 80%, at least 85%, at least 90%, at least 95%, at least 96%, at least 97%, at least 98%, at least 99%, or 100% identity with any one of SEQ ID Nos: 1-494.

72. The multivalent linker or the molecular complex of claim 71, wherein the plurality of peptide binding arms comprise a peptide binding arm comprising sequence selected from the group comprising SEQ ID Nos 1-494.

73. The multivalent linker of any one of claims 1 and 3-72 or the molecular complex of any one of claims 2-72, comprising more than one linker segment.

74. The multivalent linker of any one of claims 1 and 3-73 or the molecular complex of any one of claims 2-73, comprising the structure: (peptide binding arm)-linker segment-(peptide binding arm).

75. The multivalent linker of any one of claims 1 and 3-73 or the molecular complex of any one of claims 2-73, comprising the structure: (peptide binding arm)-linker segment-(peptide binding arm)-linker segment-(peptide binding arm).

76. The multivalent linker of any one of claims 1 and 3-75 or the molecular complex of any one of claims 2-75, wherein the peptide binding arm is a VHH of a camelid heavy chain antibody.

77. The multivalent linker of any one of claims 1 and 3-75 or the molecular complex of any one of claims 2-75, wherein the peptide binding arm is a VH of an immunoglobulin.

78. The multivalent linker of any one of claims 1 and 3-77 or the molecular complex of any one of claims 2-77, wherein the plurality of peptide binding arms are covalently linked to the linker segment.

79. The multivalent linker of any one of claims 1 and 3-78 or the molecular complex of any one of claims 2-78, wherein the plurality of peptide binding arms and the linker segment form a continuous polypeptide.

80. The multivalent linker of any one of claims 1 and 3-79 or the molecular complex of any one of claims 2-79, wherein the plurality of peptide binding arms and the linker segment are operably linked via a translational fusion.

81. The multivalent linker or the molecular complex of any one of claims 56-80, wherein the plurality of peptide binding arms, the linker segment, and the reporter or effector are each operably linked via a translational fusion.

82. The multivalent linker of any one of claims 1 and 3-81 or the molecular complex of any one of claims 2-81, wherein each of the binding arm binds to an epitope of the target antigen unit.

83. The multivalent linker of any one of claims 1 and 3-82 or the molecular complex of any one of claims 2-82, wherein the peptide binding arm binds to the epitope non-covalently.

84. A composition comprising the multivalent linker of any one of claims 1 and 3-83 or the molecular complex of any one of claims 2-83.

85. The composition of claim 84, comprising a buffer.

86. A composition comprising two or more different multivalent linkers according to any one of claims 1 and 3-83 or two or more different molecular complex of any one of claims 2-83, wherein each of the multivalent linker is linked to a different reporter.

87. The composition of claim any one of claims 84-86, wherein the target antigen unit comprises a binding domain capable of binding to a test antigen after the multivalent linker binds to the target antigen unit.

88. The composition of claim 87, further comprising a decoy molecule that comprises an epitope of the peptide binding arm(s) but does not comprise the binding domain capable of binding to the test antigen.

89. The composition of any one of claim 84-88, comprising a cryoprotectant selected from glycerol, ethylene glycol, and dimethyl sulfoxide (DMSO).

90. The composition of claim 89, wherein the cryoprotectant is glycerol, and wherein the concentration of the glycerol is up to 50% by volume.

91. The composition of claim 89, wherein the glycerol concentration is less than 30% or less than 15% by volume.

92. The composition of any one of claim 89-91, wherein the glycerol concentration is no less than 5% or no less than 10% by volume.

93. A method for detecting a test antigen in a sample, the method comprising the steps of:
A) contacting the sample with the multivalent linker of any one of claims 1 and 3-83 and a binding agent capable of specifically binding to the test antigen, and
B) removing unbound binding agent and multivalent linker, and
C) measuring the presence of the multivalent linker in the sample;
wherein the binding agent comprises the target antigen unit, and wherein the binding agent specifically binds to the test antigen.

94. A method for detecting two or more test antigens in a sample comprising contacting the sample with a first binding agent specific for a first test antigen and a second binding agent specific for a second test antigen,
wherein the first and second binding agents are each bound to a first multivalent linker and a second multivalent linker, respectively, wherein the first and/or second multivalent linkers are the multivalent linkers according to any one of claims 1 and 3-83, and wherein each multivalent linker is attached to a reporter, wherein the reporters are not the same.

95. The method of claim 94, wherein each multivalent linker specifically binds the constant region of the first or second binding agent with an apparent k_{off}(s⁻¹) rate of less than or equal to 1.0×10⁻⁵.

96. The method of claim 94 or 95, wherein the first and second binding agents are each non-covalently bound to a first multivalent linker and a second multivalent linker, respectively.

97. The method of claim 94 or 95, wherein the first and second binding agents are each linked or conjugated to a first multivalent linker and a second multivalent linker, respectively.

98. The method of any one of claims 93-97, wherein less than 5%, 4%, 3%, 2%, or 1% of the multivalent linkers bind to two or more of the binding agents.

99. The method of any one of claims 93-97, wherein less than 5%, 4%, 3%, 2%, or 1% of the first multivalent linker binds to the second binding agent, and wherein less than 5%, 4%, 3%, 2%, or 1% of the second multivalent linker binds to the first binding agent.

100. A method for detecting one or more test antigens in a sample comprising contacting the sample with one or more of the molecular complexes of any one of claims 2-83, wherein the single target antigen unit within each of the molecular complexes is comprised within a binding agent, and wherein the binding agent is capable of specifically binding to the test antigen.

101. The method of claim 100, for detecting two or more different test antigens with two or more of the molecular complexes, wherein the binding agent of each of the molecular complexes is capable of specifically binding to one of the test antigens.

102. The method of any one of claims 93-101, wherein the binding agent is an antibody or comprises an antigen binding fragment thereof.

103. The method of claim 102, wherein the first and second antibodies are of the same species.

104. The method of claim 102, wherein the first and second antibodies are rabbit IgG antibodies.

105. The method of claim 102, wherein the first and second antibodies are mouse IgG antibodies.

106. The method of claim 102, wherein the first and second antibodies are rat IgG antibodies.

107. The method of claim 102, wherein the first and second antibodies are human IgG antibodies.

108. The method of any one of claims 94-107, wherein the first multivalent linker is incubated with the first binding agent prior to contacting the sample with the first binding agent.

109. The method of claim 108, wherein the second multivalent linker is incubated with the second binding agent prior to contacting the sample with the second binding agent.

110. The method of claim 108 or 109, wherein the first binding agent is incubated with the first multivalent linker at a molar ratio of about 1:2.5.

111. The method of any one of claims 108-110, wherein the second binding agent is incubated with the second multivalent linker at a molar ratio of about 1:2.5.

112. The method of any one of claims 108-111, wherein the first binding agent stock concentration is at least 0.001 g/l.

113. The method of any one of claims 108-112, wherein the second binding agent stock concentration is at least 0.001 g/l.

114. The method of any one of claims 94-113, wherein glycerol concentration in a solution containing the first binding agent and/or the second binding agent is between 0-50% by volume.

115. The method of claim 114, wherein the glycerol concentration is less than 30%, or less than 15% by volume.

116. The method of claim 114 or 115, wherein the glycerol concentration is no less than 5% or no less than 10% by volume.

117. The method of any one of claims 93-116, wherein unbound multivalent linker are quenched by adding a decoy molecule that comprises the epitopes of the peptide binding arms but does not bind the test antigen(s).

118. The method of any one of claims 93-117, wherein unbound multivalent linker are removed from multivalent linker-binding agent complexes.

119. The method of claim 118, wherein the unbound multivalent linkers are removed by ultrafiltration.

120. The method of claim 119, wherein the unbound multivalent linkers are removed by bead depletion.

121. The method of any one of claims 118-120, wherein the unbound multivalent linkers are removed by adding unspecific polyclonal IgG, or fragments thereof.

122. The method of any one of claims 118-120, wherein the unbound multivalent linkers are removed by adding unspecific monoclonal IgG, or fragments thereof.

123. The method of any one of claims 94-122, the first multivalent linker and the first binding agent are incubated for about 30 minutes.

124. The method of any one of claims 94-122, wherein the first multivalent linker and the first binding agent are incubated for less than 10 minutes.

125. The method of any one of claims 94-124, wherein the second multivalent linker and the second binding agent are incubated for about 30 minutes.

126. The method of any one of claims 94-124, wherein the second multivalent linker and the second binding agent are incubated for less than 10 minutes.

127. The method of any one of claims 93-126, wherein the method is for western blotting, enzyme linked immunosorbent assay (ELISA), immunofluorescence detection, immunohistochemistry, flow cytometry, fluorescence assisted cell sorting (FACS), screening of antibodies (e.g., using hybridomas), spatial genomic analysis, or mass spectroscopy.

128. The method of claim 127, wherein the method is for cyclic immunofluorescence detection.

129. A molecular complex, comprising:
(a) a single target antigen unit comprising:
i) a constant or Fc region of an antibody, said constant or FC region being selected from:
1) a human IgG1, IgG2, IgG3, or IgG4;
2) a murine IgG1, IgG2a, IgG2b, IgG2c or IgG3;
3) a rat IgG1, IgG2a, IgG2b, or IgG2c; or
4) a rabbit IgG; and
(b) a multivalent linker comprising:
i) a plurality of peptide binding arms, wherein each peptide binding arm binds to the single target antigen unit, wherein the peptide binding arm is a VHH of a camelid heavy chain antibody; and
ii) at least one peptide linker segment covalently operably linked to the plurality of peptide binding arms, wherein the peptide linker segment is between 10 and 40 amino acids long, and wherein at least 80%, at least 85%, at least 90%, at least 95%, or 100%, of the amino acids in the linker segment are selected from glycine (G), alanine (A), serine (S), threonine (T), glutamic acid (E), aspartic acid (D), lysine (K) and arginine (R).

130. The molecular complex of claim 129, wherein each of the peptide binding arms of the multivalent linker is non-covalently linked to a CH2 domain, a CH3 domain, and/or a CH4 domain of the constant region or the Fc region of the antibody.

131. The molecular complex of any one of claims 129-130, wherein the linker segment comprises a (G4S) unit.

132. The molecular complex of any one of claims 129-130, wherein the linker segment comprises more than 3 (G4S) units.

133. The molecular complex of any one of claims 129-132, wherein the plurality of peptide binding arms and the peptide linker segment are operably linked via a translational fusion.


SEQUENCE LISTING (532 sequences)

SEQ ID NO: 1 - protein, 127 aa, synthetic construct
QVQLVESGGGLVQPGGSLRLSCAAAGLNFGYVNLGWFRQAPAKERDFVAGITWNGGSAYYADFVEGRFTISRDNNRRTLYLQMNDLKPEDTALYYCAANGGIGSGSGYEDDGSYAYWGQGTQVTVSS

SEQ ID NO: 2 - protein, 129 aa, synthetic construct
QVQLVESGGGLVQAGGSLRLSCAAAGLNFGYVNLGWFRQAPAKEREFVAGITWNGGSAYYGDSVKGRFTISRDNAKNAVFLQMNSLKPEDTAVYYCAAMSRRIGVQPGSPCRDYDADYWGQGTQVTVSS

SEQ ID NO: 3 - protein, 129 aa, synthetic construct
QVQLVESGGGLVQPGGSLRLSCAASGFSLDGLAVGWFRQGPGTGREAVACINSRDGTTYYAGSLKGRFTISGDSAKNTVYLQMNNLKPEDTAVYICAATDWGTYGLERLWDSISEYDHWGQGTQVTVSS

SEQ ID NO: 4 - protein, 129 aa, synthetic construct
QVQLVESGGGLVQPGGSLRLSCAGSDINRYAIGWFRQASGEEREFVAALASIGGKRYYADSVKGRFTISRDMFKNTLYLQMNDLKPEDTAVYYCAARERSFSPTDTLTVVTTARAYDYWGQGTQVTVSS

SEQ ID NO: 5 - protein, 116 aa, synthetic construct
QVQLVESGGGWVQAGGSLSLSCTASGTGFFINSMGWYRQAPGKEREMVATISSSGRSDYGDGVLGRFTISRDYANRVYLQMNSLKPEDTGNYYCRVRGDGRATDYWGQGTQVTVSS

SEQ ID NO: 6 - protein, 124 aa, synthetic construct
QVQLVESGGGLVQPGGSLRLSCAVAGRDWNLYAIGWFRQAPGKEREFVARISWIGGATVYADSVKGRFTISKDVAYNTVHLQMDALNPADTAVYYCAAPSRRVWSESQSDYDYWGQGTQVTVSS

SEQ ID NO: 7 - protein, 125 aa, synthetic construct
QVQLVESGGGLVQPGGSLRLSCTTSGVTLDYSAVAWFRQAPGKEREGVSCISSGGGSINYAESVKGRFTISRDNAKKMVFLQANSLKPEDTAVYYCAADASCPAGTLTAADFGSWGQGTQVTVSS

SEQ ID NO: 8 - protein, 125 aa, synthetic construct
QVQLVESGGGLVQPGGSLRLSCAVSGLTSDDYGIAWFRQAPGKQREGVSCISSTDGRSHYADSVLGRFTISRDNAKNTQYLQMNSLKPEDTAVYYCAAGYIIRLQSECVMSRGYWGQGTQVTVSS

SEQ ID NO: 9 - protein, 112 aa, synthetic construct
QVQLVESGGGLVQPGGSLRLSCAASGFTFKSSPMTWVRQPPGKGLEWVASISSDGSRTLYASSVKGRFTIARDNAKNTLYLQMNTLKSEDTAVYFCDYEESRGQGTQVTVSS

SEQ ID NO: 10 - protein, 112 aa, synthetic construct
QVQLVESGGGLVQPGGSLRLSCAASGFTFSSSPMNWVRQPPGKGLEWVASISSDGSRTLYAYSVKGRFTIARDNAKNTLYLQMHTLESEDTAVYYCDYDDSRGPGTQVTVSS

SEQ ID NO: 11 - protein, 112 aa, synthetic construct
QVQLVESGGGLVQPGGSLRLSCAASGFTFSSSPMNWVRQPPGKGLEWVASISSDGSRTLYRDSVKGRFTIARDNAKNTLYLQMHTLESEDTAVYYCDYDDSRGPGTQVTVSS

SEQ ID NO: 12 - protein, 128 aa, synthetic construct
QVQLVESGGGLVQPGGSLRLSCAVSGFPLDYYAIGWFRQAPGKEREGVACSSSGEGSTVYADSVKGRFTISRDDAKNTAYLQMNSLKPGDTAVYYCATRPYSGRGCPSPNTMGAYDYWGVGTQVTVSS

SEQ ID NO: 13 - protein, 118 aa, synthetic construct
QVQLVESGGGLVQPGGSLRLSCAASGFTFSSYSMSWVRQAPGKGPEWVAVINTGGGSTYYADSVKGRFTISRDNAKNTLYLQMNSLKPEDTALYYCATGAEANWRLSGNQGTQVTVSS

SEQ ID NO: 14 - protein, 117 aa, synthetic construct
QVQLVESGGGQVQPGGSLRLSCAASGFTFSGYTMSWVRQAPGKGLDWISAISSDGGSTTYADSVKGRFTISRDNARNTLYLQMNSLKPEDTAVYYCAKGPRRSYDSWGQGTQVTVSS

SEQ ID NO: 15 - protein, 117 aa, synthetic construct
QVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSS

SEQ ID NO: 16 - protein, 117 aa, synthetic construct
QVQLVESGGGLVQPGGSLRLSCAASGFTFSGYTMSWVRQAPGKGLEWVSAISSDGGSTTYADSVKGRFTISRDNAKNTLYLQMNSLKPEDTAVYYCAKGPRRSYDSWGQGTQVTVSS

SEQ ID NO: 17 - protein, 127 aa, synthetic construct
QVQLVESGGGLVQAGGSLRLSCAASGRTFGNYAMGWFRQAPGKEREGVSCISSSGGSANYADSVKGRFTISRDNAKNTVYLQMNSLKPEDTAVYYCAAPRGGGPWYCPLDYNGMNYWGKGTQVTVSS

SEQ ID NO: 18 - protein, 127 aa, synthetic construct
QVQLVESGGGLVQAGGSLRLSCAASGFTFDDYAIGWFRQAPGKQREGVSCISPSDGSIYYADSVKGRFTLSRDNAKNTVYLQMNSLKPEDTAVYYCATVSRHGSGYVCYAGGEDDHWGQGTQVTVSS

SEQ ID NO: 19 - protein, 127 aa, synthetic construct
QVQLVESGGGLVQPGGSLRLSCAASGFTLDDYAIGWWRQAPGKQREGVSCISPSDGSIYYADSVKGRFTLSRDNAKNTVYLQMNSLKPEDTAVYYCATVSRHGSGYVCYAGGEDDHWGRGTQVTVSS

SEQ ID NO: 20 - protein, 127 aa, synthetic construct
QVQLVESGGGLVQPGGSLRLSCTASGFTLDHYAIGWWRQAPGKQREGVSCISPSDGSIYYADSVKGRFTLSRDNAKNTVYLQMNSLKPEDTAVYYCATVSRHGSGYVCYAGGEDDHWGRGTQVTVSS

SEQ ID NO: 21 - protein, 124 aa, synthetic construct
QVQLVESGGGLVQPGGSLRLSCAASGFTLAYWTIGWFRQTPGKEREGVSSISGRDGSTNYADSVKGRFTISRDVSKNTVYLQMNSLKPEDTAVYYCASGGSDYGLGHWRWYDYWGQGTKVTVSS

SEQ ID NO: 22 - protein, 124 aa, synthetic construct
QVQLVESGGGLVQPGGSLRLSCAASGFTLAYWTIGWFRQTPGKEREGVSSISGRDGSTNYADSVKGRFTISRDVAKNTVYLQMNSLKPEDTAVYYCAAGGSDYGLGHWRWYDYWGQGTQVTVSS

SEQ ID NO: 23 - protein, 124 aa, synthetic construct
QVQLVESGGGLVQPGGSLRLSCVASGFTLAYWTIGWFRQTPGKEREGVSSISGRDGSTNYADSVKGRFTISRDVAKNTVYLQMNSLKPEDTAVYYCAAGGSDYGLGHWRWYDYWGQGTQVTVSS

SEQ ID NO: 24 - protein, 129 aa, synthetic construct
QVQLVESGGGLVQPGGSLKLSCTASGLGLDYYGVGWFRQAPGKEREGVSCINSSGDNIDYADSVKGRFTVSRDLTKNTVYLQMNSLKPEDTAVYYCAAEVIRRVSTVQTMCAFRPMSHWGQGTQVTVSS

SEQ ID NO: 25 - protein, 129 aa, synthetic construct
QVQLVESGGGLVRAGGSLRLSCAAPGRTFRTYAMGWFRQAPGKEREGVSCISSSGDSTNYADSVKGRFTVSRDNAKNTVYLQMNSLKPEDTAVYYCAAEEIRRVPTVQAMCAFGPMSHWGQGQQVTVSS

SEQ ID NO: 26 - protein, 129 aa, synthetic construct
QVQLVESGGGLVQPGGSLTLSCIVSGFTVDDKAIGWFRQAPGKEREGVSCISSSGGSTDYADSVKGRFTASRDNAKNTVYLHMNSLKPDDTAVYYCAAEVIRRVPTVQRMCAFGPMSHWGKGTQVTVSS

SEQ ID NO: 27 - protein, 129 aa, synthetic construct
QVQLVESGGGLVQPGGSLRLSCAVSGLTLDYYAIGWFRQAPGKEREAVSCISSSGDSTDYADSVKGRFTVSREDAKNTVYLQMNSLKREDTAVYYCAAEIIRRVPTVQAMCAFGPMSHWGRGQQVTVSS

SEQ ID NO: 28 - protein, 129 aa, synthetic construct
QVQLVESGGGLVQPGGSLRLSCAASGFTLDNYAIAWFRQAPGKEREAVSCASSSGGTTNYADSVEGRFTISRDNAKNTVYLQMNSLKPEDTAVYYCAAEKIRRVPTIQTMCAFGPMSYWGQGTQVTVSS

SEQ ID NO: 29 - protein, 129 aa, synthetic construct
QVQLVESGGGLVQPGGSLRLSCAPSGNTLDYYTIGWFRQAPGKEREAVSCISSSGGTTDYADSVKGRFTGSRDNAKNTVYLQMNSLKPEDTAVYYCAAEIVRRVPTVQTMCAFGPMSHWGQGTQVTVSS

SEQ ID NO: 30 - protein, 129 aa, synthetic construct
QVQLVESGGGLVQPGGSLRLSCAASGFSLDYYAIGWFRQAPGKEREGVSCISSSGGSTMYADSVKGRFTISRDNAKNTVYLQMNSLKPEDTAVYYCAAEVIRRVSTVQTMCAFGPMSHWGQGTQVTVSS

SEQ ID NO: 31 - protein, 129 aa, synthetic construct
QVQLVESGGGLVQPGGSLRLSCVASGFTLDYYAIGWFRQAPGKEREAVSCVSSSGGSTDYADSVKGRFTISRDNAKNMVYLQMNSLKPEDTAVYYCAAETIRREPTVQTMCAFGPMSHWGQGTQVTVSS

SEQ ID NO: 32 - protein, 129 aa, synthetic construct
QVQLVESGGGLVQPGGSLTLSCAPSGFTLDNYAIGWFRQAPGKEREAVSCISSSGDSTNYADSVKGRFTVSRDNAKNTVYLQMNSLKPEDTAVYYCAAEVIRRVATVQAMCAFGPMSHWGQGQQVTVSS

SEQ ID NO: 33 - protein, 129 aa, synthetic construct
QVQLVESGGGLVQPGGSLTLSCAPSGFTLDNYAIGWFRQAPGKEREAVSCISSSGDSTNYADSVKGRFTVSRDNAKNTVYLQMNSLKPEDTAVYYCAAETIRREPTVQTMCAFGPMSHWGQGTQVTVSS

SEQ ID NO: 34 - protein, 129 aa, synthetic construct
QVQLVESGGGLVQPGGSLTLSCAPSGFTLDNYAIGWFRQAPGKEREAVSCISSSGDSTNYADSVKGRFTVSRDNAKNTVYLQMNSLKPEDTAVYYCAAEVIRRVPTVQTMCAFGPMSHWGQGQQVTVSS

SEQ ID NO: 35 - protein, 129 aa, synthetic construct
QVQLVESGGGLVQPGGSLTLSCAPSGFTLDNYAIGWFRQAPGKEREAVSCISSSGDSTNYADSVKGRFTVSRDNAKNTVYLQMNSLKPEDTAVYYCAAEVIRRVPTVQTMCAFGPMSHWGQGTQVTVSS

SEQ ID NO: 36 - protein, 123 aa, synthetic construct
QVQLVESGGGLVQPGGSLRLSCAASGFTLPTYAIGWFRQAPGKEREGVSYISSGGGSTAYANSVNGRFTISRDFAKNTVYLQMNTLRPEDTGVYYCAATYSAPTILADDYDYWGQGTQVTVSS

SEQ ID NO: 37 - protein, 125 aa, synthetic construct
QVQLVESGGGLVQPGGSLRLSCAASGFTLDYYGIGWFRQAPGKEREGVSCIDSSDGSTYYADSVKGRFTISRDAAKNTVYLQMNSLKPEDTAIYYCAPSSYCTVVIGTGFRYDYWGQGTQVTVSS

SEQ ID NO: 38 - protein, 123 aa, synthetic construct
QVQLVESGGGLVQPGGSLRLSCVASGFTLDYYAIGWFRQAPGKEREGVSCIDSSGGKTDYADSVKGRFTISRDNARNTVYLQMNSLKPEDTAVYYCAVGEGSSCFFRTDYDQWGQGTQVTVSS

SEQ ID NO: 39 - protein, 127 aa, synthetic construct
QVQLVESGGGLVQPGGSLRLSCTASGFTLDYYAIGWFRQAPGKGREGVSCISSSGGSTNYADSVKGRFTISRDNAKNTVYLQMNSLKPEDTDVYYCAVRSRSGVGCVVADPNWYDHWGQGTQVTVSS

SEQ ID NO: 40 - protein, 125 aa, synthetic construct
QVQLVESGGGLVQSGGSLRLSCSASGFTLDYYAIGWFRQAPGKEREGVSCISSSGGSTNYADSVKGRFTISRDNAENTVYLQMNSLKPEDTAVYYCAAKPRVGCVIANPSWYDHWGQGTQVTVSS

SEQ ID NO: 41 - protein, 124 aa, synthetic construct
QVQLVESGGGLVQPGGSLRLSCAASGFTLDYYAIGWFRQAPGKEREGVSCISSDGSSTNYADSVKGRFTISRDNAKNTVYLQMNSLKPEDTAVYYCAQDLGSSWCRRHGNFGSWGQGTQVTVSS

SEQ ID NO: 42 - protein, 124 aa, synthetic construct
QVQLVESGGGLVQPGGSLRLSCAASGFTVNGHAIGWFRQAPGKEREGVSCISSDGTSTNYADSVKGRFTISRDNAKNTVFLQMNSLKPEDTAAYYCAQDLGSSWCRRHGNFGSWGQGTQVTVSS

SEQ ID NO: 43 - protein, 124 aa, synthetic construct
QVQLVESGGGLVQPGGSLRLSCAASGFTVDGHAIGWFRQAPGKEREWVSCISSDGTTTNYADSVKGRFTISRDNAKNTVFLQMNSLKPEDTAVYYCAQDLGSSWCRRHGNFGSWGQGTQVTVSS

SEQ ID NO: 44 - protein, 124 aa, synthetic construct
QVQLVESGGGLVQPGGSLRLSCAASGFTVDGHAIGWFRQAPGKERDGVSCISSDGTSTNYADSVKGRFTISRDNAKNTVYLQMNSLKPEDTAVYYCAQDLGSSWCRRHGNFGSWGQGTQVTVSS

SEQ ID NO: 45 - protein, 124 aa, synthetic construct
QVQLVESGGGLVQPGGSLRLSCAASGFTVDGHAIGWFRQAPGKEREGVSCISSDGTSTNYEDSVKGRFTISRDNAKNTVYLQMNDLKPEDTAVYYCAQDLGSSWCRRHGNFGSWGQGTQVTVSS

SEQ ID NO: 46 - protein, 124 aa, synthetic construct
QVQLVESGGGLVQPGGSLRLSCAASGFTVDGHAIGWFRQAPGKEREGVSCISSDGTSTNYEDSVKGRFTISRDNAKNTVYLQMNSLKPEDTAVYYCAQDLGSSWCRRHGNFGSWGQGTQVTVSS

SEQ ID NO: 47 - protein, 124 aa, synthetic construct
QVQLVESGGGLVQPGGSLRLSCAASGFTVDGHAIGWFRQAPGKEREGVSCISSDGTSTNYEDSVKGRFTISRDNAKNTVYLQMNGLKPEDTAVYYCAQDLGSSWCRRHGNFGSWGQGTQVTVSS

SEQ ID NO: 48 - protein, 124 aa, synthetic construct
QVQLVESGGGLVQPGGSLRLSCSASGFSLENYAIGWYRQAPGKEREGISCISSSGGRTSYVDSVKGRFTISRDNAKNTVYLQMNSLKPEDTAVYYCAAWEWLVYCPREPNYDYWGQGTQVTVSS

SEQ ID NO: 49 - protein, 124 aa, synthetic construct
QVQLVESGGGLVQPGGSLRLSCAASGFTLDNYAIGWYRQAPGKEREGVSCITSRGGRTNYADSVKGRFTISRDNAKNTVYLQMNNLKPEDTADYYCAAWAELVYCPREPNYDYWGQGTQVTVSS

SEQ ID NO: 50 - protein, 124 aa, synthetic construct
QVQLVESGGGLVQPGGSLRLSCAASGFTLDNYAIGWYRQAPGKEREGVSCISSRGGRTNYADSVKGRFTISRDNAKNTVYLQMNNLKPEDTAVYYCAAWAELVYCPREPNYDYWGQGTQVTVSS

SEQ ID NO: 51 - protein, 123 aa, synthetic construct
QVQLVESGGGLVQPGGSLRLACAVSGFTLDVYAIGWFRQAPEKEREGVSCISSGGGSTEYADSVKGRFTISRDNAKNTVYLQMNSLKPEDTAIYYCALTYGPPGLEYDQYDHWGQGTQVTVSS

SEQ ID NO: 52 - protein, 123 aa, synthetic construct
QVQLVESGGGLVQPGGSLRVSCAVSGFTLDRYAIGWFRQAPGKEREGVSCISSGGGSTEYADSVKGRFTISRDNAKNTVYLQMNSLKPEDTAVYYCAATYGPPGLGYDEYEMWGQGTQVTVSS

SEQ ID NO: 53 - protein, 123 aa, synthetic construct
QVQLVESGGGLVQPGGSLRLACAVSEFPWGNYAIGWFRQAPGKEREGVSYISSGGGSTDYADSVKGRFTISRDNAKNTVYLQMNSLKPEDTAVYYCAGDYSAPSLVAADYAHWGQGTQVTVSS

SEQ ID NO: 54 - protein, 123 aa, synthetic construct
QVQLVESGGGSVQPGGSLRLSCAVSGFTLDRYAIGWFRQAPGKEREGVSYISSGGGSTDYADSVKGRFTISRDNAKNTVYLQMNSLKPEDTAVYYCAGDYSAPSLVAADYAHWGQGTQVTVSS

SEQ ID NO: 55 - protein, 123 aa, synthetic construct
QVQLVESGGGLVQPGGSLRLSCAVSGFTLDRYAIGWFRQAPGKEREGVSYISSGGGSTDYADSVKGRFTISRDNAKNTVYLQMNSLKPEDTAVYYCAGDYSAPSLVAADYAHWGQGTQVTVSS

SEQ ID NO: 56 - protein, 123 aa, synthetic construct
QVQLVESGGGLVQIGGSLRLSCAVSGFTLDRYAIGWFRQAPGKEREGVSYISSGGGSTDYADSVKGRFTISRDNAKNTVYLQMNSLKPEDTAVYYCAGDYSAPSLVAADYAHWGQGTQVTVSS

SEQ ID NO: 57 - protein, 123 aa, synthetic construct
QVQLVESGGGLVQIGGSLRLSCAVSGFTLDRYAIGWFRQAPGKEREGVSYISSGGGSTDYADSVKGRFTISRDNAKNTVYLQMNSLKPEDTAVYYCAGDYSAPSLVPADYAHWGQGTQVTVSS

SEQ ID NO: 58 - protein, 126 aa, synthetic construct
QVQLVESGGGSVQPGGSLKLSCESSGETLNNYAIAWFRQVPGKEHEGVSYISSGGGSTNYADSVKGRFTISRDNARNTVYLQMNSLKPEDTAVYSCVATAAAPSLRYSDYGPYDYWGQGTQVTVSS

SEQ ID NO: 59 - protein, 123 aa, synthetic construct
QVQLVESGGGLVQSGGSLRLSCAASGFTYDLYAIGWFRQAPGKEREGVSYISSGGGSTDYADSVKGRFTISRDNAKNAVYLQMNSLKPEDTAVYYCAATYAGPSLDIDQYDYWGQGTQVTVSS

SEQ ID NO: 60 - protein, 126 aa, synthetic construct
QVQLVESGGGLVQPGGSLRLSCAASGFTLDYYAIGWFRQAPGKEREGVSYISSGGGSTNYADSVKGRFTISRDNAKNTVYLQMNSLKPEDTAVYYCVATLAAPSLHYSDYGPYDYWGQGTQVTVSS

SEQ ID NO: 61 - protein, 126 aa, synthetic construct
QVQLVESGGGLVQPGGSLRLSCAASGFTLDYYAIGWFRQAPGKEREGVSYISSGGGSTNYADSVKGRFAISRDNAKNTVYLQMNSLKPEDTAVYYCAATRAAPSLHYSDYGPYDYWGQGTQVTVSS

SEQ ID NO: 62 - protein, 123 aa, synthetic construct
QVQLVESGGGSVQPGGSLRLSCVTSGFTLDLYAMGWFRQAPGKEREGVSSISSGGGSTNYADSVKGRFTISRDFAKNTVYLQMNNLKPEDTAVYYCAANYSAPTIIPYYYDYWGQGTQVTVSS

SEQ ID NO: 63 - protein, 123 aa, synthetic construct
QVQLVESGGGSVQAGGSLRLSCAASGGTFSSYAMAWFRQAPGKEREGVSYVSSGGGSTKYADSVQGRFTISRDFAKNTVYLQMNDLKPEDTAVYYCAADYAAPSLTPSSYTYWGQGTQVTVSS

SEQ ID NO: 64 - protein, 123 aa, synthetic construct
QVQLVESGGGLVQPGGSLRLSCVTRQYTLDYYAIGWFRQAPGKEREGVSYVSSGGGSTNYADSVKGRFTISRDFAKNTVYLQMNDLKPEDTAVYYCAADYAAPSLTPSSYTYWGQGTQVTVSS

SEQ ID NO: 65 - protein, 123 aa, synthetic construct
QVQLVESGGGLVQPGGSLRLSCVASGFTLDYYAIGWFRQAPGKEREGVSYVSSGGGSTNYADSVKGRFTISRDFAKNTVYLQMNDLKPEDTAVYYCAADYAAPSLTPSSYTYWGQGTQVTVSS

SEQ ID NO: 66 - protein, 126 aa, synthetic construct
QVQLVESGGGLVQAGGSLRLSCAVFGRSFSGYYMGWFRQAPGKEREFVAAINWSGAITRYADSVEGRFTISRDNTKNTVFLQMNSLKPEDTAVYYCAALDRSGSYSSTDLSGYGSWGQGTQVTVSS

SEQ ID NO: 67 - protein, 126 aa, synthetic construct
QVQLVESGGGLVQAGGSLELSCAVSGSSFSGYYMGWFRQAPGKEREFVGTINWSGTITRYADSVEGRFTISRDNTKNMVFLQMNSLKPEDTAEYYCAALDRSGSYSSTDLSGYGSWGQGTQVTVSS

SEQ ID NO: 68 - protein, 124 aa, synthetic construct
QVQLVESGGGSVQAGGSLRLSCVASGRTFSITRMGWFRQAPGKEREFVVTITWSSGDTYYADSVQGRFTISRDNAENSVDLQMNSLKPEDTAVYYCAAASGTYYWTSERGYPYWGQGTQVTVSS

SEQ ID NO: 69 - protein, 125 aa, synthetic construct
QVQLVESGGGLVQAGGSLRLSCAASGGPFSSYNMGWFRQSPGKAREFVAAINWRNTSTVYPDSVKGRFTISRDNTKDTVYLQMNSLKPDDTAVYYCAAVTGTGWPLRADYLYEYWGQGAQVTVSS

SEQ ID NO: 70 - protein, 125 aa, synthetic construct
QVQLVESGGGLVQAGGSLRLSCAASGGPFSSYNMGWFRQSPGKAREFVAAINWKNTSTYYADSVKGRFTISRDNTKDTVYLQMNTLKPDDTAVYYCAAVTGTGWPLRVDYLYDYWGQGTQVTVSS

SEQ ID NO: 71 - protein, 123 aa, synthetic construct
QVQLVESGGGLVQAGGSLRLSCSASGRTFSRLFLGWFRQTPGKEPTFVAAISWSGATYYADSVKDRFTISRDNDENTVSLEMNSLKSEDTAVYYCAADIDGGLLSATSTYDYWGQGTQVTVSS

SEQ ID NO: 72 - protein, 123 aa, synthetic construct
QVQLVESGGGSVQAGGSLRLSCAASGRTFSRYFMGWFRQAPEKERAFVAAISWSGAEYYADSVKGRFTISRDNAKNTVSLQMNSLKSEDTAVYYCAADIDGGLLSATSTYDYWGQGTQVTVSS

SEQ ID NO: 73 - protein, 123 aa, synthetic construct
QVQLVESGGGSVQAGGSLRLSCAASGRTFSRYFMGWFRQAPGKERAFVAAISWSGGTYYADSVKHRFTISRDNTKNTVSLQMNSLKSEDTAVYYCAADIDGGLLSATSTYDYWGQGTQVTVSS

SEQ ID NO: 74 - protein, 128 aa, synthetic construct
QVQLVESGGGLVQAGGSLRLSCAAPGRTFRTYAMGWFRQAPGAEREFVAVISWDNDNTHYADSVKGRFTISRDNAENTVYLQMNSLKPEDTAVYYCAASTTGSYGIRWPQDWYEYDFWGQGTQVTVSS

SEQ ID NO: 75 - protein, 120 aa, synthetic construct
QVQLVESGGGLVQAGGSLRLSCVASGRTFSSSAMGWFRQAPGKEREFVAAVSWSGGDTFYADSVKGRFTISGDNAKNTVYLQMNSLEPEDTAVYYCAVDRPHTGQTWDDWGQGTQVTVSS

SEQ ID NO: 76 - protein, 121 aa, synthetic construct
QVQLVESGGGLVQAGGSLRLSCAGSGRTFSTYAMGWFRQAPGKEREFVAATSWSGETTYYADPVKGRFTISMDNAKNTVYLQMNSLKPEDTAIYYCAADTHITSMQWPSYWGQGTQVTVSS

SEQ ID NO: 77 - protein, 121 aa, synthetic construct
QVQLVESGGGLVQTGDSLRLSCAASGRTFSTYAMGWFRQAPGKEREFVAATSWSGETTYYADPVKGRFTISMDNAKNTVYLQMNSLKPEDTAIYYCAADTHITSMQWPSYWGQGTQVTVSS

SEQ ID NO: 78 - protein, 125 aa, synthetic construct
QVQLVESGGGLVQAGGSLRLSCAASGRTFSDYAMGWFRQAPGKEREFVAAINWHGSNLYYADSVKGRFTISRDNAKNMVYLQMNSLKPEDTAVYYCGASSWGGTMWKEWTKYDYWGRGTQVTVSS

SEQ ID NO: 79 - protein, 125 aa, synthetic construct
QVQLVESGGGLVQAGGSLRLSCAASGRTFGDYAMGWFRQAPGKEREFVAAINWSGSNTYYADSVKGRFTISRDNTKNMVYLQMNSLKPEDTAVYYCGASSWGGSMWKEWTKYDYWGQGTQVTVSS

SEQ ID NO: 80 - protein, 125 aa, synthetic construct
QVQLVESGGGLVQAGGSLRLSCAASGRTFSSYAMGWFRQAPGKEREFVAAINWSGSNTYYADSVKGRFTISRDNAKNMVYLQMNSLKPEDTAVYYCGASSWGGSMWKEWTRYDYWGQGTQVTVSS

SEQ ID NO: 81 - protein, 125 aa, synthetic construct
QVQLVESGGGLVQAGGSLRLSCAASGRTFSDYAMGWFRQAPGKEHEFVAAINWHGVNTYYADSVKGRFTISRDNAKNMVYLQMNKVKPEDTAVYYCGASSWGGSMWKEWTKYDYWGQGTQVTVSS

SEQ ID NO: 82 - protein, 115 aa, synthetic construct
QVQLVESGGGLVQTGGSLRLSCVASGLNFRYIAMAWYRQAPGKERELVARITRYGEPISVDSVKGRFAISRDNAKNTVWLQMDGLKNEDTADYYCNTFPSDADYWGQGTQVTVSS

SEQ ID NO: 83 - protein, 115 aa, synthetic construct
QVQLVESGGGNVQAGGSLRLSCVASGLNFRFINMGWYRQAPGKERELVARINMYGEPNSVDSVKGRFTISRDNAKNTVWLQMDGLKLEDTAVYYCNTFPSDADYWGQGTQVTVSS

SEQ ID NO: 84 - protein, 122 aa, synthetic construct
QVQLVESGGGVVQAGESLTLSCAASGNIFKINSMGWYRQPPGKERELVADITSGGVIKYADSVSGRFTISRDRAQNTVYLQMNSLKPEDTGVYYCHAVQHDPMSAMAIEDSWGQGTQVTVSS

SEQ ID NO: 85 - protein, 122 aa, synthetic construct
QVQLVESGGGLVQAGGSMRLSCVASGSHFAINDMGWYRQAVGRARELVASITPGGTIKYADSVKGRFTISRDNAKDTVYLQMNSLKPEDTGVYYCHAVQHDPMSAMAIEDSWGQGTQVTVSS

SEQ ID NO: 86 - protein, 122 aa, synthetic construct
QVQLVESGGGLVQAGGSLTLSCEASGSVVRISAMGWYRQALGKQRELVAVIDNSGTTNYADSVKGRFTISRGAAKGTVYLQMNSLKPEDTGVYYCHAVQHDPMSAMAIEDSWGQGTQVTVSS

SEQ ID NO: 87 - protein, 122 aa, synthetic construct
QVQLVESGGGLVQAGGSLRLSCAASRSVVRISAMGWYRQALGKQRELVAVIDNSGTTNYADSVKGRFTISRGAAKGTVYLQMNSLKPEDTGVYYCHAVQHDPMSAMAIEDSWGQGTQVTVSS

SEQ ID NO: 88 - protein, 122 aa, synthetic construct
QVQLVESGGGSVQAGESLRLSCAASRSVVRISAMGWYRQALGKQRELVAVIDNSGTTNYADSVKGRFTISRGAAKGTVYLQMNSLKPEDTGVYYCHAVQHDPMSAMAIEDSWGQGTQVTVSS

SEQ ID NO: 89 - protein, 122 aa, synthetic construct
QVQLGESGGGLVQTGGSLRLSCAVSGNIVSVNAMAWYRQAPGKQRDLVASVTSGGRTTYTDSVKGRFTISRVDANNTVYLQMNSLRPEDTAVYYCNAVLQTDASTWLARNYWGQGTQVTVSS

SEQ ID NO: 90 - protein, 122 aa, synthetic construct
QVQLVESGGGLVQTGGSLRLSCAVSGNIFSVNAMAWYRQALGKQRELVASITSAGSLTYTESVKGRFTISEVDANNTVYLQMNSLKPEDTAVYYCNALLSTDASTWLAKNYWGQGTQVTVSS

SEQ ID NO: 91 - protein, 125 aa, synthetic construct
QVQLVESGGGLVQPGGSLRLSCAASGHIIFSRYTMGWFRQAPGKEREFVAAISKYNNTYYADFVKGRFIISKNNAKNTVLLQMNSLKPEDTAVYYCAADGDSSRYYYRGVGYDYWGQGTQVTVSS

SEQ ID NO: 92 - protein, 124 aa, synthetic construct
QVQLVESGGGLVQAGDSLRLSCAAPGRVFSTYAMGWFRQAPGKDREFVAAISKYNNTYYGDFVKGRFIISKNNAKNTVLLQMNSLKPEDTAVYYCAADGDSSRYYYRGVGYDIWGQGTQVTVSS

SEQ ID NO: 93 - protein, 116 aa, synthetic construct
QVQLVESGGGLVQPGGSLRLSCVASGIGTIINSKGWYRQAPGKERELVATLTGDYTTNYADSVKGRFTISGDNFANVVYLQMNSLKPEDTAVYFCNVQTSGGENYWGQGTQVTVSS

SEQ ID NO: 94 - protein, 116 aa, synthetic construct
QVQLVESGGGLVQPGGSLRLSCVASGIGTIINSKGWYRQAPGKERELVATLTGDYTTNYADSVKGRFTISGDNFANVVYLQMNSLKPEDTAVYFCNVRTSGGENYWGQGTQVTVSS

SEQ ID NO: 95 - protein, 121 aa, synthetic construct
QVQLVESGGGLVQPGGSLRLSCAASGTIDNIDMMGWYRQSPGKERELVAGISTSDGRTNYDRSVKGRFIISIDKAKNMVYLQMNSLNVEDTAVYYCNARHQGGLASLRSSWGQGTQVTVSS

SEQ ID NO: 96 - protein, 121 aa, synthetic construct
QVQLVESGGGLVQTGGSLKLSCAASGGIFEINDMAWYHQAPGKQRELVASITGGGSTNYADSVKGRFTISRDDAKNMVHLQMNSLQPEDTGVYYCNAAVHQSPYILPRVYWGQGTQVTVSS

SEQ ID NO: 97 - protein, 121 aa, synthetic construct
QVQLVESGGGLVQAGGSLRLSCAASGSIFSIANMRWYRQAPGNQREWVATIDSGGNINYADFVKGRFTISRDNTKNTEYLTMNSLKPEDTAVYYCNADVKHSYSSASWNYWGQGTQVTVSS

SEQ ID NO: 98 - protein, 119 aa, synthetic construct
QVQLVESGGGLVQSGGSLRLSCAASGSILDFYAMDWYRQAPGKRRELVATISTDGSTVYADSVKGRFTISRDNAKNTVYLQMNSLKPEDTAVYYCHADSHPGSAWDDSWGQGTQVTVSS

SEQ ID NO: 99 - protein, 122 aa, synthetic construct
QVQLVESGGGLVQAGGSLSLSCAASGDITCISDMGWYRQAPGKERELVAAVTSIGITNYADSVKGRFTITRDNAKNTVYLQMNSLKPEDTAVYYCKADQCSIGGEVYGMDGWGKGTQVTVSS

SEQ ID NO: 100 - protein, 122 aa, synthetic construct
QVQLVESGGGLVQAGGSLSLSCAASGDITCISDMGWYRQAPGKERELVATVSSIGLTNYADSVKGRFTISRDNAKNTVYLQMNSLKPEDTAVYYCKADQCSIGGVIYGMDYWGKGTQVTVSS

SEQ ID NO: 101 - protein, 121 aa, synthetic construct
QVQLVESGGGVVQPGESLTLSCTASGNIFKINSMAWYRQAPGKQRELVAEITSGGSTNYANSVKGRFTISRDSAKNTVYLQMSSLKPEDTAVYYCNAEPNPGPSTPFDDYWGQGTQVTVSS

SEQ ID NO: 102 - protein, 111 aa, synthetic construct
QVQLVESGGGLVQAGGSLRLSCTASGGFVGIYTMGWYRQAPGKQRELVATITDLGNTDYANSVKGRFTISRDNAKTTAYLQMDSLKPEDTAVYYCNARNTWGQGTQVTVSS

SEQ ID NO: 103 - protein, 122 aa, synthetic construct
QVQLVESGGGLVQPGGSLRLSCVVSGSGSNFSIGAMGWYRQAPGKQRELVAAITNGGGTNYADSVKGRFTISRDAAKNTVYLQMNSLNVEDTAVYYCNARSQGGLASIRSSWGQGTQVTVSS

SEQ ID NO: 104 - protein, 119 aa, synthetic construct
QVQLVESGGDSVQAGESLRLSCVASGSTLAISAWGWYRQTPGKQRDLVAGITSGGTTNYADSVKGRFTISRDNAKNTVYLQMNSLKPEDTAVYYCNADIGPSSWLEDYWGQGTQVTVSS

SEQ ID NO: 105 - protein, 119 aa, synthetic construct
QVQLVESGGGSVHAGGSLTLSCAVSGSAFPITVMGWYRQAPGKQRALVADITSGGRTNYADSVQGRFTISRDNAKNTVYLQMSSLKPEDTAVYYCNAENGPSSWVDDYWGQGTQVTVSS

SEQ ID NO: 106 - protein, 123 aa, synthetic construct
QVQLVESGGGLVQAGESLRLSCTASGSIVSISGMGWYRQAPGKQRELVATIHSGDMTNYAASVKGRFTVSRDDAKNTMYLQMNRVRDEDTAVYYCTAQERPDPFSAMVPKEYWGQGTQVTVSS

SEQ ID NO: 107 - protein, 123 aa, synthetic construct
QVQLVESGGGLVQAGGSLRLACAASGSISVINAMGWYRQAPGKQREFVAHITNGGSTNYAASVKGRFTVSRDDAKNTMYLQMNRVRDEDTAVYYCTAQERPDPFSAMVPKEYWGQGTQVTVSS

SEQ ID NO: 108 - protein, 123 aa, synthetic construct
QVQLVESGGALVQPGGSLRLACAASGSISVINAMGWYRQAPGKQREFVAHITNGGSTNYAASVKGRFTVSRDDAKNTMYLQMNRVRDEDTAVYYCTAQERPDPFSAMVPKEYWGQGTQVTVSS

SEQ ID NO: 109 - protein, 123 aa, synthetic construct
QVQLVESGGGLVQPGGSLRLACAASGSISVINAMGWYRQAPGKQREFVAHITNGGSTNYAASVKGRFTVSRDDAKNTMYLQMNRVRDEDTAVYYCTAQERPDPFSAMVPKEYWGQGTQVTVSS

SEQ ID NO: 110 - protein, 122 aa, synthetic construct
QVQLVESGGGLVQAGGSLRLSCRASGSSFTSDGMAWYRQAPGKQRELVAGITSGGSTDYADSVKGRFAISRDNAKNTVYLQMNSLKPEDTAVYYCNIHRSQGFLTIGGRDYWGQGTQVTVSS

SEQ ID NO: 111 - protein, 118 aa, synthetic construct
QVQLVESGGGLVQAGGSLRLSCAVSGDTFSIHGIGWYRQAPGKQREQVATIAPGGATNYADSVKGRFTISRDNAKNTVYLQMNSLRPDDTAVYYCNKDLAYVSDMDYWGKGTQVTVSS

SEQ ID NO: 112 - protein, 118 aa, synthetic construct
QVQLVESGGGLVQAGGSLRLSCAASGTTFSSNVMGWYRQAPGKQREQVATISPGGTTNYANSVKGRFTISRDNAKNTVYLQMNSLRPDDTAVYYCNKDLSYVTDMDYWGKGTQVTVSS

SEQ ID NO: 113 - protein, 118 aa, synthetic construct; identical to SEQ ID NO: 112

SEQ ID NO: 114 - protein, 118 aa, synthetic construct
QVQLVESGGALVQAGGSLRLSCTVSGRTFSGSAMGWYRQAPGKQREQVATISPGGTTNYANSVKGRFTISRDNAKNTVYLQMNSLRPDDTAVYYCNKDLSYVTDMDYWGKGTQVTVSS

SEQ ID NO: 115 - protein, 123 aa, synthetic construct
QVQLVESGGGVVQAGGTLRLSCVASGSSWTINAMAWYRQTPGKQRELVASITTGENKHYADSVKGRFTISRDSAKNTVYLQMNSLKPEDTAVYYCNAVLRADPTSAVRPYDYWGQGTQVTVSS

SEQ ID NO: 116 - protein, 122 aa, synthetic construct
QVQLVESGGGKVQAGGSLRLSCTASGRTFRDYAMGWFRQAPGKEREFVAALSFSGHSTYYADSVKGRFTVSRDNAKNTMFLQMNSLKPEDTAVYYCNTGVIMGGTYFFGDYWGQGTQVTVSS

SEQ ID NO: 117 - protein, 119 aa, synthetic construct
QVQLVESGGGLVQAGGSLRLSCASSESIFSFTAMGWYRQAPGRQRELVAEIASGGSTIYADSVKGRFTISRDSAKNTVYLQMNSLKPEDTAVYYCNGERAPATRDDDYWGQGTQVTVSS

SEQ ID NO: 118 - protein, 118 aa, synthetic construct
QVQLVESGGGLVQVGGSLRLSCVGSGSMASITYMGWYRQDPGKQRELVARISSGGSTNYADSVKARFTISRDNAKNTVYLQMNNLKPEDTAVYYCNAEGGDYYPLEYWGQGTQVTVSS

SEQ ID NO: 119 - protein, 120 aa, synthetic construct
QVQLVESGGGLVQAGGSLRLSCAASGSIFSIDGMGWYRQAPGKQRELVAHITSGGSTDYADSVKGRFTISRDNAKNALYLQMNSLKPEDTAVYYCNAERPDPSSWLDAYWGQGTQVTVSS

SEQ ID NO: 120 - protein, 119 aa, synthetic construct
QVQLVESGGGVVEAGGSLRLSCAVSTSILSINTMGWYRQAPGKQRELVATMTSGGSTNYADSVKGRFTISRDNAKNTVYLQMSSLKPEDTAVYYCNAENGPSSWVDDYWGQGTQVTVSS

SEQ ID NO: 121 - protein, 119 aa, synthetic construct
QVQLVESGGGLVQPGGSLRLSCAVSTSILSINTMGWYRQAPGKQRELVATMTSGGSTNYADSVKGRFTISRDNAKNTVYLQMSSLKPEDTAVYYCNAENGPSSWVDDYWGQGTQVTVSS

SEQ ID NO: 122 - protein, 121 aa, synthetic construct
QVQLVESGGGSVQAGGSLRLSCASSGSIFSFNAIGWYRQAPEKERELVAVITSGGSTNYADSVKGRFTISRDNAKNTVYLQMNSLKPEDTAVYYCNSGREVGGTYYFGDVWGQGTQVTVSS

SEQ ID NO: 123 - protein, 122 aa, synthetic construct
QVQLVESGGGLVQPGGSLRLSCAASGSIFSINAMGWYRQAPGKERELVATITSHGTTNYADSVKGRFTISRDNAKNTAYLQMNSLKPEDTAVYYCNAQRWEGSDFSRWDEWWGQGTQVTVSS

SEQ ID NO: 124 - protein, 122 aa, synthetic construct
QVQLVESGGGLVQAGGSLRLSCEASGSISSINAMGWYRQTPLKGRELVATITTTGNTNYANSVKGRFTISRDNAKNTAYLQMNSLKPEDTAVYYCNAQRWEGSDFSRWDEWWGQGTQVTVSS

SEQ ID NO: 125 - protein, 122 aa, synthetic construct
QVQLVESGGGLVQPGSSLRLSCAASGSISSINAMGWYRQTPLKGRELVATITTTGNTNYANSVKGRFTISRDNAKNTAYLQMNSLKPEDTAVYYCNAQRWEGSDFSRWDEWWGQGTQVTVSS

SEQ ID NO: 126 - protein, 122 aa, synthetic construct
QVQLVESGGGLVQPGESLRLSCAASGSISSINAMGWYRQTPLKGRELVATITTTGNTNYANSVKGRFTISRDNAKNTAYLQMNSLKPEDTAVYYCNAQRWEGSDFSRWDEWWGQGTQVTVSS

SEQ ID NO: 127 - protein, 122 aa, synthetic construct
QVQLVESGGGLVQPGGALRLSCAASGSISSINAMGWYRQTPLKGRELVATITTTGNTNYANSVKGRFTISRDNAKNTAYLQMNSLKPEDTAVYYCNAQRWEGSDFSRWDEWWGQGTQVTVSS

SEQ ID NO: 128 - protein, 122 aa, synthetic construct
QVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSS

SEQ ID NO: 129 - protein, 122 aa, synthetic construct
QVQLVESGGGLVQPGGSLRLSCAASGTIININAMGWYRQAPGKERELVATITSGGSTNYADSVKGRFTISRDNARNTAYLQMNSLKPEDTAVYYCNAQHWEGSDYSRWDDYWGQGTQVTVSS

SEQ ID NO: 130 - protein, 122 aa, synthetic construct
QVQLVESGGGLVQPGGSLRLSCAASGNIFSINAMGWYRQAPGKERELVATITNGGSTNYGRSVKGRFTISRDNAKNTADLQMNSLKPEDTAVYYCNAVRWEGSDYSRWVDYWGQGTQVTVSS

SEQ ID NO: 131 - protein, 122 aa, synthetic construct
QVQLVESGGGSVQPGGSLRLSCTASGSTFSINAMGWYRQAPGKARELVATMTNDGGTNYARSVKGRFTISRDNAKNTAYLQMNSLKPEDTAVYYCNAVRWEGSDYSRWDDYWGQGTQVTVSS

SEQ ID NO: 132 - protein, 126 aa, synthetic construct
QVQLVESGGGLVQAGGSLRLSCAASGSTFSIDVMGWYRQAPGKQRELVATITSGGSTNYADSVKGRFTISRNNAKNTVYLQMNSLKSEDTAVYYCNAESGAQGFLTIPEGRRNPYWGQGTQVTVSS

SEQ ID NO: 133 - protein, 122 aa, synthetic construct
QVQLVESGGGLVQSGGSLRLSCAASGSTFSINVMGWYRQAPGKQRELVATITSGGSTDYAPSVKGRFTISRNSAANTVYLQMNSLRSEDTAVYYCNAEFLDLTESTRGMDYWGQGTQVTVSS

SEQ ID NO: 134 - protein, 126 aa, synthetic construct
QVQLVESGGGLVRTGESLTLSCVASGSIVSINVMGWYRQAPGKQRELVATITSGGSTDYAPSVKGRFTISRNSAANTVYLQMNSLRSEDTAVYYCYAELGTQGFLAIPEGRRNPYWGQGTQVTVSS

SEQ ID NO: 135 - protein, 126 aa, synthetic construct
QVQLVESGGGLVQSGGSLRLSCAASGSTFSINVMGWYRQAPGKQRELVATITSGGSTDYAPSVKGRFTISRNSAANTVYLQMNSLRSEDTAVYYCYAELGTQGFLAIPEGRRNPYWGQGTQVTVSS

SEQ ID NO: 136 - protein, 125 aa, synthetic construct
QVQLVESGGGLVQAGGSLRLSCAASGSVFSVNAMGWYRQAPGKQRELVATVTSTGSTNYADSVKGRFTISRDNAKNTVYLQMNSLKPEDTAVYYCNLHGTNRAMGVMPASPFDSWGRGTQVTVSS

SEQ ID NO: 137 - protein, 121 aa, synthetic construct
QVQLVESGGGLVQAGGSLRLSCAASGLTSGIGAIGWYRQAPGKQRELVAAISSDGSTKYADSVKGRFTISRDSAKNTIYLQMTSLKPEDTAVYYCNSGAWFSGEYYFGSTWGQGTQVTVSS

SEQ ID NO: 138 - protein, 121 aa, synthetic construct
QVQLVESGGGLVQAGGSLRLSCAASGSTFMINAIGWYRQAPRNQRELLAAISSDGSTKYADSVKGRFTISRDNAKNTIYLQMTSLKPEDTAVYYCNSGAWFSGEYYFGSTWGQGTQVTVSS

SEQ ID NO: 139 - protein, 122 aa, synthetic construct
QVQLVESGGGLVQAGGSLRLSCAASGSIFSINAMGWYRQAPGKQRELVAEVTTGGITNYADSVKGRFTISRDNAKGTVYLQMNSLRPEDTAVYYCNAEPRPGPGATTDPDYWGQGTQVTVSS

SEQ ID NO: 140 - protein, 121 aa, synthetic construct
QVQLVESGGGFVQAGGSLRLSCAASGSIVSFNAMGWYRQAPGKQRELVATITPSGGSTNYADSVKGRFTISTDNAKNTVYLQMNSLKPEDTAVYYCNAEFQGGLAFTRTSWGQGTQVTVSS

SEQ ID NO: 141 - protein, 123 aa, synthetic construct
QVQLVESGGGLVQAGGSLRLSCAASRSILSFNAMGWYRQAPGNQRELVASITSGGSTNYKDSVKGRFTISRDNAKNTVYLQMNSLKPEDTAVYYCNAVLRADPTSAVRPYDYWGQGTQVTVSS

SEQ ID NO: 142 - protein, 123 aa, synthetic construct
QVQLVESGGGLVQAGGSLRLSCVASGSISSTNLMGWYREAPGKQRELVATITSGRSTNYADSVKGRFTISRDNAKNTVYLQMNSLKPEDTAVYYCNAVLRADPTSAVRPYDYWGQGTQVTVSS

SEQ ID NO: 143 - protein, 119 aa, synthetic construct
QVQLVESGGGLVQAGGSLRLSCAASGSIFSIWTMGWYRQAPGKQRELVADITSGGSTNYADSVKGRFTISRDNAKNTVYLQMNSLKPGDTAVYYCKAESDEESYVRNYWGQGTQVTVSS

SEQ ID NO: 144 - protein, 119 aa, synthetic construct
QVQLVESGGGLVQPGGSLRLACAASGSISVINAMGWYRQAPGKQREFVAHITNGGSTNYAASVKGRFTVSRDNAENTLYLQMNSLKPEDTAVYYCNANEDKTYYPLDYWGQGTQVTVSS

SEQ ID NO: 145 - protein, 118 aa, synthetic construct
QVQLVESGGGLVQPGGSLTLSCAASGSIFSDFAMAWYRQAPGKQRELVARITSGGSTNYADSVKGRFTISRDNAKNTLYLQMNSLKPEDTAIYYCNALGGTYYPLDYWGQGTQVTVSS

SEQ ID NO: 146 - protein, 121 aa, synthetic construct
QVQLVESGGGLVQAGGSLRLSCAASGSPFSINAMAWYRQAQGKQRELVAAISSGGSTYYADSVKGRFTISRDNAKNTVYLQMTSLKPEDTAVYYCNTGVIMGGSYYFGDYWGQGTQVTVSS

SEQ ID NO: 147 - protein, 121 aa, synthetic construct
QVQLVESGGGLVQAGGSLRLSCAASGSPFSINAMAWYRQAQGKQRELVATISSGGSTNYADSVKGRFTISRDNAKNTVYLQMNSLKPEDTAVYYCNTGVIMGGTYFFGDYWGQGTQVTVSS

SEQ ID NO: 148 - protein, 121 aa, synthetic construct
QVQLVESGGGLVQAGGSLRLSCAASGSTFSINAIGWYRQAPGKQRELVAAISSGGSTNYADSVKGRFTISRDNAKNTVYLQMNSLKPEDTAVYYCNAGAWVSGSYYFGDYWGQGTQVTVSS

SEQ ID NO: 149 - protein, 121 aa, synthetic construct
QVQLVESGGGLVQAGGSLRLSCAASGSTFTTNAIGWFRQAPGKQREFVAAISSDGSTNYADSVKGRFTISRDNAKNTVYLQMNSLKPEDTAVYYCNTGVIMGGSYYFGDYWGQGTQVTVSS

SEQ ID NO: 150 - protein, 122 aa, synthetic construct
QVQLVESGGGLVQPGGSLRLSCAASGRTTSVMAMGWFRQAPGKEREFVAAISWSDGSTVYADSVKGRFTISRDNAKNTVYLQMNSLKPEDTAVYYCNTGVIMGGTYYFGDYWGQGTQVTVSS

SEQ ID NO: 151 - protein, 119 aa, synthetic construct
QVQLVESGGGLVQAGGSLTLSCTASRSIFSINTMGWYRQAPGKQRELVATITSGGSTAYAESVKGRFTISTDNAKNTVYLQMNGLKPEDTAVYYCNAEDAPGTYLDDYWGQGTQVTVSS

SEQ ID NO: 152 - protein, 119 aa, synthetic construct
QVQLVESGGGLVQAGGSLTLSCAASTSIFSINTMGWYRQAPGKQRELVATITSGGSLNYADSVKGRFTISRDNAKNTVSLQMNRLKPEDTAVYYCNAEDAPGTYLDDYWGQGTQVTVSS

SEQ ID NO: 153 - protein, 119 aa, synthetic construct
QVQLVESGGGLVQAGGSLRLSCAASGSIFSINAMGWYRQAPGKQRELVATITKGGSLNYADSVKGRFTISRDNAKNTVSLQMNRLKPEDTAVYYCNAEDAPGTYLDDYWGQGTQVTVSS

SEQ ID NO: 154 - protein, 119 aa, synthetic construct
QVQLVESGGGLVQAGGSLRLSCAASGSIFSIAVMGWYRQAPGKLRELVADITSGGTTNYADSVKGRFTISRDNAKNTVYLQMNSLKPEDTAVYYCNAEVAPSTVDEDYWGHGTQVTVSS

SEQ ID NO: 155 - protein, 119 aa, synthetic construct
QVQLVESGGGLVQAGGSLRLSCAASGSIFSIAVMGWYRQAPGKLRELVADITSGGTTNYADSVKGRFTISRDNAKNTVYLQMNSLKPEDTAVYYCNAEVAPSTVDEDYWGQGTQVTVSS

SEQ ID NO: 156 - protein, 124 aa, synthetic construct
QVQLVESGGGLVQAGGSLRLSCSASGSIFSINAMGWYRQAPGKQRELVAAITSGGSTNYADSVKGRFTISRDNAKNTVYLQMNSLKPEDTAVYYCKARAEEQGGLVLPREYDYWGQGTQVTVSS

SEQ ID NO: 157 - protein, 119 aa, synthetic construct
QVQLVESGGGLVQAGGSLRLSCVASGSIVSINSMGWYRQAPGKQRELVAAVTSGGSTNYADSVKGRFTISRDNAENTLYLQMNSLKPEDTAVYYCNANEDKTYYPLDYWGQGTQVTVSS

SEQ ID NO: 158 - protein, 119 aa, synthetic construct
QVQLVESGGGLVQAGGSLRLSCLASGSIFRFDVMGWYRQAPGKQRELVATITSGGSTNYADSVKGRFTISRDNAENTLYLQMNSLKPEDTAVYYCNANEDKTYYPLDYWGQGTQVTVSS

SEQ ID NO: 159 - protein, 118 aa, synthetic construct
QVQLVESGGGLVQAGGSLRLSCAASGSISSINAMGWYRQAPGKQRELVAGITSGGSTNYADSVKGRFTISRDNAKNTVYLQMNSLKPEDTAVYYCNLNQGDYYPIDYWGQGTQVTVSS

SEQ ID NO: 160 - protein, 121 aa, synthetic construct
QVQLVESGGGLVQAGGSLRLSCAVSGSLLSIDAMGWYRQAPGRQRELVARITSGGTTNYADSVKGRFTISRDNAKNTVYLQMNSLKPEDTAVYYCNLEESTSSDYYPIDYWGQGTQVTVSS

SEQ ID NO: 161 - protein, 121 aa, synthetic construct
QVQLVESGGGLVQAGGSLRLSCAVSGNIFSIDAMGWYRQAPGRQRELVARITSGGTTKYADSVKGRFTISRDNAKNTVYLQMNSLKPEDTAVYYCNLEESTSSDYYPIDYWGQGTQVTVSS

SEQ ID NO: 162 - protein, 121 aa, synthetic construct
QVQLVESGGGLVQAGGSLRLSCAVSGNIFSIDAMGWYRQAPGRQRELVARITSGGTTNYADSVKGRFTISRDNAKNTVYLQMNSLKPEDTAVYYCNLEESTSSDYYPIDYWGQGTQVTVSS

SEQ ID NO: 163 - protein, 127 aa, synthetic construct
QVQLVESGGGLVEAGKSLKILCVASGGTLSRYTMAWFRQAPGKEREFVAAINWKGDVTTYEESVRGRHSISRDNAKNTVYLQMNVLKPEDTGVYYCAADEVVGGWRRATNPDDHDYWGQGTQVTVSS

SEQ ID NO: 164 - protein, 128 aa, synthetic construct
QVQLVESGGGLVQPGGSLRLSCSASERAFSDYRVAWFRQAPGKEREFVASVNWNGDNSYYADSVKGRFTISRDNAKNAVYLQMNSLKPGDTAVYVCAQSPILSVDSQVVTSPAQYQYWGQGTQVTVSS

SEQ ID NO: 165 - protein, 129 aa, synthetic construct
QVQLVESGGGLVQAGGSLRLSCAITGGVFSRYTLGWFRQAPGKEREVVAAITWSSGVTYYTDSEKGRFIISRDSAKNVLYLQMSSLKPEDTAMYYCAADVDGSRRWGLYWAVSSDYDYWGQGTQVTVSS

SEQ ID NO: 166 - protein, 128 aa, synthetic construct
QVQLVESGGGLVQPGSSLRLSCAASGRTFNSLSMAWFRQAPGKEREFVAGISWIGNTVYSNSVKGRFSISEDTSKNTGYLQMNSLKPEDTAVYYCAATMDRSPSWVRTNMQSAAYNYWGQGTQVTVSS

SEQ ID NO: 167 - protein, 127 aa, synthetic construct
QVQLVQLQESGGGLVQAGGSLRLSCAASGRTESISSMGWFRQAPGKEREFVVGVYWIDGSTYYRGSVKDRFTISRDNVKNLVHLQMNNLKPEDTAVYYCAAASRGGSPVHPSSFPYWGQGTQVTVSS

SEQ ID NO: 168 - protein, 128 aa, synthetic construct
QVQLVESGGGLVQAGGSLRLSCVVSGRTWNNYAVAWFRQAPGKERDIVAAITLSAGTSYYADSVKGRFTISRDNVKESVFLQMNSLKPEDTAVYYCAAETSPLRYGDSYWDESTYDYWGQGTQVTVSS

SEQ ID NO: 169 - protein, 128 aa, synthetic construct
QVQLVESGGGLVQTGGSLRLSCAASGRTWNNYAVAWFRQAPGKERDIVAAITLSGGTSYYADSVKGRFTISRDNVKESVFLQMNSLKPEDTAVYYCAAETSPLRYSDSYWDESTYDYWGQGTQVTVSS

SEQ ID NO: 170 - protein, 118 aa, synthetic construct
QVQLVESGGGLVQPGGSLRLSCTASGFPFSSTWMNWYRQSPGKERQLVAGMSSTGGTTDYANSVKDRFTISRDNNKNTLYLQMNSLKPEDEGLYYCNRPAAIRENDDWGQGTQVTVSS

SEQ ID NO: 171 - protein, 118 aa, synthetic construct
QVQLVESGGGLVQPGGSLRLSCAASGFPFSSTWMNWYRQSPGKERQLVAGMSSTGGTTDYANSVKDRFTISRDNNKNTLYLQMNSLKPEDEGLYYCNRPAAIRENDDWGQGTQVTVSS

SEQ ID NO: 172 - protein, 128 aa, synthetic construct
QVQLVQLQESGGGLVQAGDSLRLSCTASTRTFSSHRMAWFRQAPGKEREFVAYISGSGGATYYAVPVRGRFAISRDNAKNTMYLQMNSLKPEDTAVYYCAATTFSGKSGVLTTTYDYWGQGTQVTVSS

SEQ ID NO: 173 - protein, 117 aa, synthetic construct
QVQLVQAGGSLRLSCVASGSTFRNYPVAWFRQAPGQEREYVAAIRWTTGSTVYADSVKGRFTMSRDNAKNTVYLQMNNLKPEDTGVYYCAARTGPIITSESRAYEFWGQGTQVTVSS

SEQ ID NO: 174 - protein, 124 aa, synthetic construct
QVQLVESGGGLVQPGGSLRLSCVASGSTFRNYPVAWFRQAPGQEREYVAAIRWTTGSTVYADSVKGRFTMSRDNAKNTVYLQMNNLKPEDTGVYYCAARTGPIITSESRAYEFWGQGTQVTVSS

SEQ ID NO: 175 - protein, 124 aa, synthetic construct
QVQLVESGGGLVQAGGSLRLSCVASGGTFSSYTMGWFRQAPGKERKFVAAITSSGGSTVYQNSVNGRFTISRDNAKNAVYLQMNNLKPEDTAVYYCAADGVGRSSSYVGNYGYWGQGTQVTVSS

SEQ ID NO: 176 - protein, 117 aa, synthetic construct
QVQLVESGGGLVQAGGSLRLSCRASGSTFSSGPIGWYRAPPGKERELVAFISSDGIIKYEDSVKGRFTIWRDNAKNTVYLSMIGLKPEDTAVYSCNLKRPFDRSEYWGQGTQVTVSS

SEQ ID NO: 177 - protein, 117 aa, synthetic construct
QVQLVESGGGLVQAGESLRLSCVVSGTTFSICGIGWYRQAPGKQRELVAFISSDGVTKYADSVKGRFTISRDNAKNTVYLSMVGLKPDDTAVYSCKLKRPFDRSEYWGQGTQVTVSS

SEQ ID NO: 178 - protein, 121 aa, synthetic construct
QVQLVESGGGLVQAGGSLRLSCTGSGITFSDNPIGWYRQAPGNQREMVAGIGSGGRTNYINAVKGRFTISRDNTKNTVYLQMNDLRPEDTAVYYCNVRALYYSDYHASEYWGQGTQVTVSS

SEQ ID NO: 179 - protein, 114 aa, synthetic construct
QVQLVESGGGLVQPGGSLRLSCAASGFTVSSYLIRWYRQATGKQREFVAAITSGGEDRNYADSVKGRFTISTDNAKNTVALQMNNLKPEDTAIYYCNILPGDFWGQGTQVTVSS

SEQ ID NO: 180 - protein, 114 aa, synthetic construct
QVQLVESGGGLVQAGGSLRLSCAASGFTVSSYLIRWYRQATGKQREFVAAITSGGEDRNYADSVKGRFTISTDNAKNTVALQMNNLKPEDTAIYYCNILPGDFWGQGTQVTVSS

SEQ ID NO: 181 - protein, 120 aa, synthetic construct
QVQLVESGGGLVQPGESLRLSCAASGSIFSFYAMGWYRQAPGKEREWVVDISRDGATKYAASVKGRFTISRDNAKNAGYLEMNSLKPEDTAVYYCYLVGALVGESRREYWGQGTQVTVSS

SEQ ID NO: 182 - protein, 113 aa, synthetic construct
QVQLVESGGGLVQPGGSLRLSCTSSTRYFNIHAMGWYRQAPGKERELVARINSGGRTSYADSVKGRFAISRDNARKTVYLQMNNLMPEDTGDYYCNLDSQINWGQGTQVTVSS

SEQ ID NO: 183 - protein, 113 aa, synthetic construct
QVQLVESGGGLVQPGGSLRLSCTSSTRYFNIHAMGWYRQAPGKERELVARINSGGRTSYADSVKGRFAISRDNARKTVYLQMNNLMPEDTGVYYCNLDSQINWGQGTQVTVSS

SEQ ID NO: 184 - protein, 113 aa, synthetic construct
QVQLVESGGGLVQPGGSLRLSCTSSTRYFNIHAMGWYRQAPGKERELVARINSGGRTSYADSVKGRFAISRDNARKTVDLQMNDLMPDDTGVYYCNLDSQINWGQGTQVTVSS

SEQ ID NO: 185 - protein, 113 aa, synthetic construct
QVQLVESGGGLVQPGGSLRLSCTSSTRYFNIHAMGWYRQAPGKERELVARINSGGRTSYADSVKGRFAISRDNDRKTVDLQMNSLMPEDAGVYYCNLDSQINWGQGTQVTVSS

SEQ ID NO: 186 - protein, 113 aa, synthetic construct
QVQLVESGGGLVQPGGSLRLSCTSSTRYFNIHAMGWYRQAPGKERELVARINSGGRTSYADSVKGRFAISRDNARKTVDLQMNRLLPEDTGVYYCNLDSQINWGQGTQVTVSS

SEQ ID NO: 187 - protein, 113 aa, synthetic construct
QVQLVESGGGLVQPGGSLRLSCTSSTRYFNIHAMGWYRQAPGKERQLVARINSGGRTSYADSVKGRFAISRDNARKTVDLQMNSLMPEDTGVYYCNLDSQINWGQGTQVTVSS

SEQ ID NO: 188 - protein, 113 aa, synthetic construct
QVQLVESGGGLVQPGGSLRLSCTSSTRYFNIHAMGWYRQAPGKERELVARINSGGRTSYADSVKGRFAISRDNARKTVDLQMNSLMPEDTGVYYCNLDSQINWGQGTQVTVSS

SEQ ID NO: 189 - protein, 113 aa, synthetic construct
QVQLVESGGGSVQPGGSLRLSCTSSTRYFNIHAMGWYRQAPGKERELVARINSGGRTSYSDSVKGRFAISRDNARKTVDLQMNSLMPEDTGVYYCNLDSQINWGQGTQVTVSS

SEQ ID NO: 190 - protein, 113 aa, synthetic construct
QVQLVESGGGLVQPGGSLRLSCTSSTRYFNIHAMGWYRQAPGKERELVARINSGGRTSYSDSVKGRFAISRDNARKTVDLQMNSLMPEDTGVYYCNLDSQINWGQGTQVTVSS

SEQ ID NO: 191 - protein, 113 aa, synthetic construct
QVQLVESGGGWVQPGGSLNLSCASSTTYFSIHAMGWYRQAPGKQRELVARINSGGRTTYADSVKGRFAISRDNARKTVWLQMNSLKPEDTGVYYCNLDSQINWGQGTQVTVSS

SEQ ID NO: 192 - protein, 113 aa, synthetic construct
QVQLVESGGGLVRPGGSLRLSCASSTTYFSIHAMGWYRQAPGKQRELVARINSGGRTTYADSVKGRFAISRDNAKKTVYLQMNSLKPEDTGVYYCNLDSQINWGQGTQVTVSS

SEQ ID NO: 193 - protein, 113 aa, synthetic construct
QVQLVESGGGLVQPGGSLRLSCASSGIAFSLFTMGWYRQAPGKQHELVARITGSGITNYADSVKGRFTISRDNVKKMVYLQMSSLEPEDTAVYYCNTELQGAWGQGTQVTVSS

SEQ ID NO: 194 - protein, 113 aa, synthetic construct
QVQLVESGGGLVQPGGSLRLSCASSGSAFSLITMGWYRQAPGKQHELVARITGSGITNYADSVKGRFTISRDNVKKMVYLQMSSLEPEDTAVYYCNTELRGAWGQGTQVTVSS

SEQ ID NO: 195 - protein, 116 aa, synthetic construct
QVQLVESGGGLVQPGGSLRLSCAASGGSFSISTVNWSWYRQAPGKQRELVATIGNIDRTAYADSVKGRFTISRDNGKNTVYLQMNSLKPEDTAVYYCNLKRLNEYWGQGTQVTVSS

SEQ ID NO: 196 - protein, 116 aa, synthetic construct
QVQLVESGGGLVQPGGSLRLSCAASGGSFSISTVNWSWYRQAPGKHRELVATIGNIDRTAYADSVKGRFTISRDNGKNTVYLQMNSLKPEDTAVYYCNLKRLNEYWGQGTQVTVSS

SEQ ID NO: 197 - protein, 120 aa, synthetic construct
QVQLVESGGGLVEPGGSLRLSCAASGRFFNIGAMDWYRQVPGNQRERVARITSDGSTFYLDSVEGRFTISRDNAKNTVYLQMNSLKSEDTAVYYCFAFIESRNGVYDEYWGQGTQVTVSS

SEQ ID NO: 198 - protein, 120 aa, synthetic construct
QVQLVESGGGLVQSGGSLRLSCAASGRFFNIGAMDWYRQVPGNQRERVARITSDGSTFYLDSVEGRFTISRDNAKNTVYLQMNSLKSEDTAVYYCFAFIESRNGVYDEYWGQGTQVTVSS

SEQ ID NO: 199 - protein, 120 aa, synthetic construct
QVQLVESGGGLVQPGGSLRLSCAASGSFFNIGAMDWYRQVPGNQRERVARITMDGSTFYLDSVKGRFTISRDNAKNTVYLQMNSLKSEDTAVYYCFAFIDSGNGVYDEFWGQGTQVTVSS

SEQ ID NO: 200 - protein, 120 aa, synthetic construct
QVQLVESGGGLVQPGGSLRLSCAASGSFFNIGAMDWYRQVPGNQRERVARITADGTPFYLPSVKGRFTISRDNAKNTVYLQMNSLKSEDTAVYYCFAFIDSENGVYDDYWGQGTQVTVSS

SEQ ID NO: 201 - protein, 120 aa, synthetic construct
QVQLVESGGGLVQPGGSLRLSCAASGSFFNIGAMDWYRQVPGNQRERVARITADGTPFYLPSVKGRFTISRDNAKNTVYLQMNSLKSEDTAVYYCFAFIDSGNGVYDDYWGQGTQVTVSS

SEQ ID NO: 202 - protein, 118 aa, synthetic construct
QVQLVESGGGLVQPGGSLRLSCAASGFDFKIYAMGWYRQAPGKQRELVASITHDGITNYADSVKGRFTISRDNAKNTVYLQMNSLKSEDTAVYYCNRVIGPIARINYWGQGTQVTVSS

SEQ ID NO: 203 - protein, 123 aa, synthetic construct
QVQLVESGGGLVQPGGSLRLSCAASGFTFSRYAMSWYRQAPGKEREWVATITSAGGSTNYANFVKGRFTISRDNAKNMAYLQMNSLKPDDTAVYLCNADRAVAWSGDYYRNYWGQGTQVTVSS

SEQ ID NO: 204 - protein, 123 aa, synthetic construct
QVQLVESGGGLVQPGGSLRLSCAASEFTFSRYAMSWYRQAPGKEREWVATITSTGGSTNYADFVKGRFTISRDNAKNTVYLQMDSLKPDDTAVYLCNADRAVQWSGDYYRNYWGQGTQVTVSS

SEQ ID NO: 205 - protein, 122 aa, synthetic construct
QVQLVESGGGLVQPGGSLRLSCAASGFSFSRYAMNWYRQAPGKGRDFVAAITSTGSTNYADSVAGRFTISRDNDKNTVYLQMNSLKPEDTAVYYCNADRAVSYSGSYYENYWGQGTQVTVSS

SEQ ID NO: 206 - protein, 123 aa, synthetic construct
QVQLVESGGGLVQPGGSLRLSCAASGFTFSRFAMSWYRQAPGKGRELVAAITAAGGSTNYADSVKGRFTISRDNAKNTVYLQMNSLKPEDTAVYYCNADRAVVYSGDYYRNYWGQGTQVTVSS

SEQ ID NO: 207 - protein, 123 aa, synthetic construct
QVQLVESGGGLVQPGGSLRLSCAASGFTFSRFAMSWYRQAPGKGRELVAAISSAGGSTNYGDSVKGRFTISRDNAKNTVYLQMNSLKPEDTAVYICNADRAVTYSGDYYRNYWGQGTQVTVSS

SEQ ID NO: 208 - protein, 122 aa, synthetic construct
QVQLVESGGGLVQPGGSLRLSCAASGRIFRLYAMGWYRQAPGKQRELVATITTGGSTNYADSVKGRFTISRDNAKNMVYLQMNDLKPEDTAVYYCAASPNIAILTSGTYQYWGQGTQVTVSS

SEQ ID NO: 209 - protein, 123 aa, synthetic construct
QVQLVESGGGLVQAGGSLRLSCDASGGTFSGYALGWFRQAPGKDREFVAATDWSGISTSYADSVKGRFTISRDNAKNMVYLQMNDLKPEDTAVYYCAASPNIAIMTSGTYQYWGQGTQVTVSS

SEQ ID NO: 210 - protein, 120 aa, synthetic construct
QVQLVESGGGLVQPGGSLRLACVISGHADIAWNMGWFRQAPGKEREVVAAITGSGEITYYSDTVKGRFTISRDNAKNTVYLQMSSLKPEDTGVYYCAAKSRTWPQKYDYWGQGTQVTVSS

SEQ ID NO: 211 - protein, 123 aa, synthetic construct
QVQLVQLQESGGGSVQAGGSLRLDCVISGHADIAWNMGWFRQAPGKEREVVAAITGSGEITYYSDSVKGRFTISRDNAKNTVYLQMSSLKPEDTGVYYCAAKSRTWPQKYDYWGQGTQVTVSS

SEQ ID NO: 212 - protein, 120 aa, synthetic construct
QVQLVESGGGSVQAGGSLRLACVISGHADIAWNMGWFRQAPGKEREVVAVITGSGEITYYSDTVKGRFTISRDNAKNTVYLQMSSLKPEDTGVYYCAAKSRTWPQKYDYWGQGTQVTVSS

SEQ ID NO: 213 - protein, 120 aa, synthetic construct
QVQLVESGGGSVQAGGSLRLACVISGHADIAWNMGWFRQAPGKEREVVAAITGSGEITYYSDTVKGRFTISRDNAKNTVYLQMSSLKPEDTGVYYCAAKSRTWPQKYDYWGQGTQVTVSS

SEQ ID NO: 214 - protein, 120 aa, synthetic construct
QVQLVESGGGSVQAGGSLRLACAISGHSDIHYNMGWFRQAPGKEREVVAAITGSGGITYYTSSVKGRFTISRDNAKNTVYLQMNSLKPEDTGVYYCAAKSRTWPQKYDYWGQGTQVTVSS

SEQ ID NO: 215 - protein, 120 aa, synthetic construct
QVQLVESGGGSVQAGGSLTLACVISGHSEVHWNMGWFRQAPGKEREVVTAITGSGGITYYADSVKGRFTISRDNAKNTVYLQMNSLKPEDTGVYYCAAKSRTWPQKYDYWGQGTQVTVSS

SEQ ID NO: 216 - protein, 126 aa, synthetic construct
QVQLVESGGGLVQPGGSLTLSCVVSGRTFDSYATGWFRQAPGKEREFVAAITWSGILTRYADSAKGRFTISRDNAKNTTYLQMNGLKAEDTAVYYCADRLRGRGNIYSDTHTYDYWGQGTQVTVSS

SEQ ID NO: 217 - protein, 126 aa, synthetic construct
QVQLVESGGGLVQAGGSLRLSCVVSGRTIDSWATGWFRQAPGKEREFVAAIAWGGTLTKYTDSAKGRFTISNDNAKSTVYLQMNGLKPEDTAVYYCADRLRGRGNIYSDTLSYDYWGQGTQVTVSS

SEQ ID NO: 218 - protein, 126 aa, synthetic construct
QVQLVESGGGLVQAGGSLRLSCVVSGRTIDSWATGWFRQAPGKEREFVAAITWSGTLTKYTDSAKGRFTISKDNAKSTVYLQMNGLKPEDTAVYYCADRLRGRGNIYSDTLSYDYWGQGTQVTVSS

SEQ ID NO: 219 - protein, 126 aa, synthetic construct
QVQLVESGGGLVQPGGSLRLSCVVSGRTIDSWATGWFRQAPGKEREFVAAITWSGTLTKYTDSAKGRFTISNDNAKSTVYLQMNGLKPEDTAVYYCADRLRGRGNIYSDTLSYDYWGQGTQVTVSS

SEQ ID NO: 220 - protein, 126 aa, synthetic construct
QVQLVESGGGLVQAGGSLRLSCVVSGRTIDSWATGWFRQAPGKEREFVAAITWSGTLTKYTDSAKGRFTISNDNAKSTVYLQMNGLKPEDTAVYYCADRLRGRGNIYSDTLSYDYWGSGTQVTVSS

SEQ ID NO: 221 - protein, 126 aa, synthetic construct
QVQLVESGGGLVQAGGSLRLSCVVSGRTIDSWATGWFRQAPGKEREFVAAITWSGTLTKYTDSAKGRFTISNDNAKSTAYLQMNGLKPEDTAVYYCADRLRGRGNIYSDTLSYDYWGQGTQVTVSS

SEQ ID NO: 222 - protein, 126 aa, synthetic construct
QVQLVESGGGLVQAGGSLRLSCVVSGRTIDSWATGWFRQAPGKEREFVAAITWSGTLTKYTDSAKGRFTISNDNAKSTVYLQMNGLKPEDTAVYYCADRLRGRGNIYSDTLSYDYWGQGVQVTVSS

SEQ ID NO: 223 - protein, 126 aa, synthetic construct
QVQLVESGGGLVQAGDSLRLSCVVSGRTIDSWATGWFRQAPGKEREFVAAITWSGTLTKYTDSAKGRFTISNDNAKSTVYLQMNGLKPEDTAVYYCADRLRGRGNIYSDTLSYDYWGQGTQVTVSS

SEQ ID NO: 224 - protein, 126 aa, synthetic construct
QVQLVESGGGLVQAGGSLRLSCVVSGRTIDSWATGWFRQAPGKEREFVAAITWSGTLTKYTDSAKGRFTISNDNAKSTVYLQMNGLKPEDTAVYYCADRLRGRGNIYSDTLSYDYWGQGTQVTVSS

SEQ ID NO: 225 - protein, 126 aa, synthetic construct
QVQLVESGGGLVQAGGSLRLSCVVSGRTVDNYATGWFRQAPGKEREFVAAIPWSGTMTKYADSAKGRFTISRDNAKNTVYLQMNSLKPEDTAVYYCADRLRGRGNIYSDTLSYDYWGQGTQVTVSS

SEQ ID NO: 226 - protein, 126 aa, synthetic construct
QVQLVESGGGLVQAGGSLRLSCVVSGRTVDNYATGWFRQAPGKEREFVAAIPWSGTMTKYADSAKGRFTISRDNAKNTVYLQMNGLKPEDTAVYYCAGRLRGRGNIYSDTLSYDYWGRGTQVTVSS

SEQ ID NO: 227 - protein, 126 aa, synthetic construct
QVQLVESGGGLVQAGGSLRLSCVVSGRTVDNYATGWFRQAPGKEREFVAAIPWSGTMTKYADSAKGRFTISRDNAKSTVYLQMNGLKPEDTAVYYCAGRLRGRGNIYSDTLSYDYWGQGTQVTVSS

SEQ ID NO: 228 - protein, 126 aa, synthetic construct
QVQLVESGGGLVQAGGSLRLSCVVSGRTVDNYATGWFRQAPGKEREFVAAIPWSGTMTKYANSAKGRFTISRDNAKSTVYLQMNGLKPEDTAVYYCAGRLRGRGNIYSDTLSYDYWGQGTQVTVSS

SEQ ID NO: 229 - protein, 126 aa, synthetic construct
QVQLVESGGGLVQAGGSLRLSCVVSGRTFDSYATGWFRQAPGKEREFVAAITWSGTMTKYADSAKGRFTISRDNAKNTTYLQMNGLKPEDTAVYYCADRLRGRGNIYSDTLSYDYWGQGTQVTVSS

SEQ ID NO: 230 - protein, 126 aa, synthetic construct
QVQLVESGGGLVQPGGSLRLSCEASGRTFDSYATGWFRQAPGKEREFVAAITWSGTMTKYADSAKGRFTISRDNAKNTTYLQMNGLKPEDTAVYYCADRLRGRGNIYSDTLSYDYWGQGTQVTVSS

SEQ ID NO: 231 - protein, 126 aa, synthetic construct
QVQLVESGGGLVQPGGSLRLSCTVSGRTFDSYATGWFRQAPGKEREFVAAITWSGTMTKYADSAKGRFTISRDNAKNTTYLQMNGLKPEDTAVYYCADRLRGRGNIYSDTLSYDYWGQGTQVTVSS

SEQ ID NO: 232 - protein, 126 aa, synthetic construct
QVQLVESGGGLVQAGGSLALSCVVSGRTVDSYATGWFRQAPGKEREFVAAITWSGTMTKYADSAKGRFTISRDDAKNTVWLQMNGLKPEDTAVYYCVDRLRGRGNIYSDTLSYDYWGQGTQVTVSS

SEQ ID NO: 233 - protein, 126 aa, synthetic construct
QVQLVESGGGLVQAGGSLALSCVVSGRTVDSYATGWFRQAPGKEREFVAAITWSGTMTKYADSAKGRFTISRDNAKSTVYLQMNGLKPEDTAVYYCVDRLRGRGNIYSDTLSYDYWGQGTQVTVSS

SEQ ID NO: 234 - protein, 123 aa, synthetic construct
QVQLVESGGGLVQAGGSLRLSCAASGRTFGNYTMGWFRQAPGKGRAFVALINWNGISTTYAGSVQGRFTISRDMAKNTVYLQMDSLKPEDTAVYYCAANLRSDSSYQGHYDYWGQGTQVTVSS

SEQ ID NO: 235 - protein, 123 aa, synthetic construct
QVQLVESGGGLVQAGGSLRLSCVVSGRTFGNYTMGWFRQAPGKGRAFVALINWNGISTTYAGSVQGRFTISRDMAKNTVYLQMDSLKPEDTAVYYCAANLRSDSSYQGHYDYWGQGTQVTVSS

SEQ ID NO: 236 - protein, 128 aa, synthetic construct
QVQLVESGGGLVQAGDSLRLSCVASGGSFHTYVMGWFRQAPGKEREFVATINYSGSNIYYANSVKGRFTISRDNAKTTVYLQMNSLKPEDTAVYYCAAQVDVYGSMWYMREPTRYDHWGQGTQVTVSS

SEQ ID NO: 237 - protein, 128 aa, synthetic construct
QVQLVESGGGLVQAGGSLRLSCVASGRTFSTYVMGWFRQAPGKEREFVATINWSGSSTYYAKSVEGRFTISRDNAKNTVYLQMDSLKPEDTAVYYCAAQVDTYGSMWHMMEPTHYEYWGQGTQVTVSS

SEQ ID NO: 238 - protein, 128 aa, synthetic construct
QVQLVESGGGLVQAGGSLRLSCVASGRTFSTYVMGWFRQAPGKEREFVATINWSGSSTYYAKSVEGRFTISRDNAKNTVYLQMDSLKPEDTAVYYCAAQVDTYGSMWHMMEPTHYDYWGQGTQVTVSS

SEQ ID NO: 239 - protein, 131 aa, synthetic construct
QVQLVQLQESGGGLVQAGGSLRLSCTVSGRTFSNYAITWFRQAPGKGRDFVAAVTWSGGATYYADSVTGRFTISRDNAKNTVYLQMNSLKPEDTAVYYCAARRDTNSGSYYYTMDYGMDYWGKGTQVTVSS

SEQ ID NO: 240 - protein, 129 aa, synthetic construct
QVQLVESGGGLVQAGGSLRLSCAASGRTFSSYTMGWFRQAPGKEREYVAAIRWSGGYTYYADSVKGRFTISRDIAKNTVYLQMNSLKPEDTAVYYCAADIIQDGLGSYYYTQGHMYDYWGQGTQVTVSS

SEQ ID NO: 241 - protein, 129 aa, synthetic construct
QVQLVESGGGLVQAGGSLRLSCAASGRTFSRYTMGWFRQVPGKEREYVAAIRWSGGYTYYADSVKGRFTISRDIAKNTGYLQMNSLKPEDTAVYYCAADIIQDGLGSYYYTQGHMYDYWGQGTQVTVSS

SEQ ID NO: 242 - protein, 132 aa, synthetic construct
QVQLVQLQESGGGLVQAGGSLRLSCAASGGTFSNYAMGWFRQAPGKERQFVAAISWTHGNTYYADSAKGRFTISRDNAKNTVYLQMNSLKPEDTAVYYCAARKSGEYSGSYYLQFDFGYDYWGQGTQVTVSS

SEQ ID NO: 243 - protein, 119 aa, synthetic construct
QVQLVESGGGLVQPGGSLRLSCAASGRTESTVAIGWFRQAPGKEREFVAAIAWSGSWTDIPDSVKGRFTISRDNAKNTVYLQMNSLKPEDTAVYYCAARGSRTYKYEYWGPGTQVTVSS

SEQ ID NO: 244 - protein, 119 aa, synthetic construct
QVQLVESGGGLVQPGGSLRLSCAASGRTESTVAIGWFRQAPGKEREFVAAIAWSGSWTDIPDSVKGRFTISRDNAKNTVYLQMNSLKPEDTAVYYCAARGSRTYKYEYWGQGTQVTVSS

SEQ ID NO: 245 - protein, 128 aa, synthetic construct
QVQLVESGGGLVQPGGSLRLSCAASGFTFSFYAMTWVRQAPGKGLEWVSRINSGGGSTSYADSVKGRFTISRDNAKNTLYLQMNSLKPEDTAVYYCALPRFASSSRYIDEIGAEYDYWGQGTQVTVSS

SEQ ID NO: 246 - protein, 126 aa, synthetic construct
QVQLVQLQESGGGLVQPGGSLRLSCAASGFTFSTSAMSWARQAPGKGLEWVSGIYSDFSTYYADSVKGRFTISRDNAKNMVYLQMNSLKPEDTAVYYCAIDSYLADSLNWRRSDYRGQGTQVTVSS

SEQ ID NO: 247 - protein, 126 aa, synthetic construct
QVQLVQLQESGGGLVQPGGSLRLSCAASGFTFSTSAMSWARQAPGKGLEWVSGIYSDDSTYYADSVKGRFTISRDNAKNTVYLQMDSLKPEDTAVYYCAIDSYLPDSLNWRRSDYRGQGTQVTVSS

SEQ ID NO: 248 - protein, 120 aa, synthetic construct
QVQLVESGGGLVQPGGSLRLSCAASGFTFSSSPMSWVRQAPGKGLEWVSSFYPHSSDTYFGDSVKGRFTIFTDNAKNTLYLQMNSLKPEDTAVYYCAADRSDYAGVFDYWGQGTQVTVSS

SEQ ID NO: 249 - protein, 130 aa, synthetic construct
QVQLVESGGGLVQPGGSLRLSCAASGFSSDYYIIVWFRQAPGKEREGASCISAISSSDGSTYYADSVKGRFTISRDNAKNTVHLLMNSLKPDDTAVYYCAAAERIHTITEPPRAPYMDYWGKGTQVTVSS

SEQ ID NO: 250 - protein, 115 aa, synthetic construct
QVQLVESGGGLVQPGGSLRLSCAASGFTVDSSYTSWYRQAPGTEREWVATITSLDGRASYANSVKGRFTISRDNAKNTVYLQMNNLKPEDTAVYYCYARDGYDRWGQGTQVTVSS

SEQ ID NO: 251 - protein, 126 aa, synthetic construct
QVQLVESGGGLVQAGGSLRLSCAASGFTFDDYAIGWFRQAPGKEREGVSCISSSDGSTYYAESVKGRFTISSDLAKNTVYLQMNSLKPEDTAVYYCGVDWDVPYCSDLLSAPYDYWGQGTQVTVSS

SEQ ID NO: 252 - protein, 126 aa, synthetic construct
QVQLVESGGGLVQAGESLRLSCAASGFTFDDYAIGWFRQAPGKEREGVSCISSSDGSTYYAESVKGRFTISSDLAKNTVYLQMNSLKPEDTAVYYCGVDWDVPYCSDLLSAPYDYWGQGTQVTVSS

SEQ ID NO: 253 - protein, 122 aa, synthetic construct
QVQLVESGGGLVQPGSSLRLSCAASGFIFDDYAIGWFRQAPGQEREGLACVSSSGGSTFYSDSVKGRFTISRDSDGNTVHLEMNNLKPEDTAIYYCATPWISTCPTNEYSFWGQGTQVTVSS

SEQ ID NO: 254 - protein, 126 aa, synthetic construct
QVQLVESGGGLVQPGGSLRLSCAASGFTLDYYAIGWFRQAPGKEREGVSCITSSDGSTKYAESVKGRFTISRDNEKNTVYLQMNSLKPEDTAVYYCATGDCHPDVVISTITGYDSWGQGTQVTVSS

SEQ ID NO: 255 - protein, 122 aa, synthetic construct
QVQLVESGGGLVPPGDSLTLSCVVSGVRLYDYAVGWFRQAPGKEREGVSCVSARDGSTEYADAVKGRFTISTDSVKSTAYLQMNNLKPEDTAIYYCATPWISTCPTNEYSFWGQGTQVTVSS

SEQ ID NO: 256 - protein, 122 aa, synthetic construct
QVQLVESGGGLVQPGVSLRLSCTASGFSLDDFAIGWVRQAPGKEREGVSCIATSDGSTHYAESVKGRFTISTDSVKSTAYLQMNNLKPEDTAIYYCVTPWISTCPTNEYSFWGQGTQVTVSS

SEQ ID NO: 257 - protein, 122 aa, synthetic construct
QVQLVESGGGLVQPGGSLRLSCTASGFSLDDFAIGWVRQAPGKEREGVSCIAASDGSTHYADSVKGRFTISTDSVKSTAYLQMNNLKPEDTAIYYCATPWISTCPTNEYSFWGQGTQVTVSS

SEQ ID NO: 258 - protein, 124 aa, synthetic construct
QVQLVESGGGLVQPGGSLRLSCAASPSGFTLDDYAMGWFRQAPGKEREGVSCISSSDGSTYYADSVKGRFTISRDNAKNTVTLQMNNLKPEDTAVYYCATPWINYCNGFEFSHWGQGTQVTVSS

SEQ ID NO: 259 - protein, 122 aa, synthetic construct
QVQLVESGGGLVQPGGSLRLSCAASGFSLDDFAIGWVRQAPGKEREGVSCIAASDGSTHYADSVKGRFTISRNNDKKTVTLLMNNLKPEDTAVYYCATPWITYCNGFEFSNWGQGTQVTVSS

SEQ ID NO: 260 - protein, 122 aa, synthetic construct
QVQLVESGGGLVQPGGSLRLSCAASGFSLDDFAIGWVRQAPGKEREGVSCIAASDGSTHYADSVKGRFTISRDNAKNTVTLQMNNLKPEDTAVYYCATPWINYCNGFEFSHWGQGTQVTVSS

SEQ ID NO: 261 - protein, 123 aa, synthetic construct
QVQLVESGGGLVQPGGSLRLSCAASGFTFSRYAMSWYRQAPGKEREWVATVTSAGGSTNYADFVKGRFTISRDNAKNTVYLQMNSLKLEDTAVYLCNADRAVTWSGDYYRNYWGQGTQVTVSS

SEQ ID NO: 262 - protein, 121 aa, synthetic construct
QVQLVESGGGLVQAGGSLRLSCAASGRTFSSYDMGWYRQAPGKERRFVAAISWSGSRPFYADSVKGRFTISRDNAKNTLYLQMSSLKPEDTAIYYCHAVPPGSWSQDSGYWGQGTQVTVSS

SEQ ID NO: 263 - protein, 118 aa, synthetic construct
QVQLVESGGGLVQAGGSLRLSCAASGRSSYDMGWYRQAPGKEREFVADIRWISGNTYYADSVKGRFTISRDNAKNTVYLQMNSLKPEDTAIYYCNAETVTDTVQSTYWGQGTQVTVSS

SEQ ID NO: 264 - protein, 119 aa, synthetic construct
QVQLVESGGGLAQAGGSLRLSCVASGGTFSSYAMGWFRQASGKAREFVAAIRRSGGSTYYADSVEGRFTISRDNAKNTVYLQMNSLTPEDTAVYYCAAGDDLVDEYEYWGQGTQVTVSS

SEQ ID NO: 265 - protein, 120 aa, synthetic construct
QVQLVESGGGSVQAGGSLRLSCAAAGRTFGSYDMGWYRQAPGKEREFVADIRWISGNTYYADSVKGRFTISRDNAKNTVYLQMNSLKPEDTAIYYCNAETVTDTVQSTYWGQGTQVTVSS

SEQ ID NO: 266 - protein, 122 aa, synthetic construct
QVQLVESGGGLVQAGGSLRLSCAVSGRTFSIYDMGWYRQAPGKEREFVAGISWGGGTIHYADSVKGRFTISRDNAKNTVYLQMNSLKPEDTAIYYCFFDSDPGGTGTLHGSWGQGTQVTVSS

SEQ ID NO: 267 - protein, 120 aa, synthetic construct
QVQLVESGGGLVQAGGSLRLSCATSGRTFKSYDMGWSRQAPGKEREFVADIRWISGNTYYADSVKGRFTISRDNAKNTVYLQMNNLKPEDTAIYYCNAETVTDTVQSTYWGQGTQVTVSS

SEQ ID NO: 268 - protein, 123 aa, synthetic construct
QVQLVESGGGLVQAGGSLRLSCAASGSIGNIDDMGWYRQAPGKQREFVADINSGGSTNYADSVKGRFTISRDSAKNTVYLQMNSLKTEDTAVYYCNADALFRDAGVYRREVVWGQGTQVTVSS

SEQ ID NO: 269 - protein, 115 aa, synthetic construct
QVQLVESGGGLVQAGGSLRLSCAASVSRFSIDIMGWYRQAPGKERELVASITVGGTTTYADSVKGRFSISRDITKNGMHLQMDSLKPDDTGVYYCNLRRSSSDYWGQGTQVTVSS

SEQ ID NO: 270 - protein, 120 aa, synthetic construct
QVQLVESGGGLVQPGDSLRLSCAASGLSLSTYAVAWFRQAPGKKREFIAGIRRIAGRTYYADSVEGRFTISRDNAENTVYLQMDSLKPEDTAVYYCAADPQDIVDAYEYWGQGTQVTVSS

SEQ ID NO: 271 - protein, 119 aa, synthetic construct
QVQLVESGGGLVQAGGSLRLSCAASRGTFSTDVMGWYRQAAGNQRELVATITAGRTTNYADSVKGRFTIARENAKNTVYLQMNSLKPEDTAVYYCNLRTDIRDNPYNYWGPGTQVTVSS

SEQ ID NO: 272 - protein, 126 aa, synthetic construct
QVQLVESGGGLVQPGGSLRLSCTASGFTFGDYDMSWVRQAPGKGPEWVSAINSGGSRTYYQDSVKGRFTISRDNAKNTLYLQMNSLKPEDTAVYYCAAGDSDASGRFTYPTDRGRWGQGTQVTVSS

SEQ ID NO: 273 - protein, 117 aa, synthetic construct
QVQLVESGGGLVQAGGSLRLSCASSRSRWSTDIMGWYRQAPGKQRELVATITVGGVTNYADSVKGRFTISRDRAKTMWYLQMNSLKPDDTAVYFCNLVSLDDSGNYWGQGTQVTVSS

SEQ ID NO: 274 - protein, 120 aa, synthetic construct
QVQLVESGGGLVQAGGSLRLSCAVSGGTFSSYDMGWYRQAPGKEREFVADIRWISGNTYYADSVKGRFTISRDNAKNTVYLQMNNLKPEDTAIYYCNAETVTDTVQSTYWGQGTQVTVSS

SEQ ID NO: 275 - protein, 121 aa, synthetic construct
QVQLVESGGGLVQEGGSLRLSCAASGRTFSSYDMGWYRQAPGKERRFVAAISWSGSRPFYADSVKGRFTISRDNAKNTLYLQMSSLKPEDTAIYYCHAVPPGSWSQDSGYWGQGTQVTVSS

SEQ ID NO: 276 - protein, 125 aa, synthetic construct
QVQLVESGGGLVQAGGSLTLSCVASGNILSGSTMGWYRQAPGKQRELVATITVDGSTNYVDSAKGRFTISRDNAKNTVYLQMNSLKPEDTGVYYCNADGLEREKVGTPYKRGNYWGQGTQVTVSS

SEQ ID NO: 277 - protein, 120 aa, synthetic construct
QVQLVESGGGLTQAGNSLRLACTASGGAFSSYDMGWYRQAPGKEREFVADIRWISGSTYYADSVKGRFTISRDNAKNTVYLQMNSLKPEDTAIYYCNAETVTDTVQSTYWGQGTQVTVSS

SEQ ID NO: 278 - protein, 116 aa, synthetic construct
QVQLVESGGGLVQAGGSLRLSCAASRSRYSTDIMGWYRQAPGKQRELVASITVGGVINYADSVKGRFTISRDTVRNTWYLQMNSLKPEDTAVYFCNLHTEYSENYWGQGTQVTVSS

SEQ ID NO: 279 - protein, 120 aa, synthetic construct
QVQLVESGGGLVQAGGSLRLSCAASGGTFSSYDMGWYRQAPGKEREFVADIRWISGNTYYADSVKGRFTISRDNAKNTVYLQMIGLKPEDTAIYYCNAETVTDTVQSTYWGQGTQVTVSS

SEQ ID NO: 280 - protein, 126 aa, synthetic construct
QVQLVESGGGLVQPGGSLRLSCTASGFTFGDYDMSWVRQAPGKGPEWVSAINSGGSSTYYQDSVKGRFTISRDNAKNTLYLQMNSLKPEDTAVYYCAAGSSDVSGSFYYPTDRGRWGQGTQVTVSS

SEQ ID NO: 281 - protein, 126 aa, synthetic construct
QVQLVESGGGLVQPGGSLRLSCAASGFTFGSSDMAWVRQAPGKGPEWVSGINSGGSRTYYQDSVKGRFTISRDNAKNTLYLQMNSLKPEDTAVYYCAAGDSDASGRFTYPTDRGRWGQGTQVTVSS

SEQ ID NO: 282 - protein, 118 aa, synthetic construct
QVQLVESGGGLVQAGGSLRLSCVASGGTFSNYAMGWFRQAPGKVREFVAAIRSGGSTYYADSVTGRFTITRDNAKNTVYLQMKSLKPEDTAVYYCAAGPDAVDEYDYSGQGTQVTVSS

SEQ ID NO: 283 - protein, 122 aa, synthetic construct
QVQLVESGGGLVQAGGSLRLSCAASGRTVDYDMGWYRQAPGREREFVASITLHGGNTYYADSVKGRFTISRDNAKNTVYLQMNSLKPEDTAIYYCTADYYSDYDGGRLLGSWGQGTQVTVSS

SEQ ID NO: 284 - protein, 121 aa, synthetic construct
QVQLVESGGGLVQAGGSLRLSCAASGRTFSSYDMGWYRQAPGKEREFVAAISWSGSRPFYADSVKGRFTISRDNAKNTLYLQMSSLKPEDTAIYYCHAVPPGSWSQDSGYWGQGTQVTVSS

SEQ ID NO: 285 - protein, 120 aa, synthetic construct
QVQLVESGGGVVQTGGSLRLSCAASGGTFSSYDMGWYRQAPGKEREFVADIRWISGNTYYADSVKGRFTISRDNAKNTVYLQMNSLKPEDTAIYYCNAETVTDTVQSTYWGQGTQVTVSS

SEQ ID NO: 286 - protein, 118 aa, synthetic construct
QVQLVESGGGLVQAGGSLRLSCAGSGRTLSSYAMGWFRQAPGKLREFVAAIRSSDSTYYADSVEGRFTISRDNAKNTVYLQMNSLKPEDTAVYYCAAGPDNSDAYDYWGQGTQVTVSS

SEQ ID NO: 287 - protein, 120 aa, synthetic construct
QVQLVESGGGLVQAGGSLRLSCAASGGTFSSYDMGWYRQAPGKEREFVADIRWISGNTYYADSVKGRFTISRDNAKNTVYLQMNSLKPEDTAIYYCNAETVTDTVQSTYWGQGTQVTVSS

SEQ ID NO: 288 - protein, 122 aa, synthetic construct
QVQLVESGGGLVQAGGSLRLSCAASGRTFGSYDMGWYRQAPGKGPEWVSITNSGGGNTYYADSVKGRFTISRDNAKNTLYLQMNSLKPEDTAVYYCATAPDWYSGSGAPDYWGQGTQVTVSS

SEQ ID NO: 289 - protein, 120 aa, synthetic construct
QVQLVESGGGLVQAGGSLKLSCAASGGTFSSYDMGWYRQAPGKEREFVADIRWISGSTYYADSVKGRFTISRDNAKNTVYLQMNSLKPEDTAIYYCNAETVTDTVQSTYWGQGTQVTVSS

SEQ ID NO: 290 - protein, 120 aa, synthetic construct
QVQLVESGGGLVQAGGSLRLSCAVSGGTFSSYDMGWYRQAPGKEREFVADIRWISGSEYYADSVKGRFTISRDNAKNTVYLQMNNLKPEDTAIYYCNAETVTDTVQSTYWGQGTQVTVSS

SEQ ID NO: 291 - protein, 117 aa, synthetic construct
QVQLVESGGGLVQAGGSLRLSCASSRSRWSTDIMGWYRQAPGKQRELVATITVGGVTTYADSVKGRFTISRDRTKNTWYLQMNSLKPDDTAVYFCNLVSLDDSGNYWGQGTQVTVSS

SEQ ID NO: 292 - protein, 120 aa, synthetic construct
QVQLVESGGGLVQAGGSLRLSCAVSGGTFSSYDMGWYRQAPGKEREFVADIRWISGSEYYADSVKGRFTISRDNAKRTVYLQMNNLKPEDTAIYYCNAETVTDTVQSTYWGQGTQVTVSS

SEQ ID NO: 293 - protein, 117 aa, synthetic construct
QVQLVESGGGLVQAGGSLRLSCAAPRSHYSTDIMGWFRQAPGKQRELVATITIGGSTNYADSVKGRFTISTDNARNTWYLQMNSLKPEDTAVYFCNLRRLGYPENYWGQGTQVTVSS

SEQ ID NO: 294 - protein, 118 aa, synthetic construct
QVQLVESGGGLVQAGGSLRLSCVASGGTFSNYAMGWFRQAPGKVREFVAAIRSGGSTYYADSVTGRFTITRDNAKNTVYLQMKSLKPEDTAVYYCAAGPDAVDEYDYWGQGTQVTVSS

SEQ ID NO: 295 - protein, 120 aa, synthetic construct
QVQLVESGGGLVQAGGSLRLSCALSGRTFGTYDMGWYRQAPGKEREFVADIRWISGNTYYADSVKGRFTISRDNAKNTVYLQMNNLKPEDTAIYYCNAETVTDTVQSTYWGQGTQVTVSS

SEQ ID NO: 296 - protein, 119 aa, synthetic construct
QVQLVESGGGLVQPGGSLRVSCAASGRTFSSYAMGWFRQAPGKQREFVAAIRSSGGSTYYADSVEGRFTISRDNAKNTVYLQMNSLKPEDTAVYYCAAGPDNSDAYDTWGQGTQVTVSS

SEQ ID NO: 297 - protein, 115 aa, synthetic construct
QVQLVESGGGLVQAGGSLRLSCTASGSRFSIDVMGWYRQAPGKQRELVATITIGGSTTYADSVKGRFSISRDRAKNAMYLQMDSLKPDDTGVYYCNLRRSSSDYWGQGTQVTVSS

SEQ ID NO: 298 - protein, 119 aa, synthetic construct
QVQLVESGGGLVQAGGSLRLSCAASGRTFSGFAMGWFRQASGKAREFVAAIRRSGGSTYYADSVKGRFTISRDNTKDMLYLQMNTLKPEDTAVYYCAAGDDLVDEYEYWGQGTQVTVSS

SEQ ID NO: 299 - protein, 120 aa, synthetic construct
QVQLVESGGGLVQAGGSLRLSCAASGGTFSSYDMGWYRQAPGKEREFVADIRWISGNTYYADSVKGRFTISRDNAKNTVYLQMNNLKPEDTAIYYCNAETVTDTVQSTYWGQGTQVTVSS

SEQ ID NO: 300 - protein, 120 aa, synthetic construct
QVQLVESGGGLVQAGGSLRLSCAVSGGTFSSYDMGWYRQAPGKEREFVADIRWSGGSTYYADSVKGRFTISRDNAKNTVYLQMNSLKPEDTAIYYCNAETVTDTVQSTYWGQGTQVTVSS

SEQ ID NO: 301 - protein, 120 aa, synthetic construct
QVQLVESGGGLVQAGGSLRLSCEASGSIFSLNVMGWYRQAPGKQRELVATITSGGSTNYADSVKGRFTISRDNAKNTVYLQMNSLKPEDTAVYYCNVPAMLREVGLEDYWGQGTQVTVSS

SEQ ID NO: 302 - protein, 126 aa, synthetic construct
QVQLVESGGGLVQPGGSLRLSCAASGFTFDKYAMSWVRQAPGKGPEWVSAINSGGSRTYYQDSVKGRFTISRDNAKNTLYLQMNSLKPEDTAVYYCAAGDSDVSGRFTYPTDRGRWGQGTQVTVSS

SEQ ID NO: 303 - protein, 117 aa, synthetic construct
QVQLVESGGGLVQAGGSLNLSCASSRSRWSTDIMGWYRQAPGKQRELVATITVGGVTNYADSVKGRFTISRDRAKTMWYLQMNSLKPDDTAVYFCNLVSLDDSGNYWGQGTQVTVSS

SEQ ID NO: 304 - protein, 119 aa, synthetic construct
QVQLVESGGGSVQAGGSLRLSCAPSARTFSSYAMGWFRQASGKAREFVAAIRRSGGSTYYADSVEGRFTISRDNAKNTVYLQMNSLTPEDTAVYYCAAGDDLVDEYEYWGQGTQVTVSS

SEQ ID NO: 305 - protein, 119 aa, synthetic construct
QVQLVESGGGSVQAGGSLRLSCAASGSFFSSYAMGWFRQAVGKQREFVAAIRSSGGSTYYADSVEGRFTISRDNAKNTVYLQMNSLKPEDTAVYYCAAGPDNSDAYDYWGQGTQVTVSS

SEQ ID NO: 306 - protein, 120 aa, synthetic construct
QVQLVESGGGLVQAGDSLRLSCAASGLSLSTYAVAWFRQAPGKKREFIAGIRRIAGRTYYADSVEGRFTISRDNAENTVYLQMDSLKPEDTAVYYCAADPQDIVDAYEYWGQGTQVTVSS

SEQ ID NO: 307 - protein, 119 aa, synthetic construct
QVQLVQLQESGGGLVQAGNSLRLSCAASGRTFSTWLGWFRQAPGKEREFVASIRMSDGLTYYSDSAEGRFTISRDNAKRTEYLQMNSLKSEDTAVYYCAAGDGIAWNYWGQGTQVTVSS

SEQ ID NO: 308 - protein, 124 aa, synthetic construct
QVQLVQLQESGGGLVQAGGSLRLSCAASGRTGSVYAMAWFRQAPEKEREFVALIRLDGGSTYYGDSVAGRFTISRDNAKNTVYLQMNSLKPEDTAVYYCAGDRSKGHVGEYDYWGQGTQVTVSS

SEQ ID NO: 309 - protein, 122 aa, synthetic construct
QVQLVQLQESGGGLVQAGGSLTLSCTASGRTLDTNPMGWFRQAPGKEREFVARINTVGFSTYYLDSAKGRFTISRDRAKNTVFLQMNSLKPEDTAVYYCAAGVDTNGDFTSRGQGTQVTVSS

SEQ ID NO: 310 - protein, 129 aa, synthetic construct
QVQLVQLQESGGGLVQPGGSLRLSCAASGFTLDDYAIGWFRQAPGKEREGVSCISSTNSYTYYSDSVKGRFTISRDNAKNTVYLQMNSLKPEDTAVYYCAGRKWGIATLDPDYYPIDLWGKGTQVTVSS

SEQ ID NO: 311 - protein, 122 aa, synthetic construct
QVQLVQLQESGGGLVQAGGSLRVSCVVSGRTFSSFAMGWFRQAPGKKREFVAAIRRSGSSTYYADSVKGRFTISRDNAKNTVYLQMNSLKPEDTGVYFCAAGSDLVDEYDYWGQGTQVTVSS

SEQ ID NO: 312 - protein, 129 aa, synthetic construct
QVQLVQLQESGGGLVQPGGSLRLSCAASGDTLDYYAIGWFRQAPGKEREGVGCISSSNSYTYYEDSVKGRFTISRDNAKNTVYLQMNSLKPEDTAVYYCAGRKWGIATMDPDYYAIDLWGKGTQVTVSS

SEQ ID NO: 313 - protein, 125 aa, synthetic construct
QVQLVQLQESGGGLVQAGGSLRLSCAASVASGRTFSNYAMGWFRQAPGKKREFVAAIRTNGISTYYAVSVEGRFTISRDNTKNTVYLQMNSLKPEDTANYYCAAGSDLVDEYDYWGQGTQVTVSS

SEQ ID NO: 314 - protein, 120 aa, synthetic construct
QVQLVESGGGLVQAGGSLRLSCAASGSAFRANVMAWYRQAPGKQRELVATISRGGTTNYADSVKGRFTISRDNSNNMVYLEMNSLKPEDTAVYYCNADGYSGSAYQRARWGQGAQVTVSS

SEQ ID NO: 315 - protein, 112 aa, synthetic construct
QVQLVESGGGLVQPGGSLTLSCTTSGIDVGVDNMGWYRQAPGKERELVAHITARGTPFIVDSVKGRFTISRDRTKNTVFLQMNSLKPEDTAVYYCNLEPRYWGQGTQVTVSS

SEQ ID NO: 316 - protein, 133 aa, synthetic construct
QVQLVESGGGLVQPGGSLTLSCTGSGFTFDNYVIGWFRQAPGKEREIVSCITRGPGSPKYADSVKGRFNVSRNGHGDSVFAVFLQMSDLKSEDTGLYYCAARRTKVSGAMLCALRGPHVYPNWGQGTQVTVSS

SEQ ID NO: 317 - protein, 121 aa, synthetic construct
QVQLVESGGGSVQPGGSLRLSCAASSGSAFRANVMAWYRQAPGKQRELVALISRSGDADYADSVKGRFTISRDNSNNMVFLQMNSLKPEDTAVYYCNAYGYSGSAYQRARWGQGTQVTVSS

SEQ ID NO: 318 - protein, 113 aa, synthetic construct
QVQLVESGGGLVQAGESLRLSCAASGIDFSMTAMGWYRQAPGKKRESVARIFGGSSTMYADSVKGRFTISRDNAKNTVYLQMNSLKAEDTGVYYCNANTYNAWGQGTQVTVSS

SEQ ID NO: 319 - protein, 117 aa, synthetic construct
QVQLVESGGGLVQAGGSLRLSCAASGSAFIANLMAWYRGKQRELVALISRDGTTNYSDSVKGRFTISRDNSNNMVYLEMNSLKPEDTAVYYCNADGYSGKTYQRARWGLGTQVTVSS

SEQ ID NO: 320 - protein, 122 aa, synthetic construct
QVQLVESGGGLVQAGGSLRVSCVASTSGSAFRANLMAWYRQAPGKQRELVALITRDGTTNYADSVKGRFTISRDDSKNMVYLEMNSLKPEDTAVYYCNADGYSGSTYQRARWGPGTQVTVSS

SEQ ID NO: 321 - protein, 123 aa, synthetic construct
QVQLVESGGGAAQAGGSLRLSCAASGRTLSTLVMAWFRQAPGKEREFVAAITVNKAKYYGDFVKGRFTVSRDNAKNTVDLQMNSLNPVDTAVYYCAAREHSSISTNAADYDYWGRGTQVTVSS

SEQ ID NO: 322 - protein, 122 aa, synthetic construct
QVQLVESGGGEVQAGGSLRVSCVASTSGSAFRANLMAWYRQAPGKERELVALITRDGTTNYADSVKGRFTISRDNSKNMVYLEMNSLKPEDTAVYYCNADGYSGSTYQRARWGQGTQVTVSS

SEQ ID NO: 323 - protein, 122 aa, synthetic construct
QVQLVESGGGLVQAGGSLRLSCAASAPGSAFRANVMAWYRQAPGKQRDLVALISRDGTSNYADSVKGRFTISRDNSKNMVYLEMNSLKPEDTAVYYCNADGHSGTTYQRARWGQGTQVTVSS

SEQ ID NO: 324 - protein, 112 aa, synthetic construct
QVQLVESGGGLAQAGGSLTLSCTTSGIDVGVDNMGWYRQAPGKERELVAHITARGTPFIVDSVKGRFTISRDRSKNTVYLQMNSLKPEDTAVYYCNLEPRYWGQGTQVTVSS

SEQ ID NO: 325 - protein, 126 aa, synthetic construct
QVQLVESGGGLVQPGGSLRLSCAVSGFAWRNLAIAWFRQPPGKEREEREGISCITTGGASTNYADSVKGRFTISRDNAKNTVYLQMNNLKPEASAVYYCAARTYSCTADQYEYDTWGQGTQVTVSS

SEQ ID NO: 326 - protein, 120 aa, synthetic construct
QVQLVESGGGLVQAGGSLRLSCAASGSAFRANVMAWYRQATGKQRELVALISRDGTTSYVDSVKGRFTISRDDSKNMVYLEMNSLKPEDTAVYYCNADGYSGSAYQRARWGQGTQVTVSS

SEQ ID NO: 327 - protein, 113 aa, synthetic construct
QVQLVESGGGLVQAGGSLRLSCVASVDMFSVTDIGWHRQAPGKQRELVALISTSGHTNYAHSVKDRATISRSGRTVYLQMNGLKVDDTGVYYCKLRHGSGDYWGQGTQVTVSS

SEQ ID NO: 328 - protein, 112 aa, synthetic construct
QVQLVESGGDLVQAGGSLTLSCTTSGIDVGVDNMGWYRQAPGKERELVAHITARGTPFIVDSVKGRFTISRDRSKNTVYLQMNSLKPEDTAVYYCNLEPRYWGQGTQVTVSS

SEQ ID NO: 329 - protein, 123 aa, synthetic construct
QVQLVESGGGLVQPGGSLRLSCVVSGLNFGRFPMGWFRQAPGKEREFVAAITVNKAKYYGDFVKGRFTVSRDNAKNTVDLQMNSLNPVDTAVYYCAAREHSSISTSAADYDYWGRGTQVTVSS

SEQ ID NO: 330 - protein, 123 aa, synthetic construct
QVQLVESGGGAVQAGGSLRLSCGASGRTFSTRNLGWFRQAPGKEREFVAAITVNKAKYYGDFVKGRFTVSRDNAKNTVDLQMNSLNPVDTAVYYCAAREHSSISTNAADYDYWGRGTQVTVSS

SEQ ID NO: 331 - protein, 124 aa, synthetic construct
QVQLVESGGGLVQAGGSLRLSCAASGSASGSAFRANVMAWYRQTLGKQRDLVAMITRGGTTTYADSVKGRFTISRDNSNNMVYLEMNSLKPEDSAVYYCNADGYSGSTYQRARWGQGTQVTVSS

SEQ ID NO: 332 - protein, 123 aa, synthetic construct
QVQLVESGGASVQPGGSLRLSCTTSGGTLASYAIGWFRQAPGKEREFVAAITVNKAKYYGDFVKGRFTVSRDNAKNTVDLQMNSLNPVDTAVYYCAAREHSSISTNAADYDYWGRGTQVTVSS

SEQ ID NO: 333 - protein, 127 aa, synthetic construct
QVQLVESGGGLVQTGGSRRLSCVGDGSGSGFNFRIIAWFRQAPGEEREGVSSISTSGTVTNYADSVKGRFIISRDNAKKTVWLQMNSLKPEDTAVYYCAACKFGGQCNLEEADWDYWGQGIQVTVSS

SEQ ID NO: 334 - protein, 113 aa, synthetic construct
QVQLVDSGGGLVQAGESLRLSCAAIGIDFNLTAMNWYRQAPGKKREVVARIFGGSSTNYADSVKGRFTISRDNAKNTVYLQMNSLKPEDTGVYYCNANTYNAWGQGTQVTVSS

SEQ ID NO: 335 - protein, 114 aa, synthetic construct
QVQLVESGGGSVQPGGSLRLSCATSGFTFGDVWMYWVRQAPGKGLEWVAIIEAVGPKYYAPSVKGRFTVSRDNARSTLYLEMNNLKLEDTARYYCSTDQDGRKRGQGTQVTVSS

SEQ ID NO: 336 - protein, 123 aa, synthetic construct
QVQLVESGGGAVQAGGSLRLSCAASGRTFSTRNLGWFRQAPGKEREFVAAITVNKATYYGDFVKGRFTVSRDNAKNTVDLQMNSLKPVDTAVYYCAARAHSSISTNAADYDYWGRGTQVTVSS

SEQ ID NO: 337 - protein, 129 aa, synthetic construct
QVQLVESGGSAVQSGGSLRLSCRISGGTLDEYAIGWFRQAPGKEREGISCISTGGDSRGYADSVRGRFTMSRDNAKNMVYLQMNSLKPEDTAVYYCAASPDRDYVCYDWGTLAPDFGSWGQGTQVTVSS

SEQ ID NO: 338 - protein, 113 aa, synthetic construct
QVQLVESGGGLVQAGESLSLSCAASGIDFSITAMGWYRQAPGKNREVVARMLDGGITTYADSVKGRFTISRDGAQNTVYLRMNSLKPEDTGVYYCNANTYNAWGQGTQVTVSS

SEQ ID NO: 339 - protein, 123 aa, synthetic construct
QVQLVESGGGAVQAGGSLRLSCAASGRTFSTRNLGWFRQAPGKEREFVAAITVNKAKYYGDFVKGRFTVSRDNAKNTVDLQMNSLNPVDTAVYYCAAREHSSISTSAADYDYWGRGTQVTVSS

SEQ ID NO: 340 - protein, 116 aa, synthetic construct
QVQLVESGGGLVQAGGSLRLSCTASGRIISTNGMRWFRQAPGKQREFVAFASSDGGTTYATAVKGRFTISRDDPKNTVYLQMDSLKPEDTATYYCQTNGPRASIIWGQGTQVTVSS

SEQ ID NO: 341 - protein, 120 aa, synthetic construct
QVQLVESGGGLVQAGGSLRLSCAASGSAFRAKIMAWYRQAPGKQRDVVAIINPDGTTNYADSVKGRFTISRDNSENMVYLEMNNLKPEDTAVYYCNADGYSGSVYQRARWGQGTQVTVSS

SEQ ID NO: 342 - protein, 113 aa, synthetic construct
QVQLVESGGGLVQAGESLRLSCAASGIDFNLTAMGWYRQAPGKKREVVARIFGGSSTNYADSVKGRFTISRDNAKNMVYLQMNSLKPEDTGVYYCNANTYNAWGQGTQVTVSS

SEQ ID NO: 343 - protein, 123 aa, synthetic construct
QVQLVESGGGAVQAGGSLRLSCAASGRTFSTRNLGWFRQAPGKEREFVAAITVNKATYYGDFVKGRFTVSRDNAKNTVDLQMNSLNPVDTAVYYCAAREHSSISTNAADYDYWGRGTQVTVSS

SEQ ID NO: 344 - protein, 127 aa, synthetic construct
QVQLVESGGGLVQTGGSRRLSCVGDGSGSGFNFRIIAWFRQAPGEEREGVSSISTSGTVTNYADSVKGRFIISRDNAKKTVWLQMNSLKPEDTAVYYCAACKFGGQCNLEEADWDYWGQGTQVTVSS

SEQ ID NO: 345 - protein, 126 aa, synthetic construct
QVQLVESGGGLVQAGGSLTLSCAASGSAAASGDAFRANIMAWYRQAPGKQRDLVALISRDGATSYADSVKGRFTISRDNDKKMVYLEMNSLQPEDTAVYYCNADGYSGSTYQRARWGQGTQVTVSS

SEQ ID NO: 346 - protein, 113 aa, synthetic construct
QVQLVESGGGLVQAGESLRLSCAAIGIDFNLTAMNWYRQAPGKKREVVARIFGGSSTNYADSVKGRFTISRDNAKNTVYLQMNSLKPEDTGVYYCNANTYNAWGQGIQVTVSS

SEQ ID NO: 347 - protein, 123 aa, synthetic construct
QVQLVESGGALVQPGGSLRLACAASGFSLDEYAVGWFRQAPGKESEGVLYISRDGLTVYADSVKGRFTISSDYAKNTLYLQMNDLKAEDTAVYFCAASSGWDGGFSNSNYKYWGQGTQVTVSS

SEQ ID NO: 348 - protein, 123 aa, synthetic construct
QVQLVESGGGLVQAGGSLTLSCAASGNIASINTMGWFRQAPGKEREFVAAITVNKAKYYGDFVKGRFTVSRDNAKNTVDLQMNSLNPVDTAVYYCAAREHSSISTSAADYDYWGRGTQVTVSS

SEQ ID NO: 349 - protein, 123 aa, synthetic construct
QVQLVESGGGLVQAGGSLRLSCEVSGFAWRNLAIGWFRQPPGKEREGVSCITTGGASTNYADSVKGRFTISRDNAKNTVYLQMNSLKPEASGVYYCAARTHSCTADQYEYIAWGQGTQVTVSS

SEQ ID NO: 350 - protein, 123 aa, synthetic construct
QVQLVESGGGAVQAGGSLRLSCAASGRTFSTRNLGWFRQAPGKEREFVAAITVNKAKYYGDFVKGRFTVSRDNAKNTVDLQMNSLNPVDTAVYYCAAREHSSISTNAADYDYWGRGTQVTVSS

SEQ ID NO: 351 - protein, 115 aa, synthetic construct
QVQLVESGGGLVQAGGSLTLSCAVSGTAAGNAMVRWYRQAPGKQRELVAWIGTDGSTNYADSVQGRFAISRDDAKNTVYLQMNSPKPEDTGVYICNTNWRLRPYWGQGTQVTVSS

SEQ ID NO: 352 - protein, 127 aa, synthetic construct
QVQLVESGGGLVQTGGSLRLSCVGSGSGSGFNFRIIAWFRQAPGKEREGVSSISTSGTGTNYADSVKGRFTISRDNAKKTVWLQMNSLKPEDTAVYYCAACAFGKNCNLEEADMGYWGKGTQVTVSS

SEQ ID NO: 353 - protein, 112 aa, synthetic construct
QVQLVESGGGLVQPGGSLTLSCTTSGIDVGVDNMGWYRQAPGKERELVAHITARGTPFIVDSVKGRFTISRDRSKNTVYLQMNSLKPEDTAVYYCNLEPRYWGQGTQVTVSS

SEQ ID NO: 354 - protein, 125 aa, synthetic construct
QVQLVESGGGLVQPGGSLTLSCAASGYTSDYRLGWFRQAPDRDREGVSCITENGSVTNYADSVKGRFTISRDNAKNTLYLQMNDLKPEDTAEYYCAASSRVFCDYANSVLAFDFWGQGTQVTVSS

SEQ ID NO: 355 - protein, 118 aa, synthetic construct
QVQLVESGGGLVQPGGSLRLSCAASGSISDIPVMGWYRQPPGKQRELVATVEDTGTSTYADFVKGRFTISRDNALNTVYLQMNSLKPEDTAVYYCNSYTSLWPRFPYWGQGTQVTVSS

SEQ ID NO: 356 - protein, 127 aa, synthetic construct
QVQLVESGGGLVQPGGSLRLSCVGDGSGSGFNFRIIAWFRQAPGEEREGVSSISTSGTVTNYADSVKGRFIISRDNAKKTVWLQMNSLKPEDTAVYYCAACKFGGQCNLEEADWDYWGEGTQVTVSS

SEQ ID NO: 357 - protein, 123 aa, synthetic construct
QVQLVESGGGLVQAGGSLRLSCGASGRTFGTRDLGWFRQAPGKEREFVAAITVNKATYYGDFVKGRFTVSRDNAKNTVDLQMNSLKPVDTAVYYCAARAHSSISTNAADYDYWGRGTQVTVSS

SEQ ID NO: 358 - protein, 133 aa, synthetic construct
QVQLVESGGGLVQPGGSLTLSCTGSGFTFDNYVIGWFRQAPGKEREIVSCITRGPGSPKYADSVKGRFNVSRNGHGDSVFAVFLQMSDLKSEDTGLYYCAARRTKVSGAMLCALRGPHVYPDWGQGTQVTVSS

SEQ ID NO: 359 - protein, 111 aa, synthetic construct
QVQLVESGGGLVQSGGSLRLSCAASGRISPTDLMSWYRQAPGGQRELVARVTSGGTANYADSVKGRFTVSRDNARNTAYLQMNSLKPEDTAVYYCTCHGSWGQGTQVTVSS

SEQ ID NO: 360 - protein, 124 aa, synthetic construct
QVQLVESGGKLVQAGGSLTLSCTASERTFFRRVLAWFRQAPGKERVFVAGLGWFSGTTDYADSVKGRFSISKDGAKNTLYLQMNNLNPEDTAVYYCAASRAYSDSFSSLDYDYWGRGTQVTVSS

SEQ ID NO: 361 - protein, 113 aa, synthetic construct
QVQLVESGGGLVQAGGSLRLSCVASVDMFSVTDIGWHRQAPGKQRELVALISSSGHTNYADSVKDRATISRSGRTVYLQMNGLKADDTGVYYCKLRHGSGDYWGQGTQVTVSS

SEQ ID NO: 362 - protein, 122 aa, synthetic construct
QVQLVESGGGLVQAGGFLRLSCAASAPGSAFRANVMAWYRQAPGKQRDLVALISRDGTSNYADSVKGRFTISRDNSKNMVYLEMNSLKPEDTAVYYCNADGHSGTTYQRARWGQGTQVTVSS

SEQ ID NO: 363 - protein, 123 aa, synthetic construct
QVQLVESGGGSVQAGDSLRLSCANNAQTGSRYPMGWFRQAPGQEREFVAAITVNKATYYGDFVKGRFTVSRDNAKNTVDLQMNSLKPVDTAVYYCAARAHSSISTNAADYDYWGRGTQVTVSS

SEQ ID NO: 364 - protein, 124 aa, synthetic construct
QVQLVESGGGLVQPGGSLRLSCAASSATSGSAFRANIMAWYRQAPGKQRDLVALISRDGTTNYADSVKGRFTISRDNSKNMVYLEMNSLKPEDTAVYYCNADGHSGSSYQRARWGQGTQVTVSS

SEQ ID NO: 365 - protein, 125 aa, synthetic construct
QVQLVESGGGLVQPGGSLTLSCAASGFTFSSYAMIWVRQTPGKGLEWVSGISNGGGSTKYADSVKGRFTISRDNAKNTLYLQMNNLKPEDTAVYYCAKPHQPATVVRPTTTYHYRGQGTQVTVSS

SEQ ID NO: 366 - protein, 123 aa, synthetic construct
QVQLVESGGGLVQPGGSLRLACAASGFSLDEYAVGWFRQVPGKEREGVSYISRGGLTVYADSVKGRFTISSDYAKNTLYLQMNDLKAEDTAVYFCAASSGWDGGFSNSNYKYWGQGTQVTVSS

SEQ ID NO: 367 - protein, 121 aa, synthetic construct
QVQLVESGGGLVQAGGSLRLSCAASGGSAFTANLMAWYRQAPGKQREWVATITRVGTTNYEDSVKGRFTISRDNSKNMVYLEMNNLKPEDTAVYYCNADGHSGSSYQRARWGQGTQVTVSS

SEQ ID NO: 368 - protein, 114 aa, synthetic construct
QVQLVESGGGWVQPGGSLRLSCAASGFPFEDSWMYWVRQAPGKGLEWVAIIEAVGPKYYANSVKGRFTISRDNAKKMVYLQMNSLKPEDTALYYCSRDQSGRVRGQGTQVTVSS

SEQ ID NO: 369 - protein, 120 aa, synthetic construct
QVQLVESGGGLVQPGDSLTLSCAASGSAFRANLMAWYRQAPGKQREWVATINRGGTTSYADSVKGRFTISRDDSKNMVYLEMNSLKPEDTAVYYCNADGYSGSTYQRARWAQGTQVTVSS

SEQ ID NO: 370 - protein, 124 aa, synthetic construct
QVQLVESGGGLVQAGGSLRLSCAASDRTIDGYAMGWFRQAPGKEREFVATISGFTGDTSYARSVKGRFTIARDNAKNMLYLQMSSLEPEDTAVYYCAARTAGIFANRDSLYAYWGQGTQVTVSS

SEQ ID NO: 371 - protein, 122 aa, synthetic construct
QVQLVESGGGLVQPGGSLNLSCVVSGFFLDYAAIGWFRQAPGKAREGVSCITFRDGNTYYVRSVKGRFTISRDNVKSTVYLQMNNLKSEDTADYYCAIGQFCGTNWREFEHYGQGTQVTVSS

SEQ ID NO: 372 - protein, 116 aa, synthetic construct
QVQLVESGGGLVQAGGSLRLSCTASGRIVSTNGMRWFRQAPGKQREFVAFASSDGGTTYATAVKGRFTISRDDPKNTVYLQMDSLKPEDTATYYCQTNGPRASIIWGQGTQVTVSS

SEQ ID NO: 373 - protein, 124 aa, synthetic construct
QVQLVESGGGLVQAGGSLRLSCAASGATSGSAFRANIMAWYRQAPGKQRDLVALISRDGTTSYADSVKGRFTISRDNSKNMVYLQMNSLKPEDTAVYYCNADGHSGSSYQRARWGQGTQVTVSS

SEQ ID NO: 374 - protein, 125 aa, synthetic construct
QVQLVESGGGLVQAGGSLRLSCADSERFHVLKAMGWYRQGPGKQRELVAVITRGGRTNYADSVKGRFTISRDNANDTVYLQMNSLKPEDTAVYYCNADLLSRPEEYYSRTSGDYWGQGTQVTVSS

SEQ ID NO: 375 - protein, 112 aa, synthetic construct
QVQLVESGGGLVQAGGSLTLSCTTSGIDVGVDNMGWYRQAPGKERELVAHITARGTPFIVDSVKGRFTISRDRSKNTVYLQMNSLKPEDTAVYYCNLEPRYWGQGTQVTVSS

SEQ ID NO: 376 - protein, 123 aa, synthetic construct
QVQLVESGGGLVQPGGSLRLACAASGFSLDEYAVGWFRQAPGKEREGVSYRSRGGLTMYADSVKGRFTFSSDYAKNTLYLQMNDLKAEDTAVYFCAASSGWDGGFSNSNYKYWGQGTQVTVSS

SEQ ID NO: 377 - protein, 122 aa, synthetic construct
QVQLVESGGGLVQPGGSLRLSCAASAPGSAFRANVMAWYRQAPGKQRDLVALISRDGTSNYADSVKGRFTISRDNSKNMVYLEMNSLKPEDTAVYYCNADGHSGTTYQRARWGQGTQVTVSS

SEQ ID NO: 378 - protein, 113 aa, synthetic construct
QVQLVESGGGLVQAGGSLRLSCVASGRTYSSTIIYWYRQAPGKQRELVASISSGGGTYYADSVKGRFTISRDNAKNTAYLQMNNLKPGDTAVYYCRWGLSNYWGQGTQVTVSS

SEQ ID NO: 379 - protein, 127 aa, synthetic construct
QVQLVESGGGLVHPGGSLRLSCVGDGSGSGFNFRIIAWFRQAPGEEREGVSSISTSGTVTNYADSVKGRFIISRDNAKKTVWLQMNSLKPEDTAVYYCAACKFGGQCNLEEADWDYWGEGTQVTVSS

SEQ ID NO: 380 - protein, 114 aa, synthetic construct
QVQLVESGGGLVQPGGSLRLSCAASGFDFSDSWMYWIRQAPGKGLEWVSTIEAVGPKYYAGSVKGRFTISRDNAKNTVYLQMNNLKPEDTALYYCARDQDGRIRGRGTQVTVSS

SEQ ID NO: 381 - protein, 126 aa, synthetic construct
QVQLVESGGGLVQSGGSLRLSCEASGSAAASGDAFRANIMAWYRQAPGKQRDLVALISRDGATSYADSVKGRFTISRDNSKNMVYLEMNSLKPEDTAVYYCNADGYSGSTYQRARWGQGTQVTVSS

SEQ ID NO: 382 - protein, 127 aa, synthetic construct
QVQLVESGGGLVQTGGSRRLSCVGDGSGSGFNFRIIAWFRQAPGEEREGVSSISTSGTVTNYADSVKGRFIISRDNAKKTVWLQMNSLKPEDTAVYYCAACKFGGQCNLEEADWDYWGEGTQVTVSS

SEQ ID NO: 383 - protein, 123 aa, synthetic construct
QVQLVESGGGLVQAGGSLRLSCAASGFTLDEYALGWFRQAPGKEREGVSYITVGGLTMYGDSVKGRFTVSRDYAKNTLYLQMNDLKPDDTAVYFCAARTGWNGGTTDSNYDYWGRGTQVTVSS

SEQ ID NO: 384 - protein, 123 aa, synthetic construct
QVQLVESGGGLVQAGGSLRLSCVASGSAGSAFRANVMAWYRQAPGKQRELVALISRSGTTSYEDSVKGRFTISRDNSNNMVYLEMNSLNPEDTAVYYCNADGYSGSVYQRARWGQGTQVTVSS

SEQ ID NO: 385 - protein, 112 aa, synthetic construct
QVQLVESGGGLVQAGGSLTLSCATSGISFSSHNVGWYRQAPGKERELVAHITARGTPFIVDSVKGRFTISKDQTKNTVYLQMNSLKLEDTGVYYCNAEPTYWGPGTQVTVSS

SEQ ID NO: 386 - protein, 120 aa, synthetic construct
QVQLVESGGGWVQAGGSLRLSCAASGNMVSTYDMSWYRQAPGKRRELVAAITAAGVTRYADSVKGRFTIARDHAKNTASLQMNSLRPEDTAVYYCYAAGNTSLTYGLTFWGQGTQVTVSS

SEQ ID NO: 387 - protein, 118 aa, synthetic construct
QVQLVESGGGLVQAGGSLRLSCAASGSVASIGAMGWYRQAPGKKRELVATIFDGSIPNYADSVKGRFTLSRDNAENTMSLQMDSLKPEDTAVYYCTARRWSSASYKYWGQGTQVTVSS

SEQ ID NO: 388 - protein, 122 aa, synthetic construct
QVQLVESGGGLVQAGGSLRLSCAASAPGSAFRANIMAWYRQAPGKQRDLVALISRDGTSNYADSVKGRFTISRDNSKNMVYLEMNSLKPEDTAVYYCNADGHSGTTYQRARWAQGTQVTVSS

SEQ ID NO: 389 - protein, 120 aa, synthetic construct
QVQLVESGGGLVQAGGSLRLSCAASGSAFRANIMAWYRQAPGKQREWVATISRVGTTNYADSVKGRFTISRDNSKNMVYLEMNSLKPEDTAVYYCNADGHSGSTYQRARWAQGTQVTVSS

SEQ ID NO: 390 - protein, 123 aa, synthetic construct
QVQLVESGGALVQPGGSLRLACAASGFSLDEYAVGWFRQAPGKEREGVSSISRGGLTSYADSVKGRFTISSDYAKNTLYLQMNDLKAEDTAVYFCAASSGWDGGFSSSNYKYWGQGTQVTVSS

SEQ ID NO: 391 - protein, 123 aa, synthetic construct
QVQLVESGGALVQPGGSLRLACAASGFSLDEYAVGWFRQVPGKEREGVSYISRGGLTVYADSVKGRFTISSDYAKNTLYLQMNDLKAEDTAVYFCAASSGWDGGFSNSNYKYWGQGTQVTVSS

SEQ ID NO: 392 - protein, 123 aa, synthetic construct
QVQLVESGGGLVQPGGSLRLACAASGFSLDEYAVGWFRQAPGKEREGVSSISRGGLTVYADSVKGRFTISSDYAKNTLYLQMNDLKAEDTAVYFCAASSGWDGGFSSSNYKYWGQGTQVTVSS

SEQ ID NO: 393 - protein, 122 aa, synthetic construct
QVQLVESGGGLVQAGGSLRLSCAASGSGSDFRANVMAWYRQADGKQRELVAMISRVGTISYADSVKGRFTISRDNSKNMVYLEMNSLKPEDTAVYYCNADGHSGSTYQRARWGQGTQVTVSS

SEQ ID NO: 394 - protein, 122 aa, synthetic construct
QVQLVESGGGLVQAGGSLRVSCVASTSGSAFRANLMAWYRQAPGKERELVALITRDGTTNYADSVKGRFTISRDNSKNMVYLEMNSLKPEDTAVYYCNADGYSGSTYQRARWGQGTQVTVSS

SEQ ID NO: 395 - protein, 113 aa, synthetic construct
QVQLVESGGGLVQAGESLRLSCAASGIDFDLTAMGWYRQAPGKKREVVARIFGGSSTNYAESVKGRFTISRDNAKNTVYLQMNSLKPEDTGVYYCNANTYNAWGQGTQVTVSS

SEQ ID NO: 396 - protein, 118 aa, synthetic construct
QVQLVESGGGLVQPGGSLRLSCVGSGSISGIHAIGWYRQAPGKERELVAYIEGSTVTNYADSVKGRFTISRDNAKNTAYLQMNSLKPEDTAVYFCNSYSSLWPRYFHWGQGTQVTVSS

SEQ ID NO: 397 - protein, 112 aa, synthetic construct
QVQLVESGGGLVQAGGSLTLSCTTSGIDVGVDNMGWYRQAPGKERELVAHITARGTPFIVDSVKGRFTISRDRTKNTVYLQMNSLKPEDTAVYYCNLEPRYWGQGTQVTVSS

SEQ ID NO: 398 - protein, 116 aa, synthetic construct
QVQLVESGGGLVQAGGSLRLSCTASGRIVSTNGMRWFRQAPGKQREFVAFASSGGGTTYATAVKDRFTISRDDPKNTVYLQMDSLKPEDTATYYCQTNGPRASIIWGQGTQVTVSS

SEQ ID NO: 399 - protein, 118 aa, synthetic construct
QVQLVESGGGLVQAGGSLRLSCAASGSISDIPVMGWYRQPPGKQRELVATVEDTGTSTYADFVKGRFTISRDNALNTVYLQMNSLKPEDTAVYYCNSYTSLWPRFPYWGQGTQVTVSS

SEQ ID NO: 400 - protein, 121 aa, synthetic construct
QVQLVESGGGLVQVGGSLRLSCAASSGSAFRANVMAWYRQAPGKQRELVALISRDGTTNYADSVKGRFTISRDNSNNMVYLEMNSLKPEDTAVYYCNADGYSGSTYQRARWAQGTQVTVSS

SEQ ID NO: 401 - protein, 112 aa, synthetic construct
QVQLVESGGGLVQAGGSLTLSCTTSGIDVGVDNMGWYRQAPGKERELVAHITARGTPFIVDSVKGRFTISRDRSKNTVYLQMNSLKPEDTAVYYCNLEPRYWGQGIQVTVSS

SEQ ID NO: 402 - protein, 123 aa, synthetic construct
QVQLVESGGGAVQAGGSLRLSCAASGRTFSTRNLGWFRQAPGKEREFVSAITVNKAKYYGDFVKGRFTVSRDNAENTVDLQMNSLNPVDTAVYYCAAREHSSISTNAADYDYCGRGTQVTVSS

SEQ ID NO: 403 - protein, 113 aa, synthetic construct
QVQLVESGGGLVQAGESLRLSCAAIGIDFNLTAMGWYRQAPGKKREVVARIFGGSSTNYADSVKGRFTISRDNAKNTVYLQMNSLKPEDTGVYYCNANTYNAWGQGTQVTVSS

SEQ ID NO: 404 - protein, 116 aa, synthetic construct
QVQLVESGGGLVQSGGSLRLSCTASGRIISTNGMRWFRQAPGKQREFVAFASSDGGTTYATAVKGRFTISRDDPKNTVYLQMDSLKPEDTATYYCQTNGPRASIIWGQGTQVTVSS

SEQ ID NO: 405 - protein, 123 aa, synthetic construct
QVQLVESGGGSVQPGGSLRLSCLVSGLTLDDVAISWFRQAPGKEREGVSCISRSGSVTNYADSVKGRFTISRDDAKNTVYLQMNSLTPEDTAVYTCAAGACTYRIGAWAMKDWGKGTQVTVSS

SEQ ID NO: 406 - protein, 120 aa, synthetic construct
QVQLVESGGGLVQAGGSLTLTCIASGDNWNVNAMAWYRQAPGKQRDLVALISRDGTTSYADSVKGRFTISRDNSKNMVYLQMNSLKPEDTAVYYCNADGHSGSSYQRARWGQGTQVTVSS

SEQ ID NO: 407 - protein, 123 aa, synthetic construct
QVQLVESGGGAVQAGGSLRLSCAASGRTFSTRNLGWFRQAPGKEREFVAAITVNKATYYGDFVKGRFTVSRDNAKNTVDLQMNSLKPVDTAVYYCAARAHSSISTNAADYDYWGQGTQVTVSS

SEQ ID NO: 408 - protein, 123 aa, synthetic construct
QVQLVESGGALVQPGGSLRLACAASGFSLDEYAVGWFRQAPGKEREGVSSISRGGLTVYADSVKGRFTISSDYAKNTLYLQMNDLKAEDTAVYFCAASSGWDGGFSSSNYKYWGQGTQVTVSS

SEQ ID NO: 409 - protein, 127 aa, synthetic construct
QVQLVESGGGLVQAGGSLRLSCVGSGSGSGFNFRIISWFRQAPGKEREGVSSISASGTGTNYAESVKGRFTISRDKDKKTVWLQMNSLKPEDTAVYYCAGCAFGKQCNLEEADMDYWGKGTQVTVSS

SEQ ID NO: 410 - protein, 121 aa, synthetic construct
QVQLVESGGGLVQAGGSLRLSCAASSGSAFRANLMAWYAQAPGKQREWVATITREGTTHYADSVKGRFTISRDNSNNMVYLEMNSLKPEDTAVYFCNADGYSGSTYQRARWGQGTQVTVSS

SEQ ID NO: 411 - protein, 114 aa, synthetic construct
QVQLVESGGGLVQPGGSLRLSCAASGFTFSDSWMYWIRQAPGKGLEWVSTIEAVGPKYYAGSVKGRFTISRDNAKNTVYLQMNNLKPEDTALYYCARDQDGRIRGRGTQVTVSS

SEQ ID NO: 412 - protein, 119 aa, synthetic construct
QVQLVESGGDLVQAGGSLRLSCAASTKISTINSMSWYRQAPETGRGLVARLSNGGVTNYADSVKGRFTISRDNAKNMVYLQMDNLKPEDTALYICNAHVLGIDRWNDYWGQGTQVTVSS

SEQ ID NO: 413 - protein, 121 aa, synthetic construct
QVQLVESGGGLVQPGGSLRLSCAASGFTLDYYAIGWFRQAPGKEREGVSCITATGDFTNYADSVKGRFTISRDNAENTVFLQMNSLKPEDSAVYYCAAYPSGRCRVKPDYWGQFTQVTVSS

SEQ ID NO: 414 - protein, 125 aa, synthetic construct
QVQLVESGGGLVQAGGSLRLSCVDSERFHVLKAMGWYRQGPGKQRELVAIITRGGRTNYADSVKGRFTISRDNANDTVYLQMNSLKPEDTAVYYCNADLLSRPEEYYSRTSGDYWGQGTQVTVSS

SEQ ID NO: 415 - protein, 114 aa, synthetic construct
QVQLVESGGGLVQPGGSLRLSCAASGFDFSDSWMYWIRQAPGKGLEWVSTIEAVGPKYYAGSVKGRFTISRDNAKNTLYLQMNNLKPEDTALYYCARDQDGRMRGRGTQVTVSS

SEQ ID NO: 416 - protein, 118 aa, synthetic construct
QVQLVESGGGLVQAGGSLRLSCAASGFNAKSYDMAWYRQAPGKQRELVATIRISGITNYADSVKGRFTISRDNANNKVYLQMNSLEPEDTALYYCKRENMYRRDPAYWGQGTQVTVSS

SEQ ID NO: 417 - protein, 124 aa, synthetic construct
QVQLVESGGGLVQPGGSLRLSCAASGSASGSAFRANVMAWYRQTLGKQRDLVAMITRGGTTTYADSVKGRFTISRDNSNNMVYLEMNSLKPEDSAVYYCNADGYSGSTYQRARWGQGTQVTVSS

SEQ ID NO: 418 - protein, 124 aa, synthetic construct
QVQLVESGGGLVQTGGSLRLSCAASGSASGSAFRANVMAWYRQTLGKQRDLVAMITRGGTTTYADSVKGRFTISRDNSNNMVYLEMNSLKPEDSAVYYCNADGYSGSTYQRARWGQGTQVTVSS

SEQ ID NO: 419 - protein, 271 aa, synthetic construct
QVQLVESGGGLVQPGGSLRLSCSASERAFSDYRVAWFRQAPGKEREFVASVNWNGDNSYYADSVKGRFTISRDNAKNAVYLQMNSLKPGDTAVYVCAQSPILSVDSQVVTSPAQYQYWGQGTQVTVSSGGGGSGGGGSGGGGSQVQLVESGGGLVQPGGSLRLSCSASERAFSDYRVAWFRQAPGKEREFVASVNWNGDNSYYADSVKGRFTISRDNAKNAVYLQMNSLKPGDTAVYVCAQSPILSVDSQVVTSPAQYQYWGQGTQVTVSS

SEQ ID NO: 420 - protein, 278 aa, synthetic construct
QVQLVESGGGLVQAGGSLRLSCAITGGVFSRYTLGWFRQAPGKEREVVAAITWSSGVTYYTDSEKGRFIISRDSAKNVLYLQMSSLKPEDTAMYYCAADVDGSRRWGLYWAVSSDYDYWGQGTQVTVSSGGGGSGGGGSGGGGSGGGGSQVQLVESGGGLVQAGGSLRLSCAITGGVFSRYTLGWFRQAPGKEREVVAAITWSSGVTYYTDSEKGRFIISRDSAKNVLYLQMSSLKPEDTAMYYCAADVDGSRRWGLYWAVSSDYDYWGQGTQVTVSS

SEQ ID NO: 421 - protein, 281 aa, synthetic construct
QVQLVESGGGLVQPGSSLRLSCAASGRTFNSLSMAWFRQAPGKEREFVAGISWIGNTVYSNSVKGRFSISEDTSKNTGYLQMNSLKPEDTAVYYCAATMDRSPSWVRTNMQSAAYNYWGQGTQVTVSSGGGGSGGGGSGGGGSGGGGSGGGGSQVQLVESGGGLVQPGSSLRLSCAASGRTFNSLSMAWFRQAPGKEREFVAGISWIGNTVYSNSVKGRFSISEDTSKNTGYLQMNSLKPEDTAVYYCAATMDRSPSWVRTNMQSAAYNYWGQGTQVTVSS

SEQ ID NO: 422 - protein, 267 aa, synthetic construct
QVQLVESGGGLVQAGGSLALSCVVSGRTVDSYATGWFRQAPGKEREFVAAITWSGTMTKYADSAKGRFTISRDDAKNTVWLQMNGLKPEDTAVYYCVDRLRGRGNIYSDTLSYDYWGQGTQVTVSSGGGGSGGGGSGGGGSQVQLVESGGGLVQAGGSLALSCVVSGRTVDSYATGWFRQAPGKEREFVAAITWSGTMTKYADSAKGRFTISRDNAKSTVYLQMNGLKPEDTAVYYCVDRLRGRGNIYSDTLSYDYWGQGTQVTVSS

SEQ ID NO: 423 - protein, 266 aa, synthetic construct
QVQLVESGGGLVQAGGSLRLSCAASGRTFGNYTMGWFRQAPGKGRAFVALINWNGISTTYAGSVQGRFTISRDMAKNTVYLQMDSLKPEDTAVYYCAANLRSDSSYQGHYDYWGQGTQVTVSSGGGGSGGGGSGGGGSGGGGSQVQLVESGGGLVQAGGSLRLSCVVSGRTFGNYTMGWFRQAPGKGRAFVALINWNGISTTYAGSVQGRFTISRDMAKNTVYLQMDSLKPEDTAVYYCAANLRSDSSYQGHYDYWGQGTQVTVSS

SEQ ID NO: 424 - protein, 281 aa, synthetic construct
QVQLVESGGGLVQAGDSLRLSCVASGGSFHTYVMGWFRQAPGKEREFVATINYSGSNIYYANSVKGRFTISRDNAKTTVYLQMNSLKPEDTAVYYCAAQVDVYGSMWYMREPTRYDHWGQGTQVTVSSGGGGSGGGGSGGGGSGGGGSGGGGSQVQLVESGGGLVQAGGSLRLSCVASGRTFSTYVMGWFRQAPGKEREFVATINWSGSSTYYAKSVEGRFTISRDNAKNTVYLQMDSLKPEDTAVYYCAAQVDTYGSMWHMMEPTHYEYWGQGTQVTVSS

SEQ ID NO: 425 - protein, 274 aa, synthetic construct
QVQLVESGGGLVQAGGSLRLSCVASGRTFSTYVMGWFRQAPGKEREFVATINWSGSSTYYAKSVEGRFTISRDNAKNTVYLQMDSLKPEDTAVYYCAAQVDTYGSMWHMMEPTHYDYWGQGTQVTVSSGGGGSGGGGSGGGGSQVQLVQLQESGGGLVQAGGSLRLSCTVSGRTFSNYAITWFRQAPGKGRDFVAAVTWSGGATYYADSVTGRFTISRDNAKNTVYLQMNSLKPEDTAVYYCAARRDTNSGSYYYTMDYGMDYWGKGTQVTVSS

SEQ ID NO: 426 - protein, 278 aa, synthetic construct
QVQLVESGGGLVQAGGSLRLSCAASGRTFSSYTMGWFRQAPGKEREYVAAIRWSGGYTYYADSVKGRFTISRDIAKNTVYLQMNSLKPEDTAVYYCAADIIQDGLGSYYYTQGHMYDYWGQGTQVTVSSGGGGSGGGGSGGGGSGGGGSQVQLVESGGGLVQAGGSLRLSCAASGRTFSRYTMGWFRQVPGKEREYVAAIRWSGGYTYYADSVKGRFTISRDIAKNTGYLQMNSLKPEDTAVYYCAADIIQDGLGSYYYTQGHMYDYWGQGTQVTVSS

SEQ ID NO: 427 - protein, 249 aa, synthetic construct
QVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSSGGGGSGGGGSGGGGSQVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSS

SEQ ID NO: 428 - protein, 276 aa, synthetic construct
QVQLVQLQESGGGLVQAGGSLRLSCAASGGTFSNYAMGWFRQAPGKERQFVAAISWTHGNTYYADSAKGRFTISRDNAKNTVYLQMNSLKPEDTAVYYCAARKSGEYSGSYYLQFDFGYDYWGQGTQVTVSSGGGGSGGGGSGGGGSGGGGSGGGGSQVQLVESGGGLVQPGGSLRLSCAASGRTESTVAIGWFRQAPGKEREFVAAIAWSGSWTDIPDSVKGRFTISRDNAKNTVYLQMNSLKPEDTAVYYCAARGSRTYKYEYWGPGTQVTVSS

SEQ ID NO: 429 - protein, 269 aa, synthetic construct
QVQLVESGGGLVQPGGSLRLSCAASGFTFSFYAMTWVRQAPGKGLEWVSRINSGGGSTSYADSVKGRFTISRDNAKNTLYLQMNSLKPEDTAVYYCALPRFASSSRYIDEIGAEYDYWGQGTQVTVSSGGGGSGGGGSGGGGSQVQLVQLQESGGGLVQPGGSLRLSCAASGFTFSTSAMSWARQAPGKGLEWVSGIYSDFSTYYADSVKGRFTISRDNAKNMVYLQMNSLKPEDTAVYYCAIDSYLADSLNWRRSDYRGQGTQVTVSS

SEQ ID NO: 430 - protein, 266 aa, synthetic construct
QVQLVQLQESGGGLVQPGGSLRLSCAASGFTFSTSAMSWARQAPGKGLEWVSGIYSDDSTYYADSVKGRFTISRDNAKNTVYLQMDSLKPEDTAVYYCAIDSYLPDSLNWRRSDYRGQGTQVTVSSGGGGSGGGGSGGGGSGGGGSQVQLVESGGGLVQPGGSLRLSCAASGFTFSSSPMSWVRQAPGKGLEWVSSFYPHSSDTYFGDSVKGRFTIFTDNAKNTLYLQMNSLKPEDTAVYYCAADRSDYAGVFDYWGQGTQVTVSS

SEQ ID NO: 431 - protein, 267 aa, synthetic construct
QVQLVESGGGLVQAGGSLALSCVVSGRTVDSYATGWFRQAPGKEREFVAAITWSGTMTKYADSAKGRFTISRDDAKNTVWLQMNGLKPEDTAVYYCVDRLRGRGNIYSDTLSYDYWGQGTQVTVSSGGGGSGGGGSGGGGSQVQLVESGGGLVQAGGSLALSCVVSGRTVDSYATGWFRQAPGKEREFVAAITWSGTMTKYADSAKGRFTISRDDAKNTVWLQMNGLKPEDTAVYYCVDRLRGRGNIYSDTLSYDYWGQGTQVTVSS

SEQ ID NO: 432 - protein, 280 aa, synthetic construct
QVQLVESGGGLVQPGGSLRLSCAASGFSSDYYIIVWFRQAPGKEREGASCISAISSSDGSTYYADSVKGRFTISRDNAKNTVHLLMNSLKPDDTAVYYCAAAERIHTITEPPRAPYMDYWGKGTQVTVSSGGGGSGGGGSGGGGSGGGGSQVQLVESGGGLVQPGGSLRLSCAASGFSSDYYIIVWFRQAPGKEREGASCISAISSSDGSTYYADSVKGRFTISRDNAKNTVHLLMNSLKPDDTAVYYCAAAERIHTITEPPRAPYMDYWGKGTQVTVSS

SEQ ID NO: 433 - protein, 279 aa, synthetic construct
QVQLVQLQESGGGLVQAGGSLRLSCAASGRTESISSMGWFRQAPGKEREFVVGVYWIDGSTYYRGSVKDRFTISRDNVKNLVHLQMNNLKPEDTAVYYCAAASRGGSPVHPSSFPYWGQGTQVTVSSGGGGSGGGGSGGGGSGGGGSGGGGSQVQLVQLQESGGGLVQAGGSLRLSCAASGRTESISSMGWFRQAPGKEREFVVGVYWIDGSTYYRGSVKDRFTISRDNVKNLVHLQMNNLKPEDTAVYYCAAASRGGSPVHPSSFPYWGQGTQVTVSS

SEQ ID NO: 434 - protein, 271 aa, synthetic construct
QVQLVESGGGLVQAGGSLRLSCVVSGRTWNNYAVAWFRQAPGKERDIVAAITLSAGTSYYADSVKGRFTISRDNVKESVFLQMNSLKPEDTAVYYCAAETSPLRYGDSYWDESTYDYWGQGTQVTVSSGGGGSGGGGSGGGGSQVQLVESGGGLVQAGGSLRLSCVVSGRTWNNYAVAWFRQAPGKERDIVAAITLSAGTSYYADSVKGRFTISRDNVKESVFLQMNSLKPEDTAVYYCAAETSPLRYGDSYWDESTYDYWGQGTQVTVSS

SEQ ID NO: 435 - protein, 276 aa, synthetic construct
QVQLVESGGGLVQTGGSLRLSCAASGRTWNNYAVAWFRQAPGKERDIVAAITLSGGTSYYADSVKGRFTISRDNVKESVFLQMNSLKPEDTAVYYCAAETSPLRYSDSYWDESTYDYWGQGTQVTVSSGGGGSGGGGSGGGGSGGGGSQVQLVESGGGLVQTGGSLRLSCAASGRTWNNYAVAWFRQAPGKERDIVAAITLSGGTSYYADSVKGRFTISRDNVKESVFLQMNSLKPEDTAVYYCAAETSPLRYSDSYWDESTYDYWGQGTQVTVSS

SEQ ID NO: 436 - protein, 251 aa, synthetic construct
QVQLVESGGGLVQPGGSLRLSCTASGFPFSSTWMNWYRQSPGKERQLVAGMSSTGGTTDYANSVKDRFTISRDNNKNTLYLQMNSLKPEDEGLYYCNRPAAIRENDDWGQGTQVTVSSGGGGSGGGGSGGGGSQVQLVESGGGLVQPGGSLRLSCTASGFPFSSTWMNWYRQSPGKERQLVAGMSSTGGTTDYANSVKDRFTISRDNNKNTLYLQMNSLKPEDEGLYYCNRPAAIRENDDWGQGTQVTVSS

SEQ ID NO: 437 - protein, 256 aa, synthetic construct
QVQLVESGGGLVQPGGSLRLSCAASGFPFSSTWMNWYRQSPGKERQLVAGMSSTGGTTDYANSVKDRFTISRDNNKNTLYLQMNSLKPEDEGLYYCNRPAAIRENDDWGQGTQVTVSSGGGGSGGGGSGGGGSGGGGSQVQLVESGGGLVQPGGSLRLSCAASGFPFSSTWMNWYRQSPGKERQLVAGMSSTGGTTDYANSVKDRFTISRDNNKNTLYLQMNSLKPEDEGLYYCNRPAAIRENDDWGQGTQVTVSS

SEQ ID NO: 438 - protein, 281 aa, synthetic construct
QVQLVQLQESGGGLVQAGDSLRLSCTASTRTFSSHRMAWFRQAPGKEREFVAYISGSGGATYYAVPVRGRFAISRDNAKNTMYLQMNSLKPEDTAVYYCAATTFSGKSGVLTTTYDYWGQGTQVTVSSGGGGSGGGGSGGGGSGGGGSGGGGSQVQLVQLQESGGGLVQAGDSLRLSCTASTRTFSSHRMAWFRQAPGKEREFVAYISGSGGATYYAVPVRGRFAISRDNAKNTMYLQMNSLKPEDTAVYYCAATTFSGKSGVLTTTYDYWGQGTQVTVSS

SEQ ID NO: 439 - protein, 259 aa, synthetic construct
QVQLVQAGGSLRLSCVASGSTFRNYPVAWFRQAPGQEREYVAAIRWTTGSTVYADSVKGRFTMSRDNAKNTVYLQMNNLKPEDTGVYYCAARTGPIITSESRAYEFWGQGTQVTVSSGGGGSGGGGSGGGGSGGGGSGGGGSQVQLVQAGGSLRLSCVASGSTFRNYPVAWFRQAPGQEREYVAAIRWTTGSTVYADSVKGRFTMSRDNAKNTVYLQMNNLKPEDTGVYYCAARTGPIITSESRAYEFWGQGTQVTVSS

SEQ ID NO: 440 - protein, 263 aa, synthetic construct
QVQLVESGGGLVQPGGSLRLSCVASGSTFRNYPVAWFRQAPGQEREYVAAIRWTTGSTVYADSVKGRFTMSRDNAKNTVYLQMNNLKPEDTGVYYCAARTGPIITSESRAYEFWGQGTQVTVSSGGGGSGGGGSGGGGSQVQLVESGGGLVQPGGSLRLSCVASGSTFRNYPVAWFRQAPGQEREYVAAIRWTTGSTVYADSVKGRFTMSRDNAKNTVYLQMNNLKPEDTGVYYCAARTGPIITSESRAYEFWGQGTQVTVSS

SEQ ID NO: 441 - protein, 268 aa, synthetic construct
QVQLVESGGGLVQAGGSLRLSCVASGGTFSSYTMGWFRQAPGKERKFVAAITSSGGSTVYQNSVNGRFTISRDNAKNAVYLQMNNLKPEDTAVYYCAADGVGRSSSYVGNYGYWGQGTQVTVSSGGGGSGGGGSGGGGSGGGGSQVQLVESGGGLVQAGGSLRLSCVASGGTFSSYTMGWFRQAPGKERKFVAAITSSGGSTVYQNSVNGRFTISRDNAKNAVYLQMNNLKPEDTAVYYCAADGVGRSSSYVGNYGYWGQGTQVTVSS

SEQ ID NO: 442 - protein, 254 aa, synthetic construct
QVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSSGGGGSGGGGSGGGGSGGGGSQVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSS

SEQ ID NO: 443 - protein, 259 aa, synthetic construct
QVQLVESGGGLVQAGGSLRLSCRASGSTFSSGPIGWYRAPPGKERELVAFISSDGIIKYEDSVKGRFTIWRDNAKNTVYLSMIGLKPEDTAVYSCNLKRPFDRSEYWGQGTQVTVSSGGGGSGGGGSGGGGSGGGGSGGGGSQVQLVESGGGLVQAGGSLRLSCRASGSTFSSGPIGWYRAPPGKERELVAFISSDGIIKYEDSVKGRFTIWRDNAKNTVYLSMIGLKPEDTAVYSCNLKRPFDRSEYWGQGTQVTVSS

SEQ ID NO: 444 - protein, 260 aa, synthetic construct
QVQLVESGGGLVQPGGSLRLSCSASERAFSDYRVAWFRQAPGKEREFVASVNWNGDNSYYADSVKGRFTISRDNAKNAVYLQMNSLKPGDTAVYVCAQSPILSVDSQVVTSPAQYQYWGQGTQVTVSSGGGGSGGGGSGGGGSQVQLVESGGGLVQAGGSLRLSCRASGSTFSSGPIGWYRAPPGKERELVAFISSDGIIKYEDSVKGRFTIWRDNAKNTVYLSMIGLKPEDTAVYSCNLKRPFDRSEYWGQGTQVTVSS

SEQ ID NO: 445 - protein, 273 aa, synthetic construct
QVQLVESGGGLVQAGGSLRLSCAITGGVFSRYTLGWFRQAPGKEREVVAAITWSSGVTYYTDSEKGRFIISRDSAKNVLYLQMSSLKPEDTAMYYCAADVDGSRRWGLYWAVSSDYDYWGQGTQVTVSSGGGGSGGGGSGGGGSGGGGSQVQLVESGGGLVQAGGSLRLSCVASGGTFSSYTMGWFRQAPGKERKFVAAITSSGGSTVYQNSVNGRFTISRDNAKNAVYLQMNNLKPEDTAVYYCAADGVGRSSSYVGNYGYWGQGTQVTVSS

SEQ ID NO: 446 - protein, 277 aa, synthetic construct
QVQLVESGGGLVQPGSSLRLSCAASGRTFNSLSMAWFRQAPGKEREFVAGISWIGNTVYSNSVKGRFSISEDTSKNTGYLQMNSLKPEDTAVYYCAATMDRSPSWVRTNMQSAAYNYWGQGTQVTVSSGGGGSGGGGSGGGGSGGGGSGGGGSQVQLVESGGGLVQPGGSLRLSCVASGSTFRNYPVAWFRQAPGQEREYVAAIRWTTGSTVYADSVKGRFTMSRDNAKNTVYLQMNNLKPEDTGVYYCAARTGPIITSESRAYEFWGQGTQVTVSS

SEQ ID NO: 447 - protein, 259 aa, synthetic construct
QVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSSGGGGSGGGGSGGGGSGGGGSGGGGSQVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSS

SEQ ID NO: 448 - protein, 239 aa, synthetic construct
QVQLVESGGGLVQPGGSLRLSCAASGFTFSSSPMNWVRQPPGKGLEWVASISSDGSRTLYRDSVKGRFTIARDNAKNTLYLQMHTLESEDTAVYYCDYDDSRGPGTQVTVSSGGGGSGGGGSGGGGSQVQLVESGGGLVQPGGSLRLSCAASGFTFSSSPMNWVRQPPGKGLEWVASISSDGSRTLYRDSVKGRFTIARDNAKNTLYLQMHTLESEDTAVYYCDYDDSRGPGTQVTVSS

SEQ ID NO: 449 - protein, 276 aa, synthetic construct
QVQLVESGGGLVQPGGSLRLSCAVSGFPLDYYAIGWFRQAPGKEREGVACSSSGEGSTVYADSVKGRFTISRDDAKNTAYLQMNSLKPGDTAVYYCATRPYSGRGCPSPNTMGAYDYWGVGTQVTVSSGGGGSGGGGSGGGGSGGGGSQVQLVESGGGLVQPGGSLRLSCAVSGFPLDYYAIGWFRQAPGKEREGVACSSSGEGSTVYADSVKGRFTISRDDAKNTAYLQMNSLKPGDTAVYYCATRPYSGRGCPSPNTMGAYDYWGVGTQVTVSS

SEQ ID NO: 450 - protein, 261 aa, synthetic construct
QVQLVESGGGLVQPGGSLRLSCAASGFTFSSYSMSWVRQAPGKGPEWVAVINTGGGSTYYADSVKGRFTISRDNAKNTLYLQMNSLKPEDTALYYCATGAEANWRLSGNQGTQVTVSSGGGGSGGGGSGGGGSGGGGSGGGGSQVQLVESGGGLVQPGGSLRLSCAASGFTFSSYSMSWVRQAPGKGPEWVAVINTGGGSTYYADSVKGRFTISRDNAKNTLYLQMNSLKPEDTALYYCATGAEANWRLSGNQGTQVTVSS

SEQ ID NO: 451 - protein, 249 aa, synthetic construct
QVQLVESGGGQVQPGGSLRLSCAASGFTFSGYTMSWVRQAPGKGLDWISAISSDGGSTTYADSVKGRFTISRDNARNTLYLQMNSLKPEDTAVYYCAKGPRRSYDSWGQGTQVTVSSGGGGSGGGGSGGGGSQVQLVESGGGQVQPGGSLRLSCAASGFTFSGYTMSWVRQAPGKGLDWISAISSDGGSTTYADSVKGRFTISRDNARNTLYLQMNSLKPEDTAVYYCAKGPRRSYDSWGQGTQVTVSS

SEQ ID NO: 452 - protein, 254 aa, synthetic construct; identical to SEQ ID NO: 442

SEQ ID NO: 453 - protein, 259 aa, synthetic construct
QVQLVESGGGLVQPGGSLRLSCAASGFTFSGYTMSWVRQAPGKGLEWVSAISSDGGSTTYADSVKGRFTISRDNAKNTLYLQMNSLKPEDTAVYYCAKGPRRSYDSWGQGTQVTVSSGGGGSGGGGSGGGGSGGGGSGGGGSQVQLVESGGGLVQPGGSLRLSCAASGFTFSGYTMSWVRQAPGKGLEWVSAISSDGGSTTYADSVKGRFTISRDNAKNTLYLQMNSLKPEDTAVYYCAKGPRRSYDSWGQGTQVTVSS

SEQ ID NO: 454 - protein, 269 aa, synthetic construct
QVQLVESGGGLVQAGGSLRLSCAASGRTFGNYAMGWFRQAPGKEREGVSCISSSGGSANYADSVKGRFTISRDNAKNTVYLQMNSLKPEDTAVYYCAAPRGGGPWYCPLDYNGMNYWGKGTQVTVSSGGGGSGGGGSGGGGSQVQLVESGGGLVQAGGSLRLSCAASGRTFGNYAMGWFRQAPGKEREGVSCISSSGGSANYADSVKGRFTISRDNAKNTVYLQMNSLKPEDTAVYYCAAPRGGGPWYCPLDYNGMNYWGKGTQVTVSS

SEQ ID NO: 455 - protein, 274 aa, synthetic construct
QVQLVESGGGLVQAGGSLRLSCAASGFTFDDYAIGWFRQAPGKQREGVSCISPSDGSIYYADSVKGRFTLSRDNAKNTVYLQMNSLKPEDTAVYYCATVSRHGSGYVCYAGGEDDHWGQGTQVTVSSGGGGSGGGGSGGGGSGGGGSQVQLVESGGGLVQAGGSLRLSCAASGFTFDDYAIGWFRQAPGKQREGVSCISPSDGSIYYADSVKGRFTLSRDNAKNTVYLQMNSLKPEDTAVYYCATVSRHGSGYVCYAGGEDDHWGQGTQVTVSS

SEQ ID NO: 456 - protein, 279 aa, synthetic construct
QVQLVESGGGLVQPGGSLRLSCAASGFTLDDYAIGWWRQAPGKQREGVSCISPSDGSIYYADSVKGRFTLSRDNAKNTVYLQMNSLKPEDTAVYYCATVSRHGSGYVCYAGGEDDHWGRGTQVTVSSGGGGSGGGGSGGGGSGGGGSGGGGSQVQLVESGGGLVQPGGSLRLSCAASGFTLDDYAIGWWRQAPGKQREGVSCISPSDGSIYYADSVKGRFTLSRDNAKNTVYLQMNSLKPEDTAVYYCATVSRHGSGYVCYAGGEDDHWGRGTQVTVSS

SEQ ID NO: 457 - protein, 269 aa, synthetic construct
QVQLVESGGGLVQPGGSLRLSCTASGFTLDHYAIGWWRQAPGKQREGVSCISPSDGSIYYADSVKGRFTLSRDNAKNTVYLQMNSLKPEDTAVYYCATVSRHGSGYVCYAGGEDDHWGRGTQVTVSSGGGGSGGGGSGGGGSQVQLVESGGGLVQPGGSLRLSCTASGFTLDHYAIGWWRQAPGKQREGVSCISPSDGSIYYADSVKGRFTLSRDNAKNTVYLQMNSLKPEDTAVYYCATVSRHGSGYVCYAGGEDDHWGRGTQVTVSS

SEQ ID NO: 458 - protein, 268 aa, synthetic construct
QVQLVESGGGLVQPGGSLRLSCAASGFTLAYWTIGWFRQTPGKEREGVSSISGRDGSTNYADSVKGRFTISRDVSKNTVYLQMNSLKPEDTAVYYCASGGSDYGLGHWRWYDYWGQGTKVTVSSGGGGSGGGGSGGGGSGGGGSQVQLVESGGGLVQPGGSLRLSCAASGFTLAYWTIGWFRQTPGKEREGVSSISGRDGSTNYADSVKGRFTISRDVSKNTVYLQMNSLKPEDTAVYYCASGGSDYGLGHWRWYDYWGQGTKVTVSS

SEQ ID NO: 459 - protein, 273 aa, synthetic construct
QVQLVESGGGLVQPGGSLRLSCAASGFTLAYWTIGWFRQTPGKEREGVSSISGRDGSTNYADSVKGRFTISRDVAKNTVYLQMNSLKPEDTAVYYCAAGGSDYGLGHWRWYDYWGQGTQVTVSSGGGGSGGGGSGGGGSGGGGSGGGGSQVQLVESGGGLVQPGGSLRLSCAASGFTLAYWTIGWFRQTPGKEREGVSSISGRDGSTNYADSVKGRFTISRDVAKNTVYLQMNSLKPEDTAVYYCAAGGSDYGLGHWRWYDYWGQGTQVTVSS

SEQ ID NO: 460 - protein, 263 aa, synthetic construct
QVQLVESGGGLVQPGGSLRLSCVASGFTLAYWTIGWFRQTPGKEREGVSSISGRDGSTNYADSVKGRFTISRDVAKNTVYLQMNSLKPEDTAVYYCAAGGSDYGLGHWRWYDYWGQGTQVTVSSGGGGSGGGGSGGGGSQVQLVESGGGLVQPGGSLRLSCVASGFTLAYWTIGWFRQTPGKEREGVSSISGRDGSTNYADSVKGRFTISRDVAKNTVYLQMNSLKPEDTAVYYCAAGGSDYGLGHWRWYDYWGQGTQVTVSS

SEQ ID NO: 461 - protein, 278 aa, synthetic construct
QVQLVESGGGLVQPGGSLKLSCTASGLGLDYYGVGWFRQAPGKEREGVSCINSSGDNIDYADSVKGRFTVSRDLTKNTVYLQMNSLKPEDTAVYYCAAEVIRRVSTVQTMCAFRPMSHWGQGTQVTVSSGGGGSGGGGSGGGGSGGGGSQVQLVESGGGLVQPGGSLKLSCTASGLGLDYYGVGWFRQAPGKEREGVSCINSSGDNIDYADSVKGRFTVSRDLTKNTVYLQMNSLKPEDTAVYYCAAEVIRRVSTVQTMCAFRPMSHWGQGTQVTVSS

SEQ ID NO: 462 - protein, 283 aa, synthetic construct
QVQLVESGGGLVRAGGSLRLSCAAPGRTFRTYAMGWFRQAPGKEREGVSCISSSGDSTNYADSVKGRFTVSRDNAKNTVYLQMNSLKPEDTAVYYCAAEEIRRVPTVQAMCAFGPMSHWGQGQQVTVSSGGGGSGGGGSGGGGSGGGGSGGGGSQVQLVESGGGLVRAGGSLRLSCAAPGRTFRTYAMGWFRQAPGKEREGVSCISSSGDSTNYADSVKGRFTVSRDNAKNTVYLQMNSLKPEDTAVYYCAAEEIRRVPTVQAMCAFGPMSHWGQGQQVTVSS

SEQ ID NO: 463 - protein, 273 aa, synthetic construct
QVQLVESGGGLVQPGGSLRLSCAVSGLTLDYYAIGWFRQAPGKEREAVSCISSSGDSTDYADSVKGRFTVSREDAKNTVYLQMNSLKREDTAVYYCAAEIIRRVPTVQAMCAFGPMSHWGRGQQVTVSSGGGGSGGGGSGGGGSQVQLVESGGGLVQPGGSLRLSCAVSGLTLDYYAIGWFRQAPGKEREAVSCISSSGDSTDYADSVKGRFTVSREDAKNTVYLQMNSLKREDTAVYYCAAEIIRRVPTVQAMCAFGPMSHWGRGQQVTVSS

SEQ ID NO: 464 - protein, 278 aa, synthetic construct
QVQLVESGGGLVQPGGSLRLSCAPSGNTLDYYTIGWFRQAPGKEREAVSCISSSGGTTDYADSVKGRFTGSRDNAKNTVYLQMNSLKPEDTAVYYCAAEIVRRVPTVQTMCAFGPMSHWGQGTQVTVSSGGGGSGGGGSGGGGSGGGGSQVQLVESGGGLVQPGGSLRLSCAPSGNTLDYYTIGWFRQAPGKEREAVSCISSSGGTTDYADSVKGRFTGSRDNAKNTVYLQMNSLKPEDTAVYYCAAEIVRRVPTVQTMCAFGPMSHWGQGTQVTVSS

SEQ ID NO: 465 - protein, 283 aa, synthetic construct
QVQLVESGGGLVQPGGSLRLSCAASGFSLDYYAIGWFRQAPGKEREGVSCISSSGGSTMYADSVKGRFTISRDNAKNTVYLQMNSLKPEDTAVYYCAAEVIRRVSTVQTMCAFGPMSHWGQGTQVTVSSGGGGSGGGGSGGGGSGGGGSGGGGSQVQLVESGGGLVQPGGSLRLSCAASGFSLDYYAIGWFRQAPGKEREGVSCISSSGGSTMYADSVKGRFTISRDNAKNTVYLQMNSLKPEDTAVYYCAAEVIRRVSTVQTMCAFGPMSHWGQGTQVTVSS

SEQ ID NO: 466 - protein, 271 aa, synthetic construct
QVQLVESGGGLVQPGGSLRLSCAASGFTFSRFAMSWYRQAPGKGRELVAAITAAGGSTNYADSVKGRFTISRDNAKNTVYLQMNSLKPEDTAVYYCNADRAVVYSGDYYRNYWGQGTQVTVSSGGGGSGGGGSGGGGSGGGGSGGGGSQVQLVESGGGLVQPGGSLRLSCAASGFTFSRFAMSWYRQAPGKGRELVAAITAAGGSTNYADSVKGRFTISRDNAKNTVYLQMNSLKPEDTAVYYCNADRAVVYSGDYYRNYWGQGTQVTVSS

SEQ ID NO: 467 - protein, 265 aa, synthetic construct
QVQLVESGGGLVQPGGSLRLACVISGHADIAWNMGWFRQAPGKEREVVAAITGSGEITYYSDTVKGRFTISRDNAKNTVYLQMSSLKPEDTGVYYCAAKSRTWPQKYDYWGQGTQVTVSSGGGGSGGGGSGGGGSGGGGSGGGGSQVQLVESGGGLVQPGGSLRLACVISGHADIAWNMGWFRQAPGKEREVVAAITGSGEITYYSDTVKGRFTISRDNAKNTVYLQMSSLKPEDTGVYYCAAKSRTWPQKYDYWGQGTQVTVSS

SEQ ID NO: 468 - protein, 271 aa, synthetic construct
QVQLVESGGGLVQPGGSLRLSCAASGFTFSRFAMSWYRQAPGKGRELVAAISSAGGSTNYGDSVKGRFTISRDNAKNTVYLQMNSLKPEDTAVYICNADRAVTYSGDYYRNYWGQGTQVTVSSGGGGSGGGGSGGGGSGGGGSGGGGSQVQLVESGGGLVQPGGSLRLSCAASGFTFSRFAMSWYRQAPGKGRELVAAISSAGGSTNYGDSVKGRFTISRDNAKNTVYLQMNSLKPEDTAVYICNADRAVTYSGDYYRNYWGQGTQVTVSS

SEQ ID NO: 469 - protein, 271 aa, synthetic construct
QVQLVESGGGLVQPGGSLRLSCAASGFTFSRYAMSWYRQAPGKEREWVATVTSAGGSTNYADFVKGRFTISRDNAKNTVYLQMNSLKLEDTAVYLCNADRAVTWSGDYYRNYWGQGTQVTVSSGGGGSGGGGSGGGGSGGGGSGGGGSQVQLVESGGGLVQPGGSLRLSCAASGFTFSRYAMSWYRQAPGKEREWVATVTSAGGSTNYADFVKGRFTISRDNAKNTVYLQMNSLKLEDTAVYLCNADRAVTWSGDYYRNYWGQGTQVTVSS

SEQ ID NO: 470 - protein, 283 aa, synthetic construct
QVQLVESGGGLVQAGGSLRLSCAASGGTFSNYAMGWFRQAPGKERQFVAAISWTHGNTYYADSAKGRFTISRDNAKNTVYLQMNSLKPEDTAVYYCAARKSGEYSGSYYLQFDFGYDYWGQGTQVTVSSGGGGSGGGGSGGGGSGGGGSGGGGSQVQLVESGGGLVQAGGSLRLSCAASGGTFSNYAMGWFRQAPGKERQFVAAISWTHGNTYYADSAKGRFTISRDNAKNTVYLQMNSLKPEDTAVYYCAARKSGEYSGSYYLQFDFGYDYWGQGTQVTVSS

SEQ ID NO: 471 - protein, 265 aa, synthetic construct
QVQLVESGGGSVQAGGSLRLDCVISGHADIAWNMGWFRQAPGKEREVVAAITGSGEITYYSDSVKGRFTISRDNAKNTVYLQMSSLKPEDTGVYYCAAKSRTWPQKYDYWGQGTQVTVSSGGGGSGGGGSGGGGSGGGGSGGGGSQVQLVESGGGSVQAGGSLRLDCVISGHADIAWNMGWFRQAPGKEREVVAAITGSGEITYYSDSVKGRFTISRDNAKNTVYLQMSSLKPEDTGVYYCAAKSRTWPQKYDYWGQGTQVTVSS

SEQ ID NO: 472 - protein, 281 aa, synthetic construct
QVQLVESGGGLVQAGGSLRLSCTVSGRTFSNYAITWFRQAPGKGRDFVAAVTWSGGATYYADSVTGRFTISRDNAKNTVYLQMNSLKPEDTAVYYCAARRDTNSGSYYYTMDYGMDYWGKGTQVTVSSGGGGSGGGGSGGGGSGGGGSGGGGSQVQLVESGGGLVQAGGSLRLSCTVSGRTFSNYAITWFRQAPGKGRDFVAAVTWSGGATYYADSVTGRFTISRDNAKNTVYLQMNSLKPEDTAVYYCAARRDTNSGSYYYTMDYGMDYWGKGTQVTVSS

SEQ ID NO: 473 - protein, 273 aa, synthetic construct
QVQLVESGGGLVQPGGSLRLSCAASGFTLDNYAIGWYRQAPGKEREGVSCISSRGGRTNYADSVKGRFTISRDNAKNTVYLQMNNLKPEDTAVYYCAAWAELVYCPREPNYDYWGQGTQVTVSSGGGGSGGGGSGGGGSGGGGSGGGGSQVQLVESGGGLVQPGGSLRLSCAASGFTLDNYAIGWYRQAPGKEREGVSCISSRGGRTNYADSVKGRFTISRDNAKNTVYLQMNNLKPEDTAVYYCAAWAELVYCPREPNYDYWGQGTQVTVSS

SEQ ID NO: 474 - protein, 246 aa, synthetic construct
QVQLVESGGGNVQAGGSLRLSCVASGLNFRFINMGWYRQAPGKERELVARINMYGEPNSVDSVKGRFTISRDNAKNTVWLQMDGLKLEDTAVYYCNTFPSDADYWGQGTQVTVSSGGGGSGGGGSGGGGSGGGGSGGGGSQVQLVESGGGNVQAGGSLRLSCVASGLNFRFINMGWYRQAPGKERELVARINMYGEPNSVDSVKGRFTISRDNAKNTVWLQMDGLKLEDTAVYYCNTFPSQVTVSS

SEQ ID NO: 475 - protein, 273 aa, synthetic construct
QVQLVESGGGLVQPGGSLRLSCAVAGRDWNLYAIGWFRQAPGKEREFVARISWIGGATVYADSVKGRFTISKDVAYNTVHLQMDALNPADTAVYYCAAPSRRVWSESQSDYDYWGQGTQVTVSSGGGGSGGGGSGGGGSGGGGSGGGGSQVQLVESGGGLVQPGGSLRLSCAVAGRDWNLYAIGWFRQAPGKEREFVARISWIGGATVYADSVKGRFTISKDVAYNTVHLQMDALNPADTAVYYCAAPSRRVWSESQSDYDYWGQGTQVTVSS

SEQ ID NO: 476 - protein, 269 aa, synthetic construct
QVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSSGGGGSGGGGSGGGGSGGGGSGGGGSQVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSS

SEQ ID NO: 477 - protein, 275 aa, synthetic construct
QVQLVESGGGLVQAGGSLRLSCAASGGPFSSYNMGWFRQSPGKAREFVAAINWKNTSTYYADSVKGRFTISRDNTKDTVYLQMNTLKPDDTAVYYCAAVTGTGWPLRVDYLYDYWGQGTQVTVSSGGGGSGGGGSGGGGSGGGGSGGGGSQVQLVESGGGLVQAGGSLRLSCAASGGPFSSYNMGWFRQSPGKAREFVAAINWKNTSTYYADSVKGRFTISRDNTKDTVYLQMNTLKPDDTAVYYCAAVTGTGWPLRVDYLYDYWGQGTQVTVSS

SEQ ID NO: 478 - protein, 261 aa, synthetic construct
QVQLVESGGGLVQAGGSLRLSCAASGTTFSSNVMGWYRQAPGKQREQVATISPGGTTNYANSVKGRFTISRDNAKNTVYLQMNSLRPDDTAVYYCNKDLSYVTDMDYWGKGTQVTVSSGGGGSGGGGSGGGGSGGGGSGGGGSQVQLVESGGGLVQAGGSLRLSCAASGTTFSSNVMGWYRQAPGKQREQVATISPGGTTNYANSVKGRFTISRDNAKNTVYLQMNSLRPDDTAVYYCNKDLSYVTDMDYWGKGTQVTVSS

SEQ ID NO: 479 - protein, 281 aa, synthetic construct
QVQLVESGGGLVQAGGSLRLSCAAPGRTFRTYAMGWFRQAPGAEREFVAVISWDNDNTHYADSVKGRFTISRDNAENTVYLQMNSLKPEDTAVYYCAASTTGSYGIRWPQDWYEYDFWGQGTQVTVSSGGGGSGGGGSGGGGSGGGGSGGGGSQVQLVESGGGLVQAGGSLRLSCAAPGRTFRTYAMGWFRQAPGAEREFVAVISWDNDNTHYADSVKGRFTISRDNAENTVYLQMNSLKPEDTAVYYCAASTTGSYGIRWPQDWYEYDFWGQGTQVTVSS

SEQ ID NO: 480 - protein, 255 aa, synthetic construct
QVQLVESGGGLVQTGGSLRLSCVASGLNFRYIAMAWYRQAPGKERELVARITRYGEPISVDSVKGRFAISRDNAKNTVWLQMDGLKNEDTADYYCNTFPSDADYWGQGTQVTVSSGGGGSGGGGSGGGGSGGGGSGGGGSQVQLVESGGGLVQTGGSLRLSCVASGLNFRYIAMAWYRQAPGKERELVARITRYGEPISVDSVKGRFAISRDNAKNTVWLQMDGLKNEDTADYYCNTFPSDADYWGQGTQVTVSS

SEQ ID NO: 481 - protein, 287 aa, synthetic construct
QVQLVESGGGLVQAGGSLKLSCGASGRTFSSYAMGWFRQASGEEREFVAALASIGGKRYYADSVKGRFTISRDMFKNTLYLQMNDLKPEDTAVYYCAARERSFSPTDTLTVVTTARAYDYWGQGTQVTVSSGGGGSGGGGSGGGGSGGGGSGGGGSQVQLVESGGGLVQAGGSLKLSCGASGRTFSSYAMGWFRQASGEEREFVAALASIGGKRYYADSVKGRFTISRDMFKNTLYLQMNDLKPEDTAVYYCAARERSFSPTDTLTVVTTARAYDYWGQGTQVTVSS

SEQ ID NO: 482 - protein, 259 aa, synthetic construct
QVQLVESGGGLVQAGGSLNLSCASSRSRWSTDIMGWYRQAPGKQRELVATITVGGVTNYADSVKGRFTISRDRAKTMWYLQMNSLKPDDTAVYFCNLVSLDDSGNYWGQGTQVTVSSGGGGSGGGGSGGGGSGGGGSGGGGSQVQLVESGGGLVQAGGSLNLSCASSRSRWSTDIMGWYRQAPGKQRELVATITVGGVTNYADSVKGRFTISRDRAKTMWYLQMNSLKPDDTAVYFCNLVSLDDSGNYWGQGTQVTVSS

SEQ ID NO: 483 - protein, 263 aa, synthetic construct
QVQLVESGGGLVQAGGSLRLSCAASRGTFSTDVMGWYRQAAGNQRELVATITAGRTTNYADSVKGRFTIARENAKNTVYLQMNSLKPEDTAVYYCNLRTDIRDNPYNYWGPGTQVTVSSGGGGSGGGGSGGGGSGGGGSGGGGSQVQLVESGGGLVQAGGSLRLSCAASRGTFSTDVMGWYRQAAGNQRELVATITAGRTTNYADSVKGRFTIARENAKNTVYLQMNSLKPEDTAVYYCNLRTDIRDNPYNYWGPGTQVTVSS

SEQ ID NO: 484 - protein, 263 aa, synthetic construct
QVQLVESGGGLVQAGGSLTLSCTASGRTLDTNPMGWFRQAPGKEREFVARINTVGFSTYYLDSAKGRFTISRDRAKNTVFLQMNSLKPEDTAVYYCAAGVDTNGDFTSRGQGTQVTVSSGGGGSGGGGSGGGGSGGGGSGGGGSQVQLVESGGGLVQAGGSLTLSCTASGRTLDTNPMGWFRQAPGKEREFVARINTVGFSTYYLDSAKGRFTISRDRAKNTVFLQMNSLKPEDTAVYYCAAGVDTNGDFTSRGQGTQVTVSS

SEQ ID NO: 485 - protein, 274 aa, synthetic construct
QVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSSGGGGSGGGGSGGGGSGGGGSGGGGSGGGGSQVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSS

SEQ ID NO: 486 - protein, 279 aa, synthetic construct
QVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSSGGGGSGGGGSGGGGSGGGGSGGGGSGGGGSGGGGSQVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSS

SEQ ID NO: 487 - protein, 278 aa, synthetic construct
QVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSSGGGGSGGGGSGGGGSGGGGSGGGGSQVQLVESGGGLVQAGGSLKLSCGASGRTFSSYAMGWFRQASGEEREFVAALASIGGKRYYADSVKGRFTISRDMFKNTLYLQMNDLKPEDTAVYYCAARERSFSPTDTLTVVTTARAYDYWGQGTQVTVSS

SEQ ID NO: 488 - protein, 283 aa, synthetic construct
QVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSSGGGGSGGGGSGGGGSGGGGSGGGGSGGGGSQVQLVESGGGLVQAGGSLKLSCGASGRTFSSYAMGWFRQASGEEREFVAALASIGGKRYYADSVKGRFTISRDMFKNTLYLQMNDLKPEDTAVYYCAARERSFSPTDTLTVVTTARAYDYWGQGTQVTVSS

SEQ ID NO: 489 - protein, 288 aa, synthetic construct
QVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSSGGGGSGGGGSGGGGSGGGGSGGGGSGGGGSGGGGSQVQLVESGGGLVQAGGSLKLSCGASGRTFSSYAMGWFRQASGEEREFVAALASIGGKRYYADSVKGRFTISRDMFKNTLYLQMNDLKPEDTAVYYCAARERSFSPTDTLTVVTTARAYDYWGQGTQVTVSS

SEQ ID NO: 490 - protein, 287 aa, synthetic construct
QVQLVESGGGLVQAGGSLKLSCGASGRTFSSYAMGWFRQASGEEREFVAALASIGGKRYYADSVKGRFTISRDMFKNTLYLQMNDLKPEDTAVYYCAARERSFSPTDTLTVVTTARAYDYWGQGTQVTVSSGSTSGSGKSSEGKGEGSTSGSGKSGQVQLVESGGGLVQAGGSLKLSCGASGRTFSSYAMGWFRQASGEEREFVAALASIGGKRYYADSVKGRFTISRDMFKNTLYLQMNDLKPEDTAVYYCAARERSFSPTDTLTVVTTARAYDYWGQGTQVTVSS

SEQ ID NO: 491 - protein, 274 aa, synthetic construct
QVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSSGGGGSGGGGSGGGGSGGGGSGGGGSGGGGSGGGGSQVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSS

SEQ ID NO: 492 - protein, 283 aa, synthetic construct
QVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSSGGGGSGGGGSGGGGSGGGGSGGGGSGGGGSGGGGSQVQLVESGGGLVQAGGSLKLSCGASGRTFSSYAMGWFRQASGEEREFVAALASIGGKRYYADSVKGRFTISRDMFKNTLYLQMNDLKPEDTAVYYCAARERSFSPTDTLTVVTTARAYDYWGQGTQVTVSS

SEQ ID NO: 493 - protein, 274 aa, synthetic construct
QVQLVESGGGSVQPGGSLRLSCEASGSIFSFNAMGWYRQFPGKERELVATILTGGSTNYARSVKGRFTISRDNARNTAYLQMNSLKPEDTAVYYCNAQHWEGSDYSRWDDYWGQGTQVTVSSGGGGSGGGGSGGGGSGGGGSGGGGSGGGGSGGGGSQVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSS

SEQ ID NO: 494 - protein, 283 aa, synthetic construct
QVQLVESGGGLVQAGGSLKLSCGASGRTFSSYAMGWFRQASGEEREFVAALASIGGKRYYADSVKGRFTISRDMFKNTLYLQMNDLKPEDTAVYYCAARERSFSPTDTLTVVTTARAYDYWGQGTQVTVSSGGGGSGGGGSGGGGSGGGGSGGGGSGGGGSGGGGSQVQLVESGGGLVQPGGSLRLSCAASEFTFSGYTMSWVRQAPGKGLEWVSAISSDGGSTTYADSVKGRFTISRDNAKNTLYLQMNSLKPEDTAVYYCAKGPRGRYDSWGQGTQVTVSS

SEQ ID NO: 495 - protein, 25 aa, synthetic construct
GSTSGSGKSSEGKGEGSTSGSGKSG

SEQ ID NO: 496 - protein, 330 aa, Homo sapiens [sequence omitted]

SEQ ID NO: 497 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 498 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 499 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 500 - protein, 324 aa, Mus musculus
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 501 - protein, 335 aa, Mus musculus
AKTTAPSVYPLVPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPHQRVPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEVLHNHLTTKTISRSLGK

SEQ ID NO: 502 - protein, 335 aa, Mus musculus
KTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 503 - protein, 334 aa, Mus musculus
KTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 504 - protein, 329 aa, Mus musculus
TTTAPSVYPLVPGCGDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 505 - protein, 326 aa, Rattus norvegicus
AETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 506 - protein, 322 aa, Rattus norvegicus
AETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGK

SEQ ID NO: 507 - protein, 333 aa, Rattus norvegicus
AQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKVERRNGGIGHKCPTCPTCHKCPVPELLGGPSVFIFPPKPKDILLISQNAKVTCVVVDVSEEEPDVQFSWFVNNVEVHTAQTQPREEQYNSTFRVVSALPIQHQDWMSGKEFKCKVNNKALPSPIEKTISKPKGLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPPGK

SEQ ID NO: 508 - protein, 329 aa, Rattus norvegicus
ARTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRIEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 509 - protein, 323 aa, Oryctolagus cuniculus
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPITHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 510 - protein, 4 aa, synthetic construct
GAGA

SEQ ID NO: 511 - protein, 4 aa, synthetic construct
GSGS

SEQ ID NO: 512 - protein, 6 aa, synthetic construct
GGAGGA

SEQ ID NO: 513 - protein, 6 aa, synthetic construct
GGSGGS

SEQ ID NO: 514 - protein, 8 aa, synthetic construct
GGGAGGGA

SEQ ID NO: 515 - protein, 8 aa, synthetic construct
GGGSGGGS

SEQ ID NO: 516 - protein, 10 aa, synthetic construct
GGGGAGGGGA

SEQ ID NO: 517 - protein, 10 aa, synthetic construct
GGGGSGGGGS

SEQ ID NO: 518 - protein, 5 aa, synthetic construct
GGGGA

SEQ ID NO: 519 - protein, 5 aa, synthetic construct
GGGGS

SEQ ID NO: 520 - protein, 10 aa, synthetic construct
GGGGAGGGGA

SEQ ID NO: 521 - protein, 15 aa, synthetic construct
GGGGAGGGGAGGGGA

SEQ ID NO: 522 - protein, 20 aa, synthetic construct
GGGGAGGGGAGGGGAGGGGA

SEQ ID NO: 523 - protein, 25 aa, synthetic construct
GGGGAGGGGAGGGGAGGGGAGGGGA

SEQ ID NO: 524 - protein, 30 aa, synthetic construct
GGGGAGGGGAGGGGAGGGGAGGGGAGGGGA

SEQ ID NO: 525 - protein, 35 aa, synthetic construct
GGGGAGGGGAGGGGAGGGGAGGGGAGGGGAGGGGA

SEQ ID NO: 526 - protein, 10 aa, synthetic construct
GGGGSGGGGS

SEQ ID NO: 527 - protein, 15 aa, synthetic construct
GGGGSGGGGSGGGGS

SEQ ID NO: 528 - protein, 20 aa, synthetic construct
GGGGSGGGGSGGGGSGGGGS

SEQ ID NO: 529 - protein, 25 aa, synthetic construct
GGGGSGGGGSGGGGSGGGGSGGGGS

SEQ ID NO: 530 - protein, 30 aa, synthetic construct
GGGGSGGGGSGGGGSGGGGSGGGGSGGGGS

SEQ ID NO: 531 - protein, 35 aa, synthetic construct
GGGGSGGGGSGGGGSGGGGSGGGGSGGGGSGGGGS

SEQ ID NO: 532 - protein, 101 aa, synthetic construct
  REPEAT 1..101: note = single repeat of (G)nX where n= 1 - 100
  VARIANT 1..100: note = up to 99 G residues in each iteration of the (G)nX where n=1-100 can be deleted
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGX